SEQUENCE LISTING (127 sequences)

SEQ ID NO: 1 - protein, 122 aa, synthetic construct
QVQLQQPGAELARPGASVKMSCKASGYSFTNYMIHWVKQRPGQGLEWIGYVNPTSGYSEYNQKFKDKATLTADKSSSTAFMQLNSLTSEDSAVYYCTRRDYYGYSHYALDYWGQGTSVTVSS

SEQ ID NO: 2 - protein, 108 aa, synthetic construct
DIVMTQSPATMSASPGEKVTLTCSARSSVSSSYLYWYQQKPGSSPKIWIYSTSNLASGVPARFSGSGSGTSYSLTISSMEAEDAASYFCHQWSSYPPTFGSGTKLEIK

SEQ ID NO: 3 - protein, 119 aa, synthetic construct
QVQLQQPGAEIVRPGASVKLSCKASGYTFTDYWMNWVKQRPGQGLEWIGAIDPSDRYTNYNQKFKDKATLTVDTSSSSAYMQLSSLTSEDSAVYFCARGGYGDYWFANWGQGTTVTVSS

SEQ ID NO: 4 - protein, 113 aa, synthetic construct
DIVMTQSPSSLAMSVGQKVTMSCKSSQNLLSSGTQKNYLAWYQQKPGQSPKPLIYFASTRESGVPDRFIGSGSGTDFTLTISSVQAEDLALYHCQQHHYFPWTFGGGTKLEIK

SEQ ID NO: 5 - protein, 116 aa, synthetic construct
QVQLQQPGAELAKPGASVKMSCKASGYSFSSYWMHWMKQRPGQGQEWIGYINPSTDYTEYNQKFKDKATLTADKSSSTAYMQLSSLTSEDSAVYYCARSRLGFDYWGQGTTLTVSS

SEQ ID NO: 6 - protein, 107 aa, synthetic construct
DIVMTQSPASLSASVGETVTITCRTSENIDSYLAWYQHKEGKSPQLLVYAATVLADGVPLRFSGSGSGTQYSLKINSLQSEDVARYYCQHYYRTPLTFGSGTKLEIK

SEQ ID NO: 7 - protein, 117 aa, synthetic construct
EVQLQQSGAELVRPGASVKMSCKASGYTLTSNNIHWVKQTPGQGLEWIGAIYPGNGDNSYNQKFTDRAILTVDTSSNTAYMQISSLTSEDSAVYFCARSFRRYFDVWGAGTSVTVSS

SEQ ID NO: 8 - protein, 108 aa, synthetic construct
DIKMTQSPAIVSASPGEKVTMTCRATSSVSSSYLQWYQQKPGSSPTLWIYSTSNLASGVPSRFSGSGSGTSYSLTISSVEAEDAATYYCQQYSSSPSTFGSGTKLEIK

SEQ ID NO: 9 - protein, 120 aa, synthetic construct
QVQLQQSGAELVKPGASVRMSCKSSGYTFTSYNIHWVRQTPGQGLEWIGAIYPGNGDNSYNQKFRGKVTLTADKSSSTAYMQLNSLTSEDSAVYYCARQTARTTRAMDYWGQGTTVTVSS

SEQ ID NO: 10 - protein, 108 aa, synthetic construct
DIVLTQSPAIMSASLGERVTMTCTASSSVSSSYLHWYQQKPGSSPKLWIYSTSNLASGVPARFSGSGSGTSYSLTISSMAAEDAATYYCHQYLRSPPTFGSGTKLEIK

SEQ ID NO: 11 - protein, 118 aa, synthetic construct
QVTLKESGPGILQPSQTLSLTCSFSGFSLSTSGMGVSWIRQPSGKGLEWLAHIFWDDDKRYNPSLKSRLTISKDTSRNQVFLKITSVDTADSATYYCARRVLSTFDVWGAGTSVTVSS

SEQ ID NO: 12 - protein, 107 aa, synthetic construct
DIQMTQTTSSLSASLGDRVTISCSASQGINNYLNWYQQKPDGTVKLLIYYTSSLHSGVPSRFSGSGSGTDFSLTISNLEPEDIATYYCQQYSKLPLTFGGGTKLEIK

SEQ ID NO: 13 - protein, 126 aa, synthetic construct
QVQLQQSGAELVKPGASVKISCKVSGYTFTDHTIHWMKQRPEQGLEWIAYIYPRDGSTNYSEKFTGYNEKFKGKATLTADTSSSTAYMQLNSLTSEDSAVYFCARGYRGFFYFDYWGQGTTLTVSS

SEQ ID NO: 14 - protein, 108 aa, synthetic construct
DIVMTQSPTLMAASPGERVTITCSVSSSIRSSNLHWYQQKSGTSPKPWIYGTSNLASGVPVRFSGSGSGTSYSLTISSMEAEDAATYYCQQWSSYPFTFGSGTKLEIK

SEQ ID NO: 15 - protein, 122 aa, synthetic construct
EVQLQQSGPELVKPGASIKISCKASGYSFTGYALNWVKQSHGKNLEWIGLINPYNGGTSYNQKFKGKATLTVDKSSSTAYMELLSLTSEDSAVYYCARWDGNPHYYYAMDYWGQGTSVTVSS

SEQ ID NO: 16 - protein, 107 aa, synthetic construct
DIVMTQSPATLSVTPGDRVSLSCRASQSISDYLHWYQQKSHESPRLLIKSGSQSMSGIPSRFSGSGSGSDFILSINSVEPEDVGMYYCQNGHSFPLTFGGGTKLEIK

SEQ ID NO: 17 - protein, 121 aa, synthetic construct
QVQLQQSGAELVRPGASVKLSCKALGYIFTDYEMHWVRQTPVHGLEWIGGIHPGSGGTVYNQKFKGKATLTADKSSSTAYMELSSLTSEDSAVYYCTRDGYYGIPDYFDYWGQGTTLTVSS

SEQ ID NO: 18 - protein, 114 aa, synthetic construct
DIVMTQSPSSLAVSVGEKVTMSCKSSQSLLYSSNQKNYLAWYQQKPGQSPKLLISWASTRESGVPDRFTGSGSGTDFTLTISSVKAEDLAVYYCQQYYNYPPYTFGAGTKLELK

SEQ ID NO: 19 - protein, 121 aa, synthetic construct
EVKLVESGGGLVEPGGSLKLSCAASGFTFSRYAMSWVRQTPEKRLEWVATISDGGGYTYYPDNVKGRFTISRDNAKNDLYLQMSHLRSEDTAMYYCASFYYGNLYYAMDYWGQGTSVTVSS

SEQ ID NO: 20 - protein, 112 aa, synthetic construct
DVVMTQTPLSLPVNLGDQASISCRSSQSLLHSNGNTYLHWSLQKPGQSPKLLIYKVSNRFSGVPDRFSGSGSGTDFTLKISRVEAEDLGVYFCSQSTHVPITFGAGTKLELK

SEQ ID NO: 21 - protein, 8 aa, synthetic construct
GYSFTNYM

SEQ ID NO: 22 - protein, 8 aa, synthetic construct
VNPTSGYS

SEQ ID NO: 23 - protein, 15 aa, synthetic construct
TRRDYYGYSHYALDY

SEQ ID NO: 24 - protein, 7 aa, synthetic construct
SSVSSSY

SEQ ID NO: 25 - skipped

SEQ ID NO: 26 - protein, 9 aa, synthetic construct
HQWSSYPPT

SEQ ID NO: 27 - protein, 8 aa, synthetic construct
GYTFTDYW

SEQ ID NO: 28 - protein, 8 aa, synthetic construct
IDPSDRYT

SEQ ID NO: 29 - protein, 12 aa, synthetic construct
ARGGYGDYWFAN

SEQ ID NO: 30 - protein, 12 aa, synthetic construct
QNLLSSGTQKNY

SEQ ID NO: 31 - skipped

SEQ ID NO: 32 - protein, 9 aa, synthetic construct
QQHHYFPWT

SEQ ID NO: 33 - protein, 8 aa, synthetic construct
GYSFSSYW

SEQ ID NO: 34 - protein, 8 aa, synthetic construct
INPSTDYT

SEQ ID NO: 35 - protein, 9 aa, synthetic construct
ARSRLGFDY

SEQ ID NO: 36 - protein, 6 aa, synthetic construct
ENIDSY

SEQ ID NO: 37 - skipped

SEQ ID NO: 38 - protein, 9 aa, synthetic construct
QHYYRTPLT

SEQ ID NO: 39 - protein, 8 aa, synthetic construct
GYTLTSNN

SEQ ID NO: 40 - protein, 8 aa, synthetic construct
IYPGNGDN

SEQ ID NO: 41 - protein, 10 aa, synthetic construct
ARSFRRYFDV

SEQ ID NO: 42 - protein, 9 aa, synthetic construct
QQYSSSPST

SEQ ID NO: 43 - protein, 8 aa, synthetic construct
GYTFTSYN

SEQ ID NO: 44 - protein, 13 aa, synthetic construct
ARQTARTTRAMDY

SEQ ID NO: 45 - protein, 9 aa, synthetic construct
HQYLRSPPT

SEQ ID NO: 46 - protein, 10 aa, synthetic construct
GFSLSTSGMG

SEQ ID NO: 47 - protein, 7 aa, synthetic construct
IFWDDDK

SEQ ID NO: 48 - protein, 10 aa, synthetic construct
ARRVLSTFDV

SEQ ID NO: 49 - protein, 6 aa, synthetic construct
QGINNY

SEQ ID NO: 50 - skipped

SEQ ID NO: 51 - protein, 9 aa, synthetic construct
QQYSKLPLT

SEQ ID NO: 52 - protein, 8 aa, synthetic construct
GYTFTDHT

SEQ ID NO: 53 - protein, 15 aa, synthetic construct
IYPRDGSTNYSEKFT

SEQ ID NO: 54 - protein, 12 aa, synthetic construct
ARGYRGFFYFDY

SEQ ID NO: 55 - protein, 7 aa, synthetic construct
SSIRSSN

SEQ ID NO: 56 - skipped

SEQ ID NO: 57 - protein, 9 aa, synthetic construct
QQWSSYPFT

SEQ ID NO: 58 - protein, 8 aa, synthetic construct
GYSFTGYA

SEQ ID NO: 59 - protein, 8 aa, synthetic construct
INPYNGGT

SEQ ID NO: 60 - protein, 15 aa, synthetic construct
ARWDGNPHYYYAMDY

SEQ ID NO: 61 - protein, 6 aa, synthetic construct
QSISDY

SEQ ID NO: 62 - skipped

SEQ ID NO: 63 - protein, 9 aa, synthetic construct
QNGHSFPLT

SEQ ID NO: 64 - protein, 8 aa, synthetic construct
GYIFTDYE

SEQ ID NO: 65 - protein, 8 aa, synthetic construct
IHPGSGGT

SEQ ID NO: 66 - protein, 14 aa, synthetic construct
TRDGYYGIPDYFDY

SEQ ID NO: 67 - protein, 12 aa, synthetic construct
QSLLYSSNQKNY

SEQ ID NO: 68 - skipped

SEQ ID NO: 69 - protein, 10 aa, synthetic construct
QQYYNYPPYT

SEQ ID NO: 70 - protein, 8 aa, synthetic construct
GFTFSRYA

SEQ ID NO: 71 - protein, 8 aa, synthetic construct
ISDGGGYT

SEQ ID NO: 72 - protein, 14 aa, synthetic construct
ASFYYGNLYYAMDY

SEQ ID NO: 73 - protein, 11 aa, synthetic construct
QSLLHSNGNTY

SEQ ID NO: 74 - skipped

SEQ ID NO: 75 - protein, 9 aa, synthetic construct
SQSTHVPIT

SEQ ID NO: 76 - protein, 122 aa, synthetic construct
QVQLVQSGAEVKKPGASVKVSCKASGYSFTNYMMHWVRQAPGQGLEWMGYVNPTSGYSEYNQKFKDRVTMTRDTSTSTVYMELSSLRSEDTAVYYCTRRDYYGYSHYALDYWGQGTLVTVSS

SEQ ID NO: 77 - protein, 122 aa, synthetic construct
QVQLVQSGAEVKKPGASVKVSCKASGYSFTNYMMHWVRQAPGQGLEWMGYVNPTSGYSEYAQKFQGRVTMTADKSTSTVYMELSSLRSEDTAVYYCTRRDYYGYSHYALDYWGQGTLVTVSS

SEQ ID NO: 78 - protein, 108 aa, synthetic construct
EIVLTQSPATLSLSPGERATLSCRASSSVSSSYLYWYQQKPGQAPRILIYSTSNRATGIPARFSGSGSGTDFTLTISSLEPEDFAVYYCHQWSSYPPTFGQGTKLEIK

SEQ ID NO: 79 - protein, 108 aa, synthetic construct
EIVLTQSPATLSLSPGERATLSCRASSSVSSSYLYWYQQKPGQSPKIWIYSTSNLATGIPARFSGSGSGTDFTLTISSLEPEDFAVYYCHQWSSYPPTFGQGTKLEIK

SEQ ID NO: 80 - protein, 119 aa, synthetic construct
QVQLVQSGAEVKKPGASVKVSCKASGYTFTDYWMNWVRQAPGQGLEWMGAIDPSDRYTNYAQKFQGRVTMTRDTSTSTVYMELSSLRSEDTAVYYCARGGYGDYWFANWGQGTLVTVSS

SEQ ID NO: 81 - protein, 119 aa, synthetic construct
QVQLVQSGAEVKKPGASVKVSCKASGYTFTDYWMNWVRQAPGQGLEWMGAIDPSDRYTNYNQKFKGRVTMTVDTSTSTVYMELSSLRSEDTAVYFCARGGYGDYWFANWGQGTLVTVSS

SEQ ID NO: 82 - protein, 113 aa, synthetic construct
DIVMTQSPDSLAVSLGERATINCKSSQNLLSSGTQKNYLAWYQQKPGQPPKPLIYFASTRESGVPDRFSGSGSGTDFTLTISSLQAEDVAVYYCQQHHYFPWTFGGGTKVEIK

SEQ ID NO: 83 - protein, 113 aa, synthetic construct
DIVMTQSPDSLAVSLGERATINCKSSQNLLSSGTQKNYLAWYQQKPGQSPKPLIYFASTRESGVPDRFSGSGSGTDFTLTISSLQAEDVAVYYCQQHHYFPWTFGGGTKVEIK

SEQ ID NO: 84 - protein, 116 aa, synthetic construct
QVQLVQSGAEVKKPGASVKVSCKASGYSFSSYWMHWMRQAPGQGQEWMGYINPSTDYTEYNQKFKDRVTMTRDTSTSTVYMELSSLRSEDTAVYYCARSRLGFDYWGQGTLVTVSS

SEQ ID NO: 85 - protein, 116 aa, synthetic construct
QVQLVQSGAEVKKPGASVKVSCKASGYSFSSYWMHWMRQAPGQGQEWMGYINPSTDYTEYNQKFKDRVTMTADKSTSTVYMELSSLRSEDTAVYYCARSRLGFDYWGQGTLVTVSS

SEQ ID NO: 86 - protein, 107 aa, synthetic construct
DIQMTQSPSSLSASVGDRVTITCRASENIDSYLAWYQHKPGKAPKLLLYAATVLESGVPSRFSGSGSGTDYTLTISSLQPEDFARYYCQHYYRTPLTFGQGTKLEIK

SEQ ID NO: 87 - protein, 107 aa, synthetic construct
DIQMTQSPSSLSASVGDRVTITCRASENIDSYLAWYQHKEGKSPKLLLYAATVLADGVPSRFSGSGSGTDYTLTISSLQPEDFARYYCQHYYRTPLTFGQGTKLEIK

SEQ ID NO: 88 - protein, 225 aa, synthetic construct
QVQLQQPGAELARPGASVKMSCKASGYSFTNYMIHWVKQRPGQCLEWIGYVNPTSGYSEYNQKFKDKATLTADKSSSTAFMQLNSLTSEDSAVYYCTRRDYYGYSHYALDYWGQGTSVTVSSASTKGPSVFPLAPSSESTSGGTAALGCLVKDYFPEPVTVSWNSGALTSGVHTFPAVLQSSGLYSLSSVVTVPSSSLGTQTYICNVNHKPSNTKVDKKVEPKSV

SEQ ID NO: 89 - protein, 214 aa, synthetic construct
DIVMTQSPATMSASPGEKVTLTCSARSSVSSSYLYWYQQKPGSSPKIWIYSTSNLASGVPARFSGSGSGTSYSLTISSMEAEDAASYFCHQWSSYPPTFGCGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTKVS

SEQ ID NO: 90 - protein, 222 aa, synthetic construct
QVQLQQPGAEIVRPGASVKLSCKASGYTFTDYWMNWVKQRPGQGLEWIGAIDPSDRYTNYNQKFKDKATLTVDTSSSSAYMQLSSLTSEDSAVYFCARGGYGDYWFANWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 91 - protein, 220 aa, synthetic construct
DIVMTQSPSSLAMSVGQKVTMSCKSSQNLLSSGTQKNYLAWYQQKPGQSPKPLIYFASTRESGVPDRFIGSGSGTDFTLTISSVQAEDLALYHCQQHHYFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 219 aa, synthetic construct
QVQLQQPGAELAKPGASVKMSCKASGYSFSSYWMHWMKQRPGQGQEWIGYINPSTDYTEYNQKFKDKATLTADKSSSTAYMQLSSLTSEDSAVYYCARSRLGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 93 - protein, 214 aa, synthetic construct
DIVMTQSPASLSASVGETVTITCRTSENIDSYLAWYQHKEGKSPQLLVYAATVLADGVPLRFSGSGSGTQYSLKINSLQSEDVARYYCQHYYRTPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 222 aa, synthetic construct
QVQLVQSGAEVKKPGASVKVSCKASGYTFTDYWMNWVRQAPGQGLEWMGAIDPSDRYTNYAQKFQGRVTMTRDTSTSTVYMELSSLRSEDTAVYYCARGGYGDYWFANWGQGTLVTVSSASTKGPSVFPLAPSSKSTSGGTAALGCLVKDYFPEPVTVSWNSGALTSGVETFPAVLQSSGLYSLSSVVTVPSSSLGTQTYICNVNHKPSNTKVDKKVEPKSC

SEQ ID NO: 95 - protein, 219 aa, synthetic construct
DIVMTQSPDSLAVSLGERATINCKSSQNLLSSGTQKNYLAWYQQKPGQSPKPLIYFASTRESGVPDRFSGSGSGTDFTLTISSLQAEDVAVYYCQQHHYFPWTFGGGTKVEIKGQPKAAPSVTLFPPSSEELQANKATLVCLISKFYPGAVTVAWKADSSPVKAGVETTTPSKKSNNKYAASSYLSLTPEQWKSHRSYSCQVTHEGSTVEKTVAPTECS

SEQ ID NO: 96 - protein, 225 aa, synthetic construct
EVQLQQSGPELVKPGASIKISCKASGYSFTGYALNWVKQSHGKNLEWIGLINPYNGGTSYNQKFKGKATLTVDKSSSTAYMELLSLTSEDSAVYYCARWDGNPHYYYAMDYWGQGTSVTVSSASTKGPSVFPLAPSSKSTSGGTAALGCLVKDYFPEPVTVSWNSGALTSGVKTFPAVLQSSGLYSLSSVVTVPSSSLGTQTYICNVNHKPSNTKVDKKVEPKSC

SEQ ID NO: 97 - protein, 214 aa, synthetic construct
DIVMTQSPATLSVTPGDRVSLSCRASQSISDYLHWYQQKSHESPRLLIKSGSQSMSGIPSRFSGSGSGSDFILSINSVEPEDVGMYYCQNGHSFPLTFGGGTKLEIKRTVAAPSVFIFPPSDEQLKSGTASVVCLLNEFYPREAKVQWKVDNALQSGNSQESVTEQESKDSTYSLSSTLTLSKADYEKHKVYACEVTHQGLSSPVTKSFNRGEC

SEQ ID NO: 98 - protein, 259 aa, synthetic construct
QAVVTQEPSLTVSPGGTVTLTCGSSTGAVTSGHYANWIQEKPGKSPRGLIGGTSKRAPGVPARFSGSLLGGKAALTISGAQPEDEAQYYCALWYSNLWVFGGGTKLTVLGGGGSGGGGSGGGGSGGGGSEVQLVESGGGLVQPGGSLKLSCAASGFTFSTYAMNWIRKAPGKGLEWVGRIRSKANSYATYYADSVKGRFTISRDDSKNTLYLQMNSLKTEDTAVYYCVRHGNFGNSYVSWFAYWGQGTLVTVSSEPKSS

SEQ ID NO: 99 - protein, 20 aa, synthetic construct
GGGGSGGGGSGGGGSGGGGS

SEQ ID NO: 100 - protein, 220 aa, synthetic construct
EVQLQQSGAELVRPGASVKMSCKASGYTLTSNNIHWVKQTPGQGLEWIGAIYPGNGDNSYNQKFTDRAILTVDTSSNTAYMQISSLTSEDSAVYFCARSFRRYFDVWGAGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 101 - protein, 215 aa, synthetic construct
DIKMTQSPAIVSASPGEKVTMTCRATSSVSSSYLQWYQQKPGSSPTLWIYSTSNLASGVPSRFSGSGSGTSYSLTISSVEAEDAATYYCQQYSSSPSTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 223 aa, synthetic construct
QVQLQQSGAELVKPGASVRMSCKSSGYTFTSYNIHWVRQTPGQGLEWIGAIYPGNGDNSYNQKFRGKVTLTADKSSSTAYMQLNSLTSEDSAVYYCARQTARTTRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 103 - protein, 215 aa, synthetic construct
DIVLTQSPAIMSASLGERVTMTCTASSSVSSSYLHWYQQKPGSSPKLWIYSTSNLASGVPARFSGSGSGTSYSLTISSMAAEDAATYYCHQYLRSPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 221 aa, synthetic construct
QVTLKESGPGILQPSQTLSLTCSFSGFSLSTSGMGVSWIRQPSGKGLEWLAHIFWDDDKRYNPSLKSRLTISKDTSRNQVFLKITSVDTADSATYYCARRVLSTFDVWGAGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 105 - protein, 214 aa, synthetic construct
DIQMTQTTSSLSASLGDRVTISCSASQGINNYLNWYQQKPDGTVKLLIYYTSSLHSGVPSRFSGSGSGTDFSLTISNLEPEDIATYYCQQYSKLPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 330 aa, synthetic construct [sequence omitted]

SEQ ID NO: 109 - protein, 107 aa, synthetic construct [sequence omitted]

SEQ ID NO: 110 - protein, 832 aa, synthetic construct
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 111 - protein, 840 aa, synthetic construct
MLHSYCANKLLEGTTLKVLRISYLVIEPVKILSEIGDLLQIPQLCFFPYSLICCFFPQFKANPPAVTFELTGETDNIFKIEQEGLLYYTKALDRETRSTHNLQVAALDANGAIVEGPVPITIEVKDVNDNRPTFLQSKYEGSVRQNSRPGKPFLYVNATDLDDPATPNGQLSYQIVIQLPMINNVMYFQINNKTGGISLTREGSQELNPAKNPSYNLVISVKDMGGQSENSFSDTTSVDIIVTENIWKAPEPVEMVENSTDPHPIKITQVRWNDPGAQYSLVDKEKLPRFPFSIDQEGDIYVTQPLDREEKDAYVFYAVAKDEYGKPLSYPLEIHVKVQDINDNPPTCPSPVTVFEVQENERLGNSIGALTAHDSDEENTANSLLNYRIVEQTPKLPMDGLFLIQTYAGMLQLAKQSLKKQDTPQYNLTIEVSDKDFKTLCFVQINVIDINDQIPIFEKSDYGNLTLAEDTNVGSTILTIQATDADEPFTGSSKILYHVIKGDSEGRLGVDTDPHTNTGYVIIKKPLDFETAAISNIVFKAENPEPLVFGVTYNASSFAKFTLFVTDVNEAPEFSQYVFQAKVSEDVAIGTKVGNVTAKDPEGLDISYSLRGDTRGWLKIDHVTGEIFSVAPLDREAGSPYRVQVVATEVGGSSLSSVSQFHLILTDVNDNPPRLAKDYMDLYFCHPLSAPGSLIFEATDDDQHLFRGPHFTFSIASESLQNDWQVSKINGTHARLSTRHTDFEEKEYVVSIRINDGGRPPLESTVSLTVTFCSCGEDGCFRPAGHQPGIPTVGMAVGILLTTLLVIGIILAVVFIRMKTDKGKDNVESAQASEVKPLRS

SEQ ID NO: 112 - protein, 827 aa, synthetic construct
MVSAQLHFLCLLTLYLTCGYGEEGKFSGPLKPMTFSIFEGQEPSQVIFQFKTNPPAVTFELTGETDGIFKIEKDGLLYHTRALDRETRAVHHLQLAALDSHGAIVDGPVPITIEVKDINDNRPTFLQSKYEGSVRQNSRPGKPFMYVNATDLDDPATPNGQLFYQIVIQLPQINDVMYFQIDSKTGAISLTPEGSQELDPVKNPSYNLVVSVKDMGGQSENSFSDTTYVDISIRENIWKAPEPVEIRENSTDPHPIKITQVQWNDPGAQYSLVNKEKLSPFPFSIDQEGNIYVTQALDREEKNSHVFFATAKDENGKPLAYPLEIYVKVIDINDNPPTCLSPVTVFEVQENEPLGNSIGIFEAHDMDEANNINSILKYKLVDQTPKVPSDGLFLIGEYEGKVQLSKQSLKKQDSPQYNLSIEVSDVDFKTLCYIQVNVIDINDQIPIFETSNYGSKTLSEDTAIGSTILIIQATDADEPFTGSSKILYKIVQGDTEGRLEVVTDPTTNAGYVKIKKPLDFETQPVSSIVFQAENPEPLVKGIEYNASSFASFELIVTDVNEVPVFPQRIFQANVSEDAAVGSRVGNVTARDPEGLTVSYSLKGNMRGWLKIDSVTGEIFSAAPLDRETESVYRVQVVATEVGGSSLSSTADFHLVLTDVNDNPPRLAKDYTGLFFCHPLSAPGSLIFEVTDDDQQSLRRPKFTFALGREGLQSDWEVSKINGTHARLSTRHTRFEEQVYNIPIRINDGGQPPMEGTVFLPVTFCQCVEGSCFRPAGRQDGIPTVGMAVGILLTTFLVIGIILAVVFIRMRKDKVENPQSPENKPLRS

SEQ ID NO: 113 - protein, 829 aa, synthetic construct
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGIPFLFLEASDRDEPGTANSDLRFHILSQAPAQPSPDMFQLEPRLGALALSPKGSTSLDHALERTYQLLVQVKDMGDQASGHQATATVEVSIIESTWVSLEPIHLAENLKVLYPHHMAQVHWSGGDVHYHLESHPPGPFEVNAEGNLYVTRELDREAQAEYLLQVRAQNSHGEDYAAPLELHVLVMDENDNVPICPPRDPTVSIPELSPPGTEVTRLSAEDADAPGSPNSHVVYQLLSPEPEDGVEGRAFQVDPTSGSVTLGVLPLRAGQNILLLVLAMDLAGAEGGFSSTCEVEVAVTDINDHAPEFITSQIGPISLPEDVEPGTLVAMLTAIDADLEPAFRLMDFAIERGDTEGTFGLDWEPDSGHVRLRLCKNLSYEAAPSHEVVVVVQSVAKLVGPGPGPGATATVTVLVERVMPPPKLDQESYEASVPISAPAGSFLLTIQPSDPISRTLRFSLVNDSEGWLCIEKFSGEVHTAQSLQGAQPGDTYTVLVEAQDTDEPRLSASAPLVIHFLKAPPAPALTLAPVPSQYLCTPRQDHGLIVSGPSKDPDLASGHGPYSFTLGPNPTVQRDWRLQTLNGSHAYLTLALHWVEPREHIIPVVVSHNAQMWQLLVRVIVCRCNVEGQCMRKVGRMKGMPTKLSAVGILVGTLVAIGIFLILIFTHWTMSRKKDPDQPADSVPLKATV

SEQ ID NO: 114 - protein, 247 aa, synthetic construct
METDTLLLWVLLLWVPGSTGQEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAHKHHHHHH

SEQ ID NO: 115 - protein, 357 aa, synthetic construct
METDTLLLWVLLLWVPGSTGNIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQHKHHHHHH

SEQ ID NO: 116 - protein, 247 aa, synthetic construct
METDTLLLWVLLLWVPGSTGDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHKHHHHHH

SEQ ID NO: 117 - protein, 225 aa, synthetic construct
QVQLVQSGAEVKKPGASVKVSCKASGYSFTNYMMHWVRQAPGQGLEWMGYVNPTSGYSEYNQKFKDRVTMTRDTSTSTVYMELSSLRSEDTAVYYCTRRDYYGYSHYALDYWGQGTLVTVSSASTKGPSVFPLAPSSKSTSGGTAALGCLVKDYFPEPVTVSWNSGALTSGVETFPAVLQSSGLYSLSSVVTVPSSSLGTQTYICNVNHKPSNTKVDKKVEPKSC

SEQ ID NO: 118 - protein, 214 aa, synthetic construct
EIVLTQSPATLSLSPGERATLSCRASSSVSSSYLYWYQQKPGQAPRILIYSTSNRATGIPARFSGSGSGTDFTLTISSLEPEDFAVYYCHQWSSYPPTFGQGTKLEIKGQPKAAPSVTLFPPSSEELQANKATLVCLISKFYPGAVTVAWKADSSPVKAGVETTTPSKKSNNKYAASSYLSLTPEQWKSHRSYSCQVTHEGSTVEKTVAPTECS

SEQ ID NO: 119 - protein, 222 aa, synthetic construct
QVQLVQSGAEVKKPGASVKVSCKASGYTFTDYWMNWVRQAPGQGLEWMGAIDPSDRYTNYNQKFKGRVTMTVDTSTSTVYMELSSLRSEDTAVYFCARGGYGDYWFANWGQGTLVTVSSASTKGPSVFPLAPSSKSTSGGTAALGCLVKDYFPEPVTVSWNSGALTSGVKTFPAVLQSSGLYSLSSVVTVPSSSLGTQTYICNVNHKPSNTKVDKKVEPKSC

SEQ ID NO: 120 - protein, 220 aa, synthetic construct
DIVMTQSPDSLAVSLGERATINCKSSQNLLSSGTQKNYLAWYQQKPGQPPKPLIYFASTRESGVPDRFSGSGSGTDFTLTISSLQAEDVAVYYCQQHHYFPWTFGGGTKVEIKRTVAAPSVFIFPPSDEQLKSGTASVVCLLNEFYPREAKVQWKVDNALQSGNSQESVTEQESKDSTYSLSSTLTLSKADYEKHKVYACEVTHQGLSSPVTKSFNRGEC

SEQ ID NO: 121 - protein, 219 aa, synthetic construct
QVQLVQSGAEVKKPGASVKVSCKASGYSFSSYWMHWMRQAPGQGQEWMGYINPSTDYTEYNQKFKDRVTMTADKSTSTVYMELSSLRSEDTAVYYCARSRLGFDYWGQGTLVTVSSASTKGPSVFPLAPSSKSTSGGTAALGCLVKDYFPEPVTVSWNSGALTSGVKTFPAVLQSSGLYSLSSVVTVPSSSLGTQTYICNVNHKPSNTKVDKKVEPKSC

SEQ ID NO: 122 - protein, 214 aa, synthetic construct
DIQMTQSPSSLSASVGDRVTITCRASENIDSYLAWYQHKEGKSPKLLLYAATVLADGVPSRFSGSGSGTDYTLTISSLQPEDFARYYCQHYYRTPLTFGQGTKLEIKRTVAAPSVFIFPPSDEQLKSGTASVVCLLNEFYPREAKVQWKVDNALQSGNSQESVTEQESKDSTYSLSSTLTLSKADYEKHKVYACEVTHQGLSSPVTKSFNRGEC

SEQ ID NO: 123 - protein, 117 aa, synthetic construct
QVQLVQSGAEVKKPGASVKVSCKASGYTLTSNNIHWVRQAPGQGLEWMGAIYPGNGDNSYAQKFTGRVTMTVDTSTNTAYMELSSLRSEDTAVYYCARSFRRYFDVWGQGTMVTVSS

SEQ ID NO: 124 - protein, 108 aa, synthetic construct
DIQMTQSPSSLSASVGDRVTITCRASSSVSSSYLQWYQQKPGKAPKLLIYSTSNLASGVPSRFSGSGSGTDFTLTISSLQPEDFATYYCQQYSSSPSTFGQGTKLEIK

SEQ ID NO: 125 - protein, 220 aa, synthetic construct
QVQLVQSGAEVKKPGASVKVSCKASGYTLTSNNIHWVRQAPGQGLEWMGAIYPGNGDNSYAQKFTGRVTMTVDTSTNTAYMELSSLRSEDTAVYYCARSFRRYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 126 - protein, 215 aa, synthetic construct
DIQMTQSPSSLSASVGDRVTITCRASSSVSSSYLQWYQQKPGKAPKLLIYSTSNLASGVPSRFSGSGSGTDFTLTISSLQPEDFATYYCQQYSSSPSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 259 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSGGGGSGGGGSEVQLVESGGGLVQPGGSLRLSCAASGFTFSTYAMNWVRQAPGKGLEWVGRIRSKANSYATYYADSVKGRFTISRDDSKNTLYLQMNSLRAEDTAVYYCVRHGNFGNSYVSWFAYWGQGTLVTVSSEPKSS

(19) United States
(12) Patent Application Publication
(10) Pub. No.: US 2026/0078179 A1
(43) Pub. Date: Mar. 19, 2026

(54) CDH17 ANTIBODIES AND USES THEREOF

(71) Applicant: NOVAROCK BIOTHERAPEUTICS, LTD., Ewing, NJ (US)

(72) Inventors: Ming LEI, Princeton, NJ (US); Han LI, Newtown, PA (US); Yi PEI, Ewing, NJ (US); Haichun HUANG, Woodland, CA (US); Di SHEN, Ewing, NJ (US); Puspa THAPA, Ewing, NJ (US); Raymond YU, East Brunswick, NJ (US)

(21) Appl. No.: 19/272,591

(22) Filed: Jul. 17, 2025

Related U.S. Application Data
(60) Provisional application No. 63/672,970, filed on Jul. 18, 2024.

(51) Int. Cl. C07K 16/28; A61K 47/68; A61P 35/00

(52) U.S. Cl. CPC C07K 16/28; A61K 47/6843; A61P 35/00; C07K 16/2809; C07K 2317/24; C07K 2317/31; C07K 2317/32; C07K 2317/55; C07K 2317/565; C07K 2317/622

(57) ABSTRACT

The present disclosure provides monoclonal and recombinant anti-CDH17 antibodies (including CDH17 biparatopic and CDH17xCD3 bispecific antibodies and CDH17-binding fragments. Such antibodies and binding fragments are useful for the treatment of cancer, cither alone or in combination with other agents.

### FIELD

The present disclosure relates to antibodies and fragments thereof which bind to Cadherin-17 (CDH17) protein. The disclosure further relates to therapeutic and diagnostic compositions comprising these antibodies and to methods of using the compositions for antibody-based immunotherapy of cancer and/or cancer diagnosis.

### RELATED APPLICATION

This application claims the benefit and priority to U.S. Patent Application No. 63/672,970, filed Jul. 18, 2024, which is incorporated herein by reference in its entirety.

### SEQUENCE LISTING

The instant application contains a sequence listing, which has been submitted in XML format via Patent Center. The contents of the XML file named “122863-5011-US-Sequence Listing” which was created on Nov. 14, 2025, and is 119,866 bytes in size, are incorporated herein by reference in their entirety.

### BACKGROUND

The cadherins (named for “calcium-dependent adhesion”) are a superfamily of conserved transmembrane proteins involved in diverse fundamental cellular processes including cell-cell adhesion (mediated by homodimerization), morphogenesis, cell recognition and signaling. By regulating contact formation and stability, cadherins play a crucial role in tissue morphogenesis and homeostasis. Cadherins are expressed in many cells and tissues and are evolutionarily conserved in vertebrates and invertebrates.

Classical cadherins (e.g., E-, N-, and P-cadherins) possess five extracellular cadherin (EC) repeats, a single transmembrane domain, and a cytoplasmic domain consisting of more than 100 amino acids. In contrast, cadherin 17 (CDH17) (also known as LI-cadherin) is a non-classical cadherin and possesses seven EC repeats, a single transmembrane domain, and a short cytoplasmic domain comprising approximately 20 amino acids (Berndorff, D. et al. J. Cell Biol. 125, 1353-1369 (1994). Due to its unique distinct structural features cadherin 17 (CDH17), along with cadherin 16 (CDH16), are classified as members of the seven-domain cadherin subfamily.

CDH17 exhibits elevated expression during embryogenesis and is present in fetal liver and the gastrointestinal tract. CDH17 localizes to the basolateral domain of hepatocytes and enterocytes, where it functions to maintain tissue integrity of epithelial tissue by mediating intercellular adhesion in a calcium-dependent manner. In normal human adult tissues, CDH17 is expressed in colon mucosa, small-intestine mucosa, appendix, and pancreatic ducts, but not acinar cells or islet (Altree-Tacha, et al. Arch Pathol Lab Med 141 (1): 144-150 (2017)). CDH17 expression is not detected in normal kidney, lung, liver, brain, adrenal gland, or skin tissues (Jacobsen, F., et al. Pathology-Research and Practice. (2024)).

CDH17 expression has been reported on the surface of neuroendocrine tumors (NETs) and various types of gastrointestinal cancers. NETs constitute a diverse group of epithelial neoplasms that most frequently occur in the gastrointestinal, pancreatic, and bronchopulmonary systems. CDH17 expression has been found in hepatocellular carcinoma (Wong, Luk et al. Biochem Biophys Res Commun 311 (3): 618-624 (2003)), gastric cancer (Dong, Yu et al. Dig Dis Sci 52 (2): 536-542 (2007), Altree-Tacha, Tyrrell et al. 2017)), pancreatic cancer (Yuan H et al., Data Brief: 25:104332 (2019)) and colorectal cancer (Tian, Han et al. Biomed Pharmacother 108:331-337 (2018)).

CDH17 plays multiple roles in cancer, including promoting tumor progression, invasion, and metastasis through facilitating cell adhesion and migration. Its involvement in cell adhesion potentially aids in the formation of tumor cell clusters and the spread of metastatic lesions to distant organs. (Bartolome, R. A., et al. Clin Cancer Res 24 (2): 433-444. (2018); Yuan, H., et al. Data Brief 25:104332. (2019); Garcia-Martinez, J. M., et al. Mol Cancer Ther 20 (1): 96-108. (2021)).

Elevated levels of CDH17 have been shown to correlate with tumor burden and poor prognosis in colorectal cancer (CRC) (Lui Ng et al., JCO 41, e14651-e14651 (2023)). In addition, the expression of CDH17 was notably reduced in colorectal cancer (CRC) cases exhibiting high microsatellite instability. Moreover, it displayed a negative correlation with immune response gene sets and the expression levels of MHC class I and II molecules (Wong, K. K. Comput Biol Chem 105:107897. (2023)).

In hepatocellular carcinoma (HCC), alternative splice isoforms and genetic polymorphisms of the CDH17 gene have been detected, correlating with a heightened risk of HCC development (Lee, N. P., et al. Biochim Biophys Acta 1806 (2): 138-145. (2010)). CDH17 expression levels revealed a significant association with lymph node metastasis (LNM) (Lee, C. W., et al. Oncol Lett 15 (1): 559-567. (2018)).

CDH17 is a promising target for therapeutic interventions in CRCs, HCC or other cancers that highly express CDH17, and targeting it may benefit patients that do not respond to checkpoint inhibitor immunotherapies. Although CDH17 is a recognized therapeutic target, to date there are no approved anti-CDH17 therapeutic antibodies. Therefore, there is an unmet need to provide effective, safe and specific anti-CDH17 antibodies that alone, or in combination with other agents, can be used for antibody-based immunotherapy.

The present disclosure provides antibodies, biparatopic antibodies and bispecific antibodies targeting CDH17 as a tumor associated antigen (TAA) for the diagnosis and/or antibody-based immunotherapy of CDH17 positive tumors either alone or in combination with other therapeutic agents.

### SUMMARY

The present disclosure provides anti-CDH17 antibodies and fragments thereof that bind to CDH17 (e.g., CDH17 present on the surface of a cancer cell). These antibodies and fragments thereof are characterized by unique sets of CDR sequences, specificity for CDH17, and are useful in antibody-based immunotherapy either as a monotherapy or as a combination therapy with other anti-cancer agents.

In some embodiments, the disclosed CDH17 antibody or antigen binding fragment of the present disclosure may be a monoclonal antibody or a recombinant antibody (e.g., a chimeric antibody, a humanized antibody, bispecific or a biparatopic antibody) which binds to human CDH17 and exhibits one or more of the following properties alone or in combination:
(a) is specific for human CDH17 with cell binding affinities ranging from 0.1 nM-10 nM;
(b) binds to an epitope within human CDH17 ECD 1-2;
(c) binds to an epitope within human CDH17 ECD 3-5;
(d) binds to an epitope within human CDH17 ECD 6-7;
(e) binds to two distinct and non-overlapping epitopes within human CDH17;
(f) binds to a first epitope within human CDH17 ECD 3-5 and to a second epitope within ECD 6-7 of human CDH17;
(g) binds to a first epitope within human CDH17 ECD 1-2 and to a second epitope within ECD 6-7 of human CDH17;
(h) binds to a first epitope within human CDH17 ECD 1-2 and to a second epitope within ECD 3-5 of human CDH17;
(i) cross-reacts with cynomolgus CDH17;
(j) does not bind to human CDH16;
(k) binds to CDH17 endogenously expressed by human cancer cells;
(l) is internalized from the surface of CDH17 positive cell after binding CDH17;
(m) is internalized and kills CDH17 positive cells with varying levels of CDH17 expression when combined with an ADC-conjugated secondary antibody or induces killing of tumor cells when directly conjugated with cytotoxic agent;
n) simultaneously engaging CDH17 on tumor cells and CD3 on T cells in a CD3/CDH17 bispecific Ab format;
(o) induces T cell activation in a CD3/CDH17 bispecific Ab format; and
(p) redirect T cells to kill CDH17-expressing tumor cells in a CD3/CDH17 bispecific Ab format.

The disclosed murine anti-CDH17 antibodies (C17_mAb 1, C17_mAb 2, C17_mAb 3, C17_mAb 4, C17_mAb 5, C17_mAb 6, C17_mAb 7, C17_mAb 8, C17_mAb 9 and C17_mAb 10) or antigen binding fragments thereof (e.g., binding fragments thereof) bind to human CDH17 and are optionally characterized by functional features that make them suitable for use as therapeutic targeting CDH17+ tumor cells.

According to some embodiments, the anti-CDH17 antibodies or binding fragments thereof comprise a set of six complementarity determining region (CDR) sequences selected from the group consisting of three CDRs of a heavy chain (HC) variable region selected from SEQ ID NOs: 1, 3, 5, 7, 9, 11, 13, 15, 17, and 19 and three CDRs of a light chain (LC) variable region selected from SEQ ID NOs: 2, 4, 6, 8, 10, 12, 14, 16, 18, and 20. Alternatively, the anti-CDH17 antibodies or binding fragments thereof comprise a heavy chain variable region and/or a light chain variable region having at least 90%, 95%, 96%, 97%, 98%, or 99% sequence identity to the heavy chain variable region of any one of SEQ ID NOs: 1, 3, 5, 7, 9, 11, 13, 15, 17, or 19 and/or the light chain variable region of any one of SEQ ID NOs: 2, 4, 6, 8, 10, 12, 14, 16, 18, or 20, respectively. In an embodiment, the anti-CDH17 antibodies or binding fragments thereof comprise 1, 2, 3, 4, 5, 6, 7, 8, 9, or 10 amino acid mutations (e.g., substitutions, deletions, insertions) in one or more framework regions and/or one or more CDR regions (e.g., 1, 2, 3, 4, 5, or 6 CDR regions) as compared to an anti-CDH17 antibody that comprises a heavy chain variable region of any one of SEQ ID NOs: 1, 3, 5, 7, 9, 11, 13, 15, 17, and 19 and/or a light chain variable region of any one of SEQ ID NOs: 2, 4, 6, 8, 10, 12, 14, 16, 18, and 20 respectively, provided that the antibody or fragment thereof retains binding to CDH17.

In some embodiments, the anti-CDH17 antibodies or binding fragments thereof comprise a heavy chain variable region comprising CDR1: SEQ ID NO: 21, CDR2: SEQ ID NO: 22, and CDR3: SEQ ID NO: 23; and/or a light chain variable region comprising CDR1: SEQ ID NO: 24 CDR2: SEQ ID NO: 25, and CDR3: SEQ ID NO: 26. In some embodiments, the anti-CDH17 antibodies or binding fragments thereof comprise a heavy chain variable region that has an amino acid sequence having at least 90%, 95%, 96%, 97%, 98%, or 99% sequence identity to SEQ ID NO: 1 and/or light chain variable region that has an amino acid sequence having at least 90%, 95%, 96%, 97%, 98%, or 99% sequence identity to SEQ ID NO: 2.

In some embodiments, the anti-CDH17 antibodies or antigen binding fragments thereof comprise a heavy chain variable region comprising CDR1: SEQ ID NO: 27, CDR2: SEQ ID NO: 28, and CDR3: SEQ ID NO: 29; and/or a light chain variable region comprising CDR1: SEQ ID NO: 30, CDR2: SEQ ID NO: 31, and CDR3: SEQ ID NO: 32. In some embodiments, the anti-CDH17 antibodies or binding fragments thereof comprise a heavy chain variable region that has an amino acid sequence having at least 90%, 95%, 96%, 97%, 98%, or 99% sequence identity to SEQ ID NO: 3 and/or light chain variable region that has an amino acid sequence having at least 90%, 95%, 96%, 97%, 98%, or 99% sequence identity to SEQ ID NO: 4.

In some embodiments, the anti-CDH17 antibodies or antigen binding fragments thereof comprise a heavy chain variable region comprising CDR1: SEQ ID NO: 33, CDR2: SEQ ID NO: 34, and CDR3: SEQ ID NO: 35; and/or a light chain variable region comprising CDR1: SEQ ID NO: 36 CDR2: SEQ ID NO: 37, and CDR3: SEQ ID NO: 38. In some embodiments, the anti-CDH17 antibodies or binding fragments thereof comprise a heavy chain variable region that has an amino acid sequence having at least 90%, 95%, 96%, 97%, 98%, or 99% sequence identity to SEQ ID NO: 5 and/or light chain variable region that has an amino acid sequence having at least 90%, 95%, 96%, 97%, 98%, or 99% sequence identity to SEQ ID NO: 6.

In some embodiments, the anti-CDH17 antibodies or antigen binding fragments thereof comprise a heavy chain variable region comprising CDR1: SEQ ID NO: 39, CDR2: SEQ ID NO: 40, and CDR3: SEQ ID NO: 41; and/or a light chain variable region comprising CDR1: SEQ ID NO: 24, CDR2: SEQ ID NO: 25, and CDR3: SEQ ID NO: 42. In some embodiments, the anti-CDH17 antibodies or binding fragments thereof comprise a heavy chain variable region that has an amino acid sequence having at least 90%, 95%, 96%, 97%, 98%, or 99% sequence identity to SEQ ID NO: 7 and/or light chain variable region that has an amino acid sequence having at least 90%, 95%, 96%, 97%, 98%, or 99% sequence identity to SEQ ID NO: 8.

In some embodiments, the anti-CDH17 antibodies or antigen binding fragments thereof comprise a heavy chain variable region comprising CDR1: SEQ ID NO: 43, CDR2: SEQ ID NO: 40, and CDR3: SEQ ID NO: 44; and/or a light chain variable region comprising CDR1: SEQ ID NO: 24, CDR2: SEQ ID NO: 25, and CDR3: SEQ ID NO: 45. In some embodiments, the anti-CDH17 antibodies or binding fragments thereof comprise a heavy chain variable region that has an amino acid sequence having at least 90%, 95%, 96%, 97%, 98%, or 99% sequence identity to SEQ ID NO: 9 and/or light chain variable region that has an amino acid sequence having at least 90%, 95%, 96%, 97%, 98%, or 99% sequence identity to SEQ ID NO: 10.

In some embodiments, the anti-CDH17 antibodies or antigen binding fragments thereof comprise a heavy chain variable region comprising CDR1: SEQ ID NO: 46, CDR2: SEQ ID NO: 47, and CDR3: SEQ ID NO: 48; and/or a light chain variable region comprising CDR1: SEQ ID NO: 49, CDR2: SEQ ID NO: 50, and CDR3: SEQ ID NO: 51. In some embodiments, the anti-CDH17 antibodies or binding fragments thereof comprise a heavy chain variable region that has an amino acid sequence having at least 90%, 95%, 96%, 97%, 98%, or 99% sequence identity to SEQ ID NO: 11 and/or light chain variable region that has an amino acid sequence having at least 90%, 95%, 96%, 97%, 98%, or 99% sequence identity to SEQ ID NO: 12.

In some embodiments, the anti-CDH17 antibodies or antigen binding fragments thereof comprise a heavy chain variable region comprising CDR1: SEQ ID NO: 52, CDR2: SEQ ID NO: 53, and CDR3: SEQ ID NO: 54; and/or a light chain variable region comprising CDR1: SEQ ID NO: 55, CDR2: SEQ ID NO: 56, and CDR3: SEQ ID NO: 57. In some embodiments, the anti-CDH17 antibodies or binding fragments thereof comprise a heavy chain variable region that has an amino acid sequence having at least 90%, 95%, 96%, 97%, 98%, or 99% 98%, or 99% sequence identity to SEQ ID NO: 13 and/or light chain variable region that has an amino acid sequence having at least 90%, 95%, 96%, 97%, 98%, or 99% 98%, or 99% sequence identity to SEQ ID NO: 14.

In some embodiments, the anti-CDH17 antibodies or antigen binding fragments thereof comprise a heavy chain variable region comprising CDR1: SEQ ID NO: 58, CDR2: SEQ ID NO: 59, and CDR3: SEQ ID NO: 60; and/or a light chain variable region comprising CDR1: SEQ ID NO: 61, CDR2: SEQ ID NO: 62, and CDR3: SEQ ID NO: 63. In some embodiments, the anti-CDH17 antibodies or binding fragments thereof comprise a heavy chain variable region that has an amino acid sequence having at least 90%, 95%, 96%, 97%, 98%, or 99% 98%, or 99% sequence identity to SEQ ID NO: 15 and/or light chain variable region that has an amino acid sequence having at least 90%, 95%, 96%, 97%, 98%, or 99% 98%, or 99% sequence identity to SEQ ID NO: 16.

In some embodiments, the anti-CDH17 antibodies or antigen binding fragments thereof comprise a heavy chain variable region comprising CDR1: SEQ ID NO: 64, CDR2: SEQ ID NO: 65, and CDR3: SEQ ID NO: 66; and/or a light chain variable region comprising CDR1: SEQ ID NO: 67, CDR2: SEQ ID NO: 68, and CDR3: SEQ ID NO: 69. In some embodiments, the anti-CDH17 antibodies or binding fragments thereof comprise a heavy chain variable region that has an amino acid sequence having at least 90%, 95%, 96%, 97%, 98%, or 99% 98%, or 99% sequence identity to SEQ ID NO: 17 and/or light chain variable region that has an amino acid sequence having at least 90%, 95%, 96%, 97%, 98%, or 99% 98%, or 99% sequence identity to SEQ ID NO: 18.

In some embodiments, the anti-CDH17 antibodies or antigen binding fragments thereof comprise a heavy chain variable region comprising CDR1: SEQ ID NO: 70, CDR2: SEQ ID NO: 71, and CDR3: SEQ ID NO: 72; and/or a light chain variable region comprising CDR1: SEQ ID NO: 73, CDR2: SEQ ID NO: 74, and CDR3: SEQ ID NO: 75. In some embodiments, the anti-CDH17 antibodies or binding fragments thereof comprise a heavy chain variable region that has an amino acid sequence having at least 90%, 95%, 96%, 97%, 98%, or 99% 98%, or 99% sequence identity to SEQ ID NO: 19 and/or light chain variable region that has an amino acid sequence having at least 90%, 95%, 96%, 97%, 98%, or 99% 98%, or 99% sequence identity to SEQ ID NO: 20.

In some embodiments, the anti-CDH17 antibodies or antigen binding fragments thereof comprise a variable heavy chain sequence having an amino acid sequence selected from SEQ ID NOs: 1, 3, 5, 7, 9, 11, 13, 15, 17 and 19. In another embodiment, the anti-CDH17 antibodies or antigen binding fragments thereof comprise a variable heavy chain sequence having at least 90%, 95%, or 99% sequence identity to SEQ ID NOs: 1, 3, 5, 7, 9, 11, 13, 15, 17, and 19 provided that the antibody or fragment thereof retains binding to CDH17.

In other embodiments, the anti-CDH17 antibodies or antigen binding fragments thereof comprise a variable light chain sequence having an amino acid sequence selected from SEQ ID NOs: 2, 4, 6, 8, 10, 12, 14, 16, 18, and 20. In another embodiment, the anti-CDH17 antibodies or antigen binding fragments thereof comprise a variable light chain sequence having at least 90%, 95%, or 99% sequence identity to SEQ ID NOs: 2, 4, 6, 8, 10, 12, 14, 16, 18, and 20 provided that the antibody or fragment thereof retains binding to CDH17.

In other embodiments, the anti-CDH17 antibodies or antigen binding fragments thereof comprise a variable heavy chain sequence having an amino acid sequence selected from SEQ ID NOs: 1, 3, 5, 7, 9, 11, 13, 15, 17, and 19, or an amino acid sequence having at least 90%, 95%, 96%, 97%, 98%, or 99% 98%, or 99% sequence identity thereto, and a variable light chain sequence having an amino acid sequence selected from SEQ ID NOs: 2, 4, 6, 8, 10, 12, 14, 16, 18, and 20, or an amino acid sequence having at least 90%, 95%, 96%, 97%, 98%, or 99% 98%, or 99% sequence identity thereto.

In some embodiments, the murine anti-CDH17 antibodies or antigen binding fragments thereof comprise a variable heavy chain sequence and a variable light chain sequence, selected from the following combinations:
(a) a variable heavy chain sequence comprising SEQ ID NO: 1, or an amino acid sequence having at least 90%, 95%, 96%, 97%, 98%, or 99% 98%, or 99% sequence identity thereto, and a variable light chain sequence comprising SEQ ID NO: 2, or an amino acid sequence having at least 90%, 95%, 96%, 97%, 98%, or 99% 98%, or 99% sequence identity thereto;
(b) a variable heavy chain sequence comprising SEQ ID NO: 3, or an amino acid sequence having at least 90%, 95%, 96%, 97%, 98%, or 99% 98%, or 99% sequence identity thereto, and a variable light chain sequence comprising SEQ ID NO: 4, or an amino acid sequence having at least 90%, 95%, 96%, 97%, 98%, or 99% 98%, or 99% sequence identity thereto;
(c) a variable heavy chain sequence comprising SEQ ID NO: 5, or an amino acid sequence having at least 90%, 95%, 96%, 97%, 98%, or 99% 98%, or 99% sequence identity thereto, and a variable light chain sequence comprising SEQ ID NO: 6, or an amino acid sequence having at least 90%, 95%, 96%, 97%, 98%, or 99% 98%, or 99% sequence identity thereto;
(d) a variable heavy chain sequence comprising SEQ ID NO: 7, or an amino acid sequence having at least 90%, 95%, 96%, 97%, 98%, or 99% 98%, or 99% sequence identity thereto, and a variable light chain sequence comprising SEQ ID NO: 8, or an amino acid sequence having at least 90%, 95%, 96%, 97%, 98%, or 99% 98%, or 99% sequence identity thereto;
(e) a variable heavy chain sequence comprising SEQ ID NO: 9, or an amino acid sequence having at least 90%, 95%, 96%, 97%, 98%, or 99% 98%, or 99% sequence identity thereto, and a variable light chain sequence comprising SEQ ID NO: 10, or an amino acid sequence having at least 90%, 95%, 96%, 97%, 98%, or 99% 98%, or 99% sequence identity thereto;
(f) a variable heavy chain sequence comprising SEQ ID NO: 11, or an amino acid sequence having at least 90%, 95%, 96%, 97%, 98%, or 99% 98%, or 99% sequence identity thereto, and a variable light chain sequence comprising SEQ ID NO: 12, or an amino acid sequence having at least 90%, 95%, 96%, 97%, 98%, or 99% 98%, or 99% sequence identity thereto;
(g) a variable heavy chain sequence comprising SEQ ID NO: 13, or an amino acid sequence having at least 90%, 95%, 96%, 97%, 98%, or 99% 98%, or 99% sequence identity thereto, and a variable light chain sequence comprising SEQ ID NO: 14, or an amino acid sequence having at least 90%, 95%, 96%, 97%, 98%, or 99% 98%, or 99% sequence identity thereto;
(h) a variable heavy chain sequence comprising SEQ ID NO: 15, or an amino acid sequence having at least 90%, 95%, 96%, 97%, 98%, or 99% 98%, or 99% sequence identity thereto, and a variable light chain sequence comprising SEQ ID NO: 16, or an amino acid sequence having at least 90%, 95%, 96%, 97%, 98%, or 99% 98%, or 99% sequence identity thereto;
(i) a variable heavy chain sequence comprising SEQ ID NO: 17, or an amino acid sequence having at least 90%, 95%, 96%, 97%, 98%, or 99% 98%, or 99% sequence identity thereto, and a variable light chain sequence comprising SEQ ID NO: 18, or an amino acid sequence having at least 90%, 95%, 96%, 97%, 98%, or 99% 98%, or 99% sequence identity thereto; and
(j) a variable heavy chain sequence comprising SEQ ID NO: 19, or an amino acid sequence having at least 90%, 95%, 96%, 97%, 98%, or 99% 98%, or 99% sequence identity thereto, and a variable light chain sequence comprising SEQ ID NO: 20, or an amino acid sequence having at least 90%, 95%, 96%, 97%, 98%, or 99% 98%, or 99% sequence identity thereto.

In some embodiments, the present disclosure provides parental murine anti-CDH17 antibodies and antigen binding fragments thereof and methods of use thereof. A skilled practitioner will recognize that the disclosed antibodies can be modified for an intended use, such as conversion into a chimeric antibody or humanized for use as a human therapeutic antibody or binding fragment. In some embodiments, the CDH17 antibody is a recombinant monoclonal antibody.

According to some embodiments, the anti-CDH17 antibodies or binding fragments thereof are humanized antibodies comprising a set of six complementarity determining region (CDR) sequences selected from the group consisting of three CDRs of a heavy chain (HC) variable region selected and three CDRs of a light chain (LC) variable region of C17_mAb 1, C17_mAb 2, C17_mAb 3, C17_mAb 4, C17_mAb 5, C17_mAb 6, C17_mAb 7, C17_mAb 8, C17_mAb 9, and C17_mAb 10.

For example, the disclosure includes a humanized version of: the C17_mAb 1 antibody (e.g., comprising CDR regions derived from the VH sequence provided in SEQ ID NO: 1 and the VL sequence provided in SEQ ID NO: 2), the C17_mAb 2 antibody (e.g., comprising CDR regions derived from the VH sequence provided in SEQ ID NO: 3 and the VL sequence provided in SEQ ID NO: 4), the C17_mAb 3 antibody (e.g., comprising CDR regions derived from the VH sequence provided in SEQ ID NO: 5 and the VL sequence provided in SEQ ID NO: 6), the C17_mAb 4 antibody (e.g., comprising CDR regions derived from the VH sequence provided in SEQ ID NO: 7 and the VL sequence provided in SEQ ID NO: 8), the C17_mAb 5 antibody (e.g., comprising the CDR regions provided in SEQ ID NO: 9 and the VL sequence provided in SEQ ID NO: 10), the C17_mAb 6 antibody (e.g., comprising CDR regions derived from the VH sequence provided in SEQ ID NO: 11 and the VL sequence provided in SEQ ID NO: 12), the C17_mAb 7 antibody (e.g., comprising CDR regions derived from the VH sequence provided in SEQ ID NO: 13 and the VL sequence provided in SEQ ID NO: 14), the C17_mAb 8 antibody (e.g., comprising CDR regions derived from the VH sequence provided in SEQ ID NO: 15 and the VL sequence provided in SEQ ID NO: 16), the C17_mAb 9 antibody (e.g., comprising CDR regions derived from the VH sequence provided in SEQ ID NO: 17 and the VL sequence provided in SEQ ID NO: 18), or, the C17_mAb 10 antibody (e.g., comprising CDR regions derived from the VH sequence provided in SEQ ID NO: 19 and the VL sequence provided in SEQ ID NO: 20).

In some embodiments, the humanized anti-CDH17 antibodies or antigen binding fragments thereof comprise a variable heavy chain sequence having an amino acid sequence selected from SEQ ID NOs: 76, 77, 80, 81, 84, 85, and 123, or an amino acid sequence having at least 90%, 95%, or 99% sequence identity thereto, and a variable light chain sequence having an amino acid sequence selected from SEQ ID NOs: 78, 79, 82, 83, 86, 87, and 124, or an amino acid sequence having at least 90%, 95%, or 99% sequence identity thereto.

In some embodiments, the humanized anti-CDH17 antibodies or antigen binding fragments thereof comprise a variable heavy chain sequence having an amino acid sequence selected from SEQ ID NOs: 76, 77, 80, 81, 84, 85, and 123. In another embodiment, the anti-CDH17 antibodies or antigen binding fragments thereof comprise a variable heavy chain sequence having at least 90%, 95%, or 99% sequence identity to SEQ ID NOs: 76, 77, 80, 81, 84, 85, and 123 provided that the antibody or fragment thereof retains binding to CDH17.

In other embodiments, the humanized anti-CDH17 antibodies or antigen binding fragments thereof comprise a variable light chain sequence having an amino acid sequence selected from SEQ ID NOs: 78, 79, 82, 83, 86, 87, and 124. In another embodiment, the anti-CDH17 antibodies or antigen binding fragments thereof comprise a variable light chain sequence having at least 90%, 95%, or 99% sequence identity to 78, 79, 82, 83, 86, 87, and 124 provided that the antibody or fragment thereof retains binding to CDH17.

In some embodiments, the humanized anti-CDH17 antibodies or antigen binding fragments thereof comprise a variable heavy chain sequence and a variable light chain sequence, selected from the following combinations:
(a) a variable heavy chain sequence comprising SEQ ID NO: 76, or an amino acid sequence having at least 90%, 95%, 96%, 97%, 98%, or 99% sequence identity thereto, and a variable light chain sequence comprising SEQ ID NO: 78, or an amino acid sequence having at least 90%, 95%, 96%, 97%, 98%, or 99% sequence identity thereto;
(b) a variable heavy chain sequence comprising SEQ ID NO: 76, or an amino acid sequence having at least 90%, 95%, 96%, 97%, 98%, or 99% sequence identity thereto, and a variable light chain sequence comprising SEQ ID NO: 79, or an amino acid sequence having at least 90%, 95%, 96%, 97%, 98%, or 99% sequence identity thereto;
(c) a variable heavy chain sequence comprising SEQ ID NO: 77, or an amino acid sequence having at least 90%, 95%, 96%, 97%, 98%, or 99% sequence identity thereto, and a variable light chain sequence comprising SEQ ID NO: 78, or an amino acid sequence having at least 90%, 95%, 96%, 97%, 98%, or 99% sequence identity thereto;
(d) a variable heavy chain sequence comprising SEQ ID NO: 77, or an amino acid sequence having at least 90%, 95%, 96%, 97%, 98%, or 99% sequence identity thereto, and a variable light chain sequence comprising SEQ ID NO: 79, or an amino acid sequence having at least 90%, 95%, 96%, 97%, 98%, or 99% sequence identity thereto;
(e) a variable heavy chain sequence comprising SEQ ID NO: 80, or an amino acid sequence having at least 90%, 95%, 96%, 97%, 98%, or 99% sequence identity thereto, and a variable light chain sequence comprising SEQ ID NO: 82, or an amino acid sequence having at least 90%, 95%, 96%, 97%, 98%, or 99% sequence identity thereto;
(f) a variable heavy chain sequence comprising SEQ ID NO:80, or an amino acid sequence having at least 90%, 95%, 96%, 97%, 98%, or 99% sequence identity thereto, and a variable light chain sequence comprising SEQ ID NO: 83, or an amino acid sequence having at least 90%, 95%, 96%, 97%, 98%, or 99% sequence identity thereto;
(g) a variable heavy chain sequence comprising SEQ ID NO: 81, or an amino acid sequence having at least 90%, 95%, 96%, 97%, 98%, or 99% sequence identity thereto, and a variable light chain sequence comprising SEQ ID NO: 82, or an amino acid sequence having at least 90%, 95%, 96%, 97%, 98%, or 99% sequence identity thereto;
(h) a variable heavy chain sequence comprising SEQ ID NO: 81, or an amino acid sequence having at least 90%, 95%, 96%, 97%, 98%, or 99% sequence identity thereto, and a variable light chain sequence comprising SEQ ID NO: 83, or an amino acid sequence having at least 90%, 95%, 96%, 97%, 98%, or 99% sequence identity thereto;
(i) a variable heavy chain sequence comprising SEQ ID NO: 84, or an amino acid sequence having at least 90%, 95%, 96%, 97%, 98%, or 99% sequence identity thereto, and a variable light chain sequence comprising SEQ ID NO: 86, or an amino acid sequence having at least 90%, 95%, 96%, 97%, 98%, or 99% sequence identity thereto;
(j) a variable heavy chain sequence comprising SEQ ID NO: 84, or an amino acid sequence having at least 90%, 95%, 96%, 97%, 98%, or 99% sequence identity thereto, and a variable light chain sequence comprising SEQ ID NO: 87, or an amino acid sequence having at least 90%, 95%, 96%, 97%, 98%, or 99% sequence identity thereto;
(k) a variable heavy chain sequence comprising SEQ ID NO: 85, or an amino acid sequence having at least 90%, 95%, 96%, 97%, 98%, or 99% sequence identity thereto, and a variable light chain sequence comprising SEQ ID NO: 86, or an amino acid sequence having at least 90%, 95%, 96%, 97%, 98%, or 99% sequence identity thereto;
(l) a variable heavy chain sequence comprising SEQ ID NO: 85, or an amino acid sequence having at least 90%, 95%, 96%, 97%, 98%, or 99% sequence identity thereto, and a variable light chain sequence comprising SEQ ID NO: 87, or an amino acid sequence having at least 90%, 95%, 96%, 97%, 98%, or 99% sequence identity thereto; and
(m) a variable heavy chain sequence comprising SEQ ID NO: 123, or an amino acid sequence having at least 90%, 95%, 96%, 97%, 98%, or 99% sequence identity thereto, and a variable light chain sequence comprising SEQ ID NO: 124, or an amino acid sequence having at least 90%, 95%, 96%, 97%, 98%, or 99% sequence identity thereto.

In some embodiments, an immunoconjugate comprising an antibody or fragment thereof that binds CDH17 attached (e.g., via a linker) to a cytotoxic agent (e.g., a payload) is provided, wherein the antibody or fragment thereof comprises a variable heavy chain sequence and a variable light chain sequence, selected from the following combinations:
(a) a variable heavy chain sequence comprising SEQ ID NO: 1, or an amino acid sequence having at least 90%, 95%, 96%, 97%, 98%, or 99% sequence identity thereto, and a variable light chain sequence comprising SEQ ID NO: 2, or an amino acid sequence having at least 90%, 95%, 96%, 97%, 98%, or 99% sequence identity thereto;
(b) a variable heavy chain sequence comprising SEQ ID NO: 3, or an amino acid sequence having at least 90%, 95%, 96%, 97%, 98%, or 99% sequence identity thereto, and a variable light chain sequence comprising SEQ ID NO: 4, or an amino acid sequence having at least 90%, 95%, 96%, 97%, 98%, or 99% sequence identity thereto;
(c) a variable heavy chain sequence comprising SEQ ID NO: 5, or an amino acid sequence having at least 90%, 95%, 96%, 97%, 98%, or 99% sequence identity thereto, and a variable light chain sequence comprising SEQ ID NO: 6, or an amino acid sequence having at least 90%, 95%, 96%, 97%, 98%, or 99% sequence identity thereto;
(d) a variable heavy chain sequence comprising SEQ ID NO: 7, or an amino acid sequence having at least 90%, 95%, 96%, 97%, 98%, or 99% sequence identity thereto, and a variable light chain sequence comprising SEQ ID NO: 8, or an amino acid sequence having at least 90%, 95%, 96%, 97%, 98%, or 99% sequence identity thereto;
(e) a variable heavy chain sequence comprising SEQ ID NO: 9, or an amino acid sequence having at least 90%, 95%, 96%, 97%, 98%, or 99% sequence identity thereto, and a variable light chain sequence comprising SEQ ID NO: 10, or an amino acid sequence having at least 90%, 95%, 96%, 97%, 98%, or 99% sequence identity thereto;
(f) a variable heavy chain sequence comprising SEQ ID NO: 11, or an amino acid sequence having at least 90%, 95%, 96%, 97%, 98%, or 99% sequence identity thereto, and a variable light chain sequence comprising SEQ ID NO: 12, or an amino acid sequence having at least 90%, 95%, 96%, 97%, 98%, or 99% sequence identity thereto;
(g) a variable heavy chain sequence comprising SEQ ID NO: 13, or an amino acid sequence having at least 90%, 95%, 96%, 97%, 98%, or 99% sequence identity thereto, and a variable light chain sequence comprising SEQ ID NO: 14, or an amino acid sequence having at least 90%, 95%, 96%, 97%, 98%, or 99% sequence identity thereto;
(h) a variable heavy chain sequence comprising SEQ ID NO: 15, or an amino acid sequence having at least 90%, 95%, 96%, 97%, 98%, or 99% sequence identity thereto, and a variable light chain sequence comprising SEQ ID NO: 16, or an amino acid sequence having at least 90%, 95%, 96%, 97%, 98%, or 99% sequence identity thereto;
(i) a variable heavy chain sequence comprising SEQ ID NO: 17, or an amino acid sequence having at least 90%, 95%, 96%, 97%, 98%, or 99% sequence identity thereto, and a variable light chain sequence comprising SEQ ID NO: 18, or an amino acid sequence having at least 90%, 95%, 96%, 97%, 98%, or 99% sequence identity thereto; and
(j) a variable heavy chain sequence comprising SEQ ID NO: 19, or an amino acid sequence having at least 90%, 95%, 96%, 97%, 98%, or 99% sequence identity thereto, and a variable light chain sequence comprising SEQ ID NO: 20, or an amino acid sequence having at least 90%, 95%, 96%, 97%, 98%, or 99% sequence identity thereto.

In some embodiments, an immunoconjugate comprising an antibody or fragment thereof that binds CDH17 attached (e.g., via a linker) to a cytotoxic agent (e.g., a payload) is provided, wherein the antibody or fragment thereof comprises a variable heavy chain sequence and a variable light chain sequence, selected from the following combinations:
(a) a variable heavy chain sequence comprising SEQ ID NO: 76, or an amino acid sequence having at least 90%, 95%, 96%, 97%, 98%, or 99% sequence identity thereto, and a variable light chain sequence comprising SEQ ID NO: 78, or an amino acid sequence having at least 90%, 95%, 96%, 97%, 98%, or 99% sequence identity thereto;
(b) a variable heavy chain sequence comprising SEQ ID NO: 76, or an amino acid sequence having at least 90%, 95%, 96%, 97%, 98%, or 99% sequence identity thereto, and a variable light chain sequence comprising SEQ ID NO: 79, or an amino acid sequence having at least 90%, 95%, 96%, 97%, 98%, or 99% sequence identity thereto;
(c) a variable heavy chain sequence comprising SEQ ID NO: 77, or an amino acid sequence having at least 90%, 95%, 96%, 97%, 98%, or 99% sequence identity thereto, and a variable light chain sequence comprising SEQ ID NO: 78, or an amino acid sequence having at least 90%, 95%, 96%, 97%, 98%, or 99% sequence identity thereto;
(d) a variable heavy chain sequence comprising SEQ ID NO: 77, or an amino acid sequence having at least 90%, 95%, 96%, 97%, 98%, or 99% sequence identity thereto, and a variable light chain sequence comprising SEQ ID NO: 79, or an amino acid sequence having at least 90%, 95%, 96%, 97%, 98%, or 99% sequence identity thereto;
(e) a variable heavy chain sequence comprising SEQ ID NO: 80, or an amino acid sequence having at least 90%, 95%, 96%, 97%, 98%, or 99% sequence identity thereto, and a variable light chain sequence comprising SEQ ID NO: 82, or an amino acid sequence having at least 90%, 95%, 96%, 97%, 98%, or 99% sequence identity thereto;
(f) a variable heavy chain sequence comprising SEQ ID NO:80, or an amino acid sequence having at least 90%, 95%, 96%, 97%, 98%, or 99% sequence identity thereto, and a variable light chain sequence comprising SEQ ID NO: 83, or an amino acid sequence having at least 90%, 95%, 96%, 97%, 98%, or 99% sequence identity thereto;
(g) a variable heavy chain sequence comprising SEQ ID NO: 81, or an amino acid sequence having at least 90%, 95%, 96%, 97%, 98%, or 99% sequence identity thereto, and a variable light chain sequence comprising SEQ ID NO: 82, or an amino acid sequence having at least 90%, 95%, 96%, 97%, 98%, or 99% sequence identity thereto;
(h) a variable heavy chain sequence comprising SEQ ID NO: 81, or an amino acid sequence having at least 90%, 95%, 96%, 97%, 98%, or 99% sequence identity thereto, and a variable light chain sequence comprising SEQ ID NO: 83, or an amino acid sequence having at least 90%, 95%, 96%, 97%, 98%, or 99% sequence identity thereto;
(i) a variable heavy chain sequence comprising SEQ ID NO: 84, or an amino acid sequence having at least 90%, 95%, 96%, 97%, 98%, or 99% sequence identity thereto, and a variable light chain sequence comprising SEQ ID NO: 86, or an amino acid sequence having at least 90%, 95%, 96%, 97%, 98%, or 99% sequence identity thereto;
(j) a variable heavy chain sequence comprising SEQ ID NO: 84, or an amino acid sequence having at least 90%, 95%, 96%, 97%, 98%, or 99% sequence identity thereto, and a variable light chain sequence comprising SEQ ID NO: 87, or an amino acid sequence having at least 90%, 95%, 96%, 97%, 98%, or 99% sequence identity thereto;
(k) a variable heavy chain sequence comprising SEQ ID NO: 85, or an amino acid sequence having at least 90%, 95%, 96%, 97%, 98%, or 99% sequence identity thereto, and a variable light chain sequence comprising SEQ ID NO: 86, or an amino acid sequence having at least 90%, 95%, 96%, 97%, 98%, or 99% sequence identity thereto;
(l) a variable heavy chain sequence comprising SEQ ID NO: 85, or an amino acid sequence having at least 90%, 95%, 96%, 97%, 98%, or 99% sequence identity thereto, and a variable light chain sequence comprising SEQ ID NO: 87, or an amino acid sequence having at least 90%, 95%, 96%, 97%, 98%, or 99% sequence identity thereto; and
(m) a variable heavy chain sequence comprising SEQ ID NO: 123, or an amino acid sequence having at least 90%, 95%, 96%, 97%, 98%, or 99% sequence identity thereto, and a variable light chain sequence comprising SEQ ID NO: 124, or an amino acid sequence having at least 90%, 95%, 96%, 97%, 98%, or 99% sequence identity thereto.

In some embodiments, an immunoconjugate comprising an antibody or binding fragment thereof that binds CDH17 covalently attached to a cytotoxic agent is provided, wherein the antibody comprises:
(a) a heavy chain variable region comprising CDR1: SEQ ID NO: 21, CDR2: SEQ ID NO: 22, and CDR3: SEQ ID NO: 23; and/or a light chain variable region comprising CDR1: SEQ ID NO: 24, CDR2: SEQ ID NO: 25, and CDR3: SEQ ID NO: 26, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 1 and/or light chain variable region that has an amino acid sequence having at least 90%, 95%, 96%, 97%, 98%, or 99% sequence identity to SEQ ID NO: 2;
(b) a heavy chain variable region comprising CDR1: SEQ ID NO: 27, CDR2: SEQ ID NO: 28, and CDR3: SEQ ID NO: 29; and/or a light chain variable region comprising CDR1: SEQ ID NO: 30, CDR2: SEQ ID NO: 31, and CDR3: SEQ ID NO: 32, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 3 and/or light chain variable region that has an amino acid sequence having at least 90%, 95%, 96%, 97%, 98%, or 99% sequence identity to SEQ ID NO: 4;
(c) a heavy chain variable region comprising CDR1: SEQ ID NO: 33, CDR2: SEQ ID NO: 34, and CDR3: SEQ ID NO: 35; and/or a light chain variable region comprising CDR1: SEQ ID NO: 36, CDR2: SEQ ID NO: 37, and CDR3: SEQ ID NO: 38, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 5 and/or light chain variable region that has an amino acid sequence having at least 90%, 95%, 96%, 97%, 98%, or 99% sequence identity to SEQ ID NO: 6;
(d) a heavy chain variable region comprising CDR1: SEQ ID NO: 39, CDR2: SEQ ID NO: 40, and CDR3: SEQ ID NO: 41; and/or a light chain variable region comprising CDR1: SEQ ID NO: 24, CDR2: SEQ ID NO: 25, and CDR3: SEQ ID NO: 42, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 7 and/or light chain variable region that has an amino acid sequence having at least 90%, 95%, 96%, 97%, 98%, or 99% sequence identity to SEQ ID NO: 8;
(e) a heavy chain variable region comprising CDR1: SEQ ID NO: 43, CDR2: SEQ ID NO: 40, and CDR3: SEQ ID NO: 44; and/or a light chain variable region comprising CDR1: SEQ ID NO: 24, CDR2: SEQ ID NO: 25, and CDR3: SEQ ID NO: 45, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 9 and/or light chain variable region that has an amino acid sequence having at least 90%, 95%, 96%, 97%, 98%, or 99% sequence identity to SEQ ID NO: 10;
(f) a heavy chain variable region comprising CDR1: SEQ ID NO: 46, CDR2: SEQ ID NO: 47, and CDR3: SEQ ID NO: 48; and/or a light chain variable region comprising CDR1: SEQ ID NO: 49, CDR2: SEQ ID NO: 50, and CDR3: SEQ ID NO: 51, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 11 and/or light chain variable region that has an amino acid sequence having at least 90%, 95%, 96%, 97%, 98%, or 99% sequence identity to SEQ ID NO: 12;
(g) a heavy chain variable region comprising CDR1: SEQ ID NO: 52, CDR2: SEQ ID NO: 53, and CDR3: SEQ ID NO: 54; and/or a light chain variable region comprising CDR1: SEQ ID NO: 55, CDR2: SEQ ID NO: 56, and CDR3: SEQ ID NO: 57, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 13 and/or light chain variable region that has an amino acid sequence having at least 90%, 95%, 96%, 97%, 98%, or 99% sequence identity to SEQ ID NO: 14;
(h) a heavy chain variable region comprising CDR1: SEQ ID NO: 58, CDR2: SEQ ID NO: 59, and CDR3: SEQ ID NO: 60; and/or a light chain variable region comprising CDR1: SEQ ID NO: 61, CDR2: SEQ ID NO: 62, and CDR3: SEQ ID NO: 63, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 15 and/or light chain variable region that has an amino acid sequence having at least 90%, 95%, 96%, 97%, 98%, or 99% sequence identity to SEQ ID NO: 16;
(i) a heavy chain variable region comprising CDR1: SEQ ID NO: 64, CDR2: SEQ ID NO: 65, and CDR3: SEQ ID NO: 66; and/or a light chain variable region comprising CDR1: SEQ ID NO: 67, CDR2: SEQ ID NO: 68, and CDR3: SEQ ID NO: 69, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 17 and/or light chain variable region that has an amino acid sequence having at least 90%, 95%, 96%, 97%, 98%, or 99% sequence identity to SEQ ID NO: 18; or
(j) a heavy chain variable region comprising CDR1: SEQ ID NO: 70, CDR2: SEQ ID NO: 71, and CDR3: SEQ ID NO: 72; and/or a light chain variable region comprising CDR1: SEQ ID NO: 73, CDR2: SEQ ID NO: 74, and CDR3: SEQ ID NO: 75, optionally wherein the anti-CDH17 antibodies or binding fragments thereof comprise a heavy chain variable region that has an amino acid sequence having at least 90%, 95%, 96%, 97%, 98%, or 99% sequence identity to SEQ ID NO: 19 and/or light chain variable region that has an amino acid sequence having at least 90%, 95%, 96%, 97%, 98%, or 99% sequence identity to SEQ ID NO: 20.

In general, a humanized CDH17 antibody or binding fragment thereof may comprise substantially all of at least one, and typically two, variable domains, in which all, or substantially all of the hypervariable loops correspond to those of a parental murine anti-CDH17 antibody disclosed herein, and all or substantially all of the framework (FR) regions derived from a suitable human consensus immunoglobulin sequence. The humanized antibody or binding fragment thereof may optionally comprise at least a portion of (including all of) a human immunoglobulin constant region (Fc). In an alternative embodiment the human Fc region may include mutations designed to enhance or eliminate effector functions. For example, the Fc region may include mutations known to eliminate FcγR-binding, such as the “LALAPA” mutations (Shields R L, et al 2001).

In some embodiments, the CDH17 antibody or antigen binding fragment comprises six (6) CDRs, all derived from the VH or VL domain of a single parental anti-CDH17 antibody disclosed herein. For example, a binding agent may comprise all six of the CDR regions of the anti-CDH17 antibody designated “C17_mAb 1.” In a representative example, an antibody or antigen binding fragment thereof may comprise the amino acid sequences of SEQ ID NOs: 21-23 and SEQ ID NOs: 24-26, representing the CDR1, CDR2 and CDR3 of the variable heavy chain region and the CDR1, CDR2 and CDR3 of the variable light chain region of the murine anti-human CDH17 antibody referred to herein as “C17_mAb 1.”

In another alternative embodiment, heavy chain CDR and light chain variable CDR sequences of a disclosed CDH17 antibody (e.g., C17_mAb 1, C17_mAb 2, C17_mAb 3, C17 mAb 4, C17_mAb 5, C17_mAb 6, C17_mAb 7, C17_mAb 8, C17_mAb 9 and C17_mAb 10) (see Table 1 and Table 2) or antigen binding fragments thereof are incorporated into a bispecific or a biparatopic antibody.

In an alternative embodiment, heavy chain and light chain variable sequences of a disclosed humanized anti-CDH17 antibody (e.g., C17_mAb 1, C17_mAb 2, C17_mAb 3 and C17_mAb 4) or antigen binding fragments thereof are incorporated into a bispecific or a biparatopic antibody. In a particular embodiment, the biparatopic antibody comprises binding specificity for two distinct non-overlapping epitopes in human CDH17.

In a particular embodiment, the biparatopic anti-CDH17 antibody is constructed in an IgG-like format (e.g., IgG1-, IgG2-, IgG3-, or IgG4-like format) and comprises a first Fab fragment with binding specificity for a first epitope “X” in human CDH17 and a second Fab fragment with binding specificity for a second epitope “Y” in of human CDH17. In a particular embodiment, the biparatopic antibody (bpAb1, bpAb 1.1, bpAb 2 and bpAb 2.1) comprises a binding specificity to two distinct and non-overlapping epitopes within human CDH17 such as ECD 3-5 corresponding to an epitope in X and ECD 6-7 corresponding to an epitope in Y of human CDH17. In an alternative embodiment, the “biparatopic antibody (bpAb 3) comprises a binding specificity to two distinct and non-overlapping epitopes within human CDH17 such as ECD 1-2 corresponding to an epitope in X and ECD 6-7 corresponding to an epitope in Y of human CDH17.

In a particular embodiment, the biparatopic anti-CDH17 antibody comprises a first antigen-binding portion having specificity to a first epitope in human CDH17 and a second antigen-binding portion having specificity to a second distinct non-overlapping epitope within human CDH17.

In some embodiments, the biparatopic antibody comprises (a) a first antigen binding portion having specificity for an epitope in ECD 3-5 of human CDH17 and a second binding portion having specificity for an epitope in ECD 6-7 of human CDH17; or (b) a first antigen binding portion having specificity for an epitope in ECD 1-2 of human CDH17 and a second binding portion having specificity for an epitope in ECD 6-7 of human CDH17.

In some embodiments, the first antigen-binding portion and the second antigen-binding portion comprise heavy chain complementarity determining regions (CDR) VH CDR1, VH CDR2, and VH CDR3, and light chain CDRs VL CDR1, VL CDR2, and VL CDR3. In some embodiments, the VH CDR1, VH CDR2, VH CDR3, VL CDR1, VL CDR2, and VL CDR3 of the first antigen-binding portion are, respectively: (i) the amino acid sequences of SEQ ID NO: 21-26; or (ii) the amino acid sequences of SEQ ID NO: 64-69; and the VH CDR1, VH CDR2, VH CDR3, VL CDR1, VL CDR2, and VL CDR3 of the second antigen-binding portion are, respectively: (i) the amino acid sequences of SEQ ID NO: 27-32; (ii) the amino acid sequences of SEQ ID NO: 33-38; (iii) the amino acid sequences of SEQ ID NO: 39-41, SEQ ID NO: 24, SEQ ID NO: 25 and SEQ ID NO: 42; (iv) the amino acid sequences of SEQ ID NO: 43, SEQ ID NO: 40, SEQ ID NO: 44, SEQ ID NO: 24, SEQ ID NO: 25 and SEQ ID NO: 45; or (v) the amino acid sequences of SEQ ID NO: 46-51. In some embodiments, the VH CDR1, VH CDR2, VH CDR3, VL CDR1, VL CDR2, and VL CDR3 of the first antigen-binding portion are, respectively, the amino acid sequences of SEQ ID NO: 58-63; and the VH CDR1, VH CDR2, VH CDR3, VL CDR1, VL CDR2, and VL CDR3 of the second antigen-binding portion are, respectively: (i) the amino acid sequences of SEQ ID NO: 27-32; (ii) the amino acid sequences of SEQ ID NO: 33-38; (iii) the amino acid sequences of SEQ ID NO: 39-41, SEQ ID NO: 24, SEQ ID NO: 25 and SEQ ID NO: 42; (iv) the amino acid sequences of SEQ ID NO: 43, SEQ ID NO: 40, SEQ ID NO: 44, SEQ ID NO: 24, SEQ ID NO: 25 and SEQ ID NO: 45; or (v) the amino acid sequences of SEQ ID NO: 46-51.

In a particular embodiment, the biparatopic anti-CDH17 antibody comprises a first Fab fragment (Arm 1) comprising a first heavy chain variable region and a first light chain variable region and a second Fab fragment (Arm 2) comprising a second heavy chain variable region and a second variable light chain region, wherein the first heavy chain variable region, the first light chain variable region, the second heavy chain variable region, and the second variable light chain region, respectively, comprise amino acid sequences that are at least 90%, at least 95%, or at least 99% identical to the amino acid sequences of:
(a) SEQ ID NO: 88, SEQ ID NO: 89, SEQ ID NO: 90, and SEQ ID NO: 91;
(b) SEQ ID NO: 88, SEQ ID NO: 89, SEQ ID NO: 92 and SEQ ID NO: 93;
(c) SEQ ID NO: 94, SEQ ID NO: 95, SEQ ID NO: 96, and SEQ ID NO: 97;
(d) SEQ ID NO: 117, SEQ ID NO: 118, SEQ ID NO: 119, and SEQ ID NO: 120; or
(e) SEQ ID NO: 117, SEQ ID NO: 118, SEQ ID NO: 121, and SEQ ID NO: 122.

In some embodiments, the first heavy chain variable region, the first light chain variable region, the second heavy chain variable region, and the second variable light chain region, respectively, comprise amino acid sequences of:
(a) SEQ ID NO: 88, SEQ ID NO: 89, SEQ ID NO: 90, and SEQ ID NO: 91;
(b) SEQ ID NO: 88, SEQ ID NO: 89, SEQ ID NO: 92 and SEQ ID NO: 93;
(c) SEQ ID NO: 94, SEQ ID NO: 95, SEQ ID NO: 96, and SEQ ID NO: 97;
(d) SEQ ID NO: 117, SEQ ID NO: 118, SEQ ID NO: 119, and SEQ ID NO: 120; or
(e) SEQ ID NO: 117, SEQ ID NO: 118, SEQ ID NO: 121, and SEQ ID NO: 122.

In some embodiments, biparatopic anti-CDH17 antibody or antigen-binding fragment thereof is or comprises a F(ab′)₂, a F(ab)₂, IgG-scFv, tandem scFv and a bispecific antibody.

In some embodiments, the biparatopic anti-CDH17 antibody is conjugated to a cytotoxin using linker technology available. In a particular embodiment, the biparatopic anti-CDH17 antibody has been directly conjugated or paired with secondary antibody that is conjugated with cleavable linker carrying payloads such as exatecan, dxd, MMAE, MMAF, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, along with their analogs or homologs. For further exploration of cytotoxin varieties, linkers, and methodologies for coupling therapeutic agents with antibodies, refer to the following references: Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

In an alternative embodiment, the CDH17 antibody or antigen binding fragments thereof is incorporated into a bispecific or a multispecific antibody. In a particular embodiment, the disclosed bispecific antibodies comprise one binding specificity for CDH17 and a second binding specificity for a human immune cell surface protein such as CD3, CD28, CD137, OX40, CD27, GITR, TNFR2, CD16 or CD40, a tumor specific antigen (TSA) or a tumor-associated antigen (TAA) including but not limited to B7-H3, Claudin 18.2, EGFR, cMet, VEGFR, MUC1, HER3, HER2, CEA, TROP2.

In some embodiments, the bispecific anti-CDH17 x CD3 antibody (BsAb 1, BsAb 2, BsAb 3, BsAb 4 and BsAb 5) antibody have an IgG-ScFv structural format. In a particular embodiment bispecific CDH17 x CD3 antibodies comprises a scFv with binding specificity for CD3 and a Fab fragment with binding specificity for an epitope of human CDH17.

In some embodiments, the bispecific T cell engager antibody comprises a first anti-CD3 scFv fragment (Arm 1) and a second Fab fragment (Arm 2) comprising a heavy chain variable region and a light chain variable region specific for CDH17. In some embodiments, the heavy chain variable region and the light chain variable region of the Fab fragment, respectively, comprise the amino acid sequences of
(a) SEQ ID NO: 100 and SEQ ID NO: 101;
(b) SEQ ID NO: 102 and SEQ ID NO: 103;
(c) SEQ ID NO: 104 and SEQ ID NO: 105; or
(d) SEQ ID NO: 125 and SEQ ID NO: 126.

In some embodiments, the anti-CD3 scFv fragment comprises an amino acid sequence of SEQ ID NO: 98 or SEQ ID NO: 127.

In a particular embodiment, the bispecific anti-CDH17xCD3 antibody comprises a first anti-CD3 scFv fragment (Arm 1) and a second Fab fragment (Arm 2) comprising a heavy chain variable region and a variable light chain region specific for CDH17 comprising the following sequence:
(a) Arm 1 scFv SEQ ID NO: 98 and Arm 2 heavy chain variable region SEQ ID NO: 100 and variable light chain region SEQ ID NO: 101;
(b) Arm 1 scFv SEQ ID NO: 98 and Arm 2 heavy chain variable region SEQ ID NO: 102 and variable light chain region SEQ ID NO: 103;
(c) Arm 1 scFv SEQ ID NO: 98 and Arm 2 heavy chain variable region SEQ ID NO: 104 and variable light chain region SEQ ID NO: 105;
(d) Arm 1 scFv SEQ ID NO: 98 and Arm 2 heavy chain variable region SEQ ID NO: 125 and variable light chain region SEQ ID NO: 126; or
(e) Arm 1 scFv SEQ ID NO: 127 and Arm 2 heavy chain variable region SEQ ID NO: 125 and variable light chain region SEQ ID NO: 126.

In some embodiments, the anti-CDH17 antibodies and antigen binding fragments thereof comprise one or more heavy chain variable region CDRs disclosed in Table 1 and/or one or more light chain variable region CDRs disclosed in Table 2.

In some embodiments, the anti-CDH17 antibodies or fragments thereof specifically bind to human cells expressing endogenous levels of CDH17 and/or to host cells engineered to overexpress human or cynomolgus monkey CDH17.

In some embodiments, the CDH17 antibodies or antigen binding fragments bind human CDH17 with affinities between (<1 nM-10 nM) (e.g., in cell based binding experiment), and do not cross react with human CDH16.

In some embodiments, the CDH17 antibodies or antigen binding fragments bind to an epitope within human CDH17 ECD 1-2 or within human CDH17 ECD 3-5 or within human CDH17 ECD 6-7.

In one embodiment, an anti-CDH17 antibody binds to the same epitope bin of ECD 1-2 as PC1 (WO2019/222428), PC3 (WO2018/115231), PC7 (WO2010/123874), and PC8 (WO2023/107558). The remaining embodiments cover 2 other additional epitope bins (within ECD3-5, ECD6-7) distinct from ECD 1-2 bin.

In some embodiments, the CDH17 antibodies or antigen binding fragments are internalized from the surface of CDH17-positive cells after binding CDH17. In particular embodiments, the CDH17 antibodies are internalized and kill human CDH17-positive cells with varying levels of endogenous CDH17 expression when combined with an ADC-conjugated secondary antibody induced killing of tumor cells when conjugated with cytotoxic agent.

In some embodiments, the anti-CDH17 antibodies are biparatopic antibodies that bind to two distinct and non-overlapping epitopes within human CDH17. In one embodiment, a biparatopic anti-CDH17 antibody binds to a first epitope within human CDH17 ECD 3-5 and to a second epitope within ECD 6-7 of human CDH17. In an alternative embodiment, a biparatopic anti-CDH17 antibody binds to a first epitope within human CDH17 ECD 1-2 and to a second epitope within ECD 6-7 of human CDH17.

In some embodiments, the anti-CDH17 antibodies or antigen binding fragments thereof are incorporated into an immunoconjugate (e.g., an ADC) comprising an anti-CDH17 antibody or antigen binding fragment thereof conjugated (e.g., via a linker) to one or more cytotoxic agents, such as chemotherapeutic agents or drugs, growth inhibitory agents, toxins (e.g., protein toxins, enzymatically active toxins of bacterial, fungal, plant, or animal origin, or fragments thereof) or a radionuclide.

In a particular embodiment, an anti-CDH17 antibody or antigen binding fragment is incorporated into an ADC comprising a cell-permeating cytotoxic agent characterized by anti-tumor activity.

In some embodiments, the anti-CDH17 antibodies or antigen binding fragments thereof are incorporated into radioimmunoconjugates (RICs) for use in radiopharmaceutical therapy (RPT) of cancer. In some embodiments, the anti-CDH17 antibodies or antigen binding fragments thereof are incorporated into an imaging agent for diagnostic and/or prognostic uses.

In some embodiments, the anti-CDH17 antibodies or antigen binding fragments thereof are incorporated into bispecific or multispecific antibodies. The CDH17 bispecific or multispecific antibodies contain at least one CDH17-specific antigen-binding domain and a second binding domain characterized by a function that promotes the activation of T cells. For example, in a particular embodiment the bispecific antibody is a CDH17/CD3 T cell engager antibody.

In some embodiments, the anti-CDH17 antibody is a full-length antibody. In some embodiments, the anti-CDH17 antibody is an antigen binding fragment. In further embodiments, the antigen binding fragment is selected from the group consisting of: Fab, Fab′, F(ab′)2, Fd, Fv, scFv and scFv-Fc fragment, a single-chain antibody, a minibody, and a diabody.

The anti-CDH17 antibodies and antigen binding fragments thereof may be used for the treatment of cancer. Methods for the treatment of cancer may comprise administering a composition or formulation that comprises an anti-CDH17 antibody, or antigen binding fragment thereof, to a subject in need of cancer immunotherapy. For example, the anti-CDH17 antibody or antigen binding fragment thereof may be administered either alone (e.g., as a monotherapy) or in combination with another immunotherapeutic agent(s) and/or chemotherapy agent(s).

The anti-CDH17 antibodies and fragments thereof can also be used in the context of a diagnostic. Leveraging CDH17 expression as a diagnostic tool can facilitate the identification and/or classification of patients, enabling a tailored therapeutic strategy for an improved clinical outcome.

Also provided are polynucleotides encoding the anti-CDH17 antibodies or fragments thereof disclosed herein. Additionally, the disclosure provides host cells transformed or transfected with the polynucleotides, and compositions and methods useful for making and using the disclosed anti-CDH17 antibodies, or antigen binding fragments, in therapeutic methods for antibody-based immunotherapy of cancer and/or cancer diagnosis.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIGS. 1A-1I provide the amino acid sequences of the VH and VL domains of the anti-CDH17 antibodies and their respective CDR sequences (IMGT numbering). Sequence identifiers are provided and the CDRs are underlined in the variable domain sequences.

FIGS. 2A-2C provide amino acid sequences of anti-CD3 scFv, human IgG1 Fc and kappa regions, isoforms of CDH17 and CDH16, and extracellular domain (ECD1-2, ECD3-5 and ECD6-7) sequences of human CDH17.

FIG. 3A shows the binding of mPC1 antibody, C17_mAb 1 and C17_mAb 4 to a CHO CDH17 cell line overexpressing human CDH17, FIG. 3B shows the binding of mPC3, mPC8, C17_mAb 1 and C17_mAb 4 antibodies to a CHO CDH17 cell line overexpressing cyno CDH17.

FIGS. 4A and 4B show the protein family tree for CDH17 (4A) and its sequence homology between its family proteins (4B). FIG. 4C shows the lack of binding by disclosed anti-CDH17 antibodies on human CDH16 protein.

FIG. 5A shows CDH17 density in various human cancer cell lines. FIGS. 5B and 5C show the binding by representative anti-CDH17 antibodies on pancreatic tumor cell line AsPC1 (FIG. 5B) and colon tumor cell line COLO205 (FIG. 5C) cells which endogenously express CDH17.

FIGS. 6A-D show the schematic structure of extracellular domains (ECD) of human CDH17 and the binding to specific ECD by disclosed antibodies determined by ELISA.

FIG. 7A illustrates cross blocking results of the disclosed anti-CDH17 antibodies.

FIG. 7B is a summary of the groups and bins of the disclosed anti-CDH17 antibodies based on the ECD ELISA (FIG. 6B-D) and cross blocking (FIG. 7A).

FIGS. 8A and 8B are diagrams of the molecular design of the disclosed biparatopic antibodies.

FIGS. 9A and 9B shows the sequences of Fab arm and Fc, Fc mutations and binding domains of disclosed biparatopic antibodies.

FIGS. 10A-E show the enhanced internalization and internalization kinetics and the cell-based binding data of anti-CDH17 biparatopic 1 (bp Ab1) compared to its mAb alone control. FIGS. 10A-B show the internalization in COLO205 wild-type (WT) and COLO205 CDH17 knock-out (KO) cells, respectively. FIG. 10C shows the cell-based binding of anti-CDH17 antibodies on COLO205 cells. FIG. 10D shows the internalization kinetics of anti-CDH17 antibodies in AsPC1 cells and FIG. 10E shows the fold change in the internalization signal at 1.5 hours over time zero.

FIGS. 11A-D show the enhanced internalization by biparatopic 1 (Bp Ab1) across tumor cells expressing varying levels of CDH17 (tumor cell CDH17 receptor density is shown in FIG. 5A). FIG. 11E shows enhanced internalization by biparatopic 2 (Bp Ab2) and 3 (Bp Ab3) compared to PC8 in COLO205 cells.

FIG. 12 shows enhanced endocytosis-derived cellular cytotoxicity by biparatopic antibody 1 (Bp Ab1) compared to the PCs, a combination of mAbs or mAbs only controls in COLO205 cells using secondary antibody labeled with MMAF.

FIGS. 13A-C illustrate the design of CDH17/CD3 bispecific antibodies and identifies the sequence and target of each arm.

FIGS. 14A and 14B show T cell activation by CDH17/CD3 bsAb using Jurkat TCR NFAT reporter cell line in the presence (FIG. 14A) and absence (FIG. 14B) of target cells.

FIGS. 15A and 15B show that biparatopic antibodies 1.1 and 2.1 (BpAb1.1 and BpAb2.1) exhibit enhanced internalization in tumor cells with medium/low CDH17 expression (Colo205 cells, FIG. 15A) and high CDH17 expression (AsPC1 cells, FIG. 15B). The CDH17 receptor densities for these cell lines are shown in FIG. 5A.

FIG. 16 shows that anti-CDH17 biparatopic antibodies 1.1 and 2.1 induced greater cell cytotoxicity than both PC8 and one of their corresponding monoclonal antibody arms, humanized C17_mAb 1.

FIGS. 17A and 17B shows target engagement of the humanized bispecific CDH17 antibodies (bsAbs) with CDH17-expressing Colo205 cells (FIG. 17A) and Jurkat T cells (FIG. 17B).

FIG. 18 provides TDCC activity of humanized CDH17xCD3 bispecific antibodies in Colo205 cells.

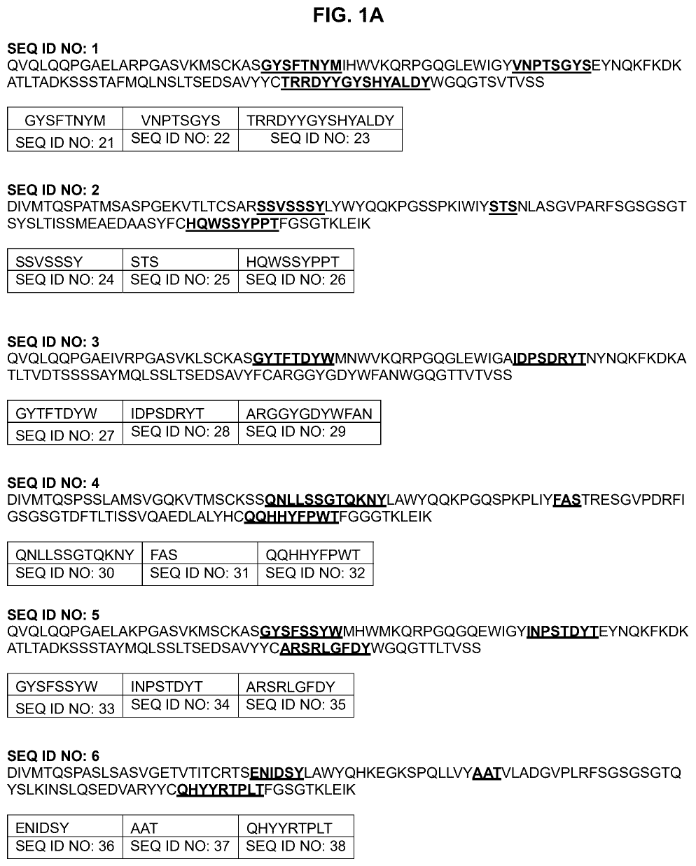

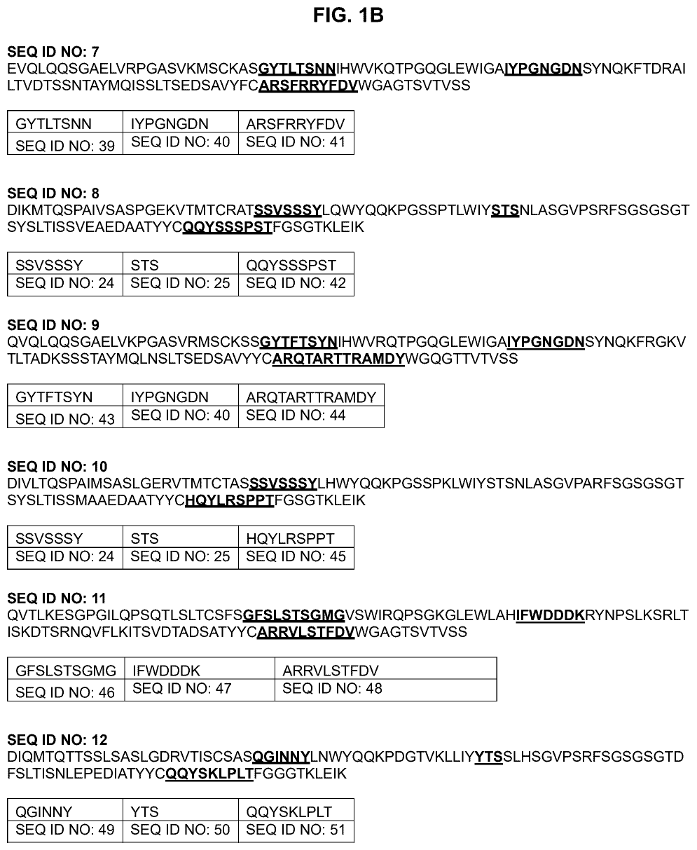

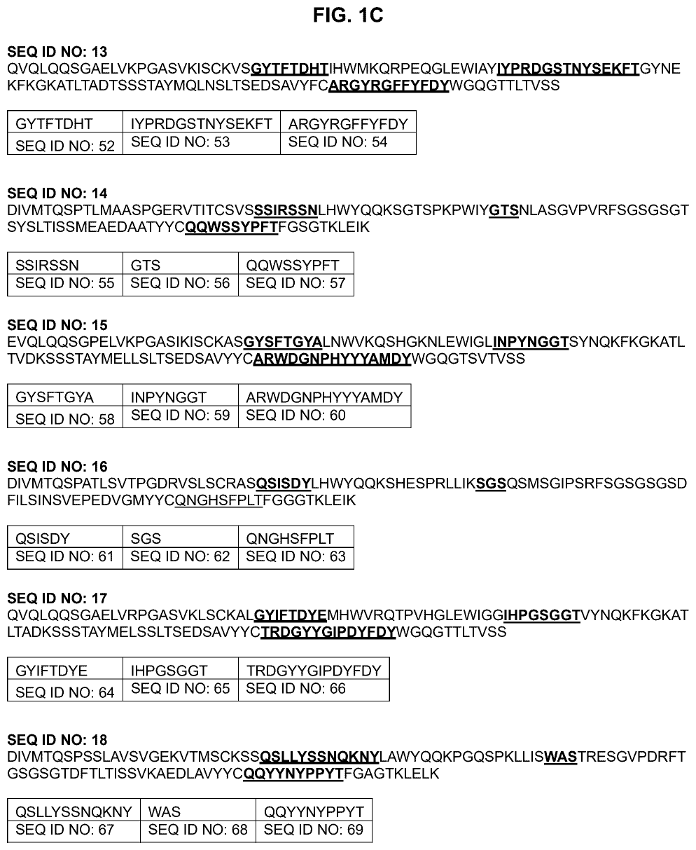

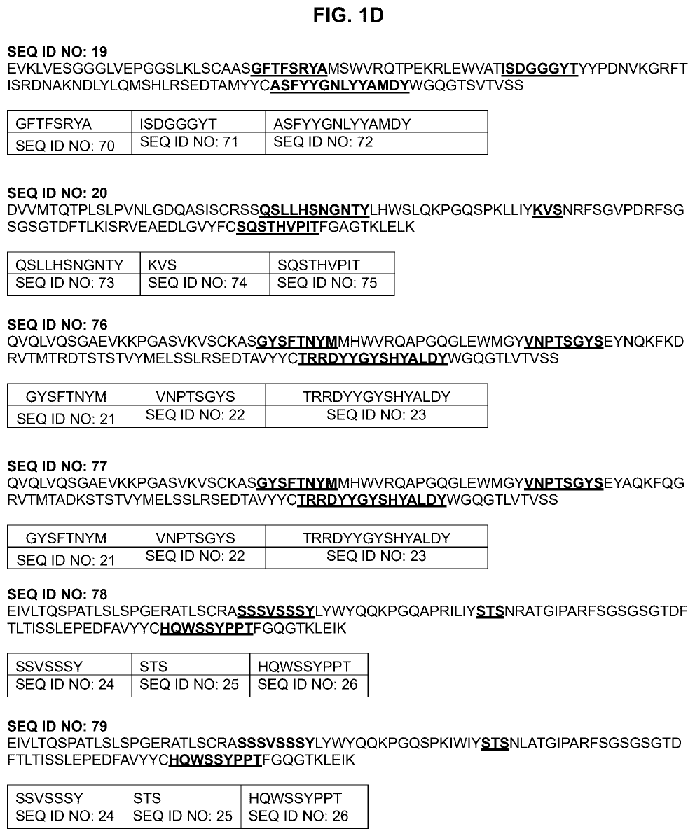

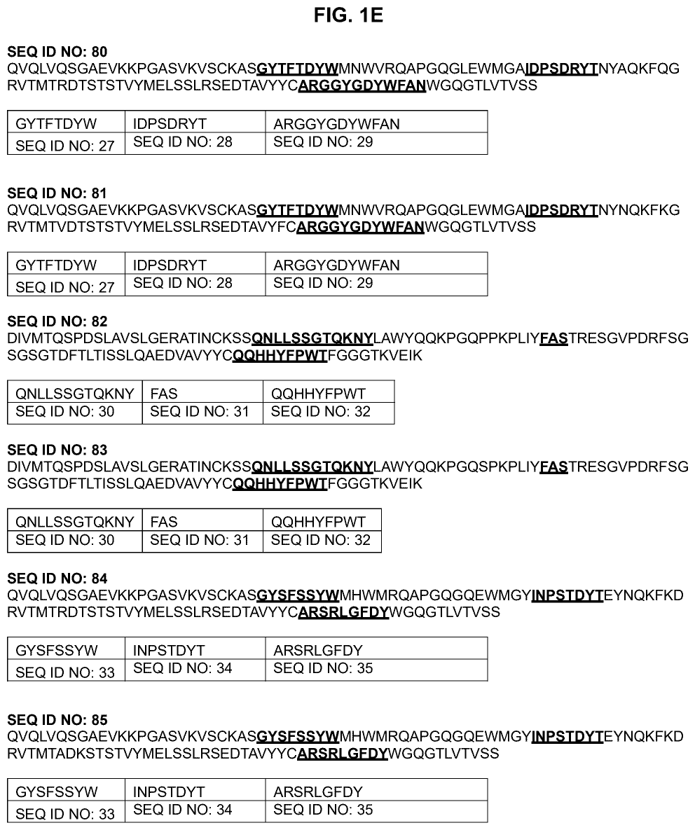

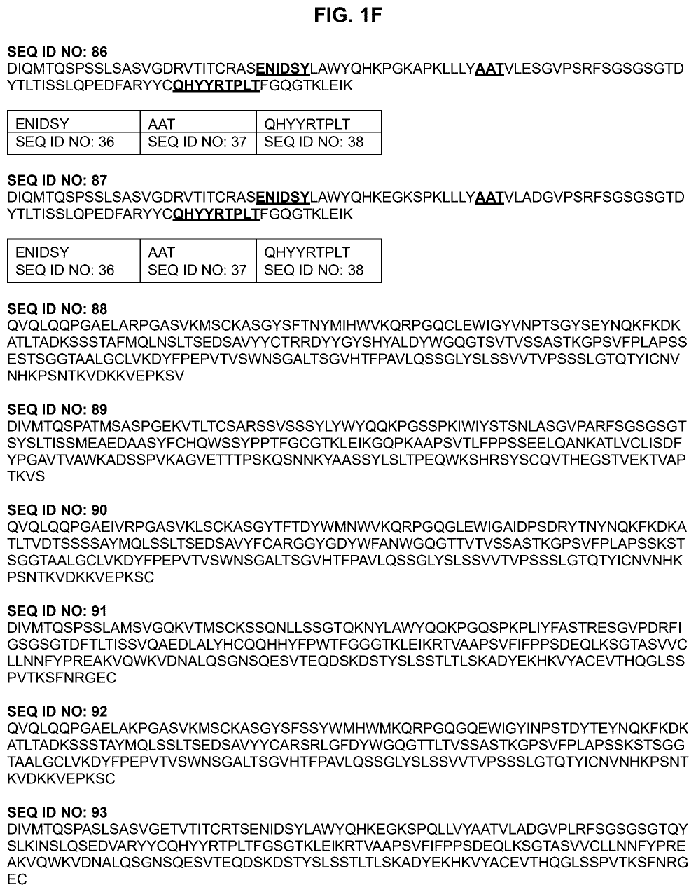

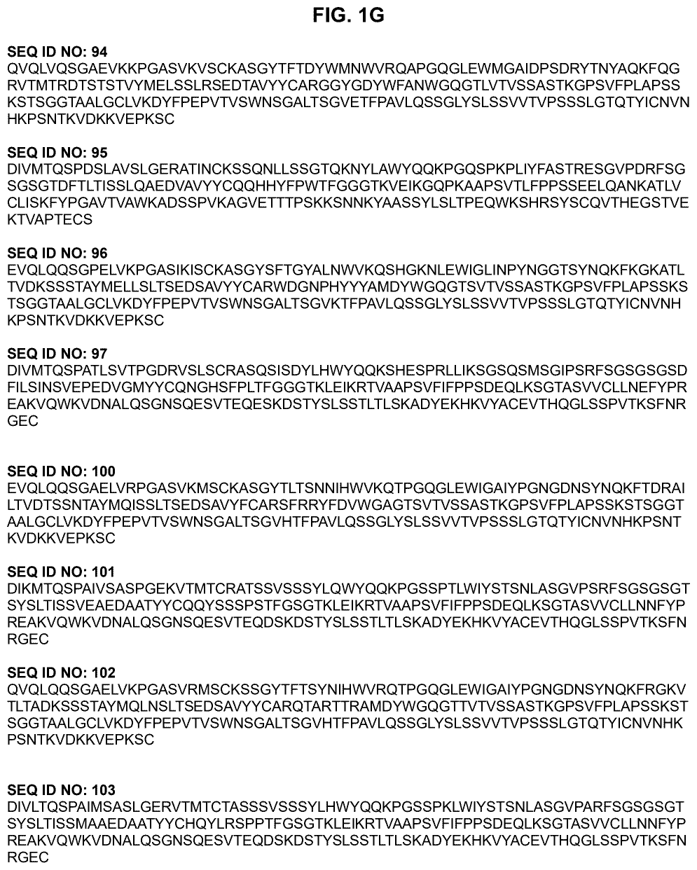

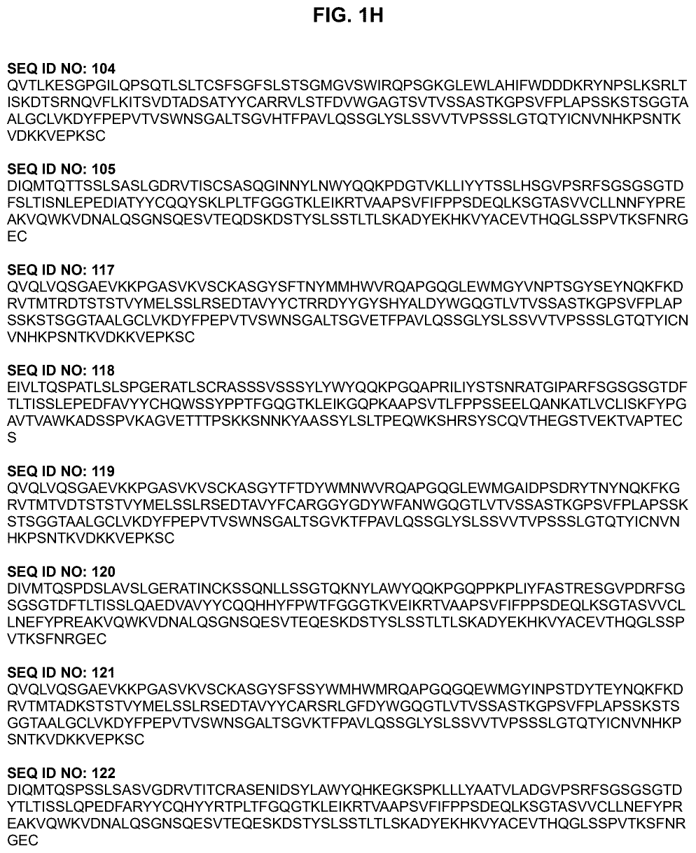

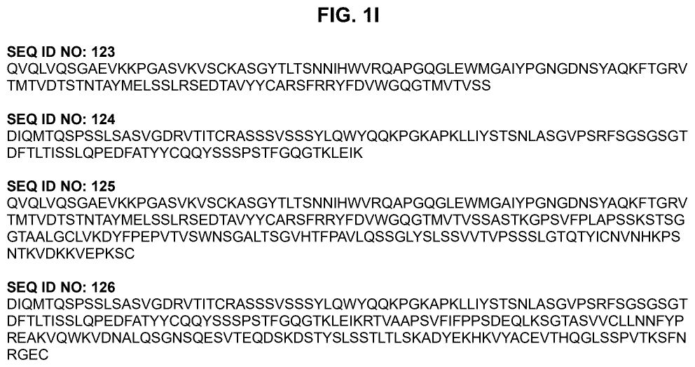

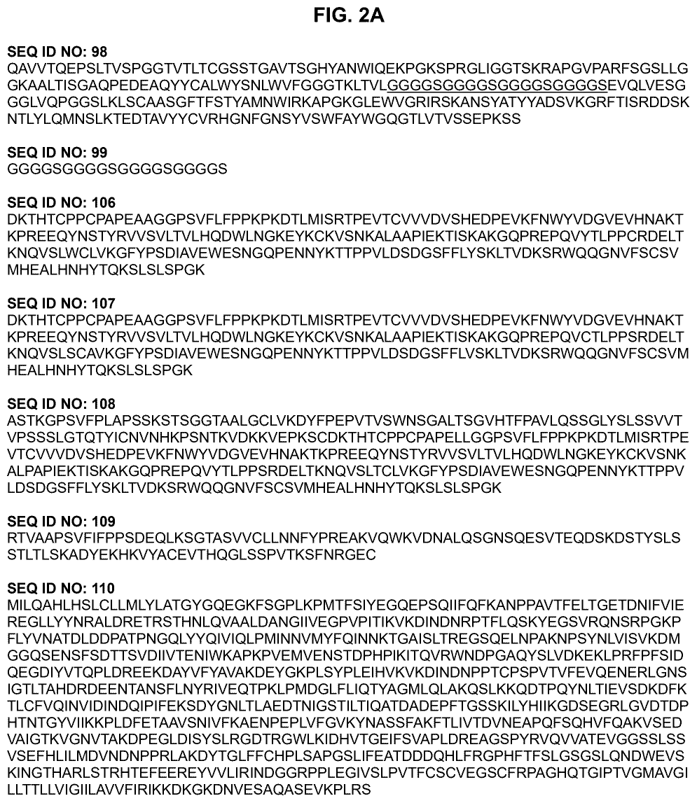

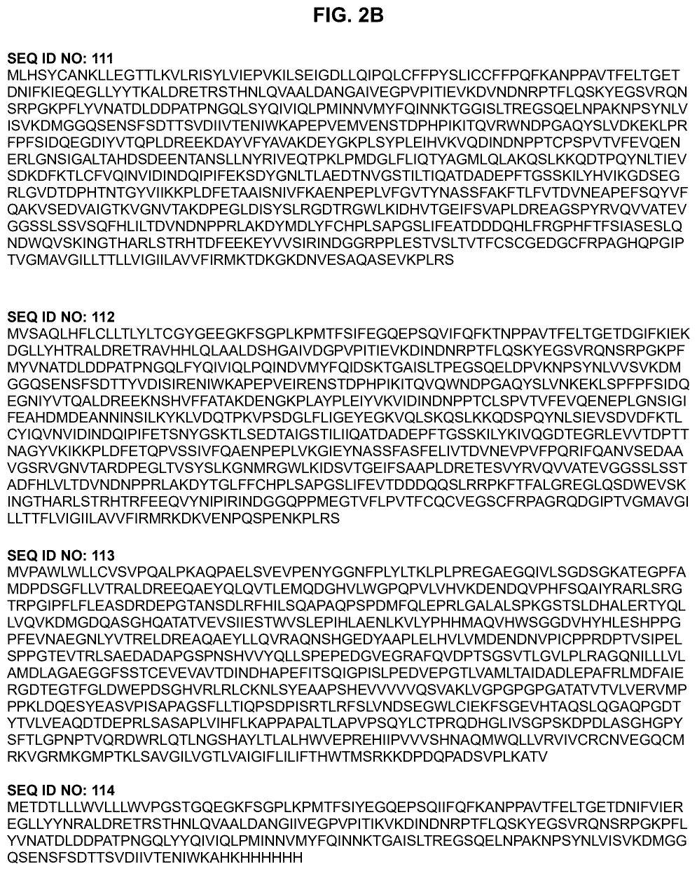

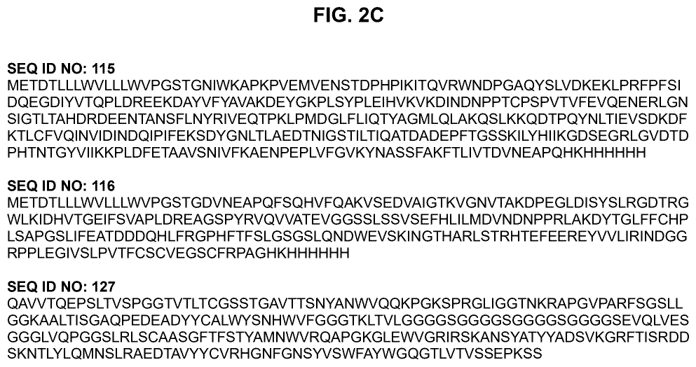

### DETAILED DESCRIPTION

So that the disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

Throughout this disclosure the following abbreviations will be used:
mAb or Mab or MAb—Monoclonal antibody.
CDR—Complementarity determining region.
VH or VH—Heavy chain variable region.
VL or VL—Light chain variable region.
FR—Antibody framework region.
TME—Tumor microenvironment.
IHC—Immunohistochemistry.
ADC—Antibody-drug conjugate.
MMAF—Monomethyl auristatin F.
MMAE—Monomethyl auristatin E.
Fab—The fragment antigen-binding region
Fc—The fragment crystallizable (Fc) region
Ab—Antibody
BsAb—Bispecific antibody
CRC—Colorectal cancer
HCC—Hepatocellular carcinoma
WT—Wild type
KO—Knock out
ECD—Extracellular domains
RT—room temperature

The term “cadherin 17”, or “CDH17” includes human CDH17, in particular the native-sequence polypeptide, isoforms, chimeric polypeptides, all homologs, fragments, and precursors of CDH17. The amino acid sequences for human, cynomolgus and murine CDH17 are provided in NCBI Reference Sequences: NP_001138135.1 (human) (SEQ ID NO: 110), XP_045254438.1 (cynomolgus monkey (SEQ ID NO: 111) and XP_006537646 (mouse) (SEQ ID NO: 112). Orthologs of CDH17 share 90.45% and 79.44% homology to the human protein in cynomolgus monkey and mouse respectively.

The term “antibody” herein is used in the broadest sense and encompasses various antibody structures, including but not limited to monoclonal antibodies, polyclonal antibodies, chimeric antibodies, humanized antibodies, and multi-specific antibodies (e.g., bispecific antibodies).

An exemplary antibody such as an IgG comprises two heavy chains and two light chains. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino terminus to carboxy terminus in the following order: FRI, CDR1, FR2, CDR2, FR3, CDR3, FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; (Kabat et al., SEQUENCES OF PROTEINS OF IMMUNOLOGICAL INTEREST, 5th Ed. Public Health Service, National Institutes of Health, Bethesda, Md., 1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; (Chothia and Lesk, J. Mol. Biol. 196:901-917, 1987) and/or those residues forming a hypervariable loop (e.g. residues 27-38 (CDR1), 56-65 (CDR2) and 105-117 (CDR3)) in light and heavy chain variable regions (Lefranc, M.-P., The Immunologist, 7, 132-136, 1999; Lefranc, M.-P, et al., Dev. Comp. Immunol., 27, 55-77, 2003).

The term “monoclonal antibody” as used herein refers to an antibody obtained from a population of substantially homogeneous antibodies, e.g., the individual antibodies comprising the population are identical and/or bind the same epitope, except for possible variant antibodies, e.g., containing naturally occurring mutations and/or arising during production of a monoclonal antibody preparation, such variants generally being present in minor amounts (e.g., variants having a C-terminal lysine deletion). In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present disclosure may be made by a variety of techniques, including but not limited to the hybridoma method, recombinant DNA methods, phage display methods, and methods utilizing transgenic animals containing all or part of the human immunoglobulin loci, such methods and other exemplary methods for making monoclonal antibodies being described herein.

The term “chimeric” antibody refers to a recombinant antibody in which a portion of the heavy and/or light chain is identical with or homologous to corresponding sequences in antibodies derived from a particular species, or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. In addition, complementarity determining region (CDR) grafting may be performed to alter certain properties of the antibody molecule including affinity or specificity. Typically, the variable domains are obtained from an antibody from an experimental animal (the “parental antibody”), such as a rodent, and the constant domain sequences are obtained from human antibodies, so that the resulting chimeric antibody can direct effector functions in a human subject and will be less likely to elicit an adverse immune response than the parental (e.g., mouse) antibody from which it is derived.

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986, Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761, 5,693,762; 6,180,370; and Selick et al., WO 90/07861, each of which is incorporated herein by reference in its entirety.

A “human antibody” is an antibody that possesses an amino-acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al, Monoclonal Antibodies and Cancer Therapy, Alan R. Liss, p. 77 (1985); Boemer et al, J. Immunol, 147 (1): 86-95 (1991). See also van Dijk and van de Winkel, Curr. Opin. Pharmacol, 5:368-74 (2001). Human antibodies can be prepared by administering the antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL, and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341:544-546)), which consist of a VH domain; (vi) isolated complementarity determining regions (CDR), and (vii) combinations of two or more isolated CDRs which may optionally be joined by a synthetic linker.

The “variable domain” (V domain) of an antibody mediates binding and confers antigen specificity of a particular antibody. However, the variability is not evenly distributed across the 110-amino acid span of the variable domains. Instead, the V regions consist of relatively invariant stretches called framework regions (FRs) of 15-30 amino acids separated by shorter regions of extreme variability referred to herein as “hypervariable regions” or CDRs that are each 9-12 amino acids long. As will be appreciated by those in the art, the exact numbering and placement of the CDRs can be different among different numbering systems. However, it should be understood that the disclosure of a variable heavy and/or variable light sequence includes the disclosure of the associated CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vLCDR1, vICDR2 and vlCDR3).

“Complementarity determining region” or “CDR” as the terms are used herein refer to short polypeptide sequences within the variable region of both heavy and light chain polypeptides that are primarily responsible for mediating specific antigen recognition. There are three CDRs (termed CDR1, CDR2, and CDR3) within each VL and each VH. Unless stated otherwise herein, CDR and framework regions are annotated according to the Kabat numbering scheme (Kabat E. A., et al., Sequences of proteins of immunological interest, In: NIH Publication No. 91-3242, US Department of Health and Human Services, Bethesda, Md, 1991).

In other embodiments, the CDRs of an antibody can be determined according to MacCallum R M et al, (1996) J Mol Biol 262:732-745, herein incorporated by reference in its entirety or according to the IMGT numbering system as described in Lefranc M-P, (1999) The Immunologist 7:132-136 and Lefranc M-P et al, (1999) Nucleic Acids Res 27:209-212, each of which is herein incorporated by reference in its entirety. See also, e.g., Martin A. “Protein Sequence and Structure Analysis of Antibody Variable Domains,” in Antibody Engineering, Kontermann and Diibel, eds., Chapter 31, pp. 422-439, Springer-Verlag, Berlin (2001), herein incorporated by reference in its entirety. In other embodiments, the CDRs of an antibody can be determined according to the AbM numbering scheme, which refers to AbM hypervariable regions, which represent a compromise between the Kabat CDRs and Chothia structural loops and are used by Oxford Molecular's AbM antibody modeling software (Oxford Molecular Group, Inc.), herein incorporated by reference in its entirety.

“Framework” or “framework region” or “FR” refers to variable domain residues other than hypervariable region (HVR) residues. The FR of a variable domain generally consists of four FR domains: FR1, FR2, FR3, and FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup Ill as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Sendee, National Institutes of Health, Bethesda, Md. (1991).

The term “effector functions,” deriving from the interaction of an antibody Fc region with certain Fc receptors, include but are not necessarily limited to Clq binding, complement dependent cytotoxicity (CDC), Fc receptor binding, FcγR-mediated effector functions such as ADCC and antibody dependent cell-mediated phagocytosis (ADCP), and down regulation of a cell surface receptor. Such effector functions generally require the Fc region to be combined with an antigen binding domain (e.g., an antibody variable domain).

The term “internalization” refers to the process by which cell surface receptors are taken up or engulfed by the cell and transported into intracellular compartments, typically endosomes. Antibody-mediated receptor internalization facilitates the targeted delivery of highly cytotoxic small molecule drug moieties directly to tumor cells by antibody-drug conjugates (ADCs). This process also plays a crucial role in regulating cellular signaling pathways by controlling the number and activity of receptors on the cell surface.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more. The amino acid residues of an antibody that contact an antigen can be determined, for example, by determining the crystal structure of the antibody in complex with the antigen or by performing hydrogen/deuterium exchange. In some embodiments, residues of an antibody that are within 5 Å the antigen are considered to contact the antigen. In some embodiments, an antibody that binds to the same epitope as a reference antibody blocks binding of the reference antibody to its antigen in a competition assay by 50% or more, and conversely, the reference antibody blocks binding of the antibody to its antigen in a competition assay by 50% or more.

The term “antigen binding fragment” refers to a molecule other than an intact antibody that comprises a portion of an intact antibody that binds the antigen to which the intact antibody binds. Examples of antigen binding fragments include but are not limited to Fv, Fab, Fab′, Fab′-SH, F(ab)2; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CHI). Pepsin treatment of an antibody yields a single large F(ab)2 fragment which roughly corresponds to two disulfide linked Fab fragments having divalent antigen-binding activity and is still capable of cross-linking antigen. Fab fragments differ from Fab′ fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antigen binding fragments originally were produced as pairs of Fab′ fragments which have hinge cysteines between them. Other chemical couplings of antigen binding fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antigen binding fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341:544-546), which consist of a VH domain; (vi) isolated complementarity determining regions (CDR), and (vii) combinations of two or more isolated CDRs which may optionally be joined by a synthetic linker.

“Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual specificity allows the antibodies to interact with high affinity with two different antigens as a single Fab or IgG molecule. According to one embodiment, the multispecific antibody in an IgG1 form binds to each epitope with an affinity of 5 μM to 0.001 pM, 3 M to 0.001 pM, 1 μM to 0.001 pM, 0.5 μM to 0.001 pM or 0.1 μM to 0.001 pM. “Monospecific” refers to the ability to bind only one epitope. Multi-specific antibodies can have structures similar to full immunoglobulin molecules and include Fc regions, for example IgG Fc regions. Such structures can include, but are not limited to, IgG-Fv, IgG-(scFv)2, DVD-Ig, (scFv)2-(scFv)2-Fc and (scFv)2-Fc-(scFv)2. In case of IgG-(scFv)2, the scFv can be attached to either the N-terminal or the C-terminal end of either the heavy chain or the light chain.

As used herein, the term “bispecific antibodies” refers to monoclonal, often human or humanized, antibodies that have binding specificities for at least two different antigens. In the disclosure, one of the binding specificities can be directed towards CDH17, the other can be for any other antigen, e.g., for a cell-surface protein, receptor, receptor subunit, tissue-specific antigen, cytokine, chemokine, cell-secreted protein, virally derived protein, virally encoded envelope protein, bacterially derived protein, or bacterial surface protein, etc.

The term “biparatopic antibody” designates a subset of bispecific antibodies wherein each antigen-binding domain identifies distinct and non-overlapping epitopes on a shared target antigen. (e.g., a TAA for example CDH17). This dual-binding capability enhances the antibody's specificity, avidity, and potentially its therapeutic efficacy. By binding to multiple epitopes on the same target molecule, biparatopic antibodies may exhibit enhanced potency in blocking signaling pathways, triggering immune responses, or facilitating the targeted delivery of therapeutic payloads, such as toxins or drugs, to diseased cells.

The term “multispecific antibody” is used in the broadest sense and specifically covers an antibody comprising a heavy chain variable domain (VH) and a light chain variable domain (VL), where the VH-VL unit has polyepitopic specificity (e.g., is capable of binding to two different epitopes on one biological molecule or each epitope on a different biological molecule). Such multispecific antibodies include, but are not limited to, full-length antibodies, antibodies having two or more VL and VH domains, bispecific diabodies and triabodies. “Polyepitopic specificity” refers to the ability to specifically bind to two or more different epitopes on the same or different target(s).

As used herein, the term “diabodies” refers to bivalent antibodies comprising two polypeptide chains, in which each polypeptide chain includes ATI and VL domains joined by a linker that is too short (e.g., a linker composed of five amino acids) to allow for intramolecular association of ATI and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one ATI domain and one VL domain joined by a linker that is exceedingly short (e.g., a linker composed of 1-2 amino acids) to permit intramolecular association of AH and AT domains within the same peptide chain.

The term an “isolated antibody” when used to describe the various antibodies disclosed herein, means an antibody that has been identified and separated and/or recovered from a cell or cell culture from which it was expressed. An isolated antibody or antigen binding fragment may include variants of the antibody or antigen binding fragment having one or more co- or post-translational modifications that arise during production, purification, and/or storage of the antibody or antigen binding fragment. Contaminant components of its natural environment are materials that would typically interfere with diagnostic or therapeutic uses for the polypeptide, and can include enzymes, hormones, and other proteinaceous or non-proteinaceous solutes. In some embodiments, an isolated antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87, 2007. In a preferred embodiment, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain.

With regards to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to CDH17 or to a CDH17 epitope without substantially binding to any other polypeptide or polypeptide epitope.

As used herein the term “specifically binds CDH17” refers to the ability of an antibody, or antigen-binding fragment to recognize and bind endogenous human CDH17 as it occurs on the surface of normal or malignant cells, but not to any other human LRR family homolog.

The term “affinity” as used herein, means the strength of the binding of an antibody to an epitope. The affinity of an antibody is given by the equilibrium dissociation constant KD, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant KA is defined by 1/KD. Methods for determining the affinity of mAbs can be found in Harlow, et al., Antibodies: A Laboratory Manual, Cold Spring Harbor Laboratory’ Press, Cold Spring Harbor, N.Y., (1988), Coligan et al., eds., Current Protocols in Immunology, Greene Publishing Assoc, and Wiley Interscience, N.Y., (1992, 1993), and Muller, Meth. Enzymol. 92:589-601 (1983), which are entirely incorporated herein by reference. One standard method well known in the art for determining the affinity of mAbs is the use of surface plasmon resonance (SPR) screening (such as by analysis with a BIAcore™ SPR analytical device).

An “epitope” is a term of art that indicates the site or sites of interaction between an antibody and its antigen(s). As described by (Janeway, C, Jr., P. Travers, et al. (2001). Immunobiology: the immune system in health and disease. Part II, Section 3-8. New York, Garland Publishing, Inc.): “An antibody generally recognizes only a small region on the surface of a large molecule such as a protein. [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C. Jr., P. Travers, et al. (2001). Immunobiology: the immune system in health and disease. Part II, Section 3-8. New York, Garland Publishing, Inc.).

The term “KD”, as used herein, refers to the equilibrium dissociation constant, which is obtained from the ratio of koff to kon (i.e., koff/kon) and is expressed as a molar concentration (M). KD values for antibodies can be determined using methods well established in the art. Preferred methods for determining the KD of an antibody include biolayer interferometry (BLI) analysis, preferably using a Fortebio Octet RED device, surface plasmon resonance, preferably using a biosensor system such as a BIACORE® surface plasmon resonance system, or flow cytometry and Scatchard analysis.

“EC₅₀” with respect to an agent and a particular activity (e.g., binding to a cell, inhibition of enzymatic activity, activation or inhibition of an immune cell), refers to the efficient concentration of the agent which produces 50% of its maximum response or effect with respect to such activity. “EC₁₀₀” with respect to an agent and a particular activity refers to the efficient concentration of the agent which produces its substantially maximum response with respect to such activity.

As used herein the term “antibody-drug conjugate” (ADC) refers to immunoconjugates consisting of recombinant monoclonal antibodies covalently linked to cytotoxic agents (known as payloads) via synthetic linkers. Immunoconjugates (antibody-drug conjugates, ADCs) are a class of highly potent antibody-based cancer therapeutics. ADCs consist of recombinant monoclonal antibodies covalently linked to cytotoxic agents (known as payloads) via synthetic linkers. ADCs combine the specificity of monoclonal antibodies and the potency of small-molecule chemotherapy drugs and facilitate the targeted delivery of highly cytotoxic small molecule drug moieties directly to tumor cells.

As used herein the term “endocytosis” refers to the process where eukaryotic cells internalize segments of the plasma membrane, cell-surface receptors, and components from the extracellular fluid. Endocytosis mechanisms include receptor-mediated endocytosis. The term “receptor-mediated endocytosis” refers to a biological mechanism by which a ligand, upon binding to its target, triggers membrane invagination and pinching, gets internalized and delivered into the cytosol or transferred to appropriate intracellular compartments.

The term “bystander effect” refers to target-cell mediated killing of antigen-negative cells adjacent to tumor cells targeted for by an antibody drug conjugate. The bystander effect is generally caused by cellular efflux of hydrophobic cytotoxic drugs, capable of diffusing out of an antigen-positive target cell and into adjacent antigen-negative cells. The presence or absence of the bystander effect can be attributed to aspects of the linker and conjugation chemistries used to produce an immunoconjugate.

As used herein the terms “antibody-based immunotherapy” and “immunotherapies” are used to broadly refer to any form of therapy that relies on the targeting specificity of an anti-CDH17 antibody, bispecific molecule, antigen-binding domain, or fusion protein comprising an anti-CDH17 antibody or antigen binding fragments or CDRs thereof, to mediate a direct or indirect effect on a CDH17 expressing cell. The terms are meant to encompass methods of treatment using naked antibodies, bispecific antibodies (including T cell engaging, NK cell engaging and other immune cell/effector cell engaging formats) antibody drug conjugates, cellular therapies using T cells (CAR-T) or NK cells (CAR-NK) engineered to comprise a CDH17-specific chimeric antigen receptor and oncolytic viruses comprising a CDH17-specific binding agent, and gene therapies by delivering the antigen binding sequences of the anti-CDH17 antibodies and express the corresponding antigen binding fragments in vivo.

### Cadherin Superfamily

The cadherin superfamily comprises calcium-dependent, membrane-associated glycoproteins. Cadherin genes encode a family of highly conserved transmembrane proteins characterized by an adhesive ectodomain consisting of tandem cadherin repeats. All members within the superfamily possess a minimum of two extracellular cadherin (EC) repeats containing calcium-binding sites within the EC linker region. Over 100 members of the human cadherin superfamily have been identified and can be classified into three subfamilies (major cadherins, protocadherins, and cadherin-related proteins), distinguishable by their protein domain composition, genomic structure, and phylogenetic analysis of protein sequences. These subfamily members include classical or type-I cadherins, atypical or type-II cadherins, desmocollins, desmogleins, protocadherins, and Flamingo cadherins. Moreover, several cadherins occupy unique positions within the superfamily, such as cadherin-13, -15, -16, -17, Dachsous, RET, FAT, MEGF1, and numerous invertebrate cadherins (Nollet, F., et al., J Mol Biol, 299(3): 551-572, 2000; Hulpiau et al., Int J. Biochem. Cell Biol., 41(2) 349-369, 2009; Hulpiau et al., Exp Cell Res, 358(1): 3-9, 2017).

The Cadherin superfamily plays pivotal roles in a myriad of biological processes essential for tissue organization, cellular communication, and organismal development. These molecules serve as adhesive bridges between adjacent cells, fostering intercellular cohesion and orchestrating complex cellular behaviors critical for embryogenesis, tissue homeostasis, and physiological function. Each tissue or cell type exhibits a distinct pattern of cadherin molecules, with their expression tightly regulated during development. Dysregulation of their expression levels or functionality is commonly observed in human malignancies, often exacerbating cancer cell invasion and metastasis (Harmon, et al., F1000 Biol Rep, 1:13, 2009; Niessen et al., Physiol Rev, 91(2): 691-731, 2011).

The expression of cadherin family members is tightly regulated in a spatial and temporal manner, exhibiting dynamic patterns across various tissues and developmental stages. Differential cadherin expression profiles govern tissue-specific adhesive interactions and cellular behaviors, thereby contributing to tissue morphogenesis and functional specialization. Dysregulated cadherin expression or function is associated with a plethora of pathological conditions, including cancer, neurodevelopmental disorders, and autoimmune diseases. Aberrant cadherin-mediated adhesion and signaling events can disrupt tissue architecture, promote tumor invasion and metastasis, and compromise immune surveillance, thereby fueling disease progression and therapeutic resistance (Polanco, J., et al. (2021). Front Mol Neurosci 14: 633719; Halbleib, J. M. and W. J. Nelson (2006). Genes Dev 20(23): 3199-3214; Harmon, R. M., et al. (2009). F1000 Biol Rep 1:13).

### Cadherin-17

The nonclassical cadherin-17 (CDH17), also known as liver-intestine (LI) cadherin, belongs to subgroup 7D of the cadherin superfamily, featuring seven cadherin-like ectodomains and a succinct cytoplasmic tail. CDH17 contains an RGD motif on its domain 6 and has been found to interact with integrin a2b1 via its RGD motif. CDH17 also contains 6 glycosylation sites throughout its extracellular domains, contributing to its structural complexity and functionality.

CDH17 is known to form trans-homodimerization to form tight junction between intestinal cells. The crystal structure of the LI-cadherin homodimer, encompassing its first four extracellular cadherin repeats (EC1-4), was reported, revealing a distinctive architecture driven by interactions between EC2 of one protein chain and EC4 of the second chain. Notably, LI-cadherin features a noncanonical calcium ion-free linker between the EC2 and EC3 domains, distinguishing it from other cadherins. Homodimer formation in the crystal structure is crucial for LI-cadherin-dependent cell adhesion, highlighting its functional relevance (Yui et al., J Biol Chem, 297(3): 101054, 2021; Gray et al., F Struct Biol Commun, 77(Pt 3): 85-94, 2021).

CDH17 plays diverse and crucial roles in various physiological processes, particularly in the liver and intestine. CDH17 primarily functions in cell-cell adhesion and tissue organization, contributing to the maintenance of epithelial integrity and barrier function. Its involvement in cell adhesion is essential for the formation and stabilization of cell-cell contacts, facilitating tissue morphogenesis, and regulating cell migration and differentiation. Beyond its adhesive functions, CDH17 is implicated in signaling pathways that regulate cellular behavior and function. Activation of CDH17 can modulate downstream signaling cascades, including those involving β-catenin, leading to alterations in gene expression and cellular processes critical for development and homeostasis. In the liver, CDH17 is implicated in bile acid metabolism and liver regeneration, contributing to hepatocyte proliferation and tissue repair. In the intestine, CDH17 is involved in the maintenance of the intestinal epithelial barrier, regulating the permeability of the intestinal mucosa and protecting against microbial invasion and inflammation (Baumgartner, W., Tissue Barriers, 1(1): e23815, 2013; Gessner et al, Annals of the New York Academy of Sciences, 915(1): 136-143, 2000).

RNA expression data indicates that CDH17 expression in normal adult human tissues appears to be primarily confined to the intestine, according to information available at https://www.proteinatlas.org/ENSG00000079112-CDH17/summary/rna. An analysis of a tissue microarray comprising 14,948 interpretable samples from 150 distinct tumor types and subtypes, as well as 76 different normal tissue types, was conducted using immunohistochemistry (IHC). In normal tissues, membranous staining of CDH17 was primarily observed in the epithelium of the intestine and pancreatic excretory ducts. Among the analyzed cancer categories, 53 out of 150 demonstrated CDH17 positivity, with 26 categories displaying at least one strongly positive case. CDH17 positivity was most frequently observed in epithelial and neuroendocrine colorectal neoplasms (50.0%-100%), other gastrointestinal adenocarcinomas (42.7%-61.6%), mucinous ovarian cancer (61.1%), pancreatic acinar cell carcinoma (28.6%), cervical adenocarcinoma (52.6%), bilio-pancreatic adenocarcinomas (40.5%-69.8%), and other neuroendocrine neoplasms (5.6%-100%). Only 9.9% of 182 pulmonary adenocarcinomas tested positive for CDH17 (Jacobsen et al., Pathology—Research and Practice, 2024). In another study, an in silico analysis evaluated 13,488 genes expressed in CRCs from the Human Protein Atlas (HPA) database to shortlist potential surface targets based on restricted expression profiles in normal tissues and moderate expression levels in CRC. Cadherin 17 (CDH17) emerging as the top-ranked target due to its high and consistent expression in CRCs. Further analysis revealed CDH17's correlation with carcinoembryonic antigen expression and its association with immune response gene sets and MHC class I and II molecules. (Wong et al, Comput Biol Chem, 105:107897, 2023).

The function of CDH17 in cancer is multifaceted. It is implicated in promoting tumor progression, invasion, and metastasis by facilitating cell adhesion, migration, and invasion processes. CDH17-mediated cell adhesion may contribute to the formation of tumor cell clusters and the establishment of metastatic lesions in distant organs. (Bartolom et al., Clin Cancer Res, 24(2): 433-444, 2018; Yuan et al., Data Brief, 25:104332, 2019; Garcia-Martinez et al, Mol Cancer Ther, 20(1): 96-108, 2021).

Increased CDH17 levels have been linked to greater tumor burden and unfavorable prognosis in colorectal cancer (CRC) (Ng et al., JCO, 41, e14651-e14651, 2023). Furthermore, reduced CDH17 expression has been observed in CRC cases with high microsatellite instability, along with a negative correlation with immune response gene sets and MHC class I and II molecule expression levels (Wong et al, Comput Biol Chem, 105:107897, 2023). Consequently, CDH17 emerges as a promising therapeutic target in CRCs, particularly for patients unresponsive to checkpoint inhibitor immunotherapies.

Additionally, CDH17 positivity in cancer cells could serve as a diagnostic marker for certain tumor types, aiding in subtype classification and prognostic assessment. Quantification of tissue CDH17 level using a standardized automated IHC platform and digital image analysis could aid in patient stratification which allows efficient treatment strategies for better survival outcomes (Zheng et al., J Surg Oncol, 123(5): 1253-1262, 2021; (Wong, Comput Biol Chem, 105:107897, 2023)).

### Anti-CDH17 Antibodies

The disclosed anti-CDH17 antibodies (C17_mAb 1, C17_mAb 2, C17_mAb 3, C17_mAb 4, C17_mAb 5, C17_mAb 6, C17_mAb 7, C17_mAb 8, C17_mAb 9 and C17_mAb10) specifically bind human CDH17. These antibodies and fragments thereof are characterized by unique sets of CDR sequences, specificity for CDH17, and are useful in cancer immunotherapy as monotherapy or in combination with other anti-cancer agents. More specifically, the disclosure relates to antibodies that bind to human CDH17, and to their use to target CDH17+ tumor cells.

The antibody of the present disclosure may be monoclonal, chimeric, humanized, bispecific, or biparatopic which binds to human CDH17 and exhibits one or more of the following properties alone or in combination: (a) is specific for human CDH17 with cell binding affinities ranging from <1 nM-10 nM; (b) binds to an epitope within human CDH17 ECD 1-2; (c) binds to an epitope within human CDH17 ECD 3-5; (d) binds to an epitope within human CDH17 ECD 6-7; (e) binds to two distinct and non-overlapping epitopes within human CDH17; (f) binds to a first epitope within human CDH17 ECD 3-5 and to a second epitope within ECD 6-7 of human CDH17; (g) binds to a first epitope within human CDH17 ECD 1-2 and to a second epitope within ECD 6-7 of human CDH17; (h) binds to a first epitope within human CDH17 ECD 1-2 and to a second epitope within ECD 3-5 of human CDH17; (i) cross-reacts with cynomolgus CDH17; (j) does not bind to human CDH16; (k) binds to CDH17 endogenously expressed by human cancer cells; (1) is internalized from the surface of CDH17 positive cell after binding CDH17; (m) is internalized and kills CDH17 positive cells with varying levels of CDH17 expression when combined with an ADC-conjugated secondary antibody or directly conjugated to cytotoxic agent (ADC) induces killing of tumor cells; n) simultaneously engaging CDH17 on tumor cells and CD3 on T cells in a CD3/CDH17 bispecific Ab format; o) induces T cell activation in CD3/CDH17 bispecific Ab format; or p) redirect T cells to kill CDH17-expressing tumor cells in a CD3/CDH17 bispecific Ab format.

Based on an in vitro assessment of maximum binding capacity, the level or rate of binding and/or internalization of CDH17 from the cell surface and the cytotoxicity (e.g., EC50) of CDH17+ tumor cells the disclosed anti-CDH17 antibodies and fragments thereof can be evaluated for suitability for use as an ADC targeting antibody or antigen binding fragment for the treatment of cancer.

In alternative embodiments, the disclosed anti-CDH17 antibodies or fragments thereof may be used in the context of a biparatopic antibody engineered to bind to distinct and non-overlapping epitopes on CDH17, enhanced potency in activating or blocking signaling pathways, triggering immune responses. In some embodiments the anti-CDH17 biparatopic antibody is an ADC. Biparatopic antibodies may exhibit enhanced internalization thus facilitating the targeted delivery of therapeutic payloads, such as toxins or drugs, to diseased cells.

In alternative embodiments, the disclosed anti-CDH17 antibodies or fragments thereof may be used in combination with other anti-CDH17 antibodies that bind to distinct and non-overlapping epitopes on CDH17, leading to enhanced internalization or therapeutic potency in activating or blocking signaling pathways, triggering immune responses, or facilitating the targeted delivery of therapeutic payloads, such as toxins or drugs, to diseased cells.

In alternative embodiments, the disclosed anti-CDH17 antibodies or fragments thereof may be used in the context of bispecific or multispecific antibodies, engineered with at least one binding specificity to CDH17. Such antibodies may be used for any of the purposes herein described. In another embodiment, a bi- or tri-specific antibody may have at least one binding specificity for CDH17, and at least one binding specificity for another ligand. In one embodiment, the other ligand has specificity for human immune cell surface proteins such as CD3, CD28, CD137, OX40, CD27, CD16 or CD40, or tumor-associated antigens including but not limited to MUC1, Claudin18.2, Her2, EGFR, Trop2, Nectin-4, LRRC15, Claudin 6, DLL3, VEGF, and c-MET.

In an embodiment, the anti-CDH17 antibodies or antigen binding fragments thereof comprise a VH having a set of CDRs (HCDR1, HCDR2, and HCDR3) disclosed in Table 1. For example, the anti-CDH17 antibodies or antigen binding fragments thereof may comprise a set of CDRs corresponding to those CDRs in one or more of the anti-CDH17 antibodies disclosed in Table 1 (e.g., the CDRs of the C17_mAb 1).

In another embodiment, the anti-CDH17 antibodies or antigen binding fragments thereof comprise a VL having a set of CDRs (LCDR1, LCDR2, and LCDR3) as disclosed in Table 2. For example, the anti-CDH17 antibodies or antigen binding fragments thereof may comprise a set of CDRs corresponding to those CDRs in one or more of the anti-CDH17 antibodies disclosed in Table 2 (e.g., the CDRs of the C17_mAb 2).

In an alternative embodiment, the anti-CDH17 antibodies or antigen binding fragments thereof comprise a VH having a set of CDRs (HCDR1, HCDR2, and HCDR3) as disclosed in Table 1, and a VL having a set of CDRs (LCDR1, LCDR2, and LCDR3) as disclosed in Table 2. In an embodiment, the antibody may be a monoclonal, chimeric, bispecific, biparatopic, humanized or human antibody, or antigen-binding portions thereof that specifically binds to human CDH17. In one embodiment, the anti-CDH17 antibody or antigen binding fragment thereof comprises all six of the CDR regions of the C17_mAb 1, C17_mAb 2, C17_mAb 3, C17_mAb 4, C17_mAb 5, C17_mAb 6, C17_mAb 7, C17_mAb 8, C17_mAb 9, or C17_mAb 10 antibody formatted as a chimeric or a humanized antibody.

In an embodiment, the anti-CDH17 antibodies or antigen binding fragments thereof comprise a VH having a set of complementarity-determining regions (CDR1, CDR2, and CDR3) selected from the group consisting of:
(i) CDR1: SEQ ID NO: 21, CDR2: SEQ ID NO: 22, CDR3: SEQ ID NO: 23;
(ii) CDR1: SEQ ID NO: 27, CDR2: SEQ ID NO: 28, CDR3: SEQ ID NO: 29;
(iii) CDR1: SEQ ID NO: 33, CDR2: SEQ ID NO: 34, CDR3: SEQ ID NO: 35;
(iv) CDR1: SEQ ID NO: 39, CDR2: SEQ ID NO: 40, CDR3: SEQ ID NO: 41;
(v) CDR1: SEQ ID NO: 43, CDR2: SEQ ID NO: 40, CDR3: SEQ ID NO: 44;
(vi) CDR1: SEQ ID NO: 46, CDR2: SEQ ID NO: 47, CDR3: SEQ ID NO: 48;
(vii) CDR1: SEQ ID NO: 52, CDR2: SEQ ID NO: 53, CDR3: SEQ ID NO: 54;
(viii) CDR1: SEQ ID NO: 58, CDR2: SEQ ID NO: 59, CDR3: SEQ ID NO: 60;
(ix) CDR1: SEQ ID NO: 64, CDR2: SEQ ID NO: 65, CDR3: SEQ ID NO: 66; and
(x) CDR1: SEQ ID NO: 70, CDR2: SEQ ID NO: 71, CDR3: SEQ ID NO: 72.

In an embodiment, the anti-CDH17 antibodies or antigen binding fragments thereof comprise a VL having a set of complementarity-determining regions (CDR1, CDR2, and CDR3) selected from the group consisting of:
(i) CDR1: SEQ ID NO: 24, CDR2: SEQ ID NO: 25, CDR3: SEQ ID NO: 26;
(ii) CDR1: SEQ ID NO: 30, CDR2: SEQ ID NO: 31, CDR3: SEQ ID NO: 32;
(iii) CDR1: SEQ ID NO: 36, CDR2: SEQ ID NO: 37, CDR3: SEQ ID NO: 38;
(iv) CDR1: SEQ ID NO: 24, CDR2: SEQ ID NO: 25, CDR3: SEQ ID NO: 42;
(v) CDR1: SEQ ID NO: 24, CDR2: SEQ ID NO: 25, CDR3: SEQ ID NO: 45;
(vi) CDR1: SEQ ID NO: 49, CDR2: SEQ ID NO: 50, CDR3: SEQ ID NO: 51;
(vii) CDR1: SEQ ID NO: 55, CDR2: SEQ ID NO: 56, CDR3: SEQ ID NO: 57;
(vii) CDR1: SEQ ID NO: 61, CDR2: SEQ ID NO: 62, CDR3: SEQ ID NO: 63;
(ix) CDR1: SEQ ID NO: 67, CDR2: SEQ ID NO: 68, CDR3: SEQ ID NO: 69; and
(x) CDR1: SEQ ID NO: 73, CDR2: SEQ ID NO: 74, CDR3: SEQ ID NO: 75.

In another embodiment, the anti-CDH17 antibodies or antigen binding fragments thereof comprise:
(a) a VH having a set of complementarity-determining regions (CDR1, CDR2, and CDR3) selected from the group consisting of:
   (i) CDR1: SEQ ID NO: 21, CDR2: SEQ ID NO: 22, CDR3: SEQ ID NO: 23;
   (ii) CDR1: SEQ ID NO: 27, CDR2: SEQ ID NO: 28, CDR3: SEQ ID NO: 29;
   (iii) CDR1: SEQ ID NO: 33, CDR2: SEQ ID NO: 34, CDR3: SEQ ID NO: 35;
   (iv) CDR1: SEQ ID NO: 39, CDR2: SEQ ID NO: 40, CDR3: SEQ ID NO: 41;
   (v) CDR1: SEQ ID NO: 43, CDR2: SEQ ID NO: 40, CDR3: SEQ ID NO: 44;
   (vi) CDR1: SEQ ID NO: 46, CDR2: SEQ ID NO: 47, CDR3: SEQ ID NO: 48;
   (vii) CDR1: SEQ ID NO: 52, CDR2: SEQ ID NO: 53, CDR3: SEQ ID NO: 54;
   (viii) CDR1: SEQ ID NO: 58, CDR2: SEQ ID NO: 59, CDR3: SEQ ID NO: 60;
   (ix) CDR1: SEQ ID NO: 64, CDR2: SEQ ID NO: 65, CDR3: SEQ ID NO: 66; and
   (x) CDR1: SEQ ID NO: 70, CDR2: SEQ ID NO: 71, CDR3: SEQ ID NO: 72; and
(b) a VL having a set of complementarity-determining regions (CDR1, CDR2, and CDR3) selected from the group consisting of:
   (i) CDR1: SEQ ID NO: 24, CDR2: SEQ ID NO: 25, CDR3: SEQ ID NO: 26;
   (ii) CDR1: SEQ ID NO: 30, CDR2: SEQ ID NO: 31, CDR3: SEQ ID NO: 32;
   (iii) CDR1: SEQ ID NO: 36, CDR2: SEQ ID NO: 37, CDR3: SEQ ID NO: 38;
   (iv) CDR1: SEQ ID NO: 24, CDR2: SEQ ID NO: 25, CDR3: SEQ ID NO: 42;
   (v) CDR1: SEQ ID NO: 24, CDR2: SEQ ID NO: 25, CDR3: SEQ ID NO: 45;
   (vi) CDR1: SEQ ID NO: 49, CDR2: SEQ ID NO: 50, CDR3: SEQ ID NO: 51;
   (vii) CDR1: SEQ ID NO: 55, CDR2: SEQ ID NO: 56, CDR3: SEQ ID NO: 57;
   (viii) CDR1: SEQ ID NO: 61, CDR2: SEQ ID NO: 62, CDR3: SEQ ID NO: 63;
   (ix) CDR1: SEQ ID NO: 67, CDR2: SEQ ID NO: 68, CDR3: SEQ ID NO: 69; and
   (x) CDR1: SEQ ID NO: 73, CDR2: SEQ ID NO: 74, CDR3: SEQ ID NO: 75.

TABLE 1
CDR Sequences of Anti-CDH17 Antibody Variable Heavy Chains

| Anti-CDH17 | | | |
|---|---|---|---|
| mAb | CDR1 | CDR2 | CDR3 |
| C17_mAb 1 | SEQ ID NO: 21 | SEQ ID NO: 22 | SEQ ID NO: 23 |
| C17_mAb 2 | SEQ ID NO: 27 | SEQ ID NO: 28 | SEQ ID NO: 29 |
| C17_mAb 3 | SEQ ID NO: 33 | SEQ ID NO: 34 | SEQ ID NO: 35 |
| C17_mAb 4 | SEQ ID NO: 39 | SEQ ID NO: 40 | SEQ ID NO: 41 |
| C17_mAb 5 | SEQ ID NO: 43 | SEQ ID NO: 40 | SEQ ID NO: 44 |
| C17_mAb 6 | SEQ ID NO: 46 | SEQ ID NO: 47 | SEQ ID NO: 48 |
| C17_mAb 7 | SEQ ID NO: 52 | SEQ ID NO: 53 | SEQ ID NO: 54 |
| C17_mAb 8 | SEQ ID NO: 58 | SEQ ID NO: 59 | SEQ ID NO: 60 |
| C17_mAb 9 | SEQ ID NO: 64 | SEQ ID NO: 65 | SEQ ID NO: 66 |
| C17_mAb 10 | SEQ ID NO: 70 | SEQ ID NO: 71 | SEQ ID NO: 72 |

TABLE 2
CDR Sequences of Anti-CDH17 Antibody Variable Light Chains

| Anti-CDH17 | | | |
|---|---|---|---|
| mAb | CDR1 | CDR2 | CDR3 |
| C17_mAb 1 | SEQ ID NO: 24 | SEQ ID NO: 25 | SEQ ID NO: 26 |
| C17_mAb 2 | SEQ ID NO: 30 | SEQ ID NO: 31 | SEQ ID NO: 32 |
| C17_mAb 3 | SEQ ID NO: 36 | SEQ ID NO: 37 | SEQ ID NO: 38 |
| C17_mAb 4 | SEQ ID NO: 24 | SEQ ID NO: 25 | SEQ ID NO: 42 |
| C17_mAb 5 | SEQ ID NO: 24 | SEQ ID NO: 25 | SEQ ID NO: 45 |
| C17_mAb 6 | SEQ ID NO: 49 | SEQ ID NO: 50 | SEQ ID NO: 51 |
| C17_mAb 7 | SEQ ID NO: 55 | SEQ ID NO: 56 | SEQ ID NO: 57 |
| C17_mAb 8 | SEQ ID NO: 61 | SEQ ID NO: 62 | SEQ ID NO: 63 |
| C17_mAb 9 | SEQ ID NO: 67 | SEQ ID NO: 68 | SEQ ID NO: 69 |
| C17_mAb 10 | SEQ ID NO: 73 | SEQ ID NO: 74 | SEQ ID NO: 75 |

In an embodiment, the anti-CDH17 antibodies or antigen binding fragments thereof comprise a combination of a VH and a VL having a set of complementarity-determining regions (CDR1, CDR2 and CDR3) selected from the group consisting of:
(i) VH: CDR1: SEQ ID NO: 21, CDR2: SEQ ID NO: 22, CDR3: SEQ ID NO: 23, and VL: CDR1: SEQ ID NO: 24, CDR2: SEQ ID NO: 25, CDR3: SEQ ID NO: 26;
(ii) VH: CDR1: SEQ ID NO: 27, CDR2: SEQ ID NO: 28, CDR3: SEQ ID NO: 29, and VL: CDR1: SEQ ID NO: 30, CDR2: SEQ ID NO: 31, CDR3: SEQ ID NO: 32;
(iii) VH: CDR1: SEQ ID NO: 33, CDR2: SEQ ID NO: 34, CDR3: SEQ ID NO: 35, and VL: CDR1: SEQ ID NO: 36, CDR2: SEQ ID NO: 37, CDR3: SEQ ID NO: 38;
(iv) VH: CDR1: SEQ ID NO: 39 CDR2: SEQ ID NO: 40, CDR3: SEQ ID NO: 41, and VL: CDR1: SEQ ID NO: 24, CDR2: SEQ ID NO: 25, CDR3: SEQ ID NO: 42;
(v) VH: CDR1: SEQ ID NO: 43, CDR2: SEQ ID NO: 40, CDR3: SEQ ID NO: 44, and VL: CDR1: SEQ ID NO: 24, CDR2: SEQ ID NO: 25, CDR3: SEQ ID NO: 45;
(vi) VH: CDR1: SEQ ID NO: 46, CDR2: SEQ ID NO: 47, CDR3: SEQ ID NO: 48; and VL: CDR1: SEQ ID NO: 49, CDR2: SEQ ID NO: 50, CDR3: SEQ ID NO: 51;
(vii) VH: CDR1: SEQ ID NO: 52, CDR2: SEQ ID NO: 53, CDR3: SEQ ID NO: 54; and VL: CDR1: SEQ ID NO: 55, CDR2: SEQ ID NO: 56, CDR3: SEQ ID NO: 57;
(viii) VH: CDR1: SEQ ID NO: 58, CDR2: SEQ ID NO: 59, CDR3: SEQ ID NO: 60-; and VL: CDR1: SEQ ID NO: 61, CDR2: SEQ ID NO: 62, CDR3: SEQ ID NO: 63;
(ix) VH: CDR1: SEQ ID NO: 64, CDR2: SEQ ID NO: 65, CDR3: SEQ ID NO: 66; and VL: CDR1: SEQ ID NO: 67, CDR2: SEQ ID NO: 68, CDR3: SEQ ID NO: 69; and
(x) VH: CDR1: SEQ ID NO: 70, CDR2: SEQ ID NO: 71, CDR3: SEQ ID NO: 72; and VL: CDR1: SEQ ID NO: 73, CDR2: SEQ ID NO: 74, CDR3: SEQ ID NO: 75.

In an embodiment, the anti-CDH17 antibodies or antigen binding fragments thereof comprise a variable heavy chain sequence selected from the group consisting of: SEQ ID NOs: 1, 3, 5, 7, 9, 11, 13, 15, 17, and 19, or an amino acid sequence having at least 90%, 95%, 96%, 97%, 98%, or 99% sequence identity thereto; and/or a variable light chain sequence selected from the group consisting of: SEQ ID NOs: 2, 4, 6, 8, 10, 12, 14, 16, 18, and 20, or an amino acid sequence having at least 90%, 95%, 96%, 97%, 98%, or 99% sequence identity thereto.

In an embodiment, the anti-CDH17 antibodies or antigen binding fragments thereof comprise a combination of a VH and a VL and a set of complementarity-determining regions (CDR1, CDR2 and CDR3) selected from the group consisting of:
(i) VH having an amino acid sequence of SEQ ID NO: 1, or an amino acid sequence having at least 90%, 95%, 96%, 97%, 98%, or 99% sequence identity thereto, and having CDR1: SEQ ID NO: 21, CDR2: SEQ ID NO: 22, CDR3: SEQ ID NO: 23, and VL having an amino acid sequence of SEQ ID NO: 2, or an amino acid sequence having at least 90%, 95%, 96%, 97%, 98%, or 99% sequence identity thereto, and having CDR1: SEQ ID NO: 24, CDR2: SEQ ID NO: 25, CDR3: SEQ ID NO: 26;
(ii) VH having an amino acid sequence of SEQ ID NO: 3, or an amino acid sequence having at least 90%, 95%, 96%, 97%, 98%, or 99% sequence identity thereto, and having CDR1: SEQ ID NO: 27, CDR2: SEQ ID NO: 28, CDR3: SEQ ID NO: 29, and
   VL having an amino acid sequence of SEQ ID NO: 4, or an amino acid sequence having at least 90%, 95%, 96%, 97%, 98%, or 99% sequence identity thereto, and having CDR1: SEQ ID NO: 30, CDR2: SEQ ID NO: 31, CDR3: SEQ ID NO: 32;
(iii) VH having an amino acid sequence of SEQ ID NO: 5, or an amino acid sequence having at least 90%, 95%, 96%, 97%, 98%, or 99% sequence identity thereto, and having CDR1: SEQ ID NO: 33, CDR2: SEQ ID NO: 34, CDR3: SEQ ID NO: 35, and
   VL having an amino acid sequence of SEQ ID NO: 6, or an amino acid sequence having at least 90%, 95%, 96%, 97%, 98%, or 99% sequence identity thereto, and having CDR1: SEQ ID NO: 36, CDR2: SEQ ID NO: 37, CDR3: SEQ ID NO: 38;
(iv) VH having an amino acid sequence of SEQ ID NO: 7, or an amino acid sequence having at least 90%, 95%, 96%, 97%, 98%, or 99% sequence identity thereto, and having CDR1: SEQ ID NO: 39 CDR2: SEQ ID NO: 40, CDR3: SEQ ID NO: 41, and
   VL having an amino acid sequence of SEQ ID NO: 8, or an amino acid sequence having at least 90%, 95%, 96%, 97%, 98%, or 99% sequence identity thereto, and having CDR1: SEQ ID NO: 24, CDR2: SEQ ID NO: 25, CDR3: SEQ ID NO: 42;
(v) VH having an amino acid sequence of SEQ ID NO: 9, or an amino acid sequence having at least 90%, 95%, 96%, 97%, 98%, or 99% sequence identity thereto, and having CDR1: SEQ ID NO: 43, CDR2: SEQ ID NO: 40, CDR3: SEQ ID NO: 44, and
   VL having an amino acid sequence of SEQ ID NO: 10, or an amino acid sequence having at least 90%, 95%, 96%, 97%, 98%, or 99% sequence identity thereto, and having CDR1: SEQ ID NO: 24, CDR2: SEQ ID NO: 25, CDR3: SEQ ID NO: 45;
(vi) VH having an amino acid sequence of SEQ ID NO: 11, or an amino acid sequence having at least 90%, 95%, 96%, 97%, 98%, or 99% sequence identity thereto, and having CDR1: SEQ ID NO: 46, CDR2: SEQ ID NO: 47, CDR3: SEQ ID NO: 48, and
   VL having an amino acid sequence of SEQ ID NO: 12, or an amino acid sequence having at least 90%, 95%, 96%, 97%, 98%, or 99% sequence identity thereto, and having CDR1: SEQ ID NO: 49, CDR2: SEQ ID NO: 50, CDR3: SEQ ID NO: 51;
(vii) VH having an amino acid sequence of SEQ ID NO: 13, or an amino acid sequence having at least 90%, 95%, 96%, 97%, 98%, or 99% sequence identity thereto, and having CDR1: SEQ ID NO: 52, CDR2: SEQ ID NO: 53, CDR3: SEQ ID NO: 54, and
   VL having an amino acid sequence of SEQ ID NO: 14, or an amino acid sequence having at least 90%, 95%, 96%, 97%, 98%, or 99% sequence identity thereto, and having CDR1: SEQ ID NO: 55, CDR2: SEQ ID NO: 56, CDR3: SEQ ID NO: 57;
(viii) VH having an amino acid sequence of SEQ ID NO: 15, or an amino acid sequence having at least 90%, 95%, 96%, 97%, 98%, or 99% sequence identity thereto, and having CDR1: SEQ ID NO: 58, CDR2: SEQ ID NO: 59, CDR3: SEQ ID NO: 60, and
   VL having an amino acid sequence of SEQ ID NO: 16, or an amino acid sequence having at least 90%, 95%, 96%, 97%, 98%, or 99% sequence identity thereto, and having CDR1: SEQ ID NO: 61, CDR2: SEQ ID NO: 62, CDR3: SEQ ID NO: 63;
(ix) VH having an amino acid sequence of SEQ ID NO: 17, or an amino acid sequence having at least 90%, 95%, 96%, 97%, 98%, or 99% sequence identity thereto, and having CDR1: SEQ ID NO: 64, CDR2: SEQ ID NO: 65, CDR3: SEQ ID NO: 66, and
   VL having an amino acid sequence of SEQ ID NO: 18, or an amino acid sequence having at least 90%, 95%, 96%, 97%, 98%, or 99% sequence identity thereto, and having CDR1: SEQ ID NO: 67, CDR2: SEQ ID NO: 68, CDR3: SEQ ID NO: 69; and
(x) VH having an amino acid sequence of SEQ ID NO: 19, or an amino acid sequence having at least 90%, 95%, 96%, 97%, 98%, or 99% sequence identity thereto, and having CDR1: SEQ ID NO: 70, CDR2: SEQ ID NO: 71, CDR3: SEQ ID NO: 72, and
   VL having an amino acid sequence of SEQ ID NO: 20, or an amino acid sequence having at least 90%, 95%, 96%, 97%, 98%, or 99% sequence identity thereto, and having CDR1: SEQ ID NO: 73, CDR2: SEQ ID NO: 74, CDR3: SEQ ID NO: 75.

In an embodiment, the anti-CDH17 antibodies or antigen binding fragments thereof comprise a pair of variable heavy chain and variable light chain sequences, selected from the following combinations: a variable heavy chain sequence comprising SEQ ID NO: 1 and a variable light chain sequence comprising SEQ ID NO: 2; a variable heavy chain sequence comprising SEQ ID NO: 3 and a variable light chain sequence comprising SEQ ID NO: 4; a variable heavy chain sequence comprising SEQ ID NO: 5 and a variable light chain sequence comprising SEQ ID NO: 6; a variable heavy chain sequence comprising SEQ ID NO: 7 and a variable light chain sequence comprising SEQ ID NO: 8; a variable heavy chain sequence comprising SEQ ID NO: 9 and a variable light chain sequence comprising SEQ ID NO: 10; a variable heavy chain sequence comprising SEQ ID NO: 11 and a variable light chain sequence comprising SEQ ID NO: 12; a variable heavy chain sequence comprising SEQ ID NO: 13 and a variable light chain sequence comprising SEQ ID NO: 14; a variable heavy chain sequence comprising SEQ ID NO: 15 and a variable light chain sequence comprising SEQ ID NO: 16; a variable heavy chain sequence comprising SEQ ID NO: 17 and a variable light chain sequence comprising SEQ ID NO: 18; and a variable heavy chain sequence comprising SEQ ID NO: 19 and a variable light chain sequence comprising SEQ ID NO: 20. The skilled person will further understand that the variable light and variable heavy chains may be independently selected, or mixed and matched, to prepare an anti-CDH17 antibody or antigen binding fragment thereof comprising a combination of variable heavy and variable light chain that is distinct from the pairings identified above.

In an alternative embodiment, the anti-CDH17 antibodies or antigen binding fragments thereof comprise a pair of variable heavy chain and variable light chain sequences, selected from the following combinations: a variable heavy chain sequence that is 90%, 95%, or 99% identical to SEQ ID NO: 1 and a variable light chain sequence that is 90%, 95%, or 99% identical to SEQ ID NO: 2; a variable heavy chain sequence that is 90%, 95%, or 99% identical to SEQ ID NO: 3 and a variable light chain sequence that is 90%, 95%, or 99% identical to SEQ ID NO: 4; a variable heavy chain sequence that is 90%, 95%, or 99% identical to SEQ ID NO: 5 and a variable light chain sequence that is 90%, 95%, or 99% identical to SEQ ID NO: 6; a variable heavy chain sequence that is 90%, 95%, or 99% identical to SEQ ID NO: 7 and a variable light chain sequence that is 90%, 95%, or 99% identical to SEQ ID NO: 8; a variable heavy chain sequence that is 90%, 95%, or 99% identical to SEQ ID NO: 9 and a variable light chain sequence that is 90%, 95%, or 99% identical to SEQ ID NO: 10; a variable heavy chain sequence that is 90%, 95%, or 99% identical to SEQ ID NO: 11 and a variable light chain sequence that is 90%, 95%, or 99% identical to SEQ ID NO: 12; a variable heavy chain sequence that is 90%, 95%, or 99% identical to SEQ ID NO: 13 and a variable light chain sequence that is 90%, 95%, or 99% identical to SEQ ID NO: 14; a variable heavy chain sequence that is 90%, 95%, or 99% identical to SEQ ID NO: 15 and a variable light chain sequence that is 90%, 95%, or 99% identical to SEQ ID NO: 16; a variable heavy chain sequence that is 90%, 95%, or 99% identical to SEQ ID NO: 17 and a variable light chain sequence that is 90%, 95%, or 99% identical to SEQ ID NO: 18; a variable heavy chain sequence that is 90%, 95%, or 99% identical to SEQ ID NO: 19 and a variable light chain sequence that is 90%, 95%, or 99% identical to SEQ ID NO: 20.

In an alternative embodiment, the anti-CDH17 antibodies or antigen binding fragments thereof comprise a pair of variable heavy chain and variable light chain sequences, selected from the following combinations: a variable heavy chain sequence that is 90%, 95%, or 99% identical to SEQ ID NO: 76 and a variable light chain sequence that is 90%, 95%, or 99% identical to SEQ ID NO: 78; a variable heavy chain sequence that is 90%, 95%, or 99% identical to SEQ ID NO: 76 and a variable light chain sequence that is 90%, 95%, or 99% identical to SEQ ID NO: 79; a variable heavy chain sequence that is 90%, 95%, or 99% identical to SEQ ID NO: 77 and a variable light chain sequence that is 90%, 95%, or 99% identical to SEQ ID NO: 78; a variable heavy chain sequence that is 90%, 95%, or 99% identical to SEQ ID NO: 77 and a variable light chain sequence that is 90%, 95%, or 99% identical to SEQ ID NO: 79; a variable heavy chain sequence that is 90%, 95%, or 99% identical to SEQ ID NO: 80 and a variable light chain sequence that is 90%, 95%, or 99% identical to SEQ ID NO: 82; a variable heavy chain sequence that is 90%, 95%, or 99% identical to SEQ ID NO: 80 and a variable light chain sequence that is 90%, 95%, or 99% identical to SEQ ID NO: 83; a variable heavy chain sequence that is 90%, 95%, or 99% identical to SEQ ID NO: 81 and a variable light chain sequence that is 90%, 95%, or 99% identical to SEQ ID NO: 82; a variable heavy chain sequence that is 90%, 95%, or 99% identical to SEQ ID NO: 81 and a variable light chain sequence that is 90%, 95%, or 99% identical to SEQ ID NO: 83; a variable heavy chain sequence that is 90%, 95%, or 99% identical to SEQ ID NO: 84 and a variable light chain sequence that is 90%, 95%, or 99% identical to SEQ ID NO: 86; a variable heavy chain sequence that is 90%, 95%, or 99% identical to SEQ ID NO: 84; a variable light chain sequence that is 90%, 95%, or 99% identical to SEQ ID NO: 87; a variable heavy chain sequence that is 90%, 95%, or 99% identical to SEQ ID NO: 85; a variable light chain sequence that is 90%, 95%, or 99% identical to SEQ ID NO: 86; a variable heavy chain sequence that is 90%, 95%, or 99% identical to SEQ ID NO: 85; a variable light chain sequence that is 90%, 95%, or 99% identical to SEQ ID NO: 87; and a variable heavy chain sequence that is 90%, 95%, or 99% identical to SEQ ID NO: 123 and a variable light chain sequence that is 90%, 95%, or 99% identical to SEQ ID NO: 124.

Advantageously, such antibodies or fragments thereof retain binding specificity for CDH17. The skilled person will further understand that the variable light and variable heavy chains may be independently selected, or mixed and matched, to prepare an anti-CDH17 antibody comprising a combination of variable heavy and variable light chain that is distinct from the pairings identified above.

In some embodiments, the antibody is a full-length antibody. In other embodiments, the antibody is an antigen binding fragment including, for example, an antigen binding fragment selected from the group consisting of: Fab, Fab′, F(ab′)2, Fv, domain antibodies (dAbs), and complementarity determining region (CDR) fragments, single-chain antibodies (scFv), chimeric antibodies, diabodies, triabodies, tetrabodies, mini-antibodies, and polypeptides that contain at least a portion of an immunoglobulin that is sufficient to confer CDH17 specific binding to the polypeptide.

In some embodiments, a variable region domain of an anti-CDH17 antibody disclosed herein may be covalently attached at a C-terminal amino acid to at least one other antibody domain or a fragment thereof. Thus, for example, a VH domain that is present in the variable region domain may be linked to an immunoglobulin CHI domain, or a fragment thereof. Similarly, a VL domain may be linked to a Cκ domain or a fragment thereof. In this way, for example, the antibody may be a Fab fragment wherein the antigen binding domain contains associated VH and VL domains covalently linked at their C-termini to a CHI and Cκ domain, respectively. The CHI domain may be extended with further amino acids, for example to provide a hinge region or a portion of a hinge region domain as found in a Fab fragment, or to provide further domains, such as antibody CH2 and CH3 domains.

Thus, in one embodiment, the antigen binding fragment comprises at least one CDR as described herein. The antigen binding fragment may comprise at least two, three, four, five, or six CDRs as described herein. The antigen binding fragment further may comprise at least one variable region domain of an antibody described herein. The variable region domain may be of any size or amino acid composition and will generally comprise at least one CDR sequence responsible for binding to human CDH17, for example, CDR-H1, CDR-H2, CDR-H3, CDR-L1, CDR-L2, and/or CDR-L3 as described herein, and which is adjacent to or in frame with one or more framework sequences.

In some embodiments, the anti-CDH17 antibody is a monoclonal antibody. In some embodiments, the anti-CDH17 antibody is a human antibody. In alternative embodiments, the anti-CDH17 antibody is a murine antibody. In some embodiments, the anti-CDH17 antibody is a chimeric antibody, a bispecific antibody, a biparatopic antibody, or a humanized antibody.

In some embodiments, the anti-CDH17 antibody is an IgG. In some embodiments, the anti-CDH17 antibody is an IgG1, IgG2, IgG3, or IgG4.

In some embodiments, the anti-CDH17 antibodies or antigen binding fragments thereof comprise one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

“Conservative modifications” refer to amino acid modifications that do not significantly affect or alter the binding characteristics of the antibody containing the amino acid sequences. Conservative modifications include amino acid substitutions, additions, and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al. (1998) Acta Physiol Scand Suppl 643:55-67; Sasaki et al. (1998) Adv Biophys 35:1-24). Amino acid substitutions to the antibodies of the disclosure may be made by known methods, for example by PCR mutagenesis (U.S. Pat. No. 4,683,195).

In some embodiments, the antibody or fragment thereof comprises a variable heavy chain sequence that comprises an amino acid sequence with at least about 95%, about 96%, about 97%, about 98%, or about 99%, sequence identity to the amino acid sequence set forth in SEQ ID NOs: 1, 3, 5, 7, 9, 11, 13, 15, 17, or 19. In other embodiments, the antibody or fragment thereof retains the binding and/or functional activity of an antibody or fragment thereof that comprises the variable heavy chain sequence of SEQ ID NOs: 1, 3, 5, 7, 9, 11, 13, 15, 17, or 19. In still further embodiments, the antibody or fragment thereof comprises the variable heavy chain sequence of SEQ ID NOs: 1, 3, 5, 7, 9, 11, 13, 15, 17, or 19, and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 1, 3, 5, 7, 9, 11, 13, 15, 17, or 19 (based on the numbering system of IMGT).

In particular embodiments, the antibody or fragment thereof comprises a variable heavy chain sequence with at least about 95%, about 96%, about 97%, about 98%, or about 99% sequence identity to the antibody or fragment thereof heavy chain variable region sequence set forth in SEQ ID NOs: 1, 3, 5, 7, 9, 11, 13, 15, 17, or 19, comprises one or more conservative amino acid substitutions in a framework region (based on the numbering system of IMGT), and retains the binding and/or functional activity of an antibody or fragment thereof that comprises a variable heavy chain sequence as set forth in SEQ ID NOs: 1, 3, 5, 7, 9, 11, 13, 15, 17, or 19 and a variable light chain sequence as set forth in SEQ ID NOs: 2, 4, 6, 8, 10, 12, 14, 16, 18, or 20.

In some embodiments, the antibody or fragment thereof comprises a variable light chain sequence that comprises an amino acid sequence with at least about 95%, about 96%, about 97%, about 98%, or about 99% sequence identity to the amino acid sequence set forth in SEQ ID NOs: 2, 4, 6, 8, 10, 12, 14, 16, 18, or 20. In other embodiments, the antibody or fragment thereof retains the binding and/or functional activity of an antibody or fragment thereof that comprises the variable light chain sequence of SEQ ID NOs: 2, 4, 6, 8, 10, 12, 14, 16, 18, or 20. In still further embodiments, the antibody or fragment thereof comprises the variable light chain sequence of SEQ ID NOS: 2, 4, 6, 8, 10, 12, 14, 16, 18, or 20 and have one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 2, 4, 6, 8, 10, 12, 14, 16, 18, or 20 (based on the numbering system of IMGT).

In particular embodiments, the antibody or fragment thereof comprises a variable light chain sequence with at least about 95%, about 96%, about 97%, about 98%, or about 99% sequence identity to light chain variable region sequence set forth in SEQ ID NOs: 2, 4, 6, 8, 10, 12, 14, 16, 18, or 20, comprises one or more conservative amino acid substitutions in a framework region (based on the numbering system of IMGT), and retains the binding and/or functional activity of an antibody or fragment thereof that comprises a variable heavy chain sequence as set forth in SEQ ID NOs: 1, 3, 5, 7, 9, 11, 13, 15, 17, or 19 and a variable light chain sequence as set forth in SEQ ID NOs: 2, 4, 6, 8, 10, 12, 14, 16, 18, or 20.

### Binding/Specificity/Species Cross-Reactivity

The disclosed antibodies (C17_mAb 1, C17_mAb 2, C17_mAb 3, C17_mAb 4, C17_mAb 5, C17_mAb 6, C17_mAb 7, C17_mAb 8, C17_mAb 9, and C17_mAb 10), humanized variants thereof, and antigen binding fragments specifically bind to human CDH17 as it occurs on the surface of normal or malignant cells. CDH17 exhibits low homology to its phylogenetically close relatives. The amino acid sequences of the human cadherin phylogenetically close protein CDH16 was obtained with NCBI Reference Sequences: NP_004053.1 SEQ ID NO: 113). The homology between the human CDH17 and human CDH16 is “29.16%” and the homology to the next most phylogenetically related protein CDH23 is “25.88%”. Based on the low homology, it is unlikely that anti-CDH17 antibodies will bind to other cadherin super family members, and there was no discernible binding to CDH17 negative cells (e.g., CHO cells and COLO205 CDH17 KO cells).

The disclosed biparatopic antibodies (biparatopic 1 and biparatopic 2) were examined for non-target specificity using a Membrane Proteome Array (MPA) platform (Integral Molecular). In this platform the antibodies were screened for off target binding to 6000 membrane protein natively expressed on unfixed human cells. The disclosed biparatopic 1 and biparatopic 2 showed no binding to any of the off-target proteins indicating high specificity for its target CDH17.

Antibodies typically bind specifically to their cognate antigen with high affinity, reflected by a dissociation constant (KD) of 10⁻⁷ to 10⁻¹¹ M or less. Any KD greater than about 10⁻⁶ M is generally considered to indicate nonspecific binding. As used herein, an antibody that “binds specifically” to an antigen refers to an antibody that binds to the antigen and substantially identical antigens with high affinity, which means having a KD of 10⁻⁷ M or less, preferably 10⁻⁸ M or less, even more preferably 5×10⁻⁹ M or less, and most preferably between 10⁻⁸ M and 10⁻¹⁰ M or less but does not bind with high affinity to an unrelated antigen. The disclosed antibodies bind to the human CDH17 with high affinity, with KD determined by BLI ranging from 1.65E-10 to 3.84E-08 M.

The term “cross-reacts,” as used herein, refers to the ability of an anti-human CDH17-specific antibody described herein to bind to CDH17 from a different species. For example, an antibody described herein may also bind CDH17 from another species (e.g., cynomolgus monkey or mouse CDH17). As used herein, cross-reactivity may be measured by detecting a specific reactivity with purified antigen in binding assays (e.g., SPR, ELISA) or binding to, or otherwise functionally interacting with, cells physiologically expressing CDH17. Methods for determining cross-reactivity include standard binding assays as described herein, for example, by BIACORE® surface plasmon resonance (SPR) analysis using a BIACORE® 2000 SPR instrument (Biacore AB, Uppsala, Sweden), Biolayer interferometry (BLI), or flow cytometric techniques.

The disclosed human CDH17 antibodies, C17_mAb 1 to C17_mAb 10 and humanized variants thereof, all bind to CDH17 from cynomolgus monkey with notable affinity: C17_mAb 1 to C17_mAb 10 bind cynoCDH17 in cell-based assays with affinities comparable to the binding affinity to huCDH17. The disclosed antibodies bind to the cynoCDH17 with affinities of 1E-10 to 7E-08 M determined by cell based binding on HEK-293-cynoCDH17 cell line. The disclosed antibodies exhibit no binding or significantly lower binding affinity to murine CDH17 compared to huCDH17 at (>6.6E-08).

### Epitope Mapping

Epitope mapping determines the antigenic regions, known as epitopes, that are recognized by antibodies. Commonly employed methods for epitope mapping include a diverse array of biochemical, structural, and computational approaches, each offering unique insights into antigen-antibody interactions. One such approach involves engineering the target antigen and screening for binding to the antibody of interest. By systematically probing individual antigen fragments, this method allows for the precise delineation of the linear epitopes along the antigen sequence.

The disclosed antibodies (C17_mAb 1, C17_mAb 2, C17_mAb 3, C17_mAb 4, C17_mAb 5, C17_mAb 6, C17_mAb 7, C17_mAb 8, C17_mAb 9 and C17_mAb 10) and antigen binding fragments and humanized variants thereof specifically bind to the extracellular domain of CDH17. Provided herein are antibodies or antigen binding fragments thereof that bind to the same or substantially the same epitope on human CDH17 as C17_mAb 1, C17_mAb 2, C17_mAb 3, C17_mAb 4, C17_mAb 5, C17_mAb 6, C17_mAb 7, C17_mAb 8, C17_mAb 9, or C17_mAb 10.

The 7 domains of CDH17 were divided into 3 sections for our studies: ECD1-2 (SEQ ID NO: 114), ECD3-5 (SEQ ID NO: 115), and ECD6-7 (SEQ ID NO: 116). Recombinant proteins were produced with sequences corresponding to the listed ECD. Using ELISA based method, the binding area to the ECD of CDH17 by the disclosed antibodies (C17_mAb 1, C17_mAb 2, C17_mAb 3, C17_mAb 4, C17_mAb 5, C17_mAb 6, C17_mAb 7, C17_mAb 8, C17_mAb 9 and C17_mAb 10) and antigen binding fragments and humanized variants thereof were evaluated.

The disclosed antibodies fall within 3 bins corresponding to ECD1-2, ECD3-5 and ECD6-7 using ELISA method. C17_mAb 8 and C17_mAb 7 are in the ECD1-2 bin with C17-mAb 7 having weak binding to ECD3-5 too. C17_mAb 1, C17_mAb 9 and C17 mAb 10 are in the ECD3-5 bin with C17_mAb 10 having weak binding to ECD6-7 bin. C17_mAb 2, C17_mAb 3, C17_mAb 4, C17_mAb 5 and C17_mAb 6 are in the ECD6-7 bin.

Using cell based cross blocking assay, there are 2-3 groups within each bin. The antibodies were grouped separately if they were in the same bin but did not block binding to each other's epitope. In ECD1-2 bin, C17_mAb 8 and C17_mAb 7 did not cross block each other's binding to ECD1-2, so there are in 2 separate groups (I and II respectively) within ECD1-2. In ECD3-5 bin, C17_mAb 1 did not cross block with C17 mAb 9 and C17_mAb 10, so it is in group I of ECD3-5 bin. C17_mAb 10 blocked C17_mAb 9 binding and are in the same group II of ECD3-5. In ECD6-7, C17_mAb 2 and C17_mAb 3 cross blocked each other but not C17_mAb 4, C17_mAb 5 and C17_mAb 6 which are in group II of ECD6-7.

### Antibody-Derived Receptor Internalization

Antibody-antigen complexes formed on the cell surface are likely to be endocytosed by the cell. The antibody-derived receptor internalization can lead to higher order receptor clustering, rapid receptor downregulation and lysosome trafficking, this process is influenced by a range of intricate factors. A key determinant is the density of target antigens on the cell surface, with higher densities potentially leading to faster uptake. The binding affinity and specificity of the antibody to its target antigen on the cell surface is also critical for efficient internalization. Additionally, the internalization process is impacted by the specific cellular machinery involved in receptor-mediated endocytosis, which can vary among different cell types. The rate of internalization can also be influenced by the antibody binding epitope. In the case of ADC which delivers the conjugated toxin through antibody-derived receptor internalization, the internalization kinetics can be influenced by the structure of the ADC, including the type of antibody used, the linker, and the cytotoxic payload. Overall, the intricate interplay of these factors determines the extent and efficiency of ADC internalization, which in turn affects the drug's ability to reach its intended intracellular target and execute its therapeutic action.

In particular embodiments, the anti-CDH17 antibodies or antigen binding fragments thereof specifically bind human CDH17 on the surface of cancer cells and induce the internalization of CDH17. Internalization of CDH17 by the disclosed anti-CDH17 antibodies was assessed by an in vitro pH dye image-based assay.

The pH dye method utilizes change in low acidic pH that occurs intracellularly for fluorescent signal to turn on. Using this method the level and rate of internalization of CDH17 from cell surface by selecting disclosed antibodies (C17_mAb 1, C17_mAb 2, C17_mAb 3, C17_mAb 7, C17_mAb 8, C17_mAb 9, and C17_mAb 10) and biparatopic antibody (Biparatopic 1, Biparatopic 2, and Biparatopic 3), antigen binding fragments and humanized variants thereof were evaluated.

The biparatopic antibodies displayed enhanced and faster internalization of CDH17 compared to their respective mAb alone or combination of mAbs condition. The enhanced internalization by the biparatopic antibodies were preserved across various cancer cell lines expressing varying levels of CDH17.

This antibody-derived endocytosis of CDH17 by the disclosed antibodies makes it ideal for ADC molecule. When combined with a toxin-conjugated secondary antibody or when directly conjugated to a toxin payload, ADCs can mediate killing of target expressing cells.

Generally, once a CDH17 ADC binds target antigen on the cancer cell surface, it is internalized and sent along the endosome/lysosome pathway for degradation. In the lysosome the payload is released either through specific cleavage of the linker by lysosome enzymes or general degradation of the antibody. The released cytotoxic compound then leaves the lysosome, accumulates to a requisite threshold level, and ultimately causes death of the targeted cancer cell.

The ADC capability of the anti-CDH17 antibodies (C17_mAb 1, C17_mAb 2, C17_mAb 3) and biparatopic antibodies (bpAb 1, bpAb 1.1. bpAb2, bpAb 2.1 and bpAb3) were demonstrated by a cytotoxicity assay using an immunotoxin conjugated secondary bound to the CDH17 antibodies wherein the toxin is released upon incorporation into cells to inhibit tumor cell growth.

Cytotoxicity was assessed in different cell lines with varying target densities and different payloads. The disclosed biparatopic 1 showed enhanced cytotoxicity compared to its respective mAb alone or combination of its mAbs. The enhanced cytotoxicity was also preserved across various cancer cell lines expressing varying levels of CDH17 and using different payload.

The biparatopic antibodies elicited enhanced internalization compared to the combinations of its mAbs showing efficient internalization can be achieved utilizing a single biparatopic molecule compared to combination of its two mAbs corresponding to each arm. This feature is highly attractive when developing an ADC as one molecule can be better regulated than two molecules to achieve the same functional efficacy during development. In some embodiments, the biparatopic antibodies disclosed herein promote targeting and/or internalization of cancer cells that express CDH17 at a detectable but intermediate to lower levels.

### T Cell Activation

By bridging tumor cells with cytotoxic T cells, bispecific T cell engager (TCE) antibodies are known to facilitate the formation of immunological synapses, leading to potent T cell activation and subsequent tumor cell killing. This process, known as redirected T cell cytotoxicity, enables T cells to recognize and eliminate cancer cells independent of major histocompatibility complex (MHC) restriction.

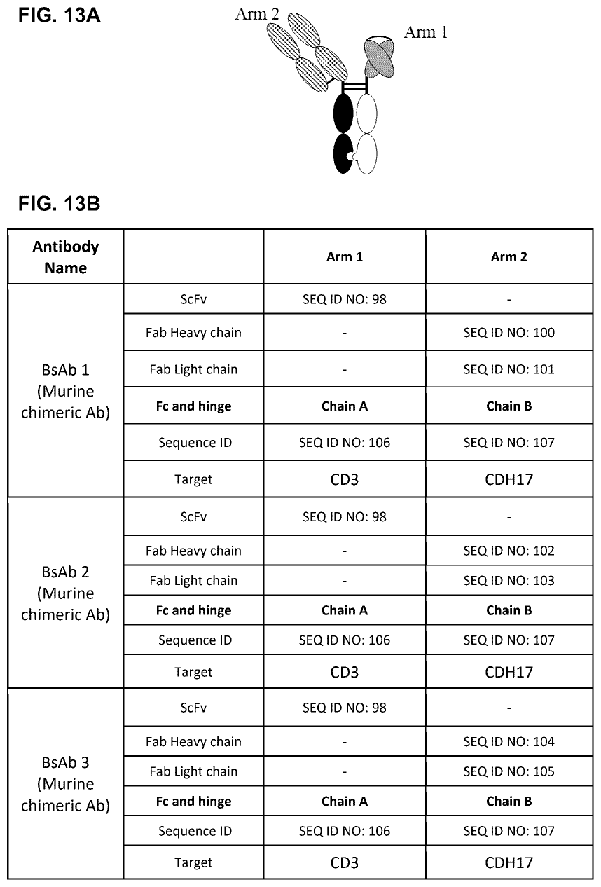

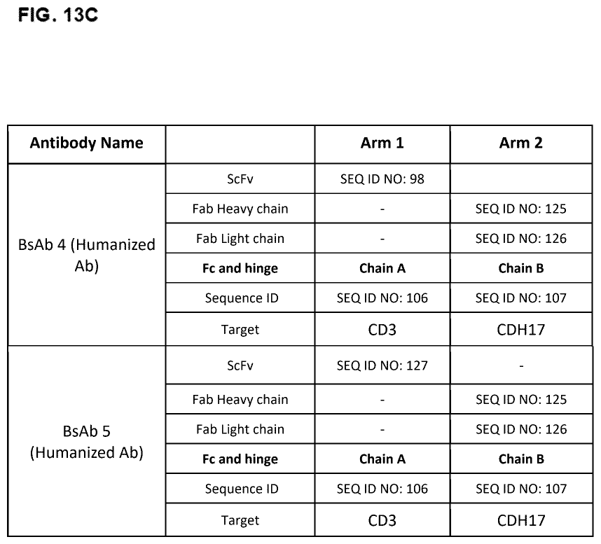

In an alternative embodiment, the disclosed anti-CDH17 antibodies were engineered to simultaneously bind to CDH17 antigen and CD3 (anti-CDH17/CD3 bispecific antibodies BsAb 1, BsAb 5). In an embodiment, the anti-CDH17/CD3 antibodies targeting CDH17 and CD3 have been synthesized in IgG-scFv format. FIG. 13 shows the composition of the bispecific antibodies, where binding arms to CD3 (Arm 1) and CDH17 (Arm 2) is shown. The scFv binding to the first antigen-binding domain (CD3) is depicted in grey, while the second antigen-binding domain (CDH17) is shown in hatched fill. To enhance the heterodimer formation of Fc for improved production, “knob in hole” mutations on CH3 described by Merchant A, et al (1998) were applied. And to eliminate FcγR-binding the “LALAPA” mutations are introduced in the Fc region (Shields R L, et al 2001). These Fc mutations are provided in FIG. 13B (code by EU numbering).

Various assay methods have been developed to measure T cell activation end points such as activation surface markers, cytokine secretion, and T cell proliferation. A reporter assay utilizing a genetically modified Jurkat T cell line with engineered NFAT reporter expression was used to evaluate the functional activity of the disclosed anti-CDH17/CD3 BsAbs. As shown herein, the CDH17 bsAbs were shown to activate the Jurkat reporter cells in a CDH17 dependent manner. Only in the presence of CDH17 expressing target cells was there activation signal by Jurkat reporter cells as stimulation of Jurkat reporter cells in the absence of target cells expressing CDH17 did not produce an activation signal.

### Chimeric and Humanized Anti-CDH17 Therapeutics

In certain embodiments, an antibody provided herein is a chimeric antibody. Certain chimeric antibodies are described (e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen binding fragments thereof.

Antibodies may be prepared as chimeric antibodies or antigen binding fragments thereof with murine variable regions and human constant regions. Human antibody constant regions may be of reported allotypes, reviewed in Jefferis et al., Human immunoglobulin allotypes: possible implications for immunogenicity. MAbs 1, 332-338 (2009). In one embodiment, the human heavy chain constant region uses a consensus human IgG1 constant region sequence (Uniprot P01857) (SEQ ID NO: 108), whereas the light chain constant region uses a consensus human kappa constant region sequence (UniProtP01834) (SEQ ID NO: 109). Human IgG1 may be chosen because it is one of the most common subtypes for chimera antibody generation and can provide effector function. Human kappa constant region may be used because a majority of parental murine antibodies are of mouse kappa light chain. For the murine antibodies with lambda light chain, a consensus human kappa constant region may be used (i.e., UniProtP01834) (SEQ ID NO: 109).

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs (or portions thereof), are derived from a non-human antibody, and FRs (or portions thereof) are derived from human antibody sequences. An antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore and/or improve antibody binding specificity or affinity. In some embodiments, some CDR residues in a humanized antibody (e.g, 1, 2, 3, 4, or 5 residues) may be mutated to improve antibody developability.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008) and framework regions derived from screening FR libraries (see, e.g., Baca et al., J. Biol. Chem. 272:10678-10684 (1997) and Rosok et al., J. Biol. Chem. 271:22611-22618 (1996).

In some embodiments, the provided bispecific and biparatopic antibodies comprises a modified Fc region wherein a naturally occurring Fc region is modified to facilitate the generation of bispecific antibody by Fc heterodimerization. For example, a Knob-in-Hole strategy (see, e.g., Intl. Publ. No. WO 2006/028936) can be employed to direct Fc heterodimerization. An amino acid with a small side chain (hole) is introduced into one Fc domain and an amino acid with a large side chain (knob) is introduced into the other Fc domain. After co-expression of the two heavy chains, a heterodimer is formed as a result of the preferential interaction of the heavy chain with a “hole” with the heavy chain with a “knob” (Ridgway, Presta et al. 1996). Exemplary Fc mutation pairs forming a knob and a hole are: T366Y/F405A, T366W/F405W, F405W/Y407A, T394W/Y407T, T394S/Y407A, T366W/T394S, F405W/T394S and T366W/T366S/L368A/Y407V.

A S354C mutation in knob chain and a Y349C mutation in hole chain were also introduced to form an inter-chain disulfide bond between heterodimerize heavy chain (Merchant A M et al. 1998). These additional mutations have been reported to enhance the heavy chain heterodimerization rate to approximately 95%.

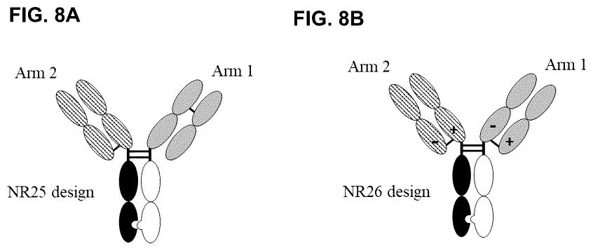

In specific embodiments, the bispecific and biparatopic antibodies may include modified VH and VL framework regions or heavy chain CHI and light chain constant regions, facilitating the accurate pairing of cognate heavy and light chains. For example, incorporating a series of mutations to replace the native disulfide bond between the heavy chain constant region and light chain constant region on one Fab arm has shown improved precision in heavy and light chain pairing for IgG-like bispecific antibodies (Chowdhury et al., 2015). In another embodiment, an electrostatic steering mechanism at the heavy chain and light chain interface may be utilized to enhance the recognition between the heavy and light chains (Yan, 2015). Introducing additional charge-charge interactions of opposite polarities between the two arms of the bispecific antibody significantly reduces the likelihood of mispairing. Since mispairing of heavy and light chains results in opposite charges, it is not conducive to forming a stable antibody. As depicted in FIG. 8, both strategies have been implemented using distinct sets of mutations in the NR25 and NR26 designs.

Antibodies of this disclosure may have amino acid substitutions in the Fc region that improve manufacturing and drug stability. An example for IgG1 Antibodies of this disclosure that are modified to improve stability, selectivity, cross-reactivity, affinity, immunogenicity or other desirable biological or biophysical property are within the scope of the disclosure.

### Biparatopic Anti-CDH17

Previous studies from Kusano-Arai et al (Kusano-Arai O, et al., Monoclon. Antib. Immunodiagn. Immunother., 37 (1): 1-11, 2018) demonstrate a synergistic cytotoxic effect of an anti-CDH17 immunotoxin cocktail comprising three antibodies specific for different epitopes of CDH17 on human gastric cancer cells. Two of the anti-CDH17 mAbs (D2101 and D2005) used to prepare the immunotoxin cocktail described in the 2018 publication were reported to bind to an epitope within EC1-2 (amino acid residues 23-236) of CDH17 while the third antibody (D2008) is reported to bind to an epitope within EC5-6 (amino acid residues 450-667) of CDH17. A subsequent publication from the same group used surface plasmon resonance (SPR) analysis using human CDH17 ectodomains as analytes and concludes that D2101 binds an epitope in EC1 of human CDH17 (Fujiwara K, et al., Ann Nucl Med.: 34 (1): 13-23 (2020)).

Using saporin as an immunotoxin that is conjugated to a secondary antibody, Kusano-Arai used an indirect method to show that a D2101 immunotoxin (D2101IT) exhibited the most efficient cytotoxicity in a human gastric cancer cell line (AGS high cell line) compared to immunotoxins comprising the anti-CDH17 mAbs D2005 and D2008. They also demonstrate that a combination of three antibodies targeting EC1-2 and EC5-6 led to enhanced cytotoxicity of the AGS gastric cancer cells.

Further examination of cytotoxicity of the three antibody cocktail on AGS expressing varying levels of CDH17: AGShigh (200 k/cell), AGSmed (50 k/cell) and AGSlow (50 k/well) demonstrates that synergistic effect of the cocktail compared to the cytotoxic effect of the CDH17 EC1-2 specific D2101IT alone is preserved even at low expressing levels of CDH17.

Kusano-Arai et al hypothesized that use of cocktail therapy that includes multiple antibodies specific for distinct epitope could lead to additional linkages between surface CDH17 molecules. These linkages may facilitate the clustering of CDH17 leading to enhanced internalization and delivery of greater amount of toxin to the target cells. It was noted that this method was a desirable strategy for low level target expressing cells that may be quite relevant in various human tumors.

The immunotoxin cocktail effect showed significant enhancement in cell killing as measured by reduced variability, the group does not show support for increased internalization or rate of internalization induced by the cocktail therapy that could explain the enhanced cell killing. It is also unclear if the synergistic effect requires all 3 mAbs to be present and if we can expect corelative reduction in synergistic effects if only 2 mAbs were used for a cocktail.

A biparatopic antibody enhances target specificity and avidity by simultaneously binding to two distinct epitopes on the target antigen. Several mechanisms of actions have been proposed for biparatopic antibodies, one of which relies on receptor crosslinking mediated actions such as increased agonism, Fc-mediated effector function, and target downregulation. Biparatopic antibodies also have the tendency to exhibit superior binding with slower dissociation from target. Considering these properties that can be exhibited by biparatopic antibodies, use of biparatopic antibodies for ADC is highly desired (Niquille et al., mAbs, 16:1, 2310890, (2024)). When used in immunotherapies, this dual-binding capability may lead to distinct mechanisms of action derived from the structural effects of biparatopic target engagement, increasing the selectivity for tumor cells expressing the target antigen while minimizing off-target effects on normal tissues, thereby improving the therapeutic index and reducing potential toxicity. A biparatopic ADC may exhibit synergistic anti-tumor activity compared to a combination of two separate ADCs, leading to improved efficacy in preclinical and clinical settings.

Biparatopic T cell engagers also represent a promising strategy for cancer immunotherapy. By simultaneously targeting two distinct epitopes on tumor antigens located on cancer cells and T cells, biparatopic T cell engagers can enhance the specificity and efficacy, potentially mitigate off-target effects and systemic toxicity by concentrating T cell activation at the tumor site. They may also counteract immunosuppressive mechanisms and enhance T cell persistence, contributing to durable anti-tumor responses.

A biparatopic antibody enhances target specificity and avidity by simultaneously binding to two distinct epitopes on the target antigen. For example, an anti-HER2 IgG1 bispecific biparatopic antibody, zanidatamab, demonstrates unique and enhanced functionalities compared to both trastuzumab and the combination of trastuzumab plus pertuzumab (tras+pert). Zanidatamab binds adjacent HER2 molecules in trans, leading to distinct HER2 reorganization characterized by polarized cell surface HER2 caps and large HER2 clusters, phenomena not observed with trastuzumab or tras+pert. Additionally, zanidatamab, but not trastuzumab nor tras+pert, elicits potent complement-dependent cytotoxicity (CDC) against high HER2-expressing tumor cells in vitro. Furthermore, zanidatamab mediates HER2 internalization and downregulation, inhibits both cell signaling and tumor growth, induces antibody-dependent cellular cytotoxicity (ADCC) and phagocytosis (ADCP), and demonstrates superior in vivo antitumor activity compared to tras+pert in a HER2-expressing xenograft model. (Weisser, N. E., et al. Nat Commun 14 (1): 1394 (2023)).

The marketed CAR-T therapy ciltacabtagene autoleucel (Carvykti) also employs the biparatopic strategy that target distinct nonoverlapping epitopes on BCMA, and has demonstrated favorable clinical outcomes. The use of dual binding domains increases avidity compared to a single binding domain, allowing a single CAR to engage with two target proteins, promoting the formation of a strong immune synapse. Remarkably, the doses needed for achieving complete remission are 10 times lower with the dual-nanobody CAR compared to those required with a single-nanobody CAR (Smith, R. Antib Ther 6 (4): 225-239 (2023)).

Anti-CDH17 antibody pairs with good binding affinity and binding specificity for distinct non-overlapping epitopes of human CDH17 were identified as potential candidates for the development of a biparatopic anti-CDH17 antibody or a biparatopic antibody drug conjugate (ADC). To maximize the efficiency of a candidate anti-CDH17 therapeutic antibody, biparatopic antibodies that could elicit a synergistic effect relative to the activity of a combination of the same antibodies used to generate the biparatopic candidate were screened. The disclosed biparatopic antibodies showed enhanced and greater synergistic effect compared to the combination of mAbs.

Biparatopic anti-CDH17 antibodies comprising two Fab arms specific for different epitopes of human CDH17 were synthesized in an IgG-like format (e.g., an IgG1-, IgG2-, IgG3-, or IgG-4 like format). In specific embodiments, a disulfide bond replacement strategy has been employed on one Fab arm of the antibody. Illustrated in FIG. 8A, Arm 1, specifically binding to CDH17 ECD3-5, incorporates engineered Fab with the replacement of the native CH1-CL disulfide bond with a disulfide bond between VH and VL. Meanwhile, Arm 2 binding to CDH17 ECD6-7 maintains the native disulfide bond. This design minimizes the likelihood of pairing between Arm 1 light chain and Arm 2 heavy chain, and vice versa for Arm 2 light chain and Arm 1 heavy chain.

In other embodiments, an engineering approach for CHI-CL, utilizing an electrostatic steering mechanism, has been implemented to retain the native cysteine for ADC payload conjugation. As depicted in FIG. 8B specific negative charged amino acids on the heavy chain and positive charged amino acids on the light chain were introduced on Arm 1 to facilitate a charge-charge interaction at the CH1-CL interface. Simultaneously, specific positive charged amino acid mutants on the heavy chain and negative charged mutants on the light chain were introduced on Arm 2 to promote correct cognation of Arm 2 heavy chain and light chain pairing, while mitigating mispairing from Arm 1 chains.

In some embodiments, the biparatopic antibody or antigen-binding fragment thereof comprise a first antigen-binding portion having specificity to a first epitope in human CDH17 and a second antigen-binding portion having specificity to a second distinct non-overlapping epitope within human CDH17.

In some embodiments, the biparatopic antibody or antigen-binding fragment thereof comprise a first antigen binding portion having specificity for an epitope in ECD 3-5 of human CDH17 and a second binding portion having specificity for an epitope in ECD 6-7 of human CDH17.

In other embodiments, the biparatopic antibody or antigen-binding fragment thereof comprise a first antigen binding portion having specificity for an epitope in ECD 1-2 of human CDH17 and a second binding portion having specificity for an epitope in ECD 6-7 of human CDH17.

In other embodiments, the biparatopic antibody or antigen-binding fragment thereof comprise a first antigen binding portion having specificity for an epitope in ECD 1-2 of human CDH17 and a second binding portion having specificity for an epitope in ECD 3-5 of human CDH17.

In some embodiments, the first antigen-binding portion and the second antigen-binding portion comprise heavy chain complementarity determining regions (CDR) VH CDR1, VH CDR2, and VH CDR3, and light chain CDRs VL CDR1, VL CDR2, and VL CDR3. In some embodiments, the VH CDR1, VH CDR2, VH CDR3, VL CDR1, VL CDR2, and VL CDR3 of the first antigen-binding portion are, respectively (i) the amino acid sequences of SEQ ID NO: 21-26; or (ii) the amino acid sequences of SEQ ID NO: 64-69; and the VH CDR1, VH CDR2, VH CDR3, VL CDR1, VL CDR2, and VL CDR3 of the second antigen-binding portion are, respectively: (i) the amino acid sequences of SEQ ID NO: 27-32; (ii) the amino acid sequences of SEQ ID NO: 33-38; (iii) the amino acid sequences of SEQ ID NO: 39-41, SEQ ID NO: 24, SEQ ID NO: 25 and SEQ ID NO: 42; (iv) the amino acid sequences of SEQ ID NO: 43, SEQ ID NO: 40, SEQ ID NO: 44, SEQ ID NO: 24, SEQ ID NO: 25 and SEQ ID NO: 45; or (v) the amino acid sequences of SEQ ID NO: 46-51.

In some embodiments, the VH CDR1, VH CDR2, VH CDR3, VL CDR1, VL CDR2, and VL CDR3 of the first antigen-binding portion are, respectively, the amino acid sequences of SEQ ID NO: 58-63; and the VH CDR1, VH CDR2, VH CDR3, VL CDR1, VL CDR2, and VL CDR3 of the second antigen-binding portion are, respectively: (i) the amino acid sequences of SEQ ID NO: 27-32; (ii) the amino acid sequences of SEQ ID NO: 33-38; (iii) the amino acid sequences of SEQ ID NO: 39-41, SEQ ID NO: 24, SEQ ID NO: 25 and SEQ ID NO: 42; (iv) the amino acid sequences of SEQ ID NO: 43, SEQ ID NO: 40, SEQ ID NO: 44, SEQ ID NO: 24, SEQ ID NO: 25 and SEQ ID NO: 45; or (v) the amino acid sequences of SEQ ID NO: 46-51.

In some embodiments, the first antigen binding portion comprises a heavy chain variable region (VH) comprising an amino acid sequence that is at least 75%, at least 80%, at least 85%, at least 90%, at least 95%, or at least 99% identical to the amino acid sequence of SEQ ID NO: 1 or 17, and a light chain variable region (VL) comprising an amino acid sequence that is at least 75%, at least 80%, at least 85%, at least 90%, at least 95%, or at least 99% identical to the amino acid sequence of SEQ ID NO: 2 or 18; and the second antigen binding portion comprises a heavy chain variable region (VH) comprising an amino acid sequence that is at least 75%, at least 80%, at least 85%, at least 90%, at least 95%, or at least 99% identical to the amino acid sequence of SEQ ID NO: 3, 5, 7, 9 or 11, and a light chain variable region (VL) comprising an amino acid sequence that is at least 75%, at least 80%, at least 85%, at least 90%, at least 95%, or at least 99% identical to the amino acid sequence of SEQ ID NO: 4, 6, 8, 10 or 12. In some embodiments, the first antigen binding portion comprises a VH comprising an amino acid sequence of SEQ ID NO: 1 or 17, and a light chain variable region (VL) comprising an amino acid sequence of SEQ ID NO: 2 or 18; and the second antigen binding portion comprises a heavy chain variable region (VH) comprising an amino acid sequence of SEQ ID NO: 3, 5, 7, 9 or 11, and a light chain variable region (VL) comprising an amino acid sequence of SEQ ID NO: 4, 6, 8, 10, or 12.

In some embodiments, the first antigen binding portion comprises a heavy chain variable region (VH) comprising an amino acid sequence that is at least 75%, at least 80%, at least 85%, at least 90%, at least 95%, or at least 99% identical to the amino acid sequence of SEQ ID NO: 15, and a light chain variable region (VL) comprising an amino acid sequence that is at least 75%, at least 80%, at least 85%, at least 90%, at least 95%, or at least 99% identical to the amino acid sequence of SEQ ID NO: 16; and the second antigen binding portion comprises a heavy chain variable region (VH) comprising an amino acid sequence that is at least 75%, at least 80%, at least 85%, at least 90%, at least 95%, or at least 99% identical to the amino acid sequence of SEQ ID NO: 3, 5, 7, 9 or 11, and a light chain variable region (VL) comprising an amino acid sequence that is at least 75%, at least 80%, at least 85%, at least 90%, at least 95%, or at least 99% identical to the amino acid sequence of SEQ ID NO: 4, 6, 8, 10 or 12. In some embodiments, the first antigen binding portion comprises a heavy chain variable region (VH) comprising an amino acid sequence of SEQ ID NO: 15, and a light chain variable region (VL) comprising an amino acid sequence of SEQ ID NO: 16; and the second antigen binding portion comprises a heavy chain variable region (VH) comprising an amino acid sequence of SEQ ID NO: 3, 5, 7, 9, or 11, and a light chain variable region (VL) comprising an amino acid sequence of SEQ ID NO: 4, 6, 8, 10, or 12.

In some embodiments, biparatopic anti-CDH17 antibody or antigen-binding fragment thereof is or comprises a F(ab′)₂, a F(ab)₂, IgG-scFv, tandem scFv and an antibody.

In some embodiments, the biparatopic anti-CDH17 antibody or antigen-binding fragment thereof comprises a first Fab fragment (Arm 1) comprising a first heavy chain variable region and a first light chain variable region and a second Fab fragment (Arm 2) comprising a second heavy chain variable region and a second variable light chain region, wherein the first heavy chain variable region, the first light chain variable region, the second heavy chain variable region, and the second variable light chain region, respectively, comprise amino acid sequences that are at least 90%, at least 95%, or at least 99% identical to the amino acid sequences of
(a) SEQ ID NO: 88, SEQ ID NO: 89, SEQ ID NO: 90, and SEQ ID NO: 91;
(b) SEQ ID NO: 88, SEQ ID NO: 89, SEQ ID NO: 92 and SEQ ID NO: 93;
(c) SEQ ID NO: 94, SEQ ID NO: 95, SEQ ID NO: 96, and SEQ ID NO: 97;
(d) SEQ ID NO: 117, SEQ ID NO: 118, SEQ ID NO: 119, and SEQ ID NO: 120; or
(e) SEQ ID NO: 117, SEQ ID NO: 118, SEQ ID NO: 121, and SEQ ID NO: 122.

In some embodiments, the first heavy chain variable region, the first light chain variable region, the second heavy chain variable region, and the second variable light chain region, respectively, comprises an amino acid sequence of
(a) SEQ ID NO: 88, SEQ ID NO: 89, SEQ ID NO: 90, and SEQ ID NO: 91;
(b) SEQ ID NO: 88, SEQ ID NO: 89, SEQ ID NO: 92 and SEQ ID NO: 93;
(c) SEQ ID NO: 94, SEQ ID NO: 95, SEQ ID NO: 96, and SEQ ID NO: 97;
(d) SEQ ID NO: 117, SEQ ID NO: 118, SEQ ID NO: 119, and SEQ ID NO: 120; or
(e) SEQ ID NO: 117, SEQ ID NO: 118, SEQ ID NO: 121, and SEQ ID NO: 122.

In some embodiments, the biparatopic anti-CDH17 antibody or antigen-binding fragment thereof is or comprises a F(ab′)₂, a F(ab)₂, IgG-scFv, tandem scFv and a bispecific antibody.

### Biparatopic Anti-CDH17 Antibody Drug Conjugates

Antibody-drug conjugates (ADCs) are a class of highly potent antibody-based cancer therapeutics. ADCs consist of recombinant antibodies covalently linked to cytotoxic agents (known as payloads) via synthetic linkers. ADCs combine the specificity of monoclonal antibodies and the potency of small-molecule chemotherapy drugs to facilitate the targeted delivery of highly cytotoxic small molecule drug moieties directly to tumor cells. CDH17 is suitable for use as ADC targets as characterized by two important properties: (i) high expression level by the target cell and limited or no expression in normal tissue, and (ii) internalization (e.g., efficient internalization) in response to antibody binding.

Utilizing a biparatopic antibody-drug conjugate (ADC) offers advantages in terms of simplicity, specificity, efficacy, and patient convenience compared to using a combination of two separate ADCs.

A biparatopic ADC enhances target specificity and avidity by simultaneously binding to two distinct epitopes on the target antigen. This dual-binding capability increases the selectivity for tumor cells overexpressing the target antigen while minimizing off-target effects on normal tissues, thereby improving the therapeutic index and reducing potential toxicity. A biparatopic ADC may exhibit synergistic anti-tumor activity compared to a combination of two separate ADCs, leading to improved efficacy in preclinical and clinical settings.

A biparatopic ADC streamlines the drug development process by consolidating two therapeutic agents into a single molecule, reducing the complexity associated with manufacturing, characterization, and regulatory approval.

Moreover, the use of a biparatopic ADC simplifies dosing regimens and treatment schedules for patients, as they only need to receive one therapeutic agent instead of multiple drugs. This enhances patient compliance and convenience while potentially reducing healthcare costs associated with drug administration and monitoring.

Examination of internalization by the disclosed CHD17 biparatopic ADC candidates using the indirect pH Dye method showed ECD 3-5 and ECD 6-7 binding mAbs have a synergistic effect of increased internalization when combined compared to mAb alone. With the increased internalization, there was also enhanced cytotoxicity of tumor cells by the biparatopics compared to mAb alone or the combination of mAbs.

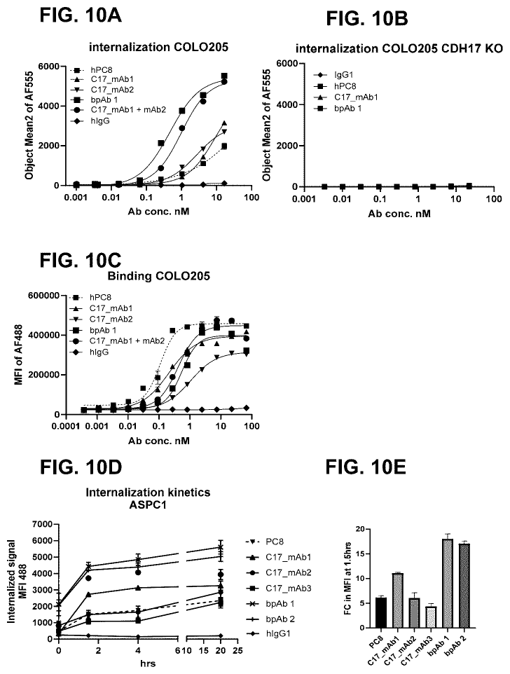

The disclosed biparatopic antibodies targeting different CDH17 epitopes show enhanced internalization and cytotoxicity compared to combinations of mAbs or mAbs alone. The combination of mAbs of our disclosed antibodies show enhanced synergistic effect compared to mAbs alone. However, the biparatopic antibodies show even greater synergistic effect that is not driven by binding as shown in FIG. 10.

The greater synergistic internalization displayed by the biparatopic is also preserved in cancer cells expressing varying levels of CDH17. The disclosed biparatopic antibodies showed greater internalization in HT29 which has low levels of CDH17 at approximately 15K/cell.

### Bispecific Anti-CDH17 T Cell Engager

Bispecific antibodies provide a recognized therapeutic strategy for redirecting immune effector cells against tumor cells. In one embodiment, a bispecific antibody targeting CDH17 and a T cell receptor component (i.e., CD3 epsilon) may stimulate T cells to initiate an anti-tumor response. In an alternative embodiment, a bispecific antibody targeting CDH17 and a T cell costimulatory receptor (e.g., CD137 or CD28) may stimulate CD8+ effector T cells to decrease tumor growth and/or kill tumor cells. Clinical development of a bispecific or multi-specific binding compound (e.g., a bispecific antibody) comprising an anti-CDH17 binding subunit may be efficacious in treating patients with various cancers that express CDH17.

In certain embodiments, the anti-CDH17 antibodies, or fragments of the antibodies can be incorporated into a bispecific antibody (bsAb) or a multi-specific antibody. These antibodies contain at least one antigen-binding antibody domain and possess functions that can be additive, synergistic, or obligative from the comprising components. In one embodiment, the bsAb comprises one binding element that binds CDH17 and a second element that binds to an immune cell surface protein such as CD3, CD28, CD137, OX40, CD27, CD16 or CD40.

Suitable anti-CD3 sequences for the CD3-binding component in a bispecific or multi-specific antibody can bind to one or more subunits of the CD3-TCR complex and activate T cells. Some of the anti-CD3 antibodies may cross-react with non-human primate CD3. The CD3-binding component can comprise complementarity-determining regions (CDRs) derived from a group of CD3 antibodies, such as OKT3, UCHT-1, SP34, and Cris-7. In an embodiment, the bispecific antibody comprises a means for binding CD3 (e.g., CDRs derived from OKT3, UCHT-1, SP34, or Cris-7). An exemplary list of the anti-CD3 antibody may be found in WO 2016/187594A1.

T cell engager (TCE) is a type of bispecific or multi-specific antibody that connects tumor cells to T cells and activates T cells. In certain embodiments, the bispecific or multi-specific antibodies containing anti-CDH17 antibodies or antigen binding fragments can also bind to the CD3 subunits on T cells. The valency for respective anti-CDH17 or anti-CD3 component can be 1 or 2. In certain embodiments, the bispecific or multi-specific antibodies contain an Fc domain. Formats of the bispecific or multi-specific antibody may be found in, for example, Labrijn et al., Bispecific antibodies: a mechanistic review of the pipeline, Nature Review Drug Discovery 18:585-608 (2019); Brinkmann et al., The making of bispecific antibodies, MAbs. February/March 2017; 9 (2): 182-212; Dickopf et al., Format and geometries matter: Structure-based design defines the functionality of bispecific antibodies, Comput Struct Biotechnol J 18, 1221-1227 (2020), and Gu et al., Biology drives the discovery of bispecific antibodies as innovative therapeutics, Antibody Therapeutics, 2020; 3:18-62.

Methods for the preparation of bispecific and trispecific antibodies may be found in, for example, Dimasi et al., Molecular engineering strategies and methods for the expression and purification of IgG1-based bispecific bivalent antibodies. Methods 154, 77-86 (2019). Wu et al., Building blocks for bispecific and trispecific antibodies. Methods. 2019 Feb. 1; 154:3-9; Sedykh et al., Bispecific antibodies: design, therapy, perspectives. Drug Des Devel Ther., 12:195-208 (2018); and Li et al. Protocol for high-throughput cloning, expression, purification, and evaluation of bispecific antibodies. STAR Protocols 3 (2022), the contents of which are incorporated herein by reference.

A bispecific TCE containing anti-CDH17 component may have the following desired features: i) binds to CDH17 with high specificity and affinity; ii) is able to bind to CD3 on T cells concurrently; iii) induces CD3-mediated signaling in T cells in a CDH17-dependent manner; iv) confers T cell-mediated, and more importantly, CDH17-dependent target cell cytotoxicity; and v) manifests good safety profile without strong cytokine release syndrome (CRS) and other toxicities.

The anti-CDH17 x CD3 antibodies targeting CDH17 and CD3 have been synthesized in IgG-ScFv format which is illustrated in FIG. 13A. The heterodimeric Fc and hinge are presented with chain A in white and chain B in black. The ScFv binding to the first antigen-binding domain (CD3) is depicted in grey, while the second antigen-binding domain (CDH17) is shown in hatched fill. To enhance the heterodimer formation of Fc for improved production, “knob in hole” mutations on CH3 Carter P. et al. (1998) were applied. And to eliminate FcγR-binding the “LALAPA” mutations are introduced in the Fc region. These Fc mutations are provided in FIG. 13B (code by EU numbering).

Suitable methods to evaluate the specificity and activity of CDH17 TCE bispecific antibodies include but are not limited to target cell and T cell binding assays, T cell signaling/activation reporter assays in the presence or absence of CDH17 target cells, CDH17 tumor associated antigen (TAA) dependent T cell activation and cytotoxicity assays, PBMC activation assays, growth inhibition of a CDH17+ tumor in an in vivo murine model comprising humanized T cells.

In a particular embodiment, a CDH17 x CD3 epsilon bispecific antibody may cause T cell dependent cytotoxicity of CDH17 positive tumor cells. The CDH17 x CD3 bispecific antibody works by binding simultaneously to CDH17 on cancer cells and CD3 receptors on T cells. This connection activates the T cells, triggering an immune response directed at the CDH17+ cancer cells. Activated T cells release cytotoxic substances and immune signaling molecules, leading to targeted destruction of the cancer cells.

In another embodiment, a CDH17 x costimulatory T cell receptor (e.g., CD137 or CD28) bispecific or multi-specific antibody may selectively activate the CD137 or CD28 co-stimulatory pathway in the tumor microenvironment. The bispecific/multi-specific antibodies bind to both human CDH17 expressed on the surfaces of cancer and human CD137 or CD28 proteins expressed on immune cells, respectively. The simultaneous binding can drive co-stimulatory agonism in CDH17 expressing tumors resulting in tumor-experienced T cell activation, expansion and ameliorated T cell exhaustion. This conditional activation of co-stimulatory pathway also prevents systemic T cell activation and reduces systemic toxicity.

In some embodiments, the bispecific T cell engager antibody comprises a first anti-CD3 scFv fragment (Arm 1) and a second Fab fragment (Arm 2) comprising a heavy chain variable region and a light chain variable region specific for CDH17. In some embodiments, the heavy chain variable region and the light chain variable region of the Fab fragment, respectively, comprise the amino acid sequences of:
(a) SEQ ID NO: 100 and SEQ ID NO: 101;
(b) SEQ ID NO: 102 and SEQ ID NO: 103;
(c) SEQ ID NO: 104 and SEQ ID NO: 105; or
(d) SEQ ID NO: 125 and SEQ ID NO: 126.

In some embodiments, the anti-CD3 scFv fragment comprises an amino acid sequence of SEQ ID NO: 98 or SEQ ID NO: 127. In some embodiments, the anti-CD3 scFv fragment, the heavy chain variable region and the light chain variable region, respectively, comprise the amino acid sequences of:
(a) SEQ ID NO: 98, SEQ ID NO: 100 and SEQ ID NO: 101;
(b) SEQ ID NO: 98, SEQ ID NO: 102 and SEQ ID NO: 103;
(c) SEQ ID NO: 98, SEQ ID NO: 104 and SEQ ID NO: 105;
(d) SEQ ID NO: 98, SEQ ID NO: 125 and SEQ ID NO: 126; or
(e) SEQ ID NO: 127, SEQ ID NO: 125 and SEQ ID NO: 126.

In some embodiments, the bispecific T cell engager antibody comprises a first antigen-binding portion having specificity to a human CDH17 protein and a second portion having specificity to human CD3, wherein the first antigen-binding portion comprises a Fab heavy chain comprising the amino acid sequence of SEQ ID NO: 125 and a Fab light chain comprising the amino acid sequence of SEQ ID NO: 126 and the second portion having specificity to human CD3 comprises an amino acid sequence selected from SEQ ID NO: 98 or SEQ ID NO: 127.

In some embodiments, the bispecific T cell engager antibody comprises a first antigen-binding portion having specificity to a human CDH17 protein and a second portion having specificity to human CD3, wherein the first antigen-binding portion comprises a Fab heavy chain comprising the amino acid sequence of SEQ ID NO: 125 and a Fab light chain comprising the amino acid sequence of SEQ ID NO: 126 and the second portion having specificity to human CD3 comprises the amino acid sequence of SEQ ID NO: 98.

In some embodiments, the bispecific T cell engager antibody comprises a first antigen-binding portion having specificity to a human CDH17 protein and a second portion having specificity to human CD3, wherein the first antigen-binding portion comprises a Fab heavy chain comprising the amino acid sequence of SEQ ID NO: 125 and a Fab light chain comprising the amino acid sequence of SEQ ID NO: 126 and the second portion having specificity to human CD3 comprises the amino acid sequence of SEQ ID NO: 127. In some embodiments, the bispecific T cell engager antibody comprises a first anti-CD3 scFv fragment (Arm 1) and a second Fab fragment (Arm 2) comprising a heavy chain variable region and a light chain variable region specific for CDH17, wherein the heavy chain variable region and the light chain variable region, respectively, comprise the amino acid sequences of SEQ ID NO: 125 and SEQ ID NO: 126; and wherein the anti-CD3 scFv fragment comprises the amino acid sequence of SEQ ID NO: 98.

In some embodiments, the bispecific T cell engager antibody comprises a first anti-CD3 scFv fragment (Arm 1) and a second Fab fragment (Arm 2) comprising a heavy chain variable region and a light chain variable region specific for CDH17, wherein the heavy chain variable region and the light chain variable region, respectively, comprise the amino acid sequences of SEQ ID NO: 125 and SEQ ID NO: 126; and wherein the anti-CD3 scFv fragment comprises the amino acid sequence of SEQ ID NO: 127.

In some embodiments, bispecific anti-CDH17 antibody or antigen-binding fragment thereof is or comprises a F(ab′)2, a F(ab)2, IgG-scFv, tandem scFv, and a bispecific antibody.

### Anti-CDH17 Target Indications/Therapeutic Strategies

Dysregulation of CDH17 has been identified as one of the important mechanisms of tumor development for gastrointestinal cancers like colon, gastric, liver, and pancreatic cancers (Berx, G. and F. van Roy (2009). Cold Spring Harb Perspect Biol 1(6): a003129).

Downregulation of CDH17 in colon cancer specimens was strongly linked to disease progression and lymph node metastasis, with consistent expression observed between primary and metastatic sites (Wong, K. K. (2023). Comput Biol Chem 105:107897). In gastric cancer, CDH17's significance was demonstrated in a clinical study involving 71 gastric cancer patients, where its expression correlated positively with histological type, tumor invasion, and lymph node metastasis.

In gastric adenocarcinoma cell lines like BGC-823 and MKN-45, CDH17 overexpression facilitated proliferation, invasion, and migration while its downregulation inhibited these processes, concurrently suppressing the NF-κB signaling pathway and downstream proteins like VEGF-C and MMP-9 (Wang, J., et al. (2013). Cancer Biol Ther 14 (3): 262-270).

In liver cancer, RT-PCR analysis of 57 cases indicated significantly elevated CDH17 levels in cancerous tissues compared to normal. Additionally, splice isoforms and gene polymorphisms of CDH17 were implicated in hepatocellular carcinogenesis risk. (Wong, B. W., et al. (2003). Biochem Biophys Res Commun 311(3): 618-624); Lee, N. P., et al. (2010). Biochim Biophys Acta 1806(2): 138-145). In intrahepatic cholangiocarcinoma, CDH17 was associated with tumor differentiation and vascular invasion. Knockdown experiments revealed its involvement in promoting MTF-1 and PLGF expression, thereby stimulating tumor angiogenesis (Zheng, B. H., et al. (2021). J Surg Oncol 123(5): 1253-1262). CDH17 immunoreactivity in esophageal adenocarcinoma correlated with clinical characteristics, with higher levels detected in well-differentiated tissues. In esophageal squamous cell carcinoma, CDH17 CpG island methylation was also enhanced (Shenoy, U. S., et al. (2022). Cell Biol Toxicol 38(1): 1-30; Ignatova, E. O., et al. (2022). World J Gastrointest Oncol 14(3): 628-645).

In pancreatic cancer, higher CDH17 expression was associated with well-differentiated carcinomas and better prognostic survival. Conversely, low CDH17 expression correlated with tumor dedifferentiation, although further research is needed to elucidate its full implications in clinicopathological features (Liu, X., et al. (2019). Cancer Letters 454:204-214).

CDH17's involvement extends beyond gastrointestinal cancers, with elevated levels observed in ovarian cancer and intraductal papillary mucinous neoplasm (IPMN). Its overexpression in ovarian epithelial carcinoma was linked to poor prognosis, while it plays a role in promoting intestinal-type differentiation in IPMN (Huang, L. P., et al. (2012). Int J Gynecol Cancer 22(7): 1170-1176; Karimi, S. S., et al. (2021). American Journal of Clinical Pathology 156 (Supplement_1): S63-S63). Overall, CDH17 holds potential for diagnosing and predicting the progression and recurrence of various cancers, including gastric, hepatocellular, and colorectal cancers.

CDH17 emerges as an optimal target for cancer immunotherapy because of its low restricted to transmembrane expression in normal tissues and high expression in colorectal, gastrointestinal, mucinous ovarian cancer, pancreatic and cervical adenocarcinoma (Jacobsen, F., et al., Pathology-Research and Practice, (2024)). In addition, elevated CDH17 expression has been unequivocally linked to the metastatic advancement observed across various neoplasms, particularly those originating from the gastrointestinal tract (Casal, J. I. and R. A. Bartolome et al., Int J Mol Sci, 20(13), (2019)).

In the competitive landscape of anti-CDH17 therapeutics, three prominent modalities have emerged: Antibody-Drug Conjugates (ADCs), Bispecific Antibodies (BsAbs), and Chimeric Antigen Receptor T cell Therapy (CAR-T). Multiple clinical agents targeting CDH17 are presently in various stages of development, predominantly focusing on gastrointestinal cancers. Among these agents, there are three bispecific antibodies: ARB-202 (CDH17 x CD3), ARB-001.T (CDH17 x CD3), and BI-905711 (CDH17 x DR5/TRAIL-R2), alongside one CAR-T therapy named CHM-2101, and Anti-CDH17-based antibody drug conjugates named TORL-3-600 (TORL Biotherapeutics).

A humanized CDH17-CD3 bispecific T-cell engager antibody, known as ARB202, was generated from the anti-CDH17 monoclonal antibody ARB102 (Lum Y et al., Mol. Pharm., 2020), by conjugating a CD3-binding scFv in IgG4-scFv format. The binding affinity (Kd [M]) of ARB202 to CDH17 and CD3 was observed at 10⁻¹⁰ and 10⁻⁹, respectively, with cellular affinities of 10⁻⁹ and 10⁻⁸. In vitro cytotoxicity assays demonstrated that ARB202-mediated tumor cell killing relied on CDH17 expression and necessitated immune synapse formation with effector T cells, while normal cells and CDH17-negative cancer cells remained resistant. In a pancreatic AsPC1 xenograft model, ARB202 (co-inoculated with human PBMC) exhibited dose-dependent inhibition of tumor growth. Consistent human IL-2 release in mice blood samples corroborated functional CDH17 and CD3 engagement (Wong, D. A., et al., Journal of Clinical Oncology, 39(3_suppl): 405-405, (2021)).

Bartolome et al showed that targeting RGD motif on CDH17 using antibodies led to reduction in cell metastasis and tumor growth via the inactivation of wnt/β-catenin pathway (Bartolome, R. A., et al., Clin Cancer Res, 24(2): 433-444, (2018)).

BI905711 is a tetravalent bispecific antibody designed to cross-link TRAILR2 with CDH17. CDH17 is used as an anchor for a tissue selective function designed to foster CDH17-dependent TRAILR2 oligomerization, leading to caspase activation and subsequent apoptosis, thereby inhibiting tumor growth in preclinical gastrointestinal cancer models. In an on-going Phase I/II study, BI 905711 was associated with a tolerable safety profile and early signs of disease control. (Garcia-Martinez et al, 2020, MCT-AACR′ 2020, Harding, J. J., et al., Journal of Clinical Oncology, 41(4_suppl): 115-115, (2023)).

A CAR T-cell therapy, designated as CHM 2101 and targeting CDH17, has advanced into clinical trials for the treatment of gastrointestinal (GI) cancers. This third-generation VHH1-CAR, which includes both CD28 and 4-1BB (VHH1-28BBz) effectively eradicated CDH17-expressing NETs and gastric, pancreatic and colorectal cancers in either tumor xenograft or autochthonous mouse models, all while avoiding toxicity to healthy tissues (Feng, Z. et al., Nat Cancer, 3, 581-594, (2022)).

Slamon, D et al from UCLA reported the development of CDH17-ADCs (WO2023/107558). In preclinical CDX tumor studies, the MMAE conjugated CDH17 antibodies were able to induce strong anti-tumor efficacies in CDH17 positive tumors, HPAFII, LS513, SNU-C1, Patu8988S. The kinetics of the internalization was also evaluated and the evaluated antibodies described as “fast internalizers” with cytotoxic effects beginning at 2 hrs, and reaching maximum effect at 24 hrs.

Overall, CDH17 holds potential for diagnosing and predicting the progression and recurrence of various cancers, including gastric, hepatocellular, and colorectal cancers.

### Anti-CDH17 Combination Strategies

Anti-CDH17 combinational immunotherapy can involve using multiple immunotherapeutic agents simultaneously or sequentially to enhance treatment efficacy. This strategy aims to address the complexity and heterogeneity of cancer, overcoming tumor evasion mechanisms and maximizing therapeutic outcomes. One common approach is the combination of checkpoint inhibitors, such as anti-PD-1/PD-L1 and anti-CTLA-4 antibodies. Additionally, combining checkpoint inhibitors with other immunotherapies, such as cancer vaccines or adoptive cell therapies, can further potentiate immune-mediated tumor destruction. Furthermore, combining immunotherapies with conventional treatments like chemotherapy or radiotherapy can induce immunogenic cell death, releasing tumor antigens and activating immune responses against cancer cells.

Combining anti-CDH17 therapies with checkpoint inhibitors emerges as a promising strategy for managing CRC patients. The expression of CDH17 was notably reduced in colorectal cancer (CRC) cases exhibiting high microsatellite instability, and its levels correlate negatively with immune response gene sets and the expression of MHC class I and II molecules in CRCs (Wong, K. K. Comput Biol Chem 105:107897, 2023). These observations imply a potential role for CDH17 in suppressing the immune response in CRC cases, while its reduced expression in MSI-H CRCs may enhance immunogenicity against MSI-H tumors, consistent with the known association between MSI-H CRC tumors and heightened immune cell infiltration due to frequent presentation of immunogenic neoantigens resulting from frameshift mutations. Consequently, metastatic MSI-H CRC patients, but not those with microsatellite stable (MSS) tumors, show responsiveness to immune checkpoint blockade therapy (Cohen, R. et, al, Target Oncol. 15 (1), 11-24. 2020). Therefore, targeting CDH17 in MSS CRCs might enhance tumor immunogenicity and lymphocytic infiltration for their eradication.

Anti-CDH17 antibodies may be combined with one or more chemotherapies. The optimal combination of CDH17-target therapy with chemotherapeutic agents requires a better understanding of the unique cell cycle interactions and the modulation of surface antigen expression by the cytotoxic partner. The combinations of ADC with cytotoxic chemotherapy are challenged by overlapping toxicities, which may be lessened with newer generation and more tumor-selective ADCs. T cell engager BsAbs are targeting immune cells which potentially can benefit from the immunogenic cell death indued by chemotherapy. The chemotherapeutic agent may upregulate the expression of surface antigens thus enhance the efficacy of targeted therapy (Wei, et al., Front Immunol, 13:1035276, 2022; Kan, et al., BMC Cancer, 15, 726, 2015).

Anti-CDH17 antibodies may be combined with one or more antiangiogenic agents. Anti-angiogenic agents could potentially enhance the penetration and exposure of tumor cells to CDH17-targeted drugs. Notably impactful outcomes, including complete responses, have been demonstrated when anetumab ravtansine or mirvetuximab soravtansine were combined with bevacizumab in preclinical models of ovarian cancer. These promising preclinical results were corroborated by a recent Phase 1b study that investigated the combination of mirvetuximab soravtansine and bevacizumab in patients with heavily treated, platinum-resistant, FRα-high ovarian cancer. Impressively, the overall response rate (ORR) of 39% surpassed the benchmark set by the pivotal AURELIA trial (27%). It is noteworthy that the expected overlapping toxicity, grade 3 thrombocytopenia, was observed in only one instance (O'Malley, et al., Gynecol. Oncol. 157, 379-385.2020; Pujade-Lauraine, E. et al. J. Clin. Oncol. 32, 1302-1308, 2014).

However, the success of combinational immunotherapy relies on careful selection of agents, dosing schedules, and patient stratification based on biomarkers and tumor characteristics. Moreover, managing potential toxicities and immune-related adverse events is crucial for optimizing treatment outcomes and patient safety.

Overall, combinational immunotherapy represents a promising approach to improve cancer treatment outcomes by exploiting the complexity of the immune system and the tumor microenvironment. Continued research efforts aimed at identifying optimal combinations and treatment regimens will further advance the field and bring us closer to achieving durable responses and long-term cancer control.

### Antibody Drug Conjugates (ADCs)

The present disclosure provides antibody-drug conjugates (ADCs) comprising an anti-CDH17 antibody or antigen-binding fragment thereof conjugated to a therapeutic moiety such as a cytotoxic agent, a chemotherapeutic drug, or a radioisotope.

Cytotoxic agents include any agent that is detrimental to the growth, viability or propagation of cells. Examples of suitable cytotoxic agents and chemotherapeutic agents that can be conjugated to anti-CDH17 antibodies in accordance with this aspect of the present disclosure include, e.g., 1-(2chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins, camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing. According to certain embodiments, the cytotoxic agent that is conjugated to an anti-CDH17 antibody is a maytansinoid such as DM1 or DM4, a tomaymycin derivative, or a dolastatin derivative. According to certain embodiments, the cytotoxic agent that is conjugated to an anti-CDH17 antibody is an auristatin such as MMAE, MMAF, or derivatives thereof. Other cytotoxic agents known in the art are contemplated within the scope of the present disclosure, including, e.g., protein toxins such ricin, C. difficile toxin, pseudomonas exotoxin, ricin, diphtheria toxin, botulinum toxin, bryodin, saporin, pokeweed toxins (i.e., phytolaccatoxin and phytolaccigenin), and others such as those set forth in Sapra et al., Pharmacol. & Therapeutics, 2013, 138:452-469.

The present disclosure also includes antibody-radionuclide conjugates (ARCs) comprising anti-CDH17 antibodies conjugated to one or more radionuclides. Exemplary radionuclides that can be used in the context of this aspect of the present disclosure include, but are not limited to, e.g., ²²⁵Ac, ²¹²Bi, ²¹³Bi, ¹³¹I, ¹⁸⁶Re, ²²⁷Th, ²²²Rn, ²²³Ra, ²²⁴Ra, and ⁹⁰Y.

The generation of antibody-drug conjugates can be accomplished by any technique known to the skilled artisan using any suitable synthetic linker and conjugation chemistry.

Linkers are classified into two broad categories: cleavable and non-cleavable. Cleavable linkers exploit the differences between normal physiologic conditions in the bloodstream and the intracellular conditions present in the cytoplasm of cancer cells (Peters, et al., Biosci Rep, 35(4): e00225, 2015). Changes in the microenvironment after an ADC-antigen complex is internalized, triggers cleavage of the linker and releases the cytotoxic payload, effectively targeting toxicity to cancer cells expressing the target antigen. Generally, non-cleavable ADCs are primarily effective against target antigen-expressing cells and are best suited to treat cancers that have high and homogenous expression of the target antigen (Kovtun, et al., Cancer Res, 66(6): 3214-3221, 2006).

Non-cleavable linkers depend solely on the process of lysosomal degradation following ADC-antigen internalization. After internalization of the ADC-antigen complex protease enzymes within the lysosome degrade the protein structure of the antibody, leaving a single amino acid (typically a cysteine or a lysine) attached to the linker and the cytotoxic agent that is released into the cytoplasm as the active drug. Published reports suggest that ADCs with an acid-labile cleavable linker may not require internalization for therapeutic potency. This finding is largely attributed to the fact that the TME is much more acidic than normal tissues and can comprise proteases capable of mediating extracellular cleavage of the chemically labile (disulphide and pH-dependent) and enzyme-labile (peptide-based) cleavable linkers, Depending on the linker type and drug combination, non-internalizing ADCs may have additional advantages by exploiting pathological features inherent to the microenvironment of many tumors such as hypoxia, necrosis, excess reducing equivalents, acidity, an abundance of both active extracellular proteases and protease-rich tumor-infiltrating myeloid cells (Staudacher, et al., Br J Cancer, 117:1736-1742, 2017).

The present disclosure comprises ADCs in which a linker connects an anti-CDH17 antibody or antigen-binding molecule to a drug or cytotoxin through an attachment at a particular amino acid within the antibody or antigen-binding molecule. Exemplary amino acid attachments that can be used in the context of this aspect of the present disclosure include, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313).

There are many techniques for chemical modification of proteins suitable for use in the conjugation of the linker-payload to a CDH17-specific antibody. Additionally, different methods of conjugation chemistry will afford different levels of control over the number and site of drug attachment and potentially impact the pharmacokinetics, toxicity and therapeutic window of an anti-CDH17 ADC. Antibody-drug conjugates can be prepared by binding the drug to an antibody in accordance with a conventional technique. Techniques for conjugating a therapeutic moiety to antibodies are well known to those of skill in the art, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Site-specific conjugation chemistry methods are intended to produce relatively homogenous ADC products without altering the binding affinity of the antibody. Generally, three strategies are used for site-specific conjugation on antibodies: i) use of engineered cysteines, ii) incorporation of unnatural amino acids, and iii) enzymatic conjugations using reaction sites of antibodies that are designed to react specifically to a bacterial enzyme (e.g., transglutaminases, glycotransferases, sortases or formyl glycine generating enzyme) that generate post-translational modifications of proteins in a site-specific manner.

Techniques for the site-specific conjugation of a therapeutic moiety to antibodies are well known and include, but are not limited to, the methods disclosed in U.S. Pat. Nos. 7,723,485; 8,937,161; 9,000,130; 9,884,127; 9,717,803; 9,669,107; 10,583,197; 10,639,291; 10,357,472; U.S. Patent Application Publication Nos.: US 2015/0283259; US 2017/0362334; US 2018/0140714; and International Publication Nos.: WO2012/153193; WO 2013/092983; WO 2013/092998; WO 2014/072482; WO 2014/202773; WO 2014/202775; WO 2015/155753; WO 2015/191883; WO 2016/102632; WO 2017/059158; WO 2018/140590, WO 2018/185526, WO 2023/025243 and WO 2023/088382.

ADCs can be designed to kill not only target antigen positive cells but also other cells in the vicinity, irrespective of the expression of the target antigen on their surface by a mechanism commonly referred to as the “bystander effect” (Kovtun et al., Cancer Res, 66 (6): 3214-21, 2006). Bystander killing occurs when a cytotoxic payload from an ADC is released, either by degradation of the ADC following internalization by the target cell or by the action of protease present in the extracellular space on cleavable linkers. In both cases, the released cytotoxic drug is then taken up by and kills surrounding or bystander cells, which themselves may or may not express the ADC target antigen.

How much a particular ADC mediates bystander killing depends largely on factors such as the extent of ADC internalization after binding to the target antigen, the presence of a non-cleavable or cleavable linker, and the hydrophobicity of the attached cytotoxic warhead. Although the bystander effect undermines the concept of the absolute target specificity of ADCs, it can be advantageous when treating solid tumors that lack homogenous expression of the target antigen.

One of skill in the art will understand how to determine if a particular anti-CDH17 antibody is suitable for development as an ADC. For example, the binding affinity of the naked antibody and an ADC prepared using the antibody can be assessed using cells engineered to overexpress the target antigen (e.g., transfected CHO cells) or human cells lines endogenously expressing CDH17 at high, medium, and low densities such as AsPC1 and Capan2 (pancreatic cancer cells), SNU16 (gastric cancer cells), COLO205 and HT29 (colorectal cancer cells). The internalization capability and the rate of internalization using the pH dye method on directly labeled antibody or using secondary antibody can be assessed in the same cell lines. In addition, in vitro cytotoxicity can be assessed in the same cell lines and bystander cytotoxicity can be evaluated using a co-culture assay with a mixture of CDH17+ cells and CDH17-cells.

The in vivo potency of selected ADCs can be evaluated in CDX and PDX tumor models expressing high, low CDH17 expression. Additionally, pharmacokinetic assessment, in vivo determination of Drug-Antibody Ratio Stability and Single-Dose Toxicity Study can be evaluated in rodents and non-human primate studies.

### Polynucleotides, Vectors and Host Cells

Other embodiments encompass isolated polynucleotides that comprise a sequence encoding an anti-CDH17 antibody or antigen binding fragment thereof, vectors, and host cells comprising the polynucleotides, and recombinant techniques for production of the antibody. The isolated polynucleotides can encode any desired form of the anti-CDH17 antibody including, for example, full length monoclonal antibodies, linear antibodies, single-chain antibodies, chimeric antibodies, humanized antibodies, bispecific antibodies, and multi-specific antibodies (e.g., formed from antigen binding fragments). The isolated polynucleotides may also encode diabodies, Fab, Fab′, F(ab)₂, or Fv fragments.

Some embodiments include isolated polynucleotides comprising sequences that encode the heavy chain variable region of an antibody or antigen binding fragment having the amino acid sequence of SEQ ID NOs: 1, 3, 5, 7, 9, 11, 13, 15, 17, or 19, or an amino acid sequence having at least 90%, 95%, 96%, 97%, 98%, or 99% sequence identity thereto. Some embodiments include isolated polynucleotides comprising sequences that encode the light chain variable region of an antibody or antigen binding fragment having the amino acid sequence of any of SEQ ID NOs: 2, 4, 6, 8, 10, 12,14, 16, 18, or 20, or an amino acid sequence having at least 90%, 95%, 96%, 97%, 98%, or 99% sequence identity thereto.

In an embodiment, the isolated polynucleotide sequence(s) encodes an antibody or antigen binding fragment having a heavy chain variable region and a light chain variable region comprising:
(a) a variable heavy chain sequence comprising SEQ ID NO: 1 and a variable light chain sequence comprising SEQ ID NO: 2;
(b) a variable heavy chain sequence comprising SEQ ID NO: 3 and a variable light chain sequence comprising SEQ ID NO: 4;
(c) a variable heavy chain sequence comprising SEQ ID NO: 5 and a variable light chain sequence comprising SEQ ID NO: 6;
(d) a variable heavy chain sequence comprising SEQ ID NO: 7 and a variable light chain sequence comprising SEQ ID NO: 8;
(e) a variable heavy chain sequence comprising SEQ ID NO: 9 and a variable light chain sequence comprising SEQ ID NO: 10;
(f) a variable heavy chain sequence comprising SEQ ID NO: 11 and a variable light chain sequence comprising SEQ ID NO: 12;
(g) a variable heavy chain sequence comprising SEQ ID NO: 13 and a variable light chain sequence comprising SEQ ID NO: 14;
(h) a variable heavy chain sequence comprising SEQ ID NO: 15 and a variable light chain sequence comprising SEQ ID NO: 16;
(i) a variable heavy chain sequence comprising SEQ ID NO: 17 and a variable light chain sequence comprising SEQ ID NO: 18; or
(j) a variable heavy chain sequence comprising SEQ ID NO: 19 and a variable light chain sequence comprising SEQ ID NO: 20.

In another embodiment, the isolated polynucleotide sequence(s) encodes an antibody or antigen binding fragment having a light chain variable region and a heavy chain variable region comprising the amino acid sequences of:
(a) a variable heavy chain sequence that is 90%, 95%, 96%, 97%, 98%, or 99% identical to SEQ ID NO: 1 and a variable light chain sequence that is 90%, 95%, 96%, 97%, 98%, or 99% identical to SEQ ID NO: 2;
(b) a variable heavy chain sequence that is 90%, 95%, 96%, 97%, 98%, or 99% identical to SEQ ID NO: 3 and a variable light chain sequence that is 90%, 95%, 96%, 97%, 98%, or 99% identical to SEQ ID NO: 4;
(c) a variable heavy chain sequence that is 90%, 95%, 96%, 97%, 98%, or 99% identical to SEQ ID NO: 5 and a variable light chain sequence that is 90%, 95%, 96%, 97%, 98%, or 99% identical to SEQ ID NO: 6;
(d) a variable heavy chain sequence that is 90%, 95%, 96%, 97%, 98%, or 99% identical to SEQ ID NO: 7 and a variable light chain sequence that is 90%, 95%, 96%, 97%, 98%, or 99% identical to SEQ ID NO: 8;
(e) a variable heavy chain sequence that is 90%, 95%, 96%, 97%, 98%, or 99% identical to SEQ ID NO: 9 and a variable light chain sequence that is 90%, 95%, 96%, 97%, 98%, or 99% identical to SEQ ID NO: 10;
(f) a variable heavy chain sequence that is 90%, 95%, 96%, 97%, 98%, or 99% identical to SEQ ID NO: 11 and a variable light chain sequence that is 90%, 95%, 96%, 97%, 98%, or 99% identical to SEQ ID NO: 12;
(g) a variable heavy chain sequence that is 90%, 95%, 96%, 97%, 98%, or 99% identical to SEQ ID NO: 13 and a variable light chain sequence that is 90%, 95%, 96%, 97%, 98%, or 99% identical to SEQ ID NO: 14;
(h) a variable heavy chain sequence that is 90%, 95%, 96%, 97%, 98%, or 99% identical to SEQ ID NO: 15 and a variable light chain sequence that is 90%, 95%, 96%, 97%, 98%, or 99% identical to SEQ ID NO: 16;
(i) a variable heavy chain sequence that is 90%, 95%, 96%, 97%, 98%, or 99% identical to SEQ ID NO: 17 and a variable light chain sequence that is 90%, 95%, 96%, 97%, 98%, or 99% identical to SEQ ID NO: 18; or
(j) a variable heavy chain sequence that is 90%, 95%, 96%, 97%, 98%, or 99% identical to SEQ ID NO: 19 and a variable light chain sequence that is 90%, 95%, 96%, 97%, 98%, or 99% identical to SEQ ID NO: 20.

In an embodiment, the isolated polynucleotide sequence(s) encodes an antibody or antigen binding fragment having a heavy chain variable region and a light chain variable region comprising:
(a) a variable heavy chain sequence comprising SEQ ID NO: 76 and a variable light chain sequence comprising SEQ ID NO: 78;
(b) a variable heavy chain sequence comprising SEQ ID NO: 76 and a variable light chain sequence comprising SEQ ID NO: 79;
(c) a variable heavy chain sequence comprising SEQ ID NO: 77 and a variable light chain sequence comprising SEQ ID NO: 78;
(d) a variable heavy chain sequence comprising SEQ ID NO: 77 and a variable light chain sequence comprising SEQ ID NO: 79;
(e) a variable heavy chain sequence comprising SEQ ID NO: 80 and a variable light chain sequence comprising SEQ ID NO: 82;
(f) a variable heavy chain sequence comprising SEQ ID NO: 80 and a variable light chain sequence comprising SEQ ID NO: 83;
(g) a variable heavy chain sequence comprising SEQ ID NO: 81 and a variable light chain sequence comprising SEQ ID NO: 82;
(h) a variable heavy chain sequence comprising SEQ ID NO: 81 and a variable light chain sequence comprising SEQ ID NO: 83;
(i) a variable heavy chain sequence comprising SEQ ID NO: 84 and a variable light chain sequence comprising SEQ ID NO: 86;
(j) a variable heavy chain sequence comprising SEQ ID NO: 84 and a variable light chain sequence comprising SEQ ID NO: 87;
(k) a variable heavy chain sequence comprising SEQ ID NO: 85 and a variable light chain sequence comprising SEQ ID NO: 86;
(l) a variable heavy chain sequence comprising SEQ ID NO: 85 and a variable light chain sequence comprising SEQ ID NO: 87; or
(m) a variable heavy chain sequence comprising SEQ ID NO: 123 and a variable light chain sequence comprising SEQ ID NO: 124.

In another embodiment, the isolated polynucleotide sequence(s) encodes an antibody or antigen binding fragment having a light chain variable region and a heavy chain variable region comprising the amino acid sequences of:
(a) a variable heavy chain sequence that is 90%, 95%, 96%, 97%, 98%, or 99% identical to SEQ ID NO: 76 and a variable light chain sequence that is 90%, 95%, 96%, 97%, 98%, or 99% identical to SEQ ID NO: 78;
(b) a variable heavy chain sequence that is 90%, 95%, 96%, 97%, 98%, or 99% identical to SEQ ID NO: 76 and a variable light chain sequence that is 90%, 95%, 96%, 97%, 98%, or 99% identical to SEQ ID NO: 79;
(c) a variable heavy chain sequence that is 90%, 95%, 96%, 97%, 98%, or 99% identical to SEQ ID NO: 77 and a variable light chain sequence that is 90%, 95%, 96%, 97%, 98%, or 99% identical to SEQ ID NO: 78;
(d) a variable heavy chain sequence that is 90%, 95%, 96%, 97%, 98%, or 99% identical to SEQ ID NO: 77 and a variable light chain sequence that is 90%, 95%, 96%, 97%, 98%, or 99% identical to SEQ ID NO: 79;
(e) a variable heavy chain sequence that is 90%, 95%, 96%, 97%, 98%, or 99% identical to SEQ ID NO:80 and a variable light chain sequence that is 90%, 95%, 96%, 97%, 98%, or 99% identical to SEQ ID NO: 82;
(f) a variable heavy chain sequence that is 90%, 95%, 96%, 97%, 98%, or 99% identical to SEQ ID NO: 80 and a variable light chain sequence that is 90%, 95%, 96%, 97%, 98%, or 99% identical to SEQ ID NO: 83;
(g) a variable heavy chain sequence that is 90%, 95%, 96%, 97%, 98%, or 99% identical to SEQ ID NO: 81 and a variable light chain sequence that is 90%, 95%, 96%, 97%, 98%, or 99% identical to SEQ ID NO: 82;
(h) a variable heavy chain sequence that is 90%, 95%, 96%, 97%, 98%, or 99% identical to SEQ ID NO: 81 and a variable light chain sequence that is 90%, 95%, 96%, 97%, 98%, or 99% identical to SEQ ID NO: 83;
(i) a variable heavy chain sequence that is 90%, 95%, 96%, 97%, 98%, or 99% identical to SEQ ID NO: 84 and a variable light chain sequence that is 90%, 95%, 96%, 97%, 98%, or 99% identical to SEQ ID NO: 86;
(j) a variable heavy chain sequence that is 90%, 95%, 96%, 97%, 98%, or 99% identical to SEQ ID NO: 84 and a variable light chain sequence that is 90%, 95%, 96%, 97%, 98%, or 99% identical to SEQ ID NO: 87;
(k) a variable heavy chain sequence that is 90%, 95%, 96%, 97%, 98%, or 99% identical to SEQ ID NO: 85 and a variable light chain sequence that is 90%, 95%, 96%, 97%, 98%, or 99% identical to SEQ ID NO: 86;
(l) a variable heavy chain sequence that is 90%, 95%, 96%, 97%, 98%, or 99% identical to SEQ ID NO: 85 and a variable light chain sequence that is 90%, 95%, 96%, 97%, 98%, or 99% identical to SEQ ID NO: 87; or
(m) a variable heavy chain sequence that is 90%, 95%, 96%, 97%, 98%, or 99% identical to SEQ ID NO: 123 and a variable light chain sequence that is 90%, 95%, 96%, 97%, 98%, or 99% identical to SEQ ID NO: 124.

The polynucleotide(s) that comprise a sequence encoding an anti-CDH17 antibody or antigen binding fragment thereof can be fused (e.g., operably linked) to one or more regulatory or control sequence, as known in the art, and can be contained in suitable expression vectors or host cells as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The anti-CDH17 antibodies or antigen binding fragments thereof can also be produced as fusion polypeptides, in which the antibody or fragment is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the host cell. For prokaryotic host cells that do not recognize and process the anti-CDH17 antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO 90/13646. In mammalian cells, mammalian signal sequences as well as viral secretory leaders, for example, the herpes simplex gD signal, can be used. The DNA for such precursor region is ligated in reading frame to DNA encoding the anti-CDH17 antibody.

Expression and cloning vectors contain a nucleic acid sequence that enables the vector to replicate in one or more selected host cells. Generally, in cloning vectors this sequence is one that enables the vector to replicate independently of the host chromosomal DNA and includes origins of replication or autonomously replicating sequences. Such sequences are well known for a variety of bacteria, yeast, and viruses. The origin of replication from the plasmid pBR322 is suitable for most Gram-negative bacteria, the 2-υ. plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

### Methods of Producing Antibodies

Anti-CDH17 antibodies or antigen binding fragments thereof may be made by any method known in the art. For example, a recipient may be immunized with soluble recombinant CDH17 protein or a fragment of a CDH17 peptide conjugated with a carrier protein thereof. Any suitable method of immunization can be used. Such methods can include adjuvants, other immune stimulants, repeat booster immunizations, and the use of one or more immunization routes. Any suitable source of human CDH17 can be used as the immunogen for the generation of the non-human or human anti-CDH17 antibodies of the compositions and methods disclosed herein.

Different forms of CDH17 antigens may be used to elicit an immune response for the identification of a biologically active anti-CDH17 antibody. Thus, the eliciting CDH17 antigen may be the entire CDH17 protein or a portion of a CDH17 protein comprising a single epitope, multiple epitopes, a single ECDs or a peptide comprising two or more ECDs alone or in combination with one or more immunogenicity enhancing agents. In some aspects, the eliciting antigen is an isolated soluble full-length protein, or a soluble protein comprising less than the full-length sequence (e.g., immunizing with a peptide comprising one or more of the truncated CDH17 proteins described herein (i.e., SEQ ID NO: 110 or SEQ ID NOs: 114 through 116). As used herein, the term “portion” refers to the minimal number of amino acids or nucleic acids, as appropriate, to constitute an immunogenic epitope of the antigen of interest. Alternatively, a genetic vector suitable for transformation of the cells of interest may be employed, including, but not limited to adenoviral vectors, plasmids, and non-viral vectors, such as cationic lipids.

It is desirable to prepare monoclonal antibodies (mAbs) from a mammalian host, such as mice, rodents, primates, humans, etc. Description of techniques for preparing such monoclonal antibodies may be found in, e.g., Sties et al. (eds.) BASIC AND CLINICAL IMMUNOLOGY (4th ed.) Lance Medical Publication, Los Altos, CA, and references cited therein; Harlow and Lane (1988) ANTIBODIES: A LABORATORY MANUAL CSH Press; Goding (1986) MONOCLONAL ANTIBODIES: PRINCIPLES AND PRACTICE (2nd ed.) Academic Press, New York, NY. Typically, spleen cells from an animal immunized with a desired antigen are immortalized, commonly by fusion with a myeloma cell. See Kohler and Milstein (196) Eur. J. Immunol. 6:511-519. Alternative methods of immortalization include transformation with Epstein Barr Virus, oncogene, or retroviruses, or other methods known in the art. See. e.g., Doyle et al. (eds. 1994 and periodic supplements) CELL AND TISSUE CULTURE: LABORATORY PROCEDURES, John Wiley and Sons, New York, NY. Colonies arising from single immortalized cells are screened for production of antibodies of the desired specificity and affinity for the antigen and yield of the monoclonal antibodies produced by such cells may be enhanced by various techniques, including injection into the peritoneal cavity of a vertebrate host. Alternatively, one may isolate DNA sequences which encode a monoclonal antibody or an antigen binding fragment thereof by screening a DNA library from human B cells according to the general protocol outlined by Huse et al. (1989) Science 246:1275-1281. Thus, antibodies may be obtained by a variety of techniques familiar to researchers skilled in the art.

Other suitable techniques involve selection of libraries of antibodies in phage, yeast, virus or similar vector. See e.g., Huse et al. supra; and Ward et al. (1989) Nature 341:544-546. The polypeptides and antibodies disclosed herein may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literatures. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10023; or made in transgenic mice, see Nils Lonberg et al. (1994), Nature 368:856-859; and Mendez et al. (1997) Nature Genetics 15:146-156; TRANSGENIC ANIMALS AND METHODS OF USE (WO 2012/62118), Medarex, Trianni, Abgenix, Ablexis, OminiAb, Harbour and other technologies.

In some embodiments, the ability of the produced antibody to bind to CDH17 and/or other related members of the Cadherin superfamily can be assessed using standard binding assays, such as surface plasmon resonance (SPR), ForteBio (BLI), ELISA, Western Blot, Immunofluorescent, or flow cytometric analysis. In some aspects, the produced antibody may also be assessed for its ability to induce antibody-mediated CDH17+ cell cytotoxicity, T cell derived cytotoxicity (TDCC), inflammatory cytokine release, or immune-induced cell death (ICD).

The antibody composition prepared from the hybridoma, or host cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gammal, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25 M salt).

Also included are nucleic acids that hybridize under low, moderate, and high stringency conditions, as defined herein, to all or a portion (e.g., the portion encoding the variable region) of the nucleotide sequence represented by isolated polynucleotide sequence(s) that encode an antibody or antigen binding fragment of the present disclosure. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, at least 96%, at least 97%, at least 98%, or at least 99% identical to the sequence of a portion or all of a nucleic acid encoding an anti-CDH17 polypeptide (e.g., a heavy chain or light chain variable region), or its complement. Hybridizing nucleic acids of the type described herein can be used, for example, as a cloning probe, a primer, e.g., a PCR primer, or a diagnostic probe.

### Antibody Compositions and Methods of Treatment

The disclosure also provides compositions including, for example, pharmaceutical compositions that comprise an anti-CDH17 antibody (i.e., a biparatopic CDH17 antibody, a CDH17 ADC, a biparatopic CDH17 ADC, a bispecific CDH17 antibody, a CDH17xCD3 bispecific antibody) or antigen binding fragment thereof for use in the treatment of patients having a cancer including, for example, colorectal adenocarcinoma, gastric adenocarcinoma, hepatic cancer, and pancreatic adenocarcinoma. In a particular embodiment, the compositions described herein are administered to cancer patients to kill tumor cells. For example, the compositions described herein can be used to treat a patient with a solid tumor characterized by the presence of cancer cells expressing or overexpressing CDH17. In some aspects, the disclosed compositions can be used to treat sarcomas, osteosarcomas, glioblastoma, ovarian cancer and pancreatic cancer.

In some aspects, the treatment of cancer represents a field where combination strategies are especially desirable since frequently the combined action of two, three, or even more cancer drugs/therapies generates synergistic effects which are considerably stronger than the impact of a mono-therapeutic approach. The agents and compositions (e.g., pharmaceutical compositions) provided herein may be used alone or in combination with conventional therapeutic regimens such as surgery, irradiation and/or chemotherapy. The agents and compositions may also be used in combination with one or more of an antineoplastic agent, a chemotherapeutic agent, a growth inhibitory agent, a cytotoxic agent, an immune checkpoint inhibitor, costimulatory molecule, kinase inhibitors, angiogenesis inhibitors, small molecule targeted therapy drugs, and multi-epitope strategies. Thus, in another embodiment of the present disclosure, a cancer treatment may be effectively combined with various other drugs.

In one treatment method, pharmaceutical compositions comprising the anti-CDH17 antibody or biparatopic anti-CDH17 antibody can further comprise a therapeutic or toxic agent, either conjugated or unconjugated to the anti-CDH17 antibody or antigen binding fragment. In a particular embodiment, an anti-CDH17 antibody is used to target an ADC with a cytotoxic payload to tumors expressing and/or overexpressing CDH17.

The disclosed CDH17 antibodies can be administered either alone or in combination with other compositions that are useful for treating cancer. In one embodiment, the disclosed antibodies can be administered either alone or in combination with other immunotherapeutics including other antibodies useful for treating cancer. For example, in an embodiment the other immunotherapeutic is an antibody against an immune checkpoint molecule selected from the group consisting of human programmed cell death protein 1 (PD-1), PD-L1 and PD-L2, lymphocyte activation gene 3 (LAG3), NKG2A, B7-H3, B7-H4, CTLA-4, GITR, VISTA, CD137, TIGIT and any combination thereof. In an alternative embodiment, the second immunotherapeutic is an antibody to a tumor specific antigen (TSA) or a tumor associated antigen (TAA). Each combination representing a separate embodiment of the disclosure.

The combination of therapeutic agents discussed herein can be administered concurrently as components of a bispecific or multi-specific binding agent or fusion protein or as a single composition in a pharmaceutically acceptable carrier. Alternatively, a combination of therapeutics can be administered concurrently as separate compositions with each agent in a pharmaceutically acceptable carrier. In another embodiment, the combination of therapeutic agents can be administered sequentially.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. In some aspects, the pharmaceutical composition is administered to a subject to treat cancer.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of the active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A composition of the present disclosure can be administered by a variety of methods known in the art. As will be appreciated by the skilled artisan, the route and/or mode of administration will vary depending upon the desired results. The active compounds can be prepared with carriers that will protect the compound against rapid releases, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

In an alternative embodiment, conventional viral and non-viral based gene transfer methods can be used to introduce nucleic acids encoding the antibodies or derivatives thereof, as described herein, in mammalian cells or target tissues. Such methods can be used to administer nucleic acids encoding the antibodies to cells in vitro. In some embodiments, the nucleic acids encoding the antibodies or derivatives thereof are administered for in vivo or ex vivo gene therapy uses. In other embodiments, gene delivery techniques are used to study the activity of the antibodies in cell-based or animal models. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding engineered polypeptides of the disclosure include lipofection, microinjection, biolistics, virosomes, liposomes, immunoliposomes, polycation or lipid: nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid: nucleic acid complexes, including targeted liposomes such as immunolipid complexes, is well known to one of skill in the art.

The use of RNA or DNA viral based systems for the delivery of nucleic acids encoding the antibodies described herein take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Dosage levels of the active ingredients in the pharmaceutical compositions may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical compositions described herein may be administered in effective amounts. An “effective amount” refers to the amount which achieves a desired reaction or the desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease.

### Non-Therapeutic Uses

The expression of CDH17 has been studied and identified by several groups to serve as a tumor biomarker and diagnostic marker for gastric cancer, liver cancer and gastrointestinal cancers (Grotzinger et al., Gut, 49:73-81, (2001); Su et al., Modern Pathology, 21, 1379-1386, (2008)). CDH17 was also identified as having higher sensitivity and specificity than existing markers such as CK20 and CDX2 in both colon and stomach adenocarcinoma (Tacha et al, Arch Pathol Lab Med (2017) 141 (1): 144-150) Examination of CDH17 expression in clinical samples also showed higher CDH17 expression to be correlative to poor survival and higher metastasis in cancer patients (Bartolome et al., Oncogene, 33, 1658-1669 (2014)). It was also reported that soluble CDH17 levels were elevated in stage II/III gastric cancer and CRC cancer (WO2010/040277).

Detection of CDH17 expression may serve as a predictive biomarker that could be utilized in the preclinical assessment of cancer patients to support personalized clinical outcomes. Such methods include contacting a biological sample from a subject with an anti-CDH17 antibody or antigen binding fragment thereof and detecting binding of the antibody CDH17. By “biological sample” is intended any biological sample obtained from an individual, cell line, tissue culture, or other source of cells potentially expressing CDH17. Methods for obtaining tissue biopsies for immunohistochemical analysis and body fluids for detection of soluble proteins in the serum or plasma of human subjects are well known in the art.

By utilizing antibodies that target CDH17, Immunohistochemistry (IHC) offers the means to assess CDH17 expression, distribution, and cellular localization. This technique proves especially useful in diagnosing and subclassifying tumors, aids in understanding disease mechanisms, predicting patient outcomes, and guiding treatment decisions.

Anti-CDH17 antibodies or antigen binding fragments are also useful in diagnostic assays to detect and/or quantify CDH17 protein, for example, detecting CDH17 expression in specific cells, tissues, or serum. The anti-CDH17 antibodies can be used diagnostically to, for example, monitor the development or progression of a disease as part of a clinical testing procedure to, e.g., determine the efficacy of a given treatment and/or prevention regimen.

It will be advantageous in some embodiments, for example, for diagnostic purposes to label the antibody with a detectable moiety. Numerous detectable labels are available, including radioisotopes, fluorescent labels, enzyme substrate labels and the like. The label may be indirectly conjugated with the antibody using various known techniques. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

Fujuwara et al., showed that radiolabeled anti-CDH17 antibodies have a potential to serve as an imaging probe for the diagnosis of primary lesions and lymph-node metastasis in gastric cancer. In their first study using 111 In-labeled anti-CDH17 antibody (111 In-D2101), they showed specificity of uptake by CDH17 expressing tumor AGS tumor cells in vivo and not MNK74 tumor cells which does not express CDH17 or normal organs. Imaging of 111 In-labeled antibody via temporal SPECT/CT in tumors showed high degree of tumor-non tumor contrast with biodistribution reaching significant incorporation of 39.2±9.5% ID/g by 96 hours. (Fujiwara et al., Annals of Nuclear Medicine, 34:13-23, (2020). In their second study, Fujiwara et al converted their antibody D2101 into scFv format and radiolabeled with ⁶⁴Cu (⁶⁴Cu-D2101). They show that ⁶⁴Cu-D2101 can also be utilized as an imaging probe with improved blood half-life and higher contrast from tumor to blood ratio compared to parental ¹¹¹In-D2101. (Fujiwara et al., Nuclear Medicine Communication, 41:688-695, (2020).

Exemplary radioisotope labels include 35S, 14C, 64CU, 1251, 3H, and 1311. The antibody can be labeled with the radioisotope, using the techniques described in, for example, Current Protocols in Immunology, Volumes 1 and 2, 1991, Coligen et al., Ed. Wiley-Interscience, New York, N.Y., Pubs. Radioactivity can be measured, for example, by scintillation counting.

Exemplary fluorescent labels include labels derived from rare earth chelates (europium chelates) or fluorescein and its derivatives, rhodamine and its derivatives, dansyl, Lissamine, phycoerythrin, and Texas Red are available. The fluorescent labels can be conjugated to the antibody via known techniques, such as those disclosed in Current Protocols in Immunology, for example. Fluorescence can be quantified using a fluorimeter. There are various well-characterized enzyme-substrate labels known in the art (see, e.g., U.S. Pat. No. 4,275,149). The enzyme generally catalyzes a chemical alteration of the chromogenic substrate that can be measured using various techniques. For example, alteration may be a color change in a substrate that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured, using a chemiluminometer, for example, or donates energy to a fluorescent acceptor.

Examples of enzymatic labels include luciferases such as firefly luciferase and bacterial luciferase (U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (such as glucose oxidase, galactose oxidase, and glucose-6-phosphate dehydrogenase), heterocydic oxidases (such as uricase and xanthine oxidase), lactoperoxidase, microperoxidase, and the like. Techniques for conjugating enzymes to antibodies are described, for example, in O'Sullivan et al., 1981, Methods for the Preparation of Enzyme-Antibody Conjugates for use in Enzyme Immunoassay, in Methods in Enzym. (J. Langone & H. Van Vunakis, eds.), Academic press, N.Y., 73:147-166.

Examples of enzyme-substrate combinations include, for example: Horseradish peroxidase (HRPO) with hydrogen peroxidase as a substrate, wherein the hydrogen peroxidase oxidizes a dye precursor such as orthophenylene diamine (OPD) or 3,3,5,5-tetramethyl benzidine hydrochloride (TMB); alkaline phosphatase (AP) with para-Nitrophenyl phosphate as chromogenic substrate; and β-D-galactosidase (β-D-Gal) with a chromogenic substrate such as p-nitrophenyl-β-D-galactosidase or fluorogenic substrate 4-methylumbelliferyl-β-D-galactosidase.

In another embodiment, the anti-CDH17 antibody or antigen binding fragment thereof is used unlabeled and detected with a labeled antibody that binds the anti-CDH17 antibody or antigen binding fragment thereof.

The antibodies and antigen binding fragments thereof described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

### Illustration of Subject Technology as Clauses

Various examples of aspects are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1: An anti-CDH17 antibody or antigen binding fragment thereof comprising a variable heavy (VH) region and a variable light (VL) region, wherein the VH region and the VL region comprise a set of complementarity-determining regions selected from:
(a) VH: CDR1: SEQ ID NO: 21, CDR2: SEQ ID NO: 22, CDR3: SEQ ID NO: 23, and VL: CDR1: SEQ ID NO: 24, CDR2: SEQ ID NO: 25, CDR3: SEQ ID NO: 26;
(b) VH: CDR1: SEQ ID NO: 27, CDR2: SEQ ID NO: 28, CDR3: SEQ ID NO: 29, and VL: CDR1: SEQ ID NO: 30, CDR2: SEQ ID NO: 31, CDR3: SEQ ID NO: 32;
(c) VH: CDR1: SEQ ID NO: 33, CDR2: SEQ ID NO: 34, CDR3: SEQ ID NO: 35, and VL: CDR1: SEQ ID NO: 36, CDR2: SEQ ID NO: 37, CDR3: SEQ ID NO: 38;
(d) VH: CDR1: SEQ ID NO: 39, CDR2: SEQ ID NO: 40, CDR3: SEQ ID NO: 41, and VL: CDR1: SEQ ID NO: 24, CDR2: SEQ ID NO: 25, CDR3: SEQ ID NO: 42;
(e) VH: CDR1: SEQ ID NO: 43, CDR2: SEQ ID NO: 40, CDR3: SEQ ID NO: 44, and VL: CDR1: SEQ ID NO: 24, CDR2: SEQ ID NO: 25, CDR3: SEQ ID NO: 45;
(f) VH: CDR1: SEQ ID NO: 46, CDR2: SEQ ID NO: 47, CDR3: SEQ ID NO: 48; and VL: CDR1: SEQ ID NO: 49, CDR2: SEQ ID NO: 50, CDR3: SEQ ID NO: 51;
(g) VH: CDR1: SEQ ID NO: 52, CDR2: SEQ ID NO: 53, CDR3: SEQ ID NO: 54; and VL: CDR1: SEQ ID NO: 55, CDR2: SEQ ID NO: 56, CDR3: SEQ ID NO: 57;
(h) VH: CDR1: SEQ ID NO: 58, CDR2: SEQ ID NO: 59, CDR3: SEQ ID NO: 60; and VL: CDR1: SEQ ID NO: 61, CDR2: SEQ ID NO: 62, CDR3: SEQ ID NO: 63;
(i) VH: CDR1: SEQ ID NO: 64, CDR2: SEQ ID NO: 65, CDR3: SEQ ID NO: 66; and VL: CDR1: SEQ ID NO: 67, CDR2: SEQ ID NO: 68, CDR3: SEQ ID NO: 69; or
(j) VH: CDR1: SEQ ID NO: 70, CDR2: SEQ ID NO: 71, CDR3: SEQ ID NO: 72; and VL: CDR1: SEQ ID NO: 73, CDR2: SEQ ID NO: 74, CDR3: SEQ ID NO: 75.

Clause 2. The anti-CDH17 antibody or antigen binding fragment thereof of Clause 1, wherein the antibody comprises:
(a) a heavy chain variable region having a sequence set forth in SEQ ID NO: 1, or an amino acid sequence having at least 90%, 95%, 96%, 97%, 98%, or 99% sequence identity thereto, and a light chain variable region having a sequence set forth in SEQ ID NO: 2, or an amino acid sequence having at least 90%, 95%, 96%, 97%, 98%, or 99% sequence identity thereto;
(b) a heavy chain variable region having a sequence set forth in SEQ ID NO: 3, or an amino acid sequence having at least 90%, 95%, 96%, 97%, 98%, or 99% sequence identity thereto, and a light chain variable region having a sequence set forth in SEQ ID NO: 4, or an amino acid sequence having at least 90%, 95%, 96%, 97%, 98%, or 99% sequence identity thereto;
(c) a heavy chain variable region having a sequence set forth in SEQ ID NO: 5, or an amino acid sequence having at least 90%, 95%, 96%, 97%, 98%, or 99% sequence identity thereto, and a light chain variable region having a sequence set forth in SEQ ID NO: 6, or an amino acid sequence having at least 90%, 95%, 96%, 97%, 98%, or 99% sequence identity thereto;
(d) a heavy chain variable region having a sequence set forth in SEQ ID NO: 7, or an amino acid sequence having at least 90%, 95%, 96%, 97%, 98%, or 99% sequence identity thereto, and a light chain variable region having a sequence set forth in SEQ ID NO: 8, or an amino acid sequence having at least 90%, 95%, 96%, 97%, 98%, or 99% sequence identity thereto;
(e) a heavy chain variable region having a sequence set forth in SEQ ID NO: 9, or an amino acid sequence having at least 90%, 95%, 96%, 97%, 98%, or 99% sequence identity thereto, and a light chain variable region having a sequence set forth in SEQ ID NO: 10, or an amino acid sequence having at least 90%, 95%, 96%, 97%, 98%, or 99% sequence identity thereto;
(f) a heavy chain variable region having a sequence set forth in SEQ ID NO: 11, or an amino acid sequence having at least 90%, 95%, 96%, 97%, 98%, or 99% sequence identity thereto, and a light chain variable region having a sequence set forth in SEQ ID NO: 12, or an amino acid sequence having at least 90%, 95%, 96%, 97%, 98%, or 99% sequence identity thereto;
(g) a heavy chain variable region having a sequence set forth in SEQ ID NO: 13, or an amino acid sequence having at least 90%, 95%, 96%, 97%, 98%, or 99% sequence identity thereto, and a light chain variable region having a sequence set forth in SEQ ID NO: 14, or an amino acid sequence having at least 90%, 95%, 96%, 97%, 98%, or 99% sequence identity thereto;
(h) a heavy chain variable region having a sequence set forth in SEQ ID NO: 15, or an amino acid sequence having at least 90%, 95%, 96%, 97%, 98%, or 99% sequence identity thereto, and a light chain variable region having a sequence set forth in SEQ ID NO: 16, or an amino acid sequence having at least 90%, 95%, 96%, 97%, 98%, or 99% sequence identity thereto;
(i) a heavy chain variable region having a sequence set forth in SEQ ID NO: 17, or an amino acid sequence having at least 90%, 95%, 96%, 97%, 98%, or 99% sequence identity thereto, and a light chain variable region having a sequence set forth in SEQ ID NO: 18, or an amino acid sequence having at least 90%, 95%, 96%, 97%, 98%, or 99% sequence identity thereto; or
(j) a heavy chain variable region having a sequence set forth in SEQ ID NO: 19, or an amino acid sequence having at least 90%, 95%, 96%, 97%, 98%, or 99% sequence identity thereto, and a light chain variable region having a sequence set forth in SEQ ID NO: 20, or an amino acid sequence having at least 90%, 95%, 96%, 97%, 98%, or 99% sequence identity thereto.

Clause 3. The anti-CDH17 antibody or antigen binding fragment thereof of Clause 1 or Clause 2, wherein the antibody comprises:
(a) a heavy chain variable region having a sequence set forth in SEQ ID NO: 1, and a light chain variable region having a sequence set forth in SEQ ID NO: 2;
(b) a heavy chain variable region having a sequence set forth in SEQ ID NO: 3, and a light chain variable region having a sequence set forth in SEQ ID NO: 4;
(c) a heavy chain variable region having a sequence set forth in SEQ ID NO: 5, and a light chain variable region having a sequence set forth in SEQ ID NO: 6;
(d) a heavy chain variable region having a sequence set forth in SEQ ID NO: 7, and a light chain variable region having a sequence set forth in SEQ ID NO: 8;
(e) a heavy chain variable region having a sequence set forth in SEQ ID NO: 9, and a light chain variable region having a sequence set forth in SEQ ID NO: 10;
(f) a heavy chain variable region having a sequence set forth in SEQ ID NO: 11, and a light chain variable region having a sequence set forth in SEQ ID NO: 12;
(g) a heavy chain variable region having a sequence set forth in SEQ ID NO: 13, and a light chain variable region having a sequence set forth in SEQ ID NO: 14;
(h) a heavy chain variable region having a sequence set forth in SEQ ID NO: 15, and a light chain variable region having a sequence set forth in SEQ ID NO: 16;
(i) a heavy chain variable region having a sequence set forth in SEQ ID NO: 17, and a light chain variable region having a sequence set forth in SEQ ID NO: 18; or
(j) a heavy chain variable region having a sequence set forth in SEQ ID NO: 19, and a light chain variable region having a sequence set forth in SEQ ID NO: 20.

Clause 4. The anti-CDH17 antibody or antigen binding fragment thereof of any one of Clauses 1-3, wherein the antibody is a murine antibody, a chimeric antibody, or a humanized antibody.

Clause 5. The anti-CDH17 antibody or antigen binding fragment thereof of any one of Clauses 1-4, wherein the antibody or antigen-binding fragment thereof is or comprises a F(ab′)2, a F(ab)2, a Fab′, a Fab, a Fv, a single chain Fv (scFv), a disulfide stabilized Fv (dsFv), an Fc-scFv, minibody, and a diabody.

Clause 6. The anti-CDH17 antibody or antigen binding fragment thereof of any one of Clauses 1-5, wherein the anti-CDH17 antibody or antigen binding fragment thereof is humanized.

Clause 7. The anti-CDH17 antibody or antigen binding fragment thereof of any one of Clauses 1-6, wherein the VH comprises an amino acid sequence that is at least 90%, 95%, 96%, 97%, 98%, or 99%, at least 95%, or at least 99% identical to an amino acid sequence selected from SEQ ID NOs: 76, 77, 80, 81, 84, 85 and 123; and wherein the VL comprises an amino acid sequence that is at least 90%, 95%, 96%, 97%, 98%, or 99%, at least 95%, or at least 99% identical to an amino acid sequence selected from SEQ ID NOs: 78, 79, 82, 83, 86, 87, and 124.

Clause 8. The anti-CDH17 antibody or antigen binding fragment thereof of any one of Clauses 1-7, wherein:
(a) the VH comprises an amino acid sequence that is at least 90%, 95%, 96%, 97%, 98%, or 99% identical to the amino acid sequence of SEQ ID NO: 76 or 77, and the VL comprises an amino acid sequence that is at least 90%, 95%, 96%, 97%, 98%, or 99% identical to the amino acid sequence of SEQ ID NO: 78 or 79;
(b) the VH comprises an amino acid sequence that is at least 90%, 95%, 96%, 97%, 98%, or 99% identical to the amino acid sequence of SEQ ID NO: 80 or 81, and the VL comprises an amino acid sequence that is at least 90%, 95%, 96%, 97%, 98%, or 99% identical to the amino acid sequence of SEQ ID NO: 82 or 83;
(c) the VH comprises an amino acid sequence that is at least 90%, 95%, 96%, 97%, 98%, or 99% identical to the amino acid sequence of SEQ ID NO: 84 or 85, and the VL comprises an amino acid sequence that is at least 90%, 95%, 96%, 97%, 98%, or 99% identical to the amino acid sequence of SEQ ID NO: 86 or 87; or
(d) the VH comprises an amino acid sequence that is at least 90%, 95%, 96%, 97%, 98%, or 99% identical to the amino acid sequence of SEQ ID NO: 123, and the VL comprises an amino acid sequence that is at least 90%, 95%, 96%, 97%, 98%, or 99% identical to the amino acid sequence of SEQ ID NO: 124.

Clause 9. The anti-CDH17 antibody or antigen binding fragment thereof of claim any one of Clauses 1-8, wherein:
(a) the VH comprises an amino acid sequence of SEQ ID NO: 76 or 77, and the VL comprises an amino acid sequence of SEQ ID NO: 78 or 79;
(b) the VH comprises an amino acid sequence of SEQ ID NO: 80 or 81, and the VL comprises an amino acid sequence of SEQ ID NO: 82 or 83;
(c) the VH comprises an amino acid sequence of SEQ ID NO: 84 or 85, and the VL comprises an amino acid sequence of SEQ ID NO: 86 or 87; or
(d) the VH comprises an amino acid sequence of SEQ ID NO: 123, and the VL comprises an amino acid sequence of SEQ ID NO: 124.

Clause 10. The anti-CDH17 antibody or antigen binding fragment thereof of any one of Clauses 1-9, further comprising a human heavy chain constant region, a human light chain constant region, or a human Fc region, or the combination thereof.

Clause 11. The anti-CDH17 antibody or antigen binding fragment thereof of any one of Clauses 1-10, wherein the antibody is or comprises a monoclonal antibody.

Clause 12. The anti-CDH17 antibody or antigen binding fragment thereof of any one of Clauses 1-11, wherein the antibody is a humanized or human monoclonal antibody.

Clause 13. The anti-CDH17 antibody or antigen binding fragment thereof of any one of Clauses 1-10, wherein the antibody is a biparatopic antibody.

Clause 14. The anti-CDH17 antibody or antigen binding fragment thereof of any one of Clauses 1-10 wherein the antibody is a bispecific antibody.

Clause 15. The anti-CDH17 antibody or antigen binding fragment thereof of any one of Clauses 1-14, wherein the anti-CDH17 antibody or antigen binding fragment thereof is conjugated to a drug moiety selected from a cytotoxin, an immunosuppressive agent, a radioisotope and a toxin.

Clause 16. The anti-CDH17 antibody or antigen binding fragment thereof of any one of Clauses 1-15, wherein
the VH CDR1 comprises the amino acid sequence of SEQ ID NO: 21,
the VH CDR2 comprises the amino acid sequence of SEQ ID NO: 22,
the VH CDR3 comprises the amino acid sequence of SEQ ID NO: 23,
the VL CDR1 comprises the amino acid sequence of SEQ ID NO: 24,
the VL CDR2 comprises the amino acid sequence of SEQ ID NO: 25, and
the VL CDR3 comprises the amino acid sequence of SEQ ID NO: 26.

Clause 17. The anti-CDH17 antibody or antigen binding fragment thereof of any one of Clauses 1-15, wherein
the VH CDR1 comprises the amino acid sequence of SEQ ID NO: 33,
the VH CDR2 comprises the amino acid sequence of SEQ ID NO: 34,
the VH CDR3 comprises the amino acid sequence of SEQ ID NO: 35,
the VL CDR1 comprises the amino acid sequence of SEQ ID NO: 36,
the VL CDR2 comprises the amino acid sequence of SEQ ID NO: 37, and
the VL CDR3 comprises the amino acid sequence of SEQ ID NO: 38.

Clause 18. The anti-CDH17 antibody or antigen binding fragment thereof of any one of Clauses 1-15, wherein
the VH CDR1 comprises the amino acid sequence of SEQ ID NO: 27,
the VH CDR2 comprises the amino acid sequence of SEQ ID NO: 28,
the VH CDR3 comprises the amino acid sequence of SEQ ID NO: 29,
the VL CDR1 comprises the amino acid sequence of SEQ ID NO: 30,
the VL CDR2 comprises the amino acid sequence of SEQ ID NO: 31, and
the VL CDR3 comprises the amino acid sequence of SEQ ID NO: 32.

Clause 19. The anti-CDH17 antibody or antigen binding fragment thereof of any one of Clauses 1-15, wherein
the VH CDR1 comprises the amino acid sequence of SEQ ID NO: 39,
the VH CDR2 comprises the amino acid sequence of SEQ ID NO: 40,
the VH CDR3 comprises the amino acid sequence of SEQ ID NO: 41,
the VL CDR1 comprises the amino acid sequence of SEQ ID NO: 24,
the VL CDR2 comprises the amino acid sequence of SEQ ID NO: 25, and
the VL CDR3 comprises the amino acid sequence of SEQ ID NO: 42.

Clause 20. A humanized anti-CDH17 antibody or antigen binding fragments thereof comprising a variable heavy (VH) region and a variable light (VL) region, wherein the VH region and the VL region comprise a set of complementarity-determining regions selected from:
(a) VH: CDR1: SEQ ID NO: 21, CDR2: SEQ ID NO: 22, CDR3: SEQ ID NO: 23, and VL: CDR1: SEQ ID NO: 24, CDR2: SEQ ID NO: 25, CDR3: SEQ ID NO: 26;
(b) VH: CDR1: SEQ ID NO: 27, CDR2: SEQ ID NO: 28, CDR3: SEQ ID NO: 29, and VL: CDR1: SEQ ID NO: 30, CDR2: SEQ ID NO: 31, CDR3: SEQ ID NO: 32;
(c) VH: CDR1: SEQ ID NO: 33, CDR2: SEQ ID NO: 34, CDR3: SEQ ID NO: 35, and VL: CDR1: SEQ ID NO: 36, CDR2: SEQ ID NO: 37, CDR3: SEQ ID NO: 38;
(d) VH: CDR1: SEQ ID NO: 39, CDR2: SEQ ID NO: 40, CDR3: SEQ ID NO: 41, and VL: CDR1: SEQ ID NO: 24, CDR2: SEQ ID NO: 25, CDR3: SEQ ID NO: 42;
(e) VH: CDR1: SEQ ID NO: 43, CDR2: SEQ ID NO: 40, CDR3: SEQ ID NO: 44, and VL: CDR1: SEQ ID NO: 24, CDR2: SEQ ID NO: 25, CDR3: SEQ ID NO: 45;
(f) VH: CDR1: SEQ ID NO: 46, CDR2: SEQ ID NO: 47, CDR3: SEQ ID NO: 48; and VL: CDR1: SEQ ID NO: 49, CDR2: SEQ ID NO: 50, CDR3: SEQ ID NO: 51;
(g) VH: CDR1: SEQ ID NO: 52, CDR2: SEQ ID NO: 53, CDR3: SEQ ID NO: 54; and VL: CDR1: SEQ ID NO: 55, CDR2: SEQ ID NO: 56, CDR3: SEQ ID NO: 57;
(h) VH: CDR1: SEQ ID NO: 58, CDR2: SEQ ID NO: 59, CDR3: SEQ ID NO: 60; and VL: CDR1: SEQ ID NO: 61, CDR2: SEQ ID NO: 62, CDR3: SEQ ID NO: 63;
(i) VH: CDR1: SEQ ID NO: 64, CDR2: SEQ ID NO: 65, CDR3: SEQ ID NO: 66; and VL: CDR1: SEQ ID NO: 67, CDR2: SEQ ID NO: 68, CDR3: SEQ ID NO: 69; or
(j) VH: CDR1: SEQ ID NO: 70, CDR2: SEQ ID NO: 71, CDR3: SEQ ID NO: 72; and VL: CDR1: SEQ ID NO: 73, CDR2: SEQ ID NO: 74, CDR3: SEQ ID NO: 75.

Clause 21. The humanized anti-CDH17 antibody or antigen binding fragments thereof of Clause 20, wherein the humanized antibody or antigen-binding fragment comprises:
(a) a variable heavy chain region (VH) comprising an amino acid sequence having at least 90%, at least 95%, or at least 99% identity to an amino acid sequence of SEQ ID NO: 76 and a variable light chain region (VL) comprising an amino acid sequence having at least 90%, at least 95%, or at least 99% identity to an amino acid sequence of SEQ ID NO: 78;
(b) a VH comprising an amino acid sequence having at least 90%, at least 95%, or at least 99% identity to an amino acid sequence of SEQ ID NO: 76 and a VL comprising an amino acid sequence having at least 90%, at least 95%, or at least 99% identity to an amino acid sequence of SEQ ID NO: 79;
(c) a VH comprising an amino acid sequence having at least 90%, at least 95%, or at least 99% identity to an amino acid sequence of SEQ ID NO: 77 and a VL comprising an amino acid sequence having at least 90%, at least 95%, or at least 99% identity to an amino acid sequence of SEQ ID NO: 78;
(d) a VH comprising an amino acid sequence having at least 90%, at least 95%, or at least 99% identity to an amino acid sequence of SEQ ID NO: 77 and a VL comprising an amino acid sequence having at least 90%, at least 95%, or at least 99% identity to an amino acid sequence of SEQ ID NO: 79;
(e) a VH comprising an amino acid sequence having at least 90%, at least 95%, or at least 99% identity to an amino acid sequence of SEQ ID NO: 80 and a VL comprising an amino acid sequence having at least 90%, at least 95%, or at least 99% identity to an amino acid sequence of SEQ ID NO: 82;
(f) a VH comprising an amino acid sequence having at least 90%, at least 95%, or at least 99% identity to an amino acid sequence of SEQ ID NO:80 and a VL comprising an amino acid sequence having at least 90%, at least 95%, or at least 99% identity to an amino acid sequence of SEQ ID NO: 83;
(g) a VH comprising an amino acid sequence having at least 90%, at least 95%, or at least 99% identity to an amino acid sequence of SEQ ID NO: 81 and a VL comprising an amino acid sequence having at least 90%, at least 95%, or at least 99% identity to an amino acid sequence of SEQ ID NO: 82;
(h) a VH comprising an amino acid sequence having at least 90%, at least 95%, or at least 99% identity to an amino acid sequence of SEQ ID NO: 81 and a VL comprising an amino acid sequence having at least 90%, at least 95%, or at least 99% identity to an amino acid sequence of SEQ ID NO: 83;
(i) a VH comprising an amino acid sequence having at least 90%, at least 95%, or at least 99% identity to an amino acid sequence of SEQ ID NO: 84 and a VL comprising an amino acid sequence having at least 90%, at least 95%, or at least 99% identity to an amino acid sequence of SEQ ID NO: 86;
(j) a VH comprising an amino acid sequence having at least 90%, at least 95%, or at least 99% identity to an amino acid sequence of SEQ ID NO: 84 and a VL comprising an amino acid sequence having at least 90%, at least 95%, or at least 99% identity to an amino acid sequence of SEQ ID NO: 87;
(k) a VH comprising an amino acid sequence having at least 90%, at least 95%, or at least 99% identity to an amino acid sequence of SEQ ID NO: 85 and a VL comprising an amino acid sequence having at least 90%, at least 95%, or at least 99% identity to an amino acid sequence of SEQ ID NO: 86;
(l) a VH comprising an amino acid sequence having at least 90%, at least 95%, or at least 99% identity to an amino acid sequence of SEQ ID NO: 85 and a VL comprising an amino acid sequence having at least 90%, at least 95%, or at least 99% identity to an amino acid sequence of SEQ ID NO: 87; or
(m) a VH comprising an amino acid sequence having at least 90%, at least 95%, or at least 99% identity to an amino acid sequence of SEQ ID NO: 123 and a VL comprising an amino acid sequence having at least 90%, at least 95%, or at least 99% identity to an amino acid sequence of SEQ ID NO: 124.

Clause 22. The humanized anti-CDH17 antibody or antigen binding fragments thereof of Clause 20 or Clause 21, wherein the humanized antibody or antigen-binding fragment comprises:
(a) a VH comprises an amino acid sequence an amino acid sequence of SEQ ID NO: 76 and a VL comprises an amino acid sequence an amino acid sequence of SEQ ID NO: 78;
(b) a VH comprises an amino acid sequence an amino acid sequence of SEQ ID NO: 76 and a VL comprises an amino acid sequence an amino acid sequence of SEQ ID NO: 79;
(c) a VH comprises an amino acid sequence an amino acid sequence of SEQ ID NO: 77 and a VL comprises an amino acid sequence an amino acid sequence of SEQ ID NO: 78;
(d) a VH comprises an amino acid sequence an amino acid sequence of SEQ ID NO: 77 and a VL comprises an amino acid sequence an amino acid sequence of SEQ ID NO: 79;
(e) a VH comprises an amino acid sequence an amino acid sequence of SEQ ID NO: 80 and a VL comprises an amino acid sequence an amino acid sequence of SEQ ID NO: 82;
(f) a VH comprises an amino acid sequence an amino acid sequence of SEQ ID NO: 80 and a VL comprises an amino acid sequence an amino acid sequence of SEQ ID NO: 83;
(g) a VH comprises an amino acid sequence an amino acid sequence of SEQ ID NO: 81 and a VL comprises an amino acid sequence an amino acid sequence of SEQ ID NO: 82;
(h) a VH comprises an amino acid sequence an amino acid sequence of SEQ ID NO: 81 and a VL comprises an amino acid sequence an amino acid sequence of SEQ ID NO: 83;
(i) a VH comprises an amino acid sequence an amino acid sequence of SEQ ID NO: 84 and a VL comprises an amino acid sequence an amino acid sequence of SEQ ID NO: 86;
(j) a VH comprises an amino acid sequence an amino acid sequence of SEQ ID NO: 84 and a VL comprises an amino acid sequence an amino acid sequence of SEQ ID NO: 87;
(k) a VH comprises an amino acid sequence an amino acid sequence of SEQ ID NO: 85 and a VL comprises an amino acid sequence an amino acid sequence of SEQ ID NO: 86;
(l) a VH comprises an amino acid sequence an amino acid sequence of SEQ ID NO: 85 and a VL comprises an amino acid sequence an amino acid sequence of SEQ ID NO: 87; or
(m) a VH comprises an amino acid sequence an amino acid sequence of SEQ ID NO: 123 and a VL comprises an amino acid sequence an amino acid sequence of SEQ ID NO: 124.

Clause 23. The humanized anti-CDH17 antibody or antigen binding fragments thereof of any one of Clauses 20-22, wherein the antibody or antigen-binding fragment thereof comprises:
(a) a heavy chain Fab region comprising an amino acid sequence that is at least 90%, at least 95%, or at least 99% identical to an amino acid sequence selected from SEQ ID NOs: 88, 90, 92, 94, 96, 100, 102, 104, 117, 119, 121, and 125; and
(b) a light chain Fab region comprising an amino acid sequence that is at least 90%, at least 95%, or at least 99% identical to an amino acid sequence selected from SEQ ID NOs: 89, 91, 93, 95, 97, 101, 118, 103, 105, 120, 122, and 126.

Clause 24. The humanized anti-CDH17 antibody or antigen binding fragments thereof of any one of Clauses 20-23, wherein the antibody or antigen-binding fragment thereof comprises:
(a) a heavy chain Fab region comprising an amino acid sequence that is at least 90%, or at least 95%, or at least 99% identical to the amino acid sequence of SEQ ID NO: 88, and a light chain Fab region comprising an amino acid sequence that is at least 90%, or at least 95%, or at least 99% identical to the amino acid sequence of SEQ ID NO: 89;
(b) a heavy chain Fab region comprising an amino acid sequence that is at least 90%, or at least 95%, or at least 99% identical to the amino acid sequence of SEQ ID NO: 90, and a light chain Fab region comprising an amino acid sequence that is at least 90%, or at least 95%, or at least 99% identical to the amino acid sequence of SEQ ID NO: 91;
(c) a heavy chain Fab region comprising an amino acid sequence that is at least 90%, or at least 95%, or at least 99% identical to the amino acid sequence of SEQ ID NO: 92, and a light chain Fab region comprising an amino acid sequence that is at least 90%, or at least 95%, or at least 99% identical to the amino acid sequence of SEQ ID NO: 93;
(d) a heavy chain Fab region comprising an amino acid sequence that is at least 90%, or at least 95%, or at least 99% identical to the amino acid sequence of SEQ ID NO: 94, and a light chain Fab region comprising an amino acid sequence that is at least 90%, or at least 95%, or at least 99% identical to the amino acid sequence of SEQ ID NO: 95;
(e) a heavy chain Fab region comprising an amino acid sequence that is at least 90%, or at least 95%, or at least 99% identical to the amino acid sequence of SEQ ID NO: 96, and a light chain Fab region comprising an amino acid sequence that is at least 90%, or at least 95%, or at least 99% identical to the amino acid sequence of SEQ ID NO: 97;
(f) a heavy chain Fab region comprising an amino acid sequence that is at least 90%, or at least 95%, or at least 99% identical to the amino acid sequence of SEQ ID NO: 100, and a light chain Fab region comprising an amino acid sequence that is at least 90%, or at least 95%, or at least 99% identical to the amino acid sequence of SEQ ID NO: 101;
(g) a heavy chain Fab region comprising an amino acid sequence that is at least 90%, or at least 95%, or at least 99% identical to the amino acid sequence of SEQ ID NO: 102, and a light chain Fab region comprising an amino acid sequence that is at least 90%, or at least 95%, or at least 99% identical to the amino acid sequence of SEQ ID NO: 103;
(h) a heavy chain Fab region comprising an amino acid sequence that is at least 90%, or at least 95%, or at least 99% identical to the amino acid sequence of SEQ ID NO: 104, and a light chain Fab region comprising an amino acid sequence that is at least 90%, or at least 95%, or at least 99% identical to the amino acid sequence of SEQ ID NO: 105;
(i) a heavy chain Fab region comprising an amino acid sequence that is at least 90%, or at least 95%, or at least 99% identical to the amino acid sequence of SEQ ID NO: 117, and a light chain Fab region comprising an amino acid sequence that is at least 90%, or at least 95%, or at least 99% identical to the amino acid sequence of SEQ ID NO: 118;
(j) a heavy chain Fab region comprising an amino acid sequence that is at least 90%, or at least 95%, or at least 99% identical to the amino acid sequence of SEQ ID NO: 119, and a light chain Fab region comprising an amino acid sequence that is at least 90%, or at least 95%, or at least 99% identical to the amino acid sequence of SEQ ID NO: 120;
(k) a heavy chain Fab region comprising an amino acid sequence that is at least 90%, or at least 95%, or at least 99% identical to the amino acid sequence of SEQ ID NO: 121, and a light chain Fab region comprising an amino acid sequence that is at least 90%, or at least 95%, or at least 99% identical to the amino acid sequence of SEQ ID NO: 122; or
(l) a heavy chain Fab region comprising an amino acid sequence that is at least 90%, or at least 95%, or at least 99% identical to the amino acid sequence of SEQ ID NO: 125, and a light chain Fab region comprising an amino acid sequence that is at least 90%, or at least 95%, or at least 99% identical to the amino acid sequence of SEQ ID NO: 126.

Clause 25. The humanized anti-CDH17 antibody or antigen binding fragments thereof of any one of Clauses 20-24, wherein the antibody or antigen-binding fragment thereof comprises:
(a) a heavy chain Fab region comprising an amino acid sequence of SEQ ID NO: 88, and a light chain Fab region comprising an amino acid sequence of SEQ ID NO: 89;
(b) a heavy chain Fab region comprising an amino acid sequence of SEQ ID NO: 90, and a light chain Fab region comprising an amino acid sequence of SEQ ID NO: 91;
(c) a heavy chain Fab region comprising an amino acid sequence of SEQ ID NO: 92, and a light chain Fab region comprising an amino acid sequence of SEQ ID NO: 93;
(d) a heavy chain Fab region comprising an amino acid sequence of SEQ ID NO: 94, and a light chain Fab region comprising an amino acid sequence of SEQ ID NO: 95;
(e) a heavy chain Fab region comprising an amino acid sequence of SEQ ID NO: 96, and a light chain Fab region comprising an amino acid sequence of SEQ ID NO: 97;
(f) a heavy chain Fab region comprising an amino acid sequence of SEQ ID NO: 100, and a light chain Fab region comprising an amino acid sequence of SEQ ID NO: 101;
(g) a heavy chain Fab region comprising an amino acid sequence of SEQ ID NO: 102, and a light chain Fab region comprising an amino acid sequence of SEQ ID NO: 103;
(h) a heavy chain Fab region comprising an amino acid sequence of SEQ ID NO: 104, and a light chain Fab region comprising an amino acid sequence of SEQ ID NO: 105;
(i) a heavy chain Fab region comprising an amino acid sequence of SEQ ID NO: 117, and a light chain Fab region comprising an amino acid sequence of SEQ ID NO: 118;
(j) a heavy chain Fab region comprising an amino acid sequence of SEQ ID NO: 119, and a light chain Fab region comprising an amino acid sequence of SEQ ID NO: 120;
(k) a heavy chain Fab region comprising an amino acid sequence of SEQ ID NO: 121, and a light chain Fab region comprising an amino acid sequence of SEQ ID NO: 122; or
(l) a heavy chain Fab region comprising an amino acid sequence of SEQ ID NO: 125, and a light chain Fab region comprising an amino acid sequence of SEQ ID NO: 126.

Clause 26. The humanized anti-CDH17 antibody or antigen binding fragments thereof of any one of Clauses 20-25.

Clause 27. The humanized antibody or antigen-binding fragment thereof of Clause 20, wherein the antibody or antigen-binding fragment thereof is conjugated to a drug moiety selected from a cytotoxin, an immunosuppressive agent, a radioisotope or a toxin.

Clause 28. A biparatopic antibody or antigen-binding fragment thereof comprising a first antigen-binding portion having specificity to a first epitope in human CDH17 and a second antigen-binding portion having specificity to a second distinct non-overlapping epitope within human CDH17, wherein first antigen-binding portion and/or the second antigen-binding portion comprises a variable heavy (VH) region and a variable light (VL) region, wherein the VH region and the VL region comprise a set of complementarity-determining regions selected from:
(a) VH: CDR1: SEQ ID NO: 21, CDR2: SEQ ID NO: 22, CDR3: SEQ ID NO: 23, and VL: CDR1: SEQ ID NO: 24, CDR2: SEQ ID NO: 25, CDR3: SEQ ID NO: 26;
(b) VH: CDR1: SEQ ID NO: 27, CDR2: SEQ ID NO: 28, CDR3: SEQ ID NO: 29, and VL: CDR1: SEQ ID NO: 30, CDR2: SEQ ID NO: 31, CDR3: SEQ ID NO: 32;
(c) VH: CDR1: SEQ ID NO: 33, CDR2: SEQ ID NO: 34, CDR3: SEQ ID NO: 35, and VL: CDR1: SEQ ID NO: 36, CDR2: SEQ ID NO: 37, CDR3: SEQ ID NO: 38;
(d) VH: CDR1: SEQ ID NO: 39, CDR2: SEQ ID NO: 40, CDR3: SEQ ID NO: 41, and VL: CDR1: SEQ ID NO: 24, CDR2: SEQ ID NO: 25, CDR3: SEQ ID NO: 42;
(e) VH: CDR1: SEQ ID NO: 43, CDR2: SEQ ID NO: 40, CDR3: SEQ ID NO: 44, and VL: CDR1: SEQ ID NO: 24, CDR2: SEQ ID NO: 25, CDR3: SEQ ID NO: 45;
(f) VH: CDR1: SEQ ID NO: 46, CDR2: SEQ ID NO: 47, CDR3: SEQ ID NO: 48; and VL: CDR1: SEQ ID NO: 49, CDR2: SEQ ID NO: 50, CDR3: SEQ ID NO: 51;
(g) VH: CDR1: SEQ ID NO: 52, CDR2: SEQ ID NO: 53, CDR3: SEQ ID NO: 54; and VL: CDR1: SEQ ID NO: 55, CDR2: SEQ ID NO: 56, CDR3: SEQ ID NO: 57;
(h) VH: CDR1: SEQ ID NO: 58, CDR2: SEQ ID NO: 59, CDR3: SEQ ID NO: 60; and VL: CDR1: SEQ ID NO: 61, CDR2: SEQ ID NO: 62, CDR3: SEQ ID NO: 63;
(i) VH: CDR1: SEQ ID NO: 64, CDR2: SEQ ID NO: 65, CDR3: SEQ ID NO: 66; and VL: CDR1: SEQ ID NO: 67, CDR2: SEQ ID NO: 68, CDR3: SEQ ID NO: 69; or
(j) VH: CDR1: SEQ ID NO: 70, CDR2: SEQ ID NO: 71, CDR3: SEQ ID NO: 72; and VL: CDR1: SEQ ID NO: 73, CDR2: SEQ ID NO: 74, CDR3: SEQ ID NO: 75.

Clause 29. The biparatopic antibody or antigen-binding fragment thereof of Clause 28, comprising:
(a) the first antigen binding portion having specificity for an epitope in ECD 3-5 of human CDH17 and a second binding portion having specificity for an epitope in ECD 6-7 of human CDH17; or
(b) the first antigen binding portion having specificity for an epitope in ECD 1-2 of human CDH17 and a second binding portion having specificity for an epitope in ECD 6-7 of human CDH17.

Clause 30. The biparatopic antibody or antigen-binding fragment thereof of any one of Clauses 27-29, comprising:
(a)
   the first antigen binding portion comprises a VH comprising an amino acid sequence of SEQ ID NO: 1 or 17, and a light chain variable region (VL) comprising an amino acid sequence of SEQ ID NO: 2 or 18; and the second antigen binding portion comprises a heavy chain variable region (VH) comprising an amino acid sequence of SEQ ID NO: 3, 5, 7, 9 or 11, and a light chain variable region (VL) comprising an amino acid sequence of SEQ ID NO: 4, 6, 8, 10 or 12; or
(b)
   the first antigen binding portion comprises a heavy chain variable region (VH) comprising an amino acid sequence of SEQ ID NO: 15, and a light chain variable region (VL) comprising an amino acid sequence of SEQ ID NO: 16; and
   the second antigen binding portion comprises a heavy chain variable region (VH) comprising an amino acid sequence of SEQ ID NO: 3, 5, 7, 9 or 11, and a light chain variable region (VL) comprising an amino acid sequence of SEQ ID NO: 4, 6, 8, 10 or 12.

Clause 31. The biparatopic antibody or antigen-binding fragment thereof of any one of Clauses 27-30, wherein biparatopic anti-CDH17 antibody or antigen-binding fragment thereof is or comprises a F(ab′)2, a F(ab)2, IgG-scFv, tandem scFv and an antibody.

Clause 32. A biparatopic anti-CDH17 antibody or antigen-binding fragment thereof, wherein the antibody comprises a first Fab fragment (Arm 1) comprising a first heavy chain variable region and a first light chain variable region and a second Fab fragment (Arm 2) comprising a second heavy chain variable region and a second variable light chain region, wherein the first heavy chain variable region, the first light chain variable region, the second heavy chain variable region, and the second variable light chain region, respectively, comprise amino acid sequences that are at least 90%, at least 95%, or at least 99% identical to the amino acid sequences of
(a) SEQ ID NO: 88, SEQ ID NO: 89, SEQ ID NO: 90, and SEQ ID NO: 91;
(b) SEQ ID NO: 88, SEQ ID NO: 89, SEQ ID NO: 92 and SEQ ID NO: 93;
(c) SEQ ID NO: 94, SEQ ID NO: 95, SEQ ID NO: 96, and SEQ ID NO: 97;
(d) SEQ ID NO: 117, SEQ ID NO: 118, SEQ ID NO: 119, and SEQ ID NO: 120; or
(e) SEQ ID NO: 117, SEQ ID NO: 118, SEQ ID NO: 121, and SEQ ID NO: 122.

Clause 33. The biparatopic anti-CDH17 antibody or antigen-binding fragment thereof of Clause 32, wherein the first heavy chain variable region, the first light chain variable region, the second heavy chain variable region, and the second variable light chain region, respectively, comprises an amino acid sequence of
(a) SEQ ID NO: 88, SEQ ID NO: 89, SEQ ID NO: 90, and SEQ ID NO: 91;
(b) SEQ ID NO: 88, SEQ ID NO: 89, SEQ ID NO: 92 and SEQ ID NO: 93;
(c) SEQ ID NO: 94, SEQ ID NO: 95, SEQ ID NO: 96, and SEQ ID NO: 97;
(d) SEQ ID NO: 117, SEQ ID NO: 118, SEQ ID NO: 119, and SEQ ID NO: 120; or
(e) SEQ ID NO: 117, SEQ ID NO: 118, SEQ ID NO: 121, and SEQ ID NO: 122.

Clause 34. The biparatopic anti-CDH17 antibody or antigen-binding fragment thereof of Clause 32 or Clause 33, wherein biparatopic anti-CDH17 antibody or antigen-binding fragment thereof is or comprises a F(ab′)2, a F(ab)2, IgG-scFv, tandem scFv and a bispecific antibody.

Clause 35. The biparatopic anti-CDH17 antibody or antigen-binding fragment thereof of any one of Clauses 27-34, further comprising a drug moiety conjugated to the biparatopic anti-CDH17 antibody or antigen-binding fragment thereof, wherein the drug moiety is selected from a cytotoxin, an immunosuppressive agent, a radioisotope or a toxin.

Clause 36. The anti-CDH17 antibody or antigen binding fragment thereof of Clause 1, wherein the antibody is a bispecific antibody.

Clause 37. The anti-CDH17 antibody or antigen binding fragment thereof of Clause 36, wherein the bispecific antibody binds to a first epitope within human CDH17 and to a second epitope within human CD3.

Clause 38. The anti-CDH17 antibody or antigen binding fragment thereof of Clause 36 or 37, wherein the second epitope is within a human CD3 epsilon subunit of a human T cell receptor complex.

Clause 39. A bispecific T cell engager antibody wherein the antibody comprising a first anti-CD3 scFv fragment (Arm 1) and a second Fab fragment (Arm 2), wherein second Fab fragment comprises a variable heavy (VH) region and a variable light (VL) region, wherein the VH region and the VL region comprise a set of complementarity-determining regions selected from:
(a) VH: CDR1: SEQ ID NO: 21, CDR2: SEQ ID NO: 22, CDR3: SEQ ID NO: 23, and VL: CDR1: SEQ ID NO: 24, CDR2: SEQ ID NO: 25, CDR3: SEQ ID NO: 26;
(b) VH: CDR1: SEQ ID NO: 27, CDR2: SEQ ID NO: 28, CDR3: SEQ ID NO: 29, and VL: CDR1: SEQ ID NO: 30, CDR2: SEQ ID NO: 31, CDR3: SEQ ID NO: 32;
(c) VH: CDR1: SEQ ID NO: 33, CDR2: SEQ ID NO: 34, CDR3: SEQ ID NO: 35, and VL: CDR1: SEQ ID NO: 36, CDR2: SEQ ID NO: 37, CDR3: SEQ ID NO: 38;
(d) VH: CDR1: SEQ ID NO: 39, CDR2: SEQ ID NO: 40, CDR3: SEQ ID NO: 41; and VL: CDR1: SEQ ID NO: 24, CDR2: SEQ ID NO: 25, CDR3: SEQ ID NO: 42;
(e) VH: CDR1: SEQ ID NO: 43, CDR2: SEQ ID NO: 40, CDR3: SEQ ID NO: 44, and VL: CDR1: SEQ ID NO: 24, CDR2: SEQ ID NO: 25, CDR3: SEQ ID NO: 45;
(f) VH: CDR1: SEQ ID NO: 46, CDR2: SEQ ID NO: 47, CDR3: SEQ ID NO: 48; and VL: CDR1: SEQ ID NO: 49, CDR2: SEQ ID NO: 50, CDR3: SEQ ID NO: 51;
(g) VH: CDR1: SEQ ID NO: 52, CDR2: SEQ ID NO: 53, CDR3: SEQ ID NO: 54; and VL: CDR1: SEQ ID NO: 55, CDR2: SEQ ID NO: 56, CDR3: SEQ ID NO: 57;
(h) VH: CDR1: SEQ ID NO: 58, CDR2: SEQ ID NO: 59, CDR3: SEQ ID NO: 60; and VL: CDR1: SEQ ID NO: 61, CDR2: SEQ ID NO: 62, CDR3: SEQ ID NO: 63;
(i) VH: CDR1: SEQ ID NO: 64, CDR2: SEQ ID NO: 65, CDR3: SEQ ID NO: 66; and VL: CDR1: SEQ ID NO: 67, CDR2: SEQ ID NO: 68, CDR3: SEQ ID NO: 69; or
(j) VH: CDR1: SEQ ID NO: 70, CDR2: SEQ ID NO: 71, CDR3: SEQ ID NO: 72; and VL: CDR1: SEQ ID NO: 73, CDR2: SEQ ID NO: 74, CDR3: SEQ ID NO: 75.

Clause 40. The bispecific T cell engager antibody of Clause 39, wherein the antibody comprising a first anti-CD3 scFv fragment (Arm 1) and a second Fab fragment (Arm 2) comprising a heavy chain variable region and a light chain variable region specific for CDH17, wherein the heavy chain variable region and the light chain variable region, respectively, comprise the amino acid sequences of:
(a) SEQ ID NO: 100 and SEQ ID NO: 101;
(b) SEQ ID NO: 102 and SEQ ID NO: 103;
(c) SEQ ID NO: 104 and SEQ ID NO: 105; or
(d) SEQ ID NO: 125 and SEQ ID NO: 126.

Clause 41. The bispecific T cell engager antibody of Clause 39 or Clause 40, wherein the anti-CD3 scFv fragment comprises an amino acid sequence of SEQ ID NO: 98 or SEQ ID NO: 127.

Clause 42. The bispecific T cell engager antibody of any one of Clauses 39-41, wherein the anti-CD3 scFv fragment the heavy chain variable region and the light chain variable region, respectively, comprise the amino acid sequences of:
(a) SEQ ID NO: 98, SEQ ID NO: 100 and SEQ ID NO: 101;
(b) SEQ ID NO: 98, SEQ ID NO: 102 and SEQ ID NO: 103;
(c) SEQ ID NO: 98, SEQ ID NO: 104 and SEQ ID NO: 105;
(d) SEQ ID NO: 98, SEQ ID NO: 125 and SEQ ID NO: 126; or
(e) SEQ ID NO: 127, SEQ ID NO: 125 and SEQ ID NO: 126.

Clause 43. A bispecific T cell engager antibody, comprising a first antigen-binding portion having specificity to a human CDH17 protein and a second portion having specificity to human CD3, wherein the first antigen-binding portion comprises a Fab heavy chain comprising the amino acid sequence of SEQ ID NO: 125 and a Fab light chain comprising the amino acid sequence of SEQ ID NO: 126 and the second portion having specificity to human CD3 has an amino acid sequence selected from SEQ ID NO: 98 or SEQ ID NO: 127.

Clause 44. The bispecific T cell engager antibody of any one of Clause 39-43, wherein biparatopic anti-CDH17 antibody or antigen-binding fragment thereof is or comprises a F(ab′)2, a F(ab)2, IgG-scFv, tandem scFv, and a bispecific antibody.

Clause 45. The anti-CDH17 antibody or antigen binding fragment thereof of Clause 1, wherein the antibody is a biparatopic antibody that binds to two distinct and non-overlapping epitopes within human CDH17.

Clause 46. The anti-CDH17 antibody or antigen binding fragment thereof of Clause 45 wherein the biparatopic antibody binds to a first epitope within human CDH17 ECD 3-5 and to a second epitope within ECD 6-7 of human CDH17.

Clause 47. The anti-CDH17 antibody or antigen binding fragment thereof of any one of Clauses 1-46, wherein the antibody binds to human and cynomolgus monkey CDH17.

Clause 48. An isolated polynucleotide encoding the antibody or antigen binding fragment thereof according to any one of Clauses 1 to 20, or the humanized anti-CDH17 antibody or antigen binding fragments thereof of any one of Clauses 20-26, or biparatopic anti-CDH17 antibody or antigen-binding fragment thereof of any one of Clauses 27-35, or and the bispecific T cell engager antibody of any one of Clauses 36-46.

Clause 49. A pharmaceutical composition comprising the antibody or antigen binding fragment thereof according to any one of Clauses 1 to 20, or the humanized anti-CDH17 antibody or antigen binding fragments thereof of any one of Clauses 20-26, or biparatopic anti-CDH17 antibody or antigen-binding fragment thereof of any one of Clauses 27-35, or and the bispecific T cell engager antibody of any one of Clauses 36-46, or the nucleic acid of clause 48 and a pharmaceutically acceptable carrier.

Clause 50. The pharmaceutical composition according to Clause 49, for use in treating a cancer.

Clause 51. A method of treating a cancer in a subject in need thereof, the method comprising administering to the subject in need thereof the pharmaceutical composition according to Clause 49.

Clause 52. The method according to Clause 51, wherein the cancer is selected from the group consisting of: colorectal adenocarcinoma, gastric adenocarcinoma, hepatic cancer, ovarian cancer, sarcomas, osteosarcomas, glioblastoma, and pancreatic adenocarcinoma.

Clause 53. A method of diagnosing a cancer in a subject, the method comprising: contacting a biological sample with the antibody or antigen binding fragment thereof according to any one of Clauses 1 to 20, or the humanized anti-CDH17 antibody or antigen binding fragments thereof of any one of Clauses 20-26, or biparatopic anti-CDH17 antibody or antigen-binding fragment thereof of any one of Clauses 27-35, or and the bispecific T cell engager antibody of any one of Clauses 36-46.

Clause 54. The isolated polynucleotide according to Clause 48 wherein the polynucleotide encodes an amino acid sequence as set forth in any one of SEQ ID NOs: 1 to 20 or SEQ ID NOS: 76 to 87 or SEQ ID NOS: 123-124.

Clause 55. A vector comprising the polynucleotide according to Clause 54.

Clause 56. A host cell comprising a polynucleotide according to Clause 54, and/or a vector according to Clause 55.

Clause 57. A method for the production of an anti-CDH17 antibody or antigen binding fragment thereof according to any one of Clauses 1 to 11, the method comprising culturing the host cell of Clause 56.

The broad scope of this disclosure is best understood with reference to the following examples, which are not intended to limit the disclosures to the specific embodiments. The specific embodiments described herein are offered by way of example only, and the disclosure is to be limited by the terms of the appended claims, along with the full scope of the equivalents to which such claims are entitled.

### EXAMPLES

### Example 1: General Methods

Methods for protein purification including immunoprecipitation, chromatography, and electrophoresis are described. See, e.g., Coligan et al. (2000) Current Protocols in Protein Science, Vol. 1, John Wiley and Sons, Inc., New York. Chemical analysis, chemical modification, post-translational modification, production of fusion proteins, and glycosylation of proteins are described. See, e.g., Coligan et al. (2000) Current Protocols in Protein Science, Vol. 2, John Wiley and Sons, Inc., New York, Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. See, e.g., Coligan et al. (2001) Current Protocols in Immunology, Vol. 1, John Wiley and Sons, Inc., New York; Harlow and Lane (1999) Using Antibodies, Cold Spring Harbor Laboratory’ Press, Cold Spring Harbor, N.Y., Harlow and Lane, supra.

Standard methods in molecular biology are described. See, e.g., Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Hybridoma or cell culture supernatant containing an anti-CDH17 antibody was purified via HiTrap protein G column (GE, cat. No. 17040401) according to the manufacturer's procedures. Briefly, supernatant was equilibrated with DPBS (Gibco, cat. No. 14190-136) for 5 CV and loaded via syringe/infusion pump (Legato 200, KDS) at ambient temperature and 3 minute residence time. The column was washed with 5 CV of DPBS and elution was performed with 4 CV of pH 2.8 elution buffer (Fisher Scientific, cat. No. PI21004). Elution was fractionated, and fractions were neutralized with IM Tris-HCL, pH 8.5 (Fisher Scientific, cat No. 50-843-270) and assayed by A280 (DropSense96, Trinean). Peak fractions were pooled, and buffer exchanged into DPBS. Centrifugal filters (EMD Millipore, cat. No. UFC803024) were equilibrated in DPBS at 4,000×g for 2 mins. Purified sample was loaded, DPBS was added, and the sample was spun at 4,000×g for 5 to 10 minute spins until total DPBS volume reached >6 DV. The final pool was analyzed by A280.

Stable cell lines expressing human CDH17 were generated by transfecting a selected host cell (i.e., CHO-K1) with pcDNA3.1-based plasmids expressing Homo sapiens CDH17NP_001138135 (isoform 1 precursor) (SEQ ID NO: 110), >XP 045254438.1 Macaca Fascicularis CDH17 (cynomolgus monkey) (SEQ ID NO: 111), or Mus musculus CDH17 XP_006537646 (mouse) (SEQ ID NO: 112) using electroporation-based transfection. Blasticidin was used to select the integrated cells. After 7-10 days of blasticidin selection, stable clones were isolated by FACS using a CDH17-specific reference antibody (see, WO 2017/095805) and an Alexa Fluor 488 or Alexa Fluor 647 conjugated secondary antibody. After expansion, the stable clones were further confirmed for human CDH17 expression by flow cytometry. Stable cell lines in which CDH17 protein expression was knocked down were generated by transfecting a selected host cell (i.e., COLO205 GFP) with a mix of three CRISPR guide RNAs against CDH17 and the Cas9 enzyme using an electroporation-based transfection. Cells were allowed to proliferate, and single-clone limited dilution was performed. After 7-20 days of selection, CDH17 KO stable clones were isolated by FACS using the CDH17-specific reference antibody PC1 and Alexa Fluor 647 conjugated secondary antibody. After expansion, the stable clones were further confirmed for the absence of expression of human CDH17 protein using a panel of CDH17 specific antibodies by flow cytometry.

The sequences for the heavy and light chain variable regions for hybridoma clones were determined as described below. Total RNA was extracted from 1-2×10⁶ hybridoma cells using the RNeasy Plus Mini Kit from Qiagen (Germantown, AID, USA). CDNA was generated by performing 5′ RACE reactions using the SMARTer RACE 573′ Kit from Takara (Mountainview, CA, USA). PCR was performed using the Q5 High-Fidelity DNA Polymerase from NEB (Ipswich, MA, USA) to amplify the variable regions from the heavy and light chains using the Takara Universal Primer Mix in combination with gene specific primers for the 3′ mouse constant region of the appropriate immunoglobulin. The amplified variable regions for the heavy and light chains were run on 2% agarose gels, the appropriate bands excised and then gel purified using the Mini Elute Gel Extraction Kit from Qiagen. The purified PCR products were cloned using the Zero Blunt PCR Cloning Kit from Invitrogen (Carlsbad, CA, USA), transformed into Stellar Competent E. Coli cells from Takara and plated onto LB Agar+50 μg/ml kanamycin plates. Direct colony Sanger sequencing was performed by GeneWiz (South Plainfield, NJ, USA). The resulting nucleotide sequences were analyzed using IMGT V-QUEST to identify productive rearrangements and analyze translated protein sequences. CDR determination was based on Kabat numbering.

Selected VH or VL chains were PCR amplified and cloned into a pcDNA3.4-based expression vector, which harbors the constant region from human IgG1 (Uniprot P01857) or human kappa light chain (UniProt P01834). Paired heavy chain- and light chain-expressing plasmids were transfected into Expi293 cells (Thermo Fisher Scientific) following provider's Expi293 expression system protocol. Five days after transfection, culture supernatants were collected by centrifugation. Chimera antibodies were purified by 1-step affinity purification using Protein A column and buffer exchanged to PBS pH 7.2.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard techniques for characterizing ligand/receptor interactions are available. See, e.g., Coligan et al. (2001) Current Protocols in Immunology, Vol. 4, John Wiley, Inc., New York. Standard methods of antibody functional characterization appropriate for the characterization of antibodies with particular mechanisms of action are also well known to those of skill in the art.

In-house CDH17-specific reference antibodies based on the human anti-CDH17 antibody, (ARB202), referred to herein as “Positive Control 1” (PC1), and on the fully human ‘BI’, referred herein as PC3 were prepared respectively based on the publicly available information (PC1 VH/VL: (SEQ ID NO: 3 and SEQ ID NO: 4, WO WO2019/222428; PC3 VH/VL (SEQ ID NO 116) and (SEQ ID NO 117) WO WO2018/115231). The PC1 and/or PC3 antibodies were used to confirm CDH17 expression by the transfectant cell lines and lack of expression of CDH17 KO cell lines used in the examples and to establish the binding and functional assays used to evaluate and characterize the anti-CDH17 specific antibodies disclosed herein.

Additional in-house CDH17-specific reference antibodies PC7 and PC8 were prepared based on publicly available information. (PC7 VH/VL: (SEQ ID NO: 38 and SEQ ID NO: 49, WO2010/123874; PC8 VH/VL (SEQ ID NO: 49 and SEQ ID NO: 50, WO2023/107558).

Internalization of CDH17-specific antibodies may be assessed by a pH dependent imaging assay that uses the co-internalization of the target bound antibody together with an anti-human IgG Fab fragment conjugated with a pHAb dye (Promega, Cat No. G9845), which is non fluorescent at neutral pH and becomes highly fluorescent at acidic pH. The detection of fluorescent signal is indicative that the mAb combined with the pH sensitive dye conjugated secondary has reached an intracellular compartment with acidic pH (i.e., late endosome or lysosome) (Nath et al., J Immunol Methods, 431:11-21).

Use of species and isotype-specific secondary ADCs to “piggyback” the effector payload on an unmodified mAb has served as a common shortcut for ADC mediated killing evaluation. Cytotoxic compounds fused to an antibody-binding domain allowing formation of a stable non-covalent ADC may represent universal tools for fast screening of a large number of antibodies. Payloads such as MMAF, MMAE, PNU, DX8951, PBD and others are readily available in secondary antibodies with both cleavable and non-cleavable formats (Kellner, et al., J Immunol Methods 371:122-133, 2011, Hellman, et al. Front Immunol, 9:2490, 2018). Therefore, to evaluate their potential ADC dependent killing activity or target expressing cells, mAbs can be pre-incubated with species and isotype-specific secondary conjugated ADCs and then added to in vitro cultured target cells. Killing may then be assessed by Cell titer Glo assays, or live-cell imaging of GFP target cells and normalized to payload alone and or isotype or untreated cells.

Software packages and databases for determining, e.g., antigenic fragments, leader sequences, protein folding, functional domains, CDR annotation, glycosylation sites, and sequence alignments, are available.

### Example 2: Generation of Anti-CDH17 Antibodies

Mouse anti-CDH17 antibodies were generated by immunizing wildtype mice with recombinant human CDH17 protein.

Wildtype mice were immunized with recombinant human CDH17 protein intraperitoneally (IP) and subcutaneously (SC). The immune response was monitored by retroorbital bleeds. The plasma was screened by ELISA, flow cytometry (FACS) and or Imaging (as described below). Mice with sufficient titers of anti-CDH17 antibodies were used for fusions. Mice were boosted intraperitoneally with recombinant human CDH17 protein and or CHO cell line expressing human CDH17 protein before sacrifice and removal of the spleens.

To select mice producing antibodies that bound CDH17 protein, sera from immunized mice were evaluated for binding to recombinant human CDH17 protein and cynomolgus CDH17 by ELISA, cell lines (CHO or Hek293) expressing human, or cynomolgus proteins, and an endogenous cell line (ASPC1) expressing human CDH17 protein by FACS. For ELISA, briefly, an ELISA plate coated with recombinant human or cynomolgus CDH17 protein was incubated with dilutions of serum from immunized mice for one hour at room temperature, the assay plate was washed, and specific antibody binding was detected with HRP-labeled anti-mouse IgG antibody. Plate was read using an ELISA plate reader (Biotek). For FACS, briefly, human CDH17-CHO cells, cynomolgus CDH17-Hek293 cells, and an endogenous cell line (ASPC1) expressing human CDH17 protein were incubated with dilutions of serum from immunized mice. Cells were washed, and specific antibody binding was detected with Alexa Fluor 647 labeled goat anti mouse IgG antibody (Invitrogen). Flow cytometric analyses were performed on a flow cytometer (Intellicyte, IQue plus, Sartorius). For imaging assay, briefly, human CDH17-CHO cells, cynomolgus CDH17-Hek293 cells, and an endogenous cell line (ASPC1) expressing human CDH17 protein were incubated with dilutions of serum from immunized mice. Cells were washed, fixed with paraformaldehyde, then washed. Specific antibody binding was detected with secondary Alexa Fluor 488 goat anti-mouse antibody and Hoechst (Invitrogen). Plates were scanned and analyzed on an imaging machine (Cytation 5, Biotek).

To generate hybridomas producing mouse antibodies of the present disclosure, splenocytes were isolated from an immunized mouse and fused to an appropriate immortalized cell line, such as a mouse myeloma cell line. The resulting hybridomas were screened for the production of antigen-specific antibodies. For example, single cell suspensions of splenocytes from immunized mice were fused to equal number of Sp2/0 non-secreting mouse IgG myeloma cells (ATCC, CRL 1581) by electrofusion. Cells were plated in flat bottom 96-well tissue culture plates, followed by 2 weeks of incubation in selection medium (HAT medium), then switched to hybridoma culture media.

10-14 days after cell plating, supernatants from individual wells were initially screened for human CDH17 by Imaging. Briefly, CDH17-CHO or ASPC-1 cells were incubated with hybridoma supernatants. Cells were washed, fixed with paraformaldehyde, then washed. Specific antibody binding was detected with secondary Alexa488 goat anti-mouse antibody and Hoechst (Invitrogen). Plates were scanned and analyzed on an imaging machine (Cytation 5, Biotek). The positive hybridomas were then screened for CDH17 binding specificity by ELISA and FACS as described above. The CDH17 antibody secreting hybridomas were transferred to 24-well plates. The positive hybridomas from 24-well plates were then screened for CDH17 binding specificity by ELISA, FACS or Imaging as described above again. The positive hybridomas were subcloned by single cell sorting using a Sony cell sorter (Sony Inc.). The stable subclones were then screened by ELISA, Imaging and FACS as described above. The positive subclones were cultured in vitro to generate small amounts of antibodies for purification and further characterization.

### Example 3: Binding of Anti-CDH17 Antibodies to Human, Cyno, and Mouse CDH17

The binding affinities of the parental murine antibody clones for the disclosed anti-CDH17 antibodies to human, cynomolgus monkey, and mouse CDH17 were evaluated using cell-based binding assays. Briefly, CHO cell lines overexpressing human CDH17, cynomolgus CDH17, or mouse CDH17 were incubated with pre-diluted antibodies (C17_mAb1-10) for 2 hours at 4° C. Cells were then fixed with 4% PFA for 20 minutes at room temperature and washed twice. Specific antibody binding was detected using AF488-labeled goat anti-mouse IgG. After incubation with the secondary antibody for 1 hour at room temperature and two washes, cells were analyzed for AF488 fluorescence intensity using an iQue (Sartorius).

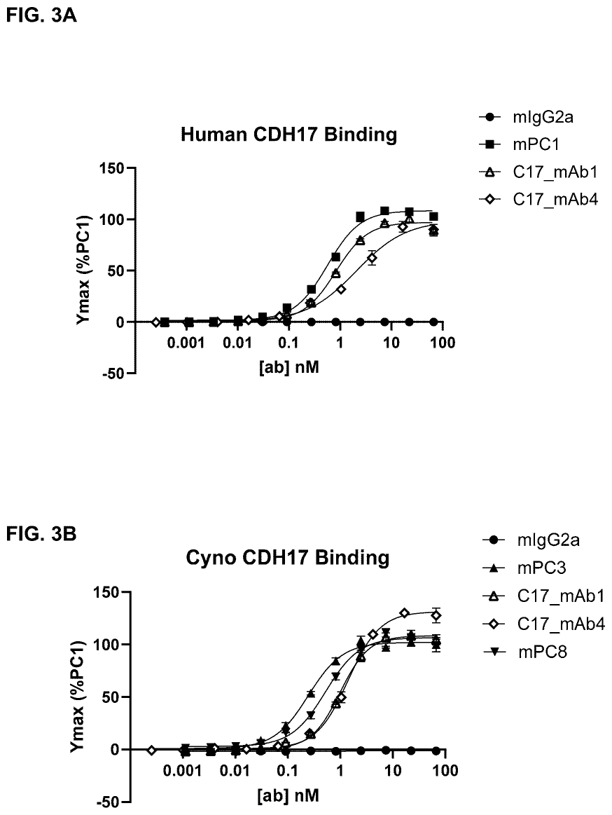

FIGS. 3A-B show that the positive controls (PCs) as well as C17_mAb1 and C17_mAb4 bind to human CDH17 (3A) and cynomolgus CDH17 (3B) in a dose-dependent manner, validating the expression of these targets in the cell lines.

All ten parental anti-CDH17 murine antibodies (C17_mAb1 to C17_mAb10) bound to human CDH17 expressed on CHO cells in a dose-dependent manner, exhibiting EC50 values ranging from 0.4 nM to 5 nM. The positive control PC1 also bound to human CDH17 with an EC50 of 0.56 nM.

The same panel of ten parental murine antibodies also demonstrated dose-dependent binding to cynomolgus CDH17 expressed on CHO cells, with EC50 values ranging from 0.1 nM to >66.7 nM. The positive controls (PC1 and PC8) bound to cynomolgus CDH17 with EC50 values of 0.25 nM and 0.52 nM, respectively.

No binding was observed to mouse CDH17. This lack of binding may be explained by the lower sequence homology of mouse CDH17 compared to human CDH17. Orthologs of CDH17 share 90.45% homology between human and cynomolgus monkey, but only 79.44% homology between human and mouse.

In summary, initial characterization showed that the ten parental murine anti-CDH17 antibodies (C17_mAb1 to C17_mAb10) bind human CDH17 with high affinity and exhibit comparable cross-reactivity to cynomolgus CDH17, except for C17_mAb9. A summary of their relative binding strengths is presented in Table 3.

TABLE 3
Relative Binding Affinities of Antibodies to CDH17

| | Human | Cyno | Mouse |
|---|---|---|---|
| Antibody name | (EC₅₀, nM) | (EC₅₀, nM) | (EC₅₀, nM) |
| PC1 | +++ | ND | ND |
| PC3 | ND | +++ | ND |
| PC7 | ND | ND | ND |
| PC8 | ND | +++ | ND |
| C17_mAb 1 | +++ | +++ | − |
| C17_mAb 2 | ++ | ++ | − |
| C17_mAb 3 | ++ | ++ | − |
| C17_mAb 4 | ++ | ++ | − |
| C17_mAb 5 | ++ | ++ | − |
| C17_mAb 6 | ++ | ++ | − |
| C17_mAb 7 | +++ | +++ | − |
| C17_mAb 8 | ++ | +++ | − |
| C17_mAb 9 | ++ | − | − |
| C17_mAb 10 | ++ | ++ | − |

−: >66.67 nM
+: >10 nM
++: 1-10 nM
+++: <1 nM
ND: Not determined

### Example 4: CDH17 Specificity

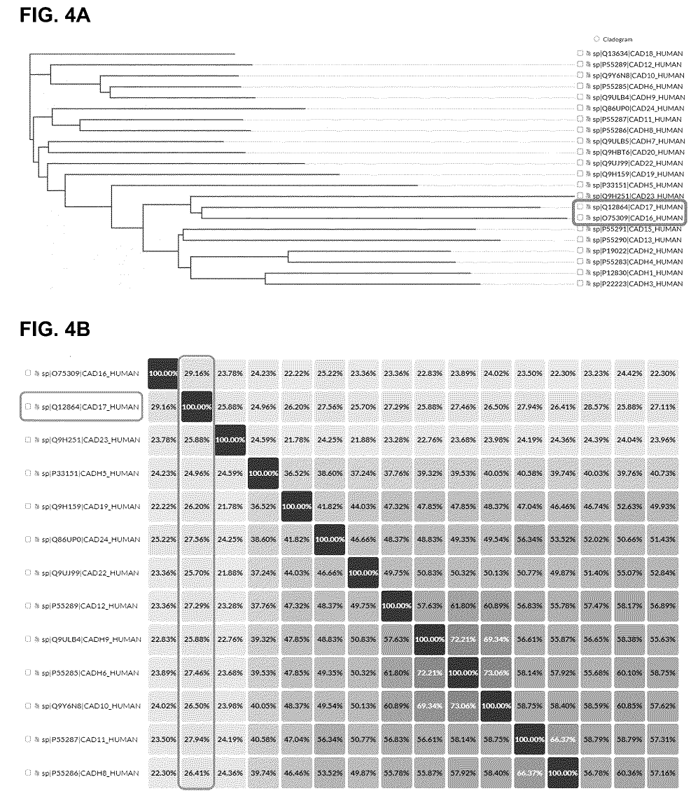

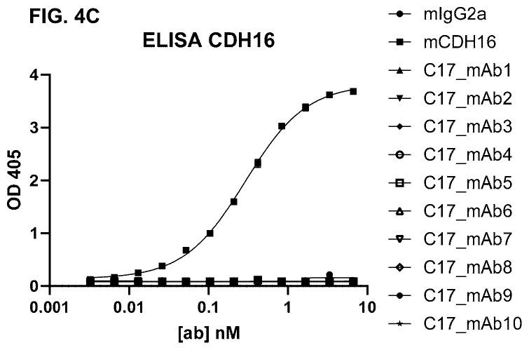

Using multiple sequence alignment tool in Uniprot.org, the family tree and sequence homology of CDH17 with its family members CDH1-23 was examined. FIG. 4A shows a cladogram illustrating the multiple sequence alignment tree of CDH17 and its family members. FIG. 4B shows the percentage of sequence homology between CDH17 and its family members. The closest family member to CDH17 is CDH16 with sequence homology of ˜29.16% similarity.

Binding to CDH16 was examined by ELISA. Briefly, recombinant human CDH16 protein was coated on 384 well plate overnight in 4° C. Plates were washed thrice and blocked with PBS/5% chicken serum for 1 hr RT. Plates were washed before addition of pre-diluted anti-CDH17 antibodies and incubated for 1 hr RT. Plates were washed and anti-mouse IgG HRP was used to detect anti-CDH17 bound to recombinant proteins. Plates were washed again before ABTS was added and incubated for 20 min RT before being read with BioTek plate reader.

FIG. 4C shows positive control (Sino Bio, Cat #10915-MM09) binding to human CDH16 protein while no binding was observed by any of the disclosed murine chimeric anti-CDH17 antibodies. The disclosed anti-CDH17 antibodies do not cross bind to CDH16.

Biparatopic antibodies (biparatopic 1 and biparatopic 2) were examined for non-target specificity using a Membrane Proteome Array (MPA) platform (Integral Molecular). In this platform the antibodies were screened for off target binding to 6000 membrane protein natively expressed on unfixed human cells. The disclosed biparatopics 1 and biparatopic 2 showed no binding to any of the off-target proteins indicating high specificity for its target CDH17.

### Example 5: Binding of Anti-CDH17 Antibodies to Tumor Cells Endogenously Expressing CDH17

The binding affinity of the parental anti-CDH17 antibodies to endogenously expressed human CDH17 in AsPC1 (high) and COLO205 (moderate) tumor cell lines was examined using cell-based flow cytometry assay.

To examine CDH17 density on cancer cells, human cancer cells as indicated were stained with saturation concentration of PC8 hIgG for 30 min at 4° C. before being fixed for 20 min with 4% PFA at RT. Cells were then washed twice before addition of anti-human IgG conjugated to AF647 and incubated for 30 min RT. Cells were washed twice before being read on the flow cytometer (Attune Nxt, Invitrogen). The density of CDH17 was calibrated by incorporating the median fluorescent intensity signal (MFI) and the receptor density per cell was calculated using a standard curve of AF647 Quantum MESF beads following manufacturer's instructions (Bangs Laboratories Inc., Cat. No. 5647).

For binding assay, colorectal cancer cells COLO205 and pancreatic cancer cells AsPC1 endogenously expressing CDH17 were incubated with prediluted antibodies at 4° C. for 2 hours. Cells were then fixed with 4% PFA before being washed twice with PBS. Anti-CDH17 antibodies were then detected by incubation with goat anti-mouse IgG conjugated to AF488. Cells were acquired for AF488 fluorescent intensity in the flow cytometer iQue (Satorius).

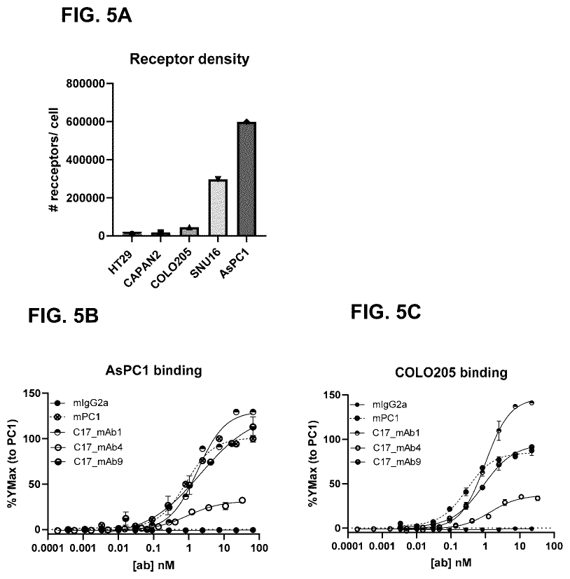

FIG. 5A shows the relative CDH17 density in the various human cancer cell lines ranging from 10,654-599,898 receptors per cell. FIG. 5B-C shows binding activities of select anti-CDH17 antibodies on AsPC1 and COLO205 cells. Binding to human CDH17 is dose dependent and the binding profile of anti-CDH17 is maintained across both cell lines.

### Example 6: Binding Domain Mapping of Anti-CDH17 Antibodies

To determine the binding sites of the disclosed anti-CDH17 antibodies, His-tagged recombinant proteins corresponding to ECD1-2 (SEQ ID NO: 114), ECD3-5 (SEQ ID NO: 115), and ECD6-7 (SEQ ID NO: 116) of human CDH17 were generated using HEK293 cells. The purified proteins were coated onto 384-well plates overnight at 4° C. Plates were then washed three times and blocked with PBS containing 5% chicken serum for 1 hour at room temperature. After washing, pre-diluted anti-CDH17 antibodies were added and incubated for 1 hour at room temperature. Plates were washed again, and bound antibodies were detected using HRP-conjugated anti-mouse IgG. Following a final wash, ABTS substrate was added and incubated for 20 minutes before absorbance was measured using a BioTek plate reader.

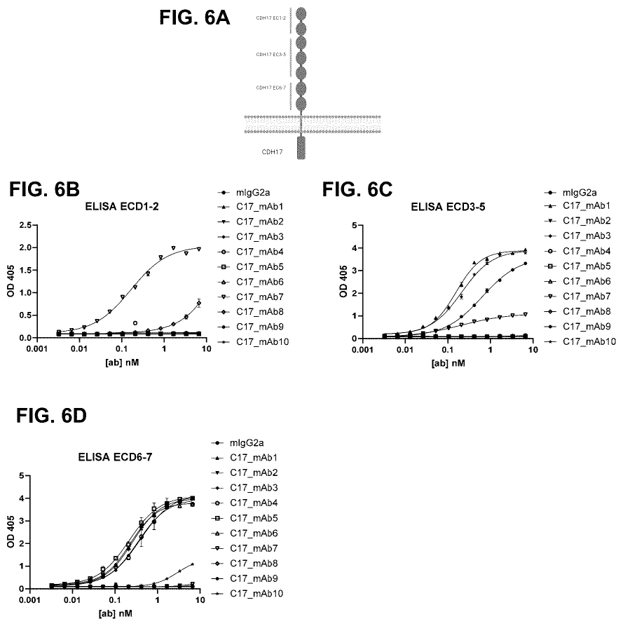

FIG. 6A shows a schematic of the CDH17 structure, which contains seven extracellular domains, and illustrates the grouping of EC1-2, EC3-5, and EC6-7. FIGS. 6B-D show the binding specificities of the parental murine anti-CDH17 antibodies to the defined ECDs of CDH17. The disclosed antibodies collectively span binding to ECD1-7 of CDH17, with some antibodies binding to more than one defined ECD at varying levels.

Table 4 below summarizes the ECD binding profiles of the anti-CDH17 antibodies as determined by the ELISA results shown in FIGS. 6B-D.

TABLE 4
ECD Binding of anti-CDH17 Antibodies

| | Primary Binding site | Secondary Binding Site |
|---|---|---|
| C17_mAb 1 | D 3/5 | N.B. |
| C17_mAb 2 | D 6/7 | N.B. |
| C17_mAb 3 | D 6/7 | N.B. |
| C17_mAb 4 | D 6/7 | N.B. |
| C17_mAb 5 | D 6/7 | N.B. |
| C17_mAb 6 | D 6/7 | N.B. |
| C17_mAb 7 | D 1/2 | D 3/5 |
| C17_mAb 8 | D 1/2 | N.B. |
| C17_mAb 9 | D 3/5 | N.B. |
| C17_mAb 10 | D 3/5 | D 6/7 |

Strong binding was designated at OD405 >1 while weak binding was referred when binding was observed but OD405 was 0.2-1. N.B.=No Binding. Next, cross-epitope binning of the anti-CDH17 antibodies was examined using a cell-based binding assay. The disclosed murine antibodies were labeled with AF488 (ThermoFisher, Cat #A20181) according to the manufacturer's instructions. AsPC1 cells, which endogenously express CDH17, were incubated with unlabeled anti-CDH17 antibodies for 2 hours at 4° C., followed by addition of AF488-labeled anti-CDH17 antibodies. Cells were then incubated for another 2 hours at 4° C., fixed with 4% PFA for 20 minutes at room temperature, and washed twice before acquisition on an iQue flow cytometer to measure AF488 fluorescence intensity.

Each bin was defined based on known ECD binding specificities as shown in Table 4. For example, antibodies specific to ECD1-2 were grouped into one bin, antibodies specific to ECD3-5 into another, and antibodies specific to ECD6-7 into a third.

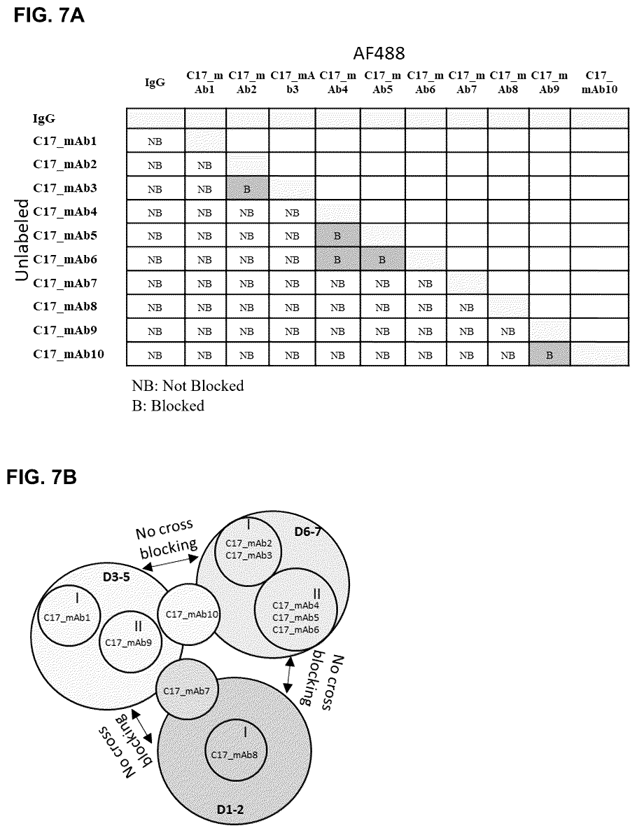

FIG. 7A shows whether individual antibodies listed in the left column were able to block the AF488-labeled antibodies listed in the top row. The MFI for AF488 was normalized to the IgG control (unlabeled), with 100% indicating maximum binding to its epitope. Within each bin, if an antibody blocked the binding of another antibody by less than 50%, it was designated as blocked (B) and placed in the same group. If they did not block each other by more than 50%, it was designated as not blocked (NB) and they were grouped separately.

FIG. 7B is a schematic diagram outlining how the antibodies were binned and grouped. In summary, antibodies within each bin did not cross-block antibodies from other bins; for example, ECD1-2 binding antibodies did not cross-block ECD3-5 or ECD6-7 binding antibodies, and a similar trend was observed for ECD3-5 and ECD6-7 bins.

Furthermore, within each bin, there were two groups where antibodies belonging to a specific bin did not block each other. For example, within the ECD6-7 bin, there were two groups: C17_mAb2 and C17_mAb3 blocked each other but did not block C17_mAb4, C17_mAb5, or C17_mAb6.

In summary, the disclosed anti-CDH17 antibodies formed three distinct bins, with antibodies within each bin not cross-blocking antibodies from other bins. Additionally, within each ECD bin, there were two groups of antibodies that did not block each other.

### Example 7: Humanization of Anti-CDH17 Antibodies

To enable therapeutic development, the variable regions of four selected parental murine anti-CDH17 antibodies (C17_mAb1 to C17_mAb4) were humanized. For each clone, the complementarity-determining regions (CDRs) from the parental murine heavy chain (HC) and light chain (LC) were grafted onto human germline framework sequences identified based on highest sequence homology and structural compatibility. Back-mutations were introduced where necessary to restore binding affinity or structural stability, as determined through in silico modeling and analysis.

Table 5 summarizes the parental clones and their corresponding humanized heavy chain (Hz HC) and light chain (Hz LC) variants generated. These humanized antibodies were subsequently used to construct biparatopic antibodies (bpAbs) and bispecific antibodies (BsAbs) for further functional evaluation. The sequence IDs for the humanized heavy and light chains are also listed in Table 5.

TABLE 5
Lead Panel of Parental antibodies
and Humanized anti-CDH17 mAbs

| | VH SEQ | VL SEQ |
|---|---|---|
| Clone name | ID NO: | ID NO: |
| C17_mAb 1 (Parental) | 1 | 2 |
| C17_mAb 1_Hz21 | 76 | 78 |
| C17_mAb 1_Hz23 | 76 | 79 |
| C17_mAb 1_Hz31 | 77 | 78 |
| C17_mAb 1_Hz33 | 77 | 79 |
| C17_mAb 2 (Parental) | 3 | 4 |
| C17_mAb 2_Hz11 | 80 | 82 |
| C17_mAb 2_Hz12 | 80 | 83 |
| C17_mAb 2_Hz31 | 81 | 82 |
| C17_mAb 2_Hz32 | 81 | 83 |
| C17_mAb 3 (Parental) | 5 | 6 |
| C17_mAb 3_Hz31 | 84 | 86 |
| C17_mAb 3_Hz33 | 84 | 87 |
| C17_mAb 3_Hz41 | 85 | 86 |
| C17_mAb 3_Hz43 | 85 | 87 |
| C17_mAb 4 (Parental) | 7 | 8 |
| C17_mAb 4_Hz31 | 123 | 124 |

For the humanized antibodies, BLI assays were performed using the corresponding recombinant protein domains to measure their binding affinities. Specifically, the binding of humanized Fabs or full-length antibodies to hCDH17 domains D1-2, D3-5, or D6-7 was assessed using the OCTET system, the data is summarized in Table 6.

TABLE 6
BLI-Measured Binding Affinity of Lead Parental
and Humanized Anti-CDH17 Antibodies

| Name | KD (M) | kon(1/Ms) | kdis(1/s) |
|---|---|---|---|
| C17_mAb1_hIgG1 | 1.38E−08 | 7.82E+04 | 1.08E−03 |
| C17_mAb1_Hz21 | 1.98E−08 | 8.15E+04 | 1.62E−03 |
| C17_mAb1_Hz23 | 1.52E−08 | 1.12E+05 | 1.70E−03 |
| C17_mAb1_Hz31 | 3.34E−08 | 7.37E+04 | 2.46E−03 |
| C17_mAb1_Hz33 | 2.98E−08 | 8.79E+04 | 2.62E−03 |
| C17_mAb2_hIgG1 | 3.61E−10 | 4.95E+04 | 1.79E−05 |
| C17_mAb2_Hz11 | <4.35E−11 | 2.30E+04 | <1E−06 |
| C17_mAb2_Hz12 | <2.42E−11 | 4.13E+04 | <1E−06 |
| C17_mAb2_Hz31 | <2.29E−11 | 4.36E+04 | <1E−06 |
| C17_mAb2_Hz32 | <2.62E−11 | 3.81E+04 | <1E−06 |
| C17_mAb3_hIgG1 | 1.33E−08 | 2.14E+04 | 2.84E−04 |
| C17_mAb3_Hz31 | 2.60E−08 | 2.33E+04 | 6.05E−04 |
| C17_mAb3_Hz33 | 1.91E−08 | 1.52E+04 | 2.90E−04 |
| C17_mAb3_Hz41 | 1.15E−08 | 3.77E+04 | 4.32E−04 |
| C17_mAb3_Hz43 | 5.55E−09 | 3.36E+04 | 1.87E−04 |
| C17_mAb4_Fab | 7.50E−10 | 8.37E+05 | 6.28E−04 |
| C17_mAb4_Hz31_Fab | 6.90E−10 | 1.57E+06 | 1.08E−03 |

Cell based binding by humanized anti-CDH17 antibodies was examined in COLO205 and AsPC1 cells. Table 7 shows the binding affinity of humanized anti-CDH17 mAbs on COLO205 and AsPC1 cells. Corresponding Parental antibodies (antibodies prior to humanization) were used as controls The binding data for C17_mAb4 and C17_mAb4_Hz31 were generated using Fab constructs, while all other binding data were obtained using full-length antibodies.

TABLE 7
Binding Affinity of Humanized Anti-
CDH17 Antibodies by Cell Based Assay

| | | AsPC1 |
|---|---|---|
| | COLO205 | binding |
| Clone name | binding EC50 | EC50 |
| C17_mAb 1 | 0.4301 | 3.57 |
| C17_mAb 1_Hz21 | 0.4407 | 1.472 |
| C17_mAb 1_Hz23 | 0.3162 | 1.854 |
| C17_mAb 1_Hz31 | 0.4859 | 1.638 |
| C17_mAb 1_Hz33 | 0.4808 | 2.064 |
| C17_mAb 2 | 1.597 | 3.421 |
| C17_mAb 2_Hz11 | 0.7668 | 1.553 |
| C17_mAb 2_Hz12 | 0.7867 | 1.599 |
| C17_mAb 2_Hz31 | 1.845 | 2.474 |
| C17_mAb 2_Hz32 | 1.807 | 2.7 |
| C17_mAb 3 | 5.543 | 4.214 |
| C17_mAb 3_Hz31 | 2.095 | 3.256 |
| C17_mAb 3_Hz33 | 1.884 | 3.922 |
| C17_mAb 3_Hz41 | 2.637 | 3.428 |
| C17_mAb 3_Hz43 | 1.873 | 3.245 |
| *C17_mAb 4 | 0.19 | NA |
| *C17_mAb 4_Hz31 | 0.43 | NA |

In summary, the results suggest that the selected humanized versions of the anti-CDH17 antibodies retained the binding characteristics of their corresponding parental murine antibodies.

### Example 8: Molecular Design and Production of Anti-CDH17 Biparatopics Antibodies

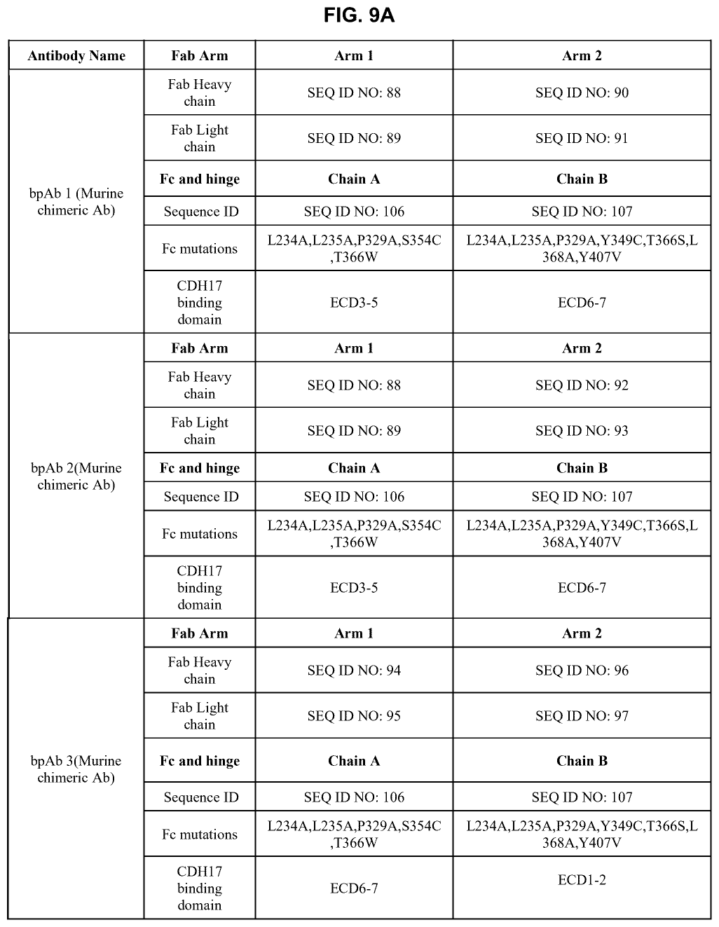

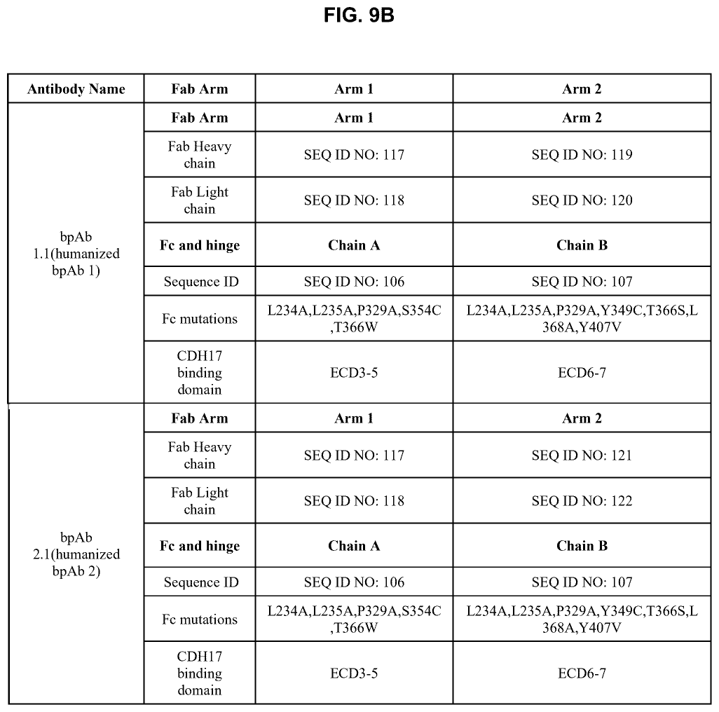

Biparatopic anti-CDH17 antibodies binding to two non-overlapping epitopes on the antigen, were synthesized in IgG-like format. FIGS. 8A-B illustrate the engineered Fc and Fab for biparatopic antibodies with two designs: NR25 and NR26. The heterodimeric Fc and hinge region are presented with chain A in white and chain B in black. The Fab binding to the first CDH17-binding domain (Arm 1) is depicted in grey, while the second CDH17-binding domain (Arm 2) is shown in hatched fill. To enhance the heterodimer formation of Fc for improved production, “knob in hole” mutations on CH3 were applied (Carter P. et al. (1998)). And to eliminate FcγR-binding the “LALAPA” mutations are introduced in the Fc region. These Fc mutations are provided in FIG. 9 (code by EU numbering). Additionally, to facilitate the correct pairing of the light chain and heavy on each Fab arm, engineering on Fab was performed through the disulfide shift strategy (NR25 design) or electrostatic-steering (NR26 design) depicted in FIG. 8A-B. The sequences of the Fab for each arm of the chimeric biparatopic anti-CDH17 antibodies comprising murine HC and LC sequences are outlined in FIG. 9A as BpAb1, BpAb2 and BpAb3. The sequences of the Fab for each arm of anti-CDH17 biparatopic antibodies prepared using humanized HC and LC variants are provided in FIG. 9B as BpAb1.1 and BpAb2.1.

The cloning and expression of the antibodies followed these steps: DNA segment encoding the antibody heavy and light chains were synthesized using codons optimized for human/mammalian expression, then sub-cloned into the mammalian expression vector pcDNA 3.4 (ThermoFisher), and transiently expressed in Expi293 cells (ThermoFisher) for production under the condition of 37° C. in CO₂ incubator. Transfected cells were harvested after 5-6 days, with the culture medium collected. The clarified culture medium was loaded onto a MabSelect SuRe (Cytiva) protein-A column and eluted with IgG elution buffer at pH 2.8 (ThermoFisher) to recover Fc-containing products. Subsequently, the product underwent additional purification steps using KappaSelect (Cytiva) and LambdaFabSelect (Cytiva) to remove non-desired products. SDS-PAGE (BioRad), size exclusion HPLC (Agilent 1100 series) analysis with SE-HPLC column (TOSO,G3000SWXL) were performed to detect and confirm the size and purity of the antibodies. Alternatively, the biparatopics antibodies could be purified from the supernatant of stable CHO expression cell lines.

### Example 9: Murine Anti-CDH17 Biparatopic Antibody Has Enhanced Internalization and Internalization Kinetics Which Is Not Due to Increased Binding

Three experiments were conducted to characterize murine anti-CDH17 biparatopic antibodies and to compare them with PC7, PC8 or their respective mAbs alone and a combination of respective mAbs.

The first experiment is the internalization of anti-CDH17 antibodies using a pH sensitive dye in image-based assay in COLO205 and COLO205 CDH17 KO (FIGS. 10A-B). The second, is cell based binding experiment in COLO205 (FIG. 10C). The third, is internalization kinetics of anti-CDH17 antibodies assessed using AF488 labeled antibodies in AsPC1 (FIGS. 10D-E).

In the first study, the internalization of anti-CDH17 in COLO205 or COLO205 CDH17 KO cells was assessed using pH sensitive dye in image-based assay. This assay measures the co-internalization of anti-CDH17 bound to its antigen and pH dye labeled anti-mouse IgG (Fab) bound to anti-CDH17. The pH dye is not fluorescent in neutral pH (˜7.2) and becomes florescent at acidic pH. Thus, upon co-internalization, the acidic compartments within a cell will induce a fluorescent signal that can be read by imaging cells in a cell imager.

Briefly, in the internalization assay, cells were seeded on imaging plates during preparation of the antibody mix. Anti-CDH17 antibodies were incubated with pH dye labeled anti-mouse IgG (Fab) at molar ratio to 1:3 for 30 mins before being serially diluted. Prediluted antibody cocktail was incubated with cells for 16 hrs in 37° C. Cells were stained with nuclear stain to identify cells and washed before being imaged in the Cytation 5 (BioTek) cell imager.

In the binding assay, tumor cells were incubated with serially diluted anti-CDH17 antibodies for 2 hr in 4° C. before being fixed. Cells were washed twice and incubated with AF488 labeled goat anti-mouse IgG antibody for 1 hr at room temperature. Cells were washed twice before being acquired on iQue for AF488 fluorescent intensity.

In the internalization kinetic study, selected antibodies were labeled with AF488 using a labeling kit (Invitrogen, Cat #A20181). Tumor cells were seeded in plates and chilled on ice for 1-2 hr before addition of prediluted AF488 labeled antibodies. Plates were further chilled for 1 hour before being placed in 37° C., 5% CO₂ to start internalization time course. At indicated timepoints internalization was stopped by fixation of cells with 4% PFA for 20 min and cells were washed thrice with 1×DPBS. After final wash cells were incubated with anti-AF488 quencher for 2 hr before being imaged on cytation imager.

FIG. 10A shows greater internalization by the anti-CDH17 biparatopic antibody bpAb 1 compared to PC8 and their respective mAb alone or combination of respective mAbs. Internalization by anti-CDH17 antibodies is also dependent on CDH17 expression as no internalization is observed in COLO205 CDH17 KO cells as shown in FIG. 10B.

FIG. 10C shows binding activity by anti-CDH17 biparatopic antibodies in dose dependent manner to endogenously expressed CDH17. Binding activity of biparatopics is not stronger than PC8, C17_mAb 1 mAb alone or combination of C17_mAb 1+C17_mAb 2 mAbs.

FIG. 10D shows internalization kinetics of anti-CDH17 biparatopic antibodies (bpAb1 and bpA2) is faster than PC8 and their respective mAb alone. Data shown in FIG. 10E is the bar graph of fold change in MFI at 1.5 hours after normalization of 0 hr timepoint for each antibody.

In summary, anti-CDH17 biparatopic antibodies exhibit greater internalization and faster internalization kinetics compared to PC8 and their respective mAb alone or combination of respective mAbs. The biparatopic antibody does not show increased binding, so the observed enhancement in activity is unlikely due to stronger binding. Instead, it may result from its unique ability to bind two non-overlapping epitopes.

### Example 10: Enhanced Internalization by Murine Anti-CDH17 Biparatopic Antibodies is Maintained Across Varying and Lower Levels of CDH17 Expressing Tumor Cell Lines

Internalization activity of anti-CDH17 in endogenously expressing tumor cell lines was accessed using pH sensitive dye in the image-based assay described above in Example 9.

Cells were seeded on imaging plates during preparation of the antibody mixes. Anti-CDH17 antibodies were incubated with pH dye labeled anti-mouse IgG (Fab) at molar ratio to 1:3 for 30 mins before being serially diluted. The pre-diluted antibody cocktail was incubated with cells for 16 hrs in 37° C. Cells were stained with nuclear stain to identify cells and washed before being imaged in the Cytation 5 (BioTek) cell imager.

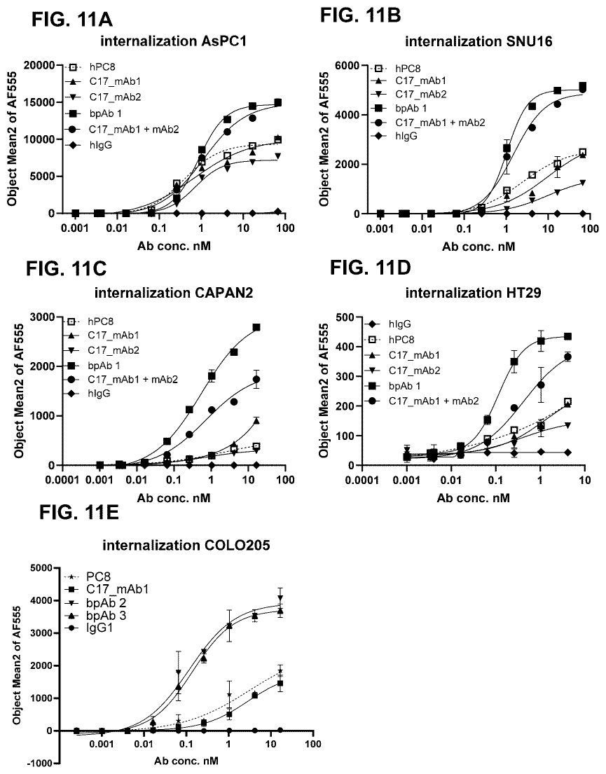

FIG. 11 shows that the anti-CDH17 biparatopic mAb1 induces enhanced internalization compared to PC8, as well as compared to their respective individual mAbs or the combination of those mAbs. This enhanced internalization is consistently observed across cell lines with varying levels of CDH17 expression, including high-expression cell lines ASPC1 (FIG. 11A) and SNU16 (FIG. 11B), low-expression cell lines CAPAN2 (FIG. 11C) and HT29 (FIG. 11D), and a medium/low-expression cell line Colo205 (FIG. 11E). Notably, the enhancement is more apparent in lower-expressing cell lines such as HT29. FIG. 11E also presents representative data for biparatopic 2 and biparatopic 3, both showing enhanced internalization compared to PC8 and CDH17_mAb1 in Colo205 cells.

The anti-CDH17 biparatopic antibodies exhibit enhanced internalization, with this increase being particularly significant in lower CDH17-expressing cell lines such as CAPAN2 (FIG. 11 C) and HT29 (FIG. 11D).

### Example 11: Enhanced Cell Cytotoxicity by Murine Anti-CDH17 Biparatopic Antibodies in COLO205 Cells

Endocytosis induced cell cytotoxicity by CDH17 specific antibodies bound to CDH17 expressing COLO205 cells was measured using a co-internalization of anti-CDH17 bound to its target with an anti-human Fab-αHFc-CL-MMAF antibody.

Tumor cells were seeded on plates while antibody cocktail was being prepared. Anti-CDH17 antibodies were incubated with Fab-αHFc-CL-MMAF at molar ratio of 1:6 for 30 mins at room temperature (RT) before being serially diluted. Cells were then incubated with pre-diluted antibodies for 96 hrs in 37° C., 5% CO₂. To measure cell viability, cell titer glo (Promega) was added and luminesce was measured immediately by Neo2 plate reader (BioTek). Media or MMAF alone (at 250 mM) was used as negative and positive controls for normalization as 0%-100% killing respectively.

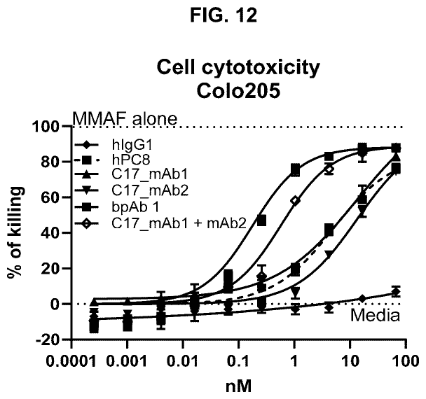

FIG. 12 shows improved cell cytotoxicity by anti-CDH17 biparatopic compared to PC8 and their respective mAb alone or combination of respective mAbs.

Anti-CDH17 biparatopic antibodies have enhanced cell cytotoxicity in COLO205 cells.

### Example 12: Molecular Design and Production of Anti-CDH17xCD3 Bispecific Antibodies

The anti-CDH17xCD3 antibodies targeting CDH17 and CD3 were synthesized in IgG-scFv format. The IgG-scFv format is illustrated in FIG. 13A which presents the heterodimeric Fc and hinge with chain A in white and chain B in black. The scFv binding to the first antigen-binding domain (CD3) is depicted in grey, while the second antigen-binding domain (CDH17) is shown in hatched fill. To enhance the heterodimer formation of Fc for improved production, “knob in hole” mutations on CH3 (Carter P. et al. (1998)) were applied. To eliminate FcγR-binding, the “LALAPA” mutations were introduced in the Fc region. These Fc mutations are provided in FIG. 13B (code by EU numbering). The sequences of the Fab for each arm of bispecific anti-CDH17 antibodies comprising murine HC and LC sequences are outlined in FIG. 13 as BsAb1, BsAb2 and BsAb3. The sequences of the Fab for each arm of anti-CDH17 bispecific prepared using humanized HC and LC variants are provided in FIG. 13C as BsAb4 and BsAb5.

The cloning and expression of the antibodies followed these steps: DNA segment encoding the antibody heavy and light chains were synthesized using codons optimized for human/mammalian expression, then sub-cloned into the mammalian expression vector pcDNA 3.4 (ThermoFisher), and transiently expressed in Expi293 cells (ThermoFisher) for production under the condition of 37° C. in CO₂ incubator. Transfected cells were harvested after 5-6 days, with the culture medium collected. The clarified culture medium was loaded onto a MabSelect SuRe (Cytiva) protein-A column and eluted with IgG elution buffer at pH 2.8 (ThermoFisher) to recover Fc-containing products. Subsequently, the product underwent additional purification steps using CaptureSelect™ CH1-XL MiniChrom Column to remove the homodimer of chain B. Additional gel filtration chromatography would be applied if necessary to remove the aggregation. SDS-PAGE (BioRad), size exclusion HPLC (Agilent 1100 series) analysis with SE-HPLC column (TOSO,G3000SWXL) were performed to detect and confirm the size and purity of the antibodies. Alternatively, the bispecific antibodies could be purified from the supernatant of stable CHO expression cell lines.

### Example 13: CDH17xCD3-meditated T Cell Activation

T cell activation by murine CDH17xCD3 bsAb was assessed using Jurkat TCR NFAT reporter cell line in the presence and absence of target cells. Jurkat TCR NFAT reporter cell line (Promega) is a luciferase-based reporter cell line used as a surrogate for T cell activation under the NFAT promoter.

In this assay, Jurkat T cells were incubated with and without target cells (COLO205) at a ratio of 2:1, target cells: effector cell. These cells were then added into pre-diluted antibodies and incubated in 37° C., 5% CO₂ incubator for 16 hours. One-glo luciferase was added and incubated for 5 mins at RT before the relative luminesce unit (RLU) was read in BioTek plate reader.

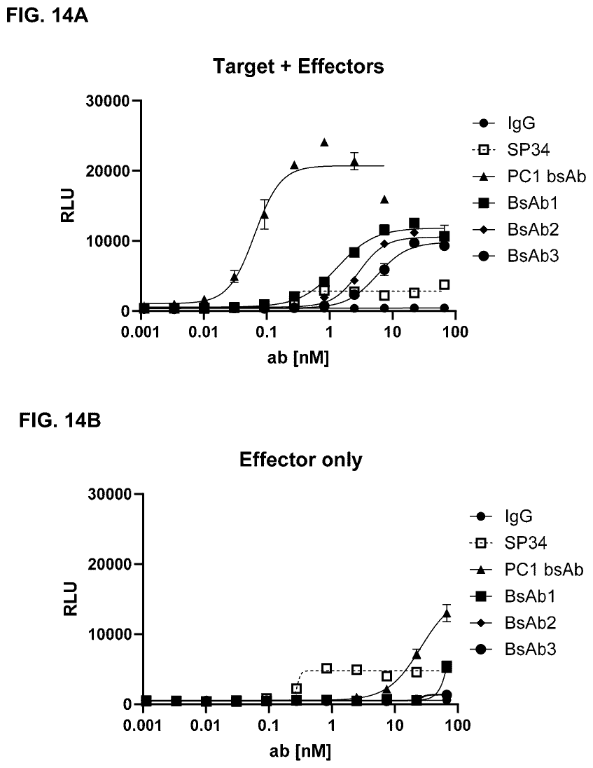

FIG. 14A shows that, in the presence of target cells, CDH17 x CD3 bsAb induced T cell signaling in a dose dependent manner. Without target cells (FIG. 14B), minimal background signaling is observed. These results illustrate that CDH17 x CD3 bsAb induces T cell activation in the presence of target cells.

Further in vivo investigations could evaluate the efficacy of CDH17 x CD3 bsAb by assessing tumor growth inhibition, immune cell infiltration, and cytokine levels, utilizing human embryonic stem cell CD34 humanized mice or human PBMC humanized mice implanted with CDH17-expressing colon, gastric, or liver xenograft models. Histopathological analysis could further elucidate immune cell infiltration into tumors. Such studies yield vital insights into the therapeutic potential of T cell-engaging CDH17-CD3 bispecific antibodies for cancer treatment in vivo.

### Example 14: Enhanced Internalization by Humanized Anti-CDH17 Biparatopic Antibodies in CDH17 Expressing Tumor Cell Lines

To evaluate whether the humanized biparatopic antibodies retained their enhanced internalization properties, the internalization activity of humanized anti-CDH17 biparatopic antibodies was assessed in tumor cell lines endogenously expressing CDH17, alongside their corresponding murine bispecific antibody versions and the positive control PC8.

Cells were seeded onto imaging plates during preparation of the antibody mixes. Anti-CDH17 antibodies were incubated with pH dye-labeled anti-mouse IgG (Fab) at a molar ratio of 1:3 for 30 minutes before being serially diluted. The pre-diluted antibody cocktails were then incubated with cells for 16 hours at 37° C. Cells were stained with a nuclear stain to identify cell nuclei, washed, and subsequently imaged using the Cytation 5 (BioTek) cell imager.

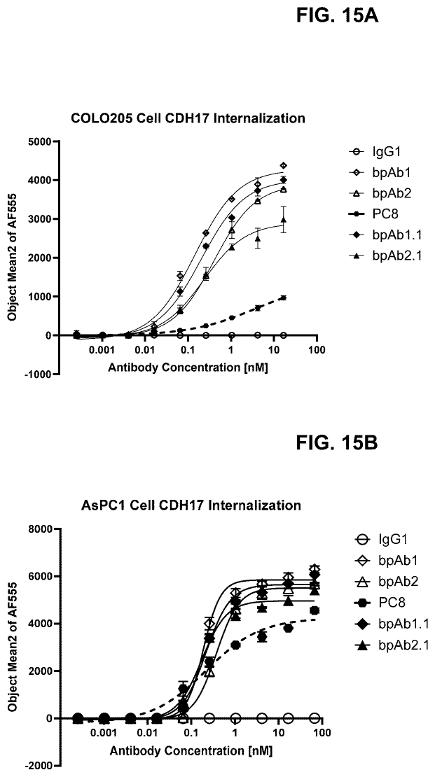

FIG. 15A shows that humanized anti-CDH17 biparatopic antibodies Ab1.1 and Ab2.1 exhibited enhanced internalization compared to PC8 and similar internalization to their respective chimeric bpAb1 and bpAb2 in Colo205 cells. This enhanced internalization was consistent across cell lines with varying levels of CDH17 expression. FIG. 15B demonstrates similar findings in AsPC1 cells, which have high CDH17 expression, where Ab1.1 and Ab2.1 showed enhanced internalization compared to PC8 and comparable activity to their chimeric counterparts.

### Example 15: Enhanced Cell Cytotoxicity by Humanized Anti-CDH17 Biparatopic Antibodies in COLO205 Cells

Endocytosis-induced cell cytotoxicity by humanized anti-CDH17 biparatopic antibodies was measured using a co-internalization assay in which anti-CDH17 antibodies bound to their targets were internalized together with an anti-human Fab-αHFc-CL-MMAF conjugate in CDH17-expressing Colo205 cells.

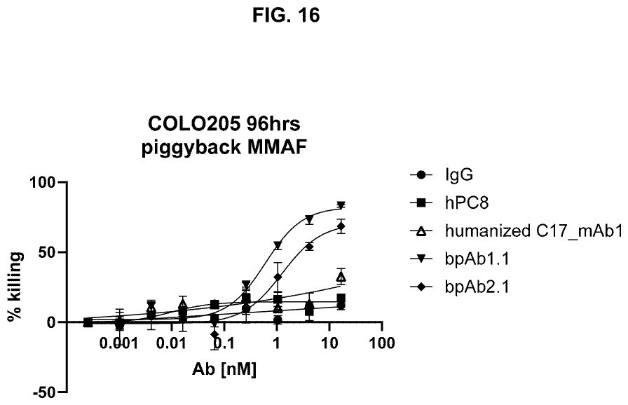

Tumor cells were seeded onto plates while the antibody cocktails were being prepared. Anti-CDH17 antibodies were incubated with Fab-αHFc-CL-MMAF at a molar ratio of 1:6 for 30 minutes at room temperature before being serially diluted. Cells were then incubated with the pre-diluted antibody mixtures for 96 hours at 37° C. in 5% CO₂. To measure cell viability, CellTiter-Glo (Promega) was added, and luminescence was measured immediately using a Neo2 plate reader (BioTek). Media or MMAF alone (at 250 nM) were used as negative and positive controls, corresponding to 0% and 100% killing, respectively, for normalization. FIG. 16 shows that anti-CDH17 biparatopic antibodies 1.1 and 2.1 induced greater cell cytotoxicity than both PC8 and their corresponding monoclonal antibody arm, C17_mAb1.

### Example 16: Target Engagement of Humanized CDH17xCD3 Bispecific Antibodies

The target engagement of humanized CDH17xCD3 bispecific antibodies (bsAbs) was assessed by evaluating their binding to both CDH17-expressing tumor cells and Jurkat T cells. Briefly, In the binding assay, target cells were incubated with serially diluted anti-CDH17 antibodies and control antibodies for 2 hr in 4° C. before being fixed. Cells were washed twice and incubated with AF488 labeled goat anti-mouse IgG antibody for 1 hr at room temperature. Cells were washed twice before being acquired on iQue for AF488 fluorescent intensity.

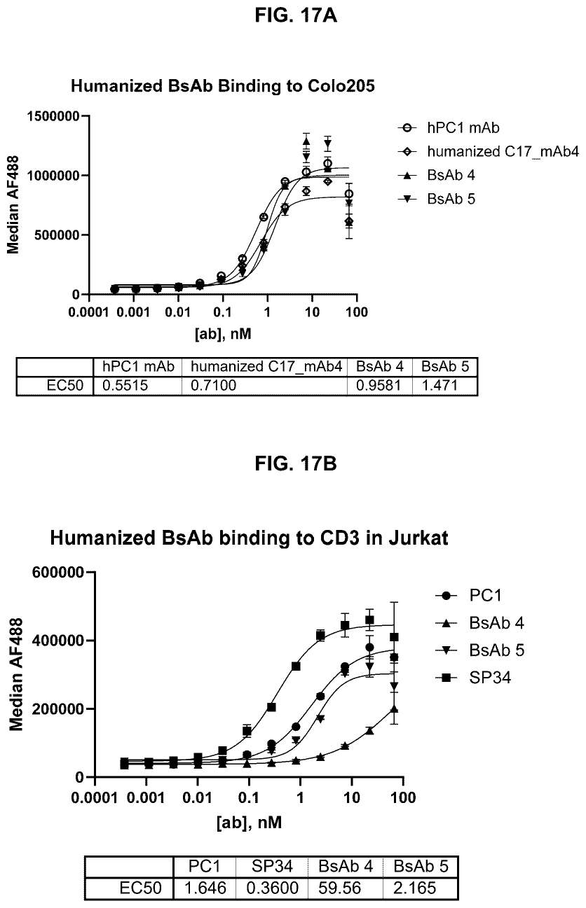

FIG. 17A shows binding of the bsAbs to Colo205 cells, which endogenously express CDH17. BsAb4 and BsAb5 demonstrated robust binding, with EC50 values of 0.96 nM and 1.47 nM, respectively, comparable to the humanized C17_mAb4 parental antibody (EC₅₀ 0.71 nM) and the positive control hPC1 mAb (EC50 0.55 nM).

FIG. 17B demonstrates bsAb binding to CD3 on Jurkat T cells. Both BsAb4 and BsAb5 bound effectively to CD3, with binding profiles comparable to the positive control PC1 and SP34, a known CD3-binding antibody. These data confirm that the humanized CDH17xCD3 bsAbs are capable of simultaneously engaging CDH17 on tumor cells and CD3 on T cells, supporting their intended bispecific mode of action.

### Example 17: TDCC Activity of Humanized CDH17xCD3 Bispecific Antibodies

The T cell-dependent cellular cytotoxicity (TDCC) activity of the humanized CDH17xCD3 bispecific antibodies (bsAbs) was evaluated using a co-culture assay with Colo205 tumor cells and human peripheral blood mononuclear cells (PBMCs) as effector cells at an effector-to-target (E: T) ratio of 20:1. In this three-day assay, tumor cells and PBMCs were incubated with serial dilutions of the bsAbs, and cytotoxicity was assessed by measuring % killing relative to controls.

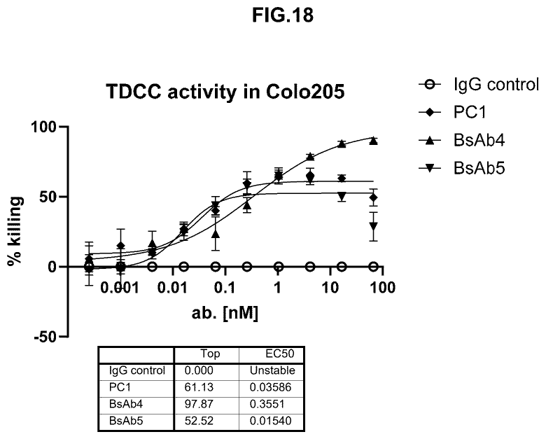

FIG. 18 shows that both BsAb4 and BsAb5 induced dose-dependent T cell-mediated cytotoxicity (TDCC) in a three-day Colo205 cell co-culture assay with PBMCs at a 20:1 E:T ratio.

BsAb4 demonstrated the highest maximal killing activity (Top: 97.87%) with an EC50 of 0.3561 nM. BsAb5 also showed significant cytotoxicity with a Top value of 52.52% and an EC50 of 0.0154 nM. The positive control antibody (PC1) exhibited moderate cytotoxicity (Top: 61.13%, EC50: 0.0359 nM), while the IgG control showed no detectable cytotoxic activity.

These results demonstrate that the humanized CDH17xCD3 bispecific antibodies effectively redirect T cells to kill CDH17-expressing tumor cells. In particular, BsAb4 showed strong potency and high maximal killing capacity, suggesting its potential as an effective therapeutic candidate for targeting CDH17-positive tumors.

Specific embodiments provided herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

In cases where numerical values are indicated herein, the skilled person will understand that the technical effect of the feature in question is ensured within an interval of accuracy, which typically encompasses a deviation of the numerical value given of ±10% or of ±5%. At the very least, and not as an attempt to limit the application of the doctrine of equivalents to the scope of the claims, each numerical parameter should at least be construed considering the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained.

The terms “a,” “an,” “the” and similar referents used in the context of the description herein (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the specification and does not pose a limitation on the scope of the claims. No language in the specification should be construed as indicating any non-claimed element essential to the practice of the description.

Unless specifically stated or obvious from context, as used herein, the term “or” is understood to be inclusive and covers both “or” and “and.”

Groupings of alternative elements or embodiments provided herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments are described herein, including the best mode known for carrying out methods provided herein. Of course, variations on these described embodiments will become apparent upon reading the foregoing description. One can be expected to employ such variations as appropriate and can be practiced other than as specifically described herein. Accordingly, this description includes all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

It is to be understood that the embodiments provided herein are illustrative of the principles of the description herein. Other modifications that can be employed are within the scope of the description. Thus, by way of example, but not of limitation, alternative configurations can be utilized in accordance with the teachings herein. Accordingly, the presented information is not limited to that precisely as shown and described.

While the present description has been described and illustrated herein by references to various specific materials, procedures, and examples, it is understood that the description is not restricted to the particular combinations of materials and procedures selected for that purpose. Numerous variations of such details can be implied as will be appreciated. It is intended that the specification and examples be considered as exemplary only, with the true scope and spirit of the specification being indicated by the following claims. All references, patents, and patent applications referred to in this application are herein incorporated by reference in their entirety.

Unless defined otherwise, all technical and scientific terms used herein have the same meaning as commonly understood. Although other probes, compositions, methods, and kits similar, or equivalent, to those described herein can be used in the practice described herein, the materials and methods are described herein. It is to be understood that the terminology used herein is for the purpose of describing embodiments only and is not intended to be limiting.

Unless specifically stated or obvious from context, as used herein, the term “about” is understood as within a range of normal tolerance, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

Any aspect or embodiment described herein can be combined with any other aspect or embodiment as described herein.

What is claimed is:

1. An antibody or antigen-binding fragment thereof that has binding specificity to a human Cadherin-17 (CDH17) protein, wherein the antibody or the fragment thereof comprises heavy chain complementarity determining regions (CDR) VH CDR1, VH CDR2, and VH CDR3 and light chain CDRs VL CDR1, VL CDR2, and VL CDR3, and wherein the VH CDR1, VH CDR2, VH CDR3, VL CDR1, VL CDR2, and VL CDR3, respectively, comprise:
(a) the amino acid sequences of SEQ ID NO: 21-26;
(b) the amino acid sequences of SEQ ID NO: 27-32;
(c) the amino acid sequences of SEQ ID NO: 33-38;
(d) the amino acid sequences of SEQ ID NO: 39-41, SEQ ID NO: 24, SEQ ID NO: 25 and SEQ ID NO: 42;
(e) the amino acid sequences of SEQ ID NO: 43, SEQ ID NO: 40, SEQ ID NO: 44, SEQ ID NO: 24, SEQ ID NO: 25 and SEQ ID NO: 45;
(f) the amino acid sequences of SEQ ID NO: 46-51;
(g) the amino acid sequences of SEQ ID NO: 52-57;
(h) the amino acid sequences of SEQ ID NO: 58-63;
(i) the amino acid sequences of SEQ ID NO: 64-69; or
(j) the amino acid sequences of SEQ ID NO: 70-75.

2. The antibody or antigen-binding fragment thereof of claim 1, wherein the antibody or antigen-binding fragment thereof is or comprises a F(ab′)₂, a F(ab)₂, a Fab′, a Fab, a Fv, a single chain Fv (scFv), a disulfide stabilized Fv (dsFv), an Fc-scFv, minibody, and a diabody.

3. The antibody or antigen-binding fragment thereof of claim 1, wherein the antibody or antigen-binding fragment thereof comprises:
a heavy chain variable region (VH) comprising an amino acid sequence that is at least 75%, at least 80%, at least 85%, at least 90%, at least 95%, or at least 99% identical to an amino acid sequence selected from SEQ ID NOs: 1, 3, 5, 7, 9, 11, 13, 15, 17, and 19; and
a light chain variable region (VL) comprising an amino acid sequence that is at least 75%, at least 80%, at least 85%, at least 90%, at least 95%, or at least 99% identical to an amino acid sequence selected from SEQ ID NOs: 2, 4, 6, 8, 10, 12, 14, 16, 18, and 20.

4. The antibody or antigen-binding fragment thereof of claim 3, wherein:
(a) the VH comprises an amino acid sequence that is at least 90% identical to the amino acid sequence of SEQ ID NO: 1, and the VL comprises an amino acid sequence that is at least 90% identical to the amino acid sequence of SEQ ID NO: 2;
(b) the VH comprises an amino acid sequence that is at least 90% identical to the amino acid sequence of SEQ ID NO: 3, and the VL comprises an amino acid sequence that is at least 90% identical to the amino acid sequence of SEQ ID NO: 4;
(c) the VH comprises an amino acid sequence that is at least 90% identical to the amino acid sequence of SEQ ID NO: 5, and the VL comprises an amino acid sequence that is at least 90% identical to the amino acid sequence of SEQ ID NO: 6;
(d) the VH comprises an amino acid sequence that is at least 90% identical to the amino acid sequence of SEQ ID NO: 7, and the VL comprises an amino acid sequence that is at least 90% identical to the amino acid sequence of SEQ ID NO: 8;
(e) the VH comprises an amino acid sequence that is at least 90% identical to the amino acid sequence of SEQ ID NO: 9, and the VL comprises an amino acid sequence that is at least 90% identical to the amino acid sequence of SEQ ID NO: 10;
(f) the VH comprises an amino acid sequence that is at least 90% identical to the amino acid sequence of SEQ ID NO: 11, and the VL comprises an amino acid sequence that is at least 90% identical to the amino acid sequence of SEQ ID NO: 12;
(g) the VH comprises an amino acid sequence that is at least 90% identical to the amino acid sequence of SEQ ID NO: 13, and the VL comprises an amino acid sequence that is at least 90% identical to the amino acid sequence of SEQ ID NO: 14;
(h) the VH comprises an amino acid sequence that is at least 90% identical to the amino acid sequence of SEQ ID NO: 15, and the VL comprises an amino acid sequence that is at least 90% identical to the amino acid sequence of SEQ ID NO: 16;
(i) the VH comprises an amino acid sequence that is at least 90% identical to the amino acid sequence of SEQ ID NO: 17, and the VL comprises an amino acid sequence that is at least 90% identical to the amino acid sequence of SEQ ID NO: 18; or
(j) the VH comprises an amino acid sequence that is at least 90% identical to the amino acid sequence of SEQ ID NO: 19, and the VL comprises an amino acid sequence that is at least 90% identical to the amino acid sequence of SEQ ID NO: 20.

5. The antibody or antigen-binding fragment thereof of claim 1, wherein the antibody or antigen-binding fragment thereof is humanized.

6. The antibody or antigen-binding fragment thereof of claim 5, wherein the VH comprises an amino acid sequence that is at least 90%, at least 95%, or at least 99% identical to an amino acid sequence selected from SEQ ID NOs: 76, 77, 80, 81, 84, 85, and 123; and wherein the VL comprises an amino acid sequence that is at least 90%, at least 95%, or at least 99% identical to an amino acid sequence selected from SEQ ID NOs: 78, 79, 82, 83, 86, 87, and 124.

7. The antibody or antigen-binding fragment thereof of claim 6, wherein the antibody or antigen-binding fragment comprises:
(a) a variable heavy chain region (VH) comprising an amino acid sequence having at least 90% identity to an amino acid sequence of SEQ ID NO: 76 and a variable light chain region (VL) comprising an amino acid sequence having at least 90% identity to an amino acid sequence of SEQ ID NO: 78;
(b) a VH comprising an amino acid sequence having at least 90% identity to an amino acid sequence of SEQ ID NO: 76 and a VL comprising an amino acid sequence having at least 90% identity to an amino acid sequence of SEQ ID NO: 79;
(c) a VH comprising an amino acid sequence having at least 90% identity to an amino acid sequence of SEQ ID NO: 77 and a VL comprising an amino acid sequence having at least 90% identity to an amino acid sequence of SEQ ID NO: 78;
(d) a VH comprising an amino acid sequence having at least 90% identity to an amino acid sequence of SEQ ID NO: 77 and a VL comprising an amino acid sequence having at least 90% identity to an amino acid sequence of SEQ ID NO: 79;
(e) a VH comprising an amino acid sequence having at least 90% identity to an amino acid sequence of SEQ ID NO: 80 and a VL comprising an amino acid sequence having at least 90% identity to an amino acid sequence of SEQ ID NO: 82;
(f) a VH comprising an amino acid sequence having at least 90% identity to an amino acid sequence of SEQ ID NO:80 and a VL comprising an amino acid sequence having at least 90% identity to an amino acid sequence of SEQ ID NO: 83;
(g) a VH comprising an amino acid sequence having at least 90% identity to an amino acid sequence of SEQ ID NO: 81 and a VL comprising an amino acid sequence having at least 90% identity to an amino acid sequence of SEQ ID NO: 82;
(h) a VH comprising an amino acid sequence having at least 90% identity to an amino acid sequence of SEQ ID NO: 81 and a VL comprising an amino acid sequence having at least 90% identity to an amino acid sequence of SEQ ID NO: 83;
(i) a VH comprising an amino acid sequence having at least 90% identity to an amino acid sequence of SEQ ID NO: 84 and a VL comprising an amino acid sequence having at least 90% identity to an amino acid sequence of SEQ ID NO: 86;
(j) a VH comprising an amino acid sequence having at least 90% identity to an amino acid sequence of SEQ ID NO: 84 and a VL comprising an amino acid sequence having at least 90% identity to an amino acid sequence of SEQ ID NO: 87;
(k) a VH comprising an amino acid sequence having at least 90% identity to an amino acid sequence of SEQ ID NO: 85 and a VL comprising an amino acid sequence having at least 90% identity to an amino acid sequence of SEQ ID NO: 86;
(l) a VH comprising an amino acid sequence having at least 90% identity to an amino acid sequence of SEQ ID NO: 85 and a VL comprising an amino acid sequence having at least 90% identity to an amino acid sequence of SEQ ID NO: 87; or
(m) a VH comprising an amino acid sequence having at least 90% identity to an amino acid sequence of SEQ ID NO: 123 and a VL comprising an amino acid sequence having at least 90% identity to an amino acid sequence of SEQ ID NO: 124.

8. The antibody or antigen-binding fragment thereof of claim 7, wherein the antibody or antigen-binding fragment thereof comprises:
(a) a heavy chain Fab region comprising an amino acid sequence that is at least 90% identical to an amino acid sequence selected from SEQ ID NOs: 88, 90, 92, 94, 96, 100, 102, 104, 117, 119, 121, and 125; and
(b) a light chain Fab region comprising an amino acid sequence that is at least 90% identical to an amino acid sequence selected from SEQ ID NOs: 89, 91, 93, 95, 97, 101, 118, 103, 105, 120, 122, and 126.

9. The antibody or antigen-binding fragment thereof of claim 8, wherein the antibody or antigen-binding fragment thereof comprises:
(a) a heavy chain Fab region comprising an amino acid sequence that is at least 90% identical to the amino acid sequence of SEQ ID NO: 88, and a light chain Fab region comprising an amino acid sequence that is at least 90% identical to the amino acid sequence of SEQ ID NO: 89;
(b) a heavy chain Fab region comprising an amino acid sequence that is at least 90% identical to the amino acid sequence of SEQ ID NO: 90, and a light chain Fab region comprising an amino acid sequence that is at least 90% identical to the amino acid sequence of SEQ ID NO: 91;
(c) a heavy chain Fab region comprising an amino acid sequence that is at least 90% identical to the amino acid sequence of SEQ ID NO: 92, and a light chain Fab region comprising an amino acid sequence that is at least 90% identical to the amino acid sequence of SEQ ID NO: 93;
(d) a heavy chain Fab region comprising an amino acid sequence that is at least 90% identical to the amino acid sequence of SEQ ID NO: 94, and a light chain Fab region comprising an amino acid sequence that is at least 90% identical to the amino acid sequence of SEQ ID NO: 95;
(e) a heavy chain Fab region comprising an amino acid sequence that is at least 90% identical to the amino acid sequence of SEQ ID NO: 96, and a light chain Fab region comprising an amino acid sequence that is at least 90% identical to the amino acid sequence of SEQ ID NO: 97;
(f) a heavy chain Fab region comprising an amino acid sequence that is at least 90% identical to the amino acid sequence of SEQ ID NO: 100, and a light chain Fab region comprising an amino acid sequence that is at least 90% identical to the amino acid sequence of SEQ ID NO: 101;
(g) a heavy chain Fab region comprising an amino acid sequence that is at least 90% identical to the amino acid sequence of SEQ ID NO: 102, and a light chain Fab region comprising an amino acid sequence that is at least 90% identical to the amino acid sequence of SEQ ID NO: 103;
(h) a heavy chain Fab region comprising an amino acid sequence that is at least 90% identical to the amino acid sequence of SEQ ID NO: 104, and a light chain Fab region comprising an amino acid sequence that is at least 90% identical to the amino acid sequence of SEQ ID NO: 105;
(i) a heavy chain Fab region comprising an amino acid sequence that is at least 90% identical to the amino acid sequence of SEQ ID NO: 117, and a light chain Fab region comprising an amino acid sequence that is at least 90% identical to the amino acid sequence of SEQ ID NO: 118;
(j) a heavy chain Fab region comprising an amino acid sequence that is at least 90% identical to the amino acid sequence of SEQ ID NO: 119, and a light chain Fab region comprising an amino acid sequence that is at least 90% identical to the amino acid sequence of SEQ ID NO: 120;
(k) a heavy chain Fab region comprising an amino acid sequence that is at least 90% identical to the amino acid sequence of SEQ ID NO: 121, and a light chain Fab region comprising an amino acid sequence that is at least 90% identical to the amino acid sequence of SEQ ID NO: 122; or
(l) a heavy chain Fab region comprising an amino acid sequence that is at least 90% identical to the amino acid sequence of SEQ ID NO: 125, and a light chain Fab region comprising an amino acid sequence that is at least 90% identical to the amino acid sequence of SEQ ID NO: 126.

10. The antibody or antigen-binding fragment thereof of claim 1, wherein the antibody or antigen-binding fragment thereof is conjugated to a drug moiety selected from a cytotoxin, an immunosuppressive agent, a radioisotope, or a toxin.

11. A biparatopic antibody or antigen-binding fragment thereof comprising a first antigen-binding portion having specificity to a first epitope in human CDH17 and a second antigen-binding portion having specificity to a second distinct non-overlapping epitope within human CDH17, wherein the biparatopic antibody comprises:
(a) a first antigen binding portion having specificity for an epitope in ECD 3-5 of human CDH17 and a second binding portion having specificity for an epitope in ECD 6-7 of human CDH17; or
(b) a first antigen binding portion having specificity for an epitope in ECD 1-2 of human CDH17 and a second binding portion having specificity for an epitope in ECD 6-7 of human CDH17.

12. The biparatopic antibody or antigen-binding fragment thereof of claim 11, wherein the antibody comprises a first Fab fragment (Arm 1) comprising a first heavy chain variable region and a first light chain variable region and a second Fab fragment (Arm 2) comprising a second heavy chain variable region and a second variable light chain region,
wherein the first heavy chain variable region, the first light chain variable region, the second heavy chain variable region, and the second variable light chain region, respectively, comprise amino acid sequences that are at least 90% identical to the amino acid sequences of
(a) SEQ ID NO: 88, SEQ ID NO: 89, SEQ ID NO: 90, and SEQ ID NO: 91;
(b) SEQ ID NO: 88, SEQ ID NO: 89, SEQ ID NO: 92 and SEQ ID NO: 93;
(c) SEQ ID NO: 94, SEQ ID NO: 95, SEQ ID NO: 96, and SEQ ID NO: 97;
(d) SEQ ID NO: 117, SEQ ID NO: 118, SEQ ID NO: 119, and SEQ ID NO: 120; or
(e) SEQ ID NO: 117, SEQ ID NO: 118, SEQ ID NO: 121, and SEQ ID NO: 122.

13. The biparatopic anti-CDH17 antibody or antigen-binding fragment thereof of claim 11, wherein the biparatopic anti-CDH17 antibody or antigen-binding fragment thereof is or comprises a F(ab′)₂, a F(ab)₂, IgG-scFv, tandem scFv, or a bispecific antibody.

14. The biparatopic anti-CDH17 antibody or antigen-binding fragment thereof of claim 11, further comprising a drug moiety conjugated to the biparatopic anti-CDH17 antibody or antigen-binding fragment thereof, wherein the drug moiety is selected from a cytotoxin, an immunosuppressive agent, a radioisotope, or a toxin.

15. A bispecific T cell engager antibody wherein the antibody comprises a first anti-CD3 scFv fragment (Arm 1) and a second Fab fragment (Arm 2) comprising a heavy chain variable region and a light chain variable region specific for CDH17, wherein the heavy chain variable region and the light chain variable region, respectively, comprise the amino acid sequences of:
(a) SEQ ID NO: 100 and SEQ ID NO: 101;
(b) SEQ ID NO: 102 and SEQ ID NO: 103;
(c) SEQ ID NO: 104 and SEQ ID NO: 105; or
(d) SEQ ID NO: 125 and SEQ ID NO: 126.

16. The bispecific T cell engager antibody of claim 15, wherein the anti-CD3 scFv fragment comprises an amino acid sequence of SEQ ID NO: 98 or SEQ ID NO: 127.

17. The bispecific T cell engager antibody of claim 15, wherein the anti-CD3 scFv fragment, the heavy chain variable region, and the light chain variable region, respectively, comprise the amino acid sequences of:
(a) SEQ ID NO: 98, SEQ ID NO: 100 and SEQ ID NO: 101;
(b) SEQ ID NO: 98, SEQ ID NO: 102 and SEQ ID NO: 103;
(c) SEQ ID NO: 98, SEQ ID NO: 104 and SEQ ID NO: 105;
(d) SEQ ID NO: 98, SEQ ID NO: 125 and SEQ ID NO: 126; or
(e) SEQ ID NO: 127, SEQ ID NO: 125 and SEQ ID NO: 126.

18. A bispecific T cell engager antibody wherein the antibody comprises a first anti-CD3 scFv fragment (Arm 1) and a second Fab fragment (Arm 2) comprising a heavy chain variable region and a light chain variable region specific for CDH17, wherein the heavy chain variable region and the light chain variable region, respectively, comprise the amino acid sequences of SEQ ID NO: 125 and SEQ ID NO: 126; and
wherein the anti-CD3 scFv fragment comprises the amino acid sequence of SEQ ID NO: 98.

19. A bispecific T cell engager antibody wherein the antibody comprises a first anti-CD3 scFv fragment (Arm 1) and a second Fab fragment (Arm 2) comprising a heavy chain variable region and a light chain variable region specific for CDH17, wherein the heavy chain variable region and the light chain variable region, respectively, comprise the amino acid sequences of SEQ ID NO: 125 and SEQ ID NO: 126; and
wherein the anti-CD3 scFv fragment comprises the amino acid sequence of SEQ ID NO: 127.

20. The bispecific T cell engager antibody of claim 15, wherein the biparatopic anti-CDH17 antibody or antigen-binding fragment thereof is or comprises a F(ab′)₂, a F(ab)₂, IgG-scFv, tandem scFv, and a bispecific antibody.

21. A nucleic acid encoding the antibody or antigen-binding fragment thereof of claim 1.

22. An expression vector comprising the nucleic acid of claim 21.

23. A host cell comprising the nucleic acid of claim 21.

24. A pharmaceutical composition comprising a pharmacologically acceptable carrier and the antibody or antigen-binding fragment thereof of claim 1.

25. A method of treating cancer in a patient in need thereof, comprising administering to the patient the antibody or antigen-binding fragment thereof of claim 1.
